(19) United States
(12) Patent Application Publication
(10) Pub. No.: US 2022/0273751 A1
(43) Pub. Date: Sep. 1, 2022

(54) GPCR HETEROMER INHIBITORS AND USES THEREOF

(71) Applicant: GPCR THERAPEUTICS, INC., Seoul (KR)

(72) Inventors: DongSeung SEEN, Seoul (KR); Eunhee KIM, Seoul (KR); SoHui KIM, Seoul (KR); HyeonGyu SEO, Seoul (KR); Jiyeong LEE, Seoul (KR); Chang Soo YANG, Seoul (KR); DongJin LEE, Seoul (KR); Jiwon YANG, Seoul (KR)

(73) Assignee: GPCR THERAPEUTICS, INC., Seoul (KR)

(21) Appl. No.: 17/609,777

(22) PCT Filed: May 14, 2020
(86) PCT No.: PCT/IB2020/054552

§ 371 Date: Nov. 8, 2021

Related U.S. Application Data
(60) Provisional application No. 63/022,845, filed on May 11, 2020.
(60) Provisional application No. 62/849,755, filed on May 17, 2019.

(51) Int. Cl. A61K 38/00; C07K 16/28; A61K 31/404; A61K 47/68; A61P 35/02; G01N 33/53

(52) U.S. Cl. CPC A61K 38/005; C07K 16/2866; A61K 31/404; A61K 47/68; A61P 35/02; C07K 16/286; G01N 33/53; C07K 2317/76

(57) ABSTRACT

This invention relates to inhibitors of CXC receptor 4 (CXCR4)-G protein-coupled receptor (GPCR) heteromers (CXCR4-GPCR heteromers) associated with cancers, where CXCR4 forms a functional heteromer with other G protein-coupled receptors (GPCRx). More specifically, this invention relates to ADRB2 that form heteromers with CXCR4, which upon co-stimulation with CXCR4 agonists and ADRB2 agonists leads to enhanced signaling downstream of CXCR4. This invention also provides for pharmaceutical compositions and kits comprising a CXCR4 inhibitor and an ADRB2 inhibitor, and methods for treating and using the same, and in the diagnosis and/or therapy for cancer.

### CROSS REFERENCE TO RELATED APPLICATIONS

This application claims the benefit of U.S. Provisional Patent Application No. 63/022,845, filed on May 11, 2020 and U.S. Provisional Patent Application No. 62/849,755, filed on May 17, 2019, the disclosure of each of which is incorporated by reference herein in its entirety.

### SEQUENCE LISTING

This application incorporates by reference a Sequence Listing submitted with this application in ASCII text format, entitled “14462-012-228_SEQ_LISTING.txt,” was created on May 11, 2020, and is 1,516 bytes in size.

### FIELD

The present invention relates to the field of cancer therapy. In particular, provided herein are pharmaceutical compositions comprising a combination of a CXCR4 inhibitor and an ADRB2 inhibitor, and methods for suppressing, methods for treating, pharmaceutical kits for use, and pharmaceutical compositions for use, using the same.

### BACKGROUND OF THE INVENTION

G protein-coupled receptors (GPCRs) are seven-transmembrane domain cell surface receptors that are coupled to G proteins. GPCRs mediate diverse sensory and physiological responses by perceiving stimuli including light, odorants, hormones, neurotransmitters, chemokines, small lipid molecules, and nucleotides. There are approximately 800 GPCR genes in human genome, and more than half of them are predicted to encode sensory receptors such as olfactory, visual, and taste receptors (Bjarnadottir, T. K., et al.; (2006) Comprehensive repertoire and phylogenetic analysis of the G protein-coupled receptors in human and mouse, Genomics 88, 263-273). The remaining 350 GPCRs have physiologically important roles in embryonic development, behavior, mood, cognition, regulation of blood pressure, heart rate, and digestive processes, regulation of immune system and inflammation, maintenance of homeostasis, and growth and metastasis of cancers (Filmore, D. (2004) It's a GPCR world. Modern Drug Discovery American Chemical Society 2004 (November), 24-28; Overington, J. P., et al., (2006) How many drug targets are there? Nat Rev Drug Discov 5, 993-996). GPCRs are associated with many diseases and are the targets of approximately 40% of all prescription drugs (Filmore, D. (2004)).

CXC receptor 4 (“CXCR4”) is a member of the chemokine receptor family GPCR. CXCR4 is expressed on most of the hematopoietic cell types, bone marrow stem cells, endothelial progenitor cells, vascular endothelial cells, neurons and neuronal stem cells, microglia and astrocytes (Klein, R. S., et al., (2004) Immune and nervous system CXCL12 and CXCR4: parallel roles in patterning and plasticity, Trends Immunol 25, 306-314; Griffith, J. W., et. al., (2014) Chemokines and chemokine receptors: positioning cells for host defense and immunity, Annu Rev Immunol 32, 659-702). CXCR4 responds to its ligand CXC Motif Chemokine ligand 12 (CXCL12), also known as Stromal cell-derived factor 1 (SDF-1), and has essential roles in the embryonic development of the hematopoietic, cardiovascular, and nervous systems (Griffith, J. W., et. al., (2014)). CXCR4 was discovered as a co-receptor for human immunodeficiency virus (HIV), and has important roles in the homing of hematopoietic stem cells (HSCs) to the bone marrow, inflammation, immune surveillance of tissues, and tissue regeneration in adult (Chatterjee, S., et al., (2014) The intricate role of CXCR4 in cancer, Adv Cancer Res 124, 31-82). CXCR4 is implicated in various immune and autoimmune diseases, such as HIV infection, ischaemia, wound healing, rheumatoid arthritis, systemic lupus erythematosus (SLE), interstitial pneumonias, vascular disease, multiple sclerosis, pulmonary fibrosis, and allergic airway disease (Chu, T. et al., (2017) CXCL12/CXCR4/CXCR7 Chemokine Axis in the Central Nervous System: Therapeutic Targets for Remyelination in Demyelinating Diseases, Neuroscientist 23, 627-648; Debnath, B., et al., (2013) Small molecule inhibitors of CXCR4, Theranostics 3, 47-75; and Domanska, U. M., et al., (2013) A review on CXCR4/CXCL12 axis in oncology: no place to hide, Eur J Cancer 49, 219-230). CXCR4 has also been associated with a variety of different cancers and considered to have multiple potential roles in malignancy. CXCR4 is overexpressed in more than 23 human cancers, including breast cancer, lung cancer, brain cancer, kidney cancer (or renal cell carcinoma), pancreatic cancer, ovarian cancer, prostate cancer, melanoma, leukemia, multiple myeloma, gastrointestinal cancers, and soft tissue sarcomas, and regarded as a poor prognosis marker (Domanska, U. M., et al., (2013); Chatterjee, S., et al., (2014); and Furusato, B., et al., (2010) CXCR4 and cancer, Pathol Int 60, 497-505).

The formation of heteromers of CXCR4 and a GPCR have been studied within a limited number of GPCR families, such as chemokine, adrenergic, and opioid receptor families. Considering the major role and increased expression of CXCR4 in a variety of pathological conditions, there is a great potential for the existence of different CXCR4-GPCRx heteromers that confer unique features to a specific disease.

Adrenoceptor Beta 2, sometimes referred to as beta-2 adrenergic receptor or β2 adrenoreceptor (“ADRB2”), is a cell membrane-spanning beta-adrenergic receptor that interacts with epinephrine, a hormone and neurotransmitter (ligand synonym, adrenaline) whose signaling, via a downstream L-type calcium channel interaction, mediates physiologic responses such as smooth muscle relaxation and bronchodilation (Gregorio, G. G., et al., (2017) Single-molecule analysis of ligand efficacy in beta2AR-G-protein activation, Nature 547, 68-73). Formation of a heteromer of CXCR4 an ADRB2 (CXCR4-ADRB2 heteromer), and the co-expression of CXCR4 and ADRB2 (P2-AR) on cardiac myocytes, and the physical association of CXCR4 with ADRB2 using co-immunoprecipitation and bioluminescence resonance energy transfer (LaRocca, T. J., et al. (2010) beta2-Adrenergic receptor signaling in the cardiac myocyte is modulated by interactions with CXCR4, J Cardiovasc Pharmacol 56, 548-559; Nakai, A., et al., (2014) Control of lymphocyte egress from lymph nodes through beta2-adrenergic receptors, J Exp Med 211, 2583-2598).

Considering the major role and increased expression of CXCR4 in a variety of pathological conditions, there is a great potential for a CXCR4-ADRB2 heteromer to confer unique features to a specific disease. Thus, there exists in the art a need for identifying and developing inhibitors of a CXCR4-ADRB2 heteromer for use as CXCR4-ADRB2 heteromer-targeting cancer therapeutics with higher efficacy and lower side effects, for example relative to CXCR4 inhibitor monotherapy.

Provided herein are inhibitors of a CXCR4-ADRB2 heteromer, or combination of inhibitors of the CXCR4-ADRB2 heteromer, and pharmaceutical compositions or pharmaceutical kits comprising the same, and methods for treating and using the same, wherein an enhanced downstream signaling results from functional CXCR4-ADRB2 heteromer formation (e.g., enhanced signaling downstream relative to CXCR4, or enhanced signaling downstream relative to ADRB2); wherein the presence of said CXCR4-ADRB2 heteromer in a subject, such as the cell of a subject, is associated with diseases, such as cancer.

### SUMMARY OF THE INVENTION

In one aspect, provided herein are methods for suppressing enhanced downstream signaling resulting from a CXCR4-ADRB2 heteromer in a cell of a subject suffering from cancer, the method including administering to the subject: (a) a CXCR4 inhibitor, wherein the CXCR4 inhibitor is burixafor; and (b) an ADRB2 inhibitor; wherein: (i) the enhanced downstream signaling results from the CXCR4-ADRB2 heteromer; and (ii) the administered combination of inhibitors suppresses the enhanced downstream signaling from said CXCR4-ADRB2 heteromer in the cancer subject.

In another aspect, provided herein are methods for treating cancer in a subject having a cell containing a CXCR4-ADRB2 heteromer, the method including administering to the subject: (a) a CXCR4 inhibitor, wherein the CXCR4 inhibitor is burixafor; and (b) an ADRB2 inhibitor; wherein: (i) enhanced downstream signaling results from the CXCR4-ADRB2 heteromer; and (ii) the administered combination of inhibitors suppresses the enhanced downstream signaling from said CXCR4-ADRB2 heteromer in the cancer subject.

In another aspect, provided herein are methods for treating cancer in a subject having a cell containing a CXCR4-ADRB2 heteromer, the method including: (a) determining whether the subject's cell contains a CXCR4-ADRB2 heteromer, wherein enhanced downstream signaling results from the CXCR4-ADRB2 heteromer; and (b) if the subject's cell contains said CXCR4-ADRB2 heteromer, then administering to the cancer subject: (i) a CXCR4 inhibitor, wherein the CXCR4 inhibitor is burixafor; and (ii) an ADRB2 inhibitor.

In another aspect, provided herein are methods for treating cancer in a subject having a cell containing a CXCR4-ADRB2 heteromer, wherein enhanced downstream signaling results from the CXCR4-ADRB2 heteromer, the method including: (1) determining whether the subject has the cell containing the CXCR4-ADRB2 heteromer by: obtaining or having obtained a biological sample from the subject; and performing or having performed an assay on the biological sample to determine if: (i) the subject's cell contains said CXCR4-ADRB2 heteromer; or (ii) a combination of a CXCR4 inhibitor and an ADRB2 inhibitor: alters heteromer-specific properties or function of said CXCR4-ADRB2 heteromer in the subject's derived cell(s); alters heteromer-specific properties of the subject's derived cell(s) containing said CXCR4-ADRB2 heteromer; or decreases cancer progression in the subject having cells containing said CXCR4-ADRB2 heteromer; and (2) if the subject has a cell containing said CXCR4-ADRB2 heteromer, then administering to the cancer subject a combination of a CXCR4 inhibitor and an ADRB2 inhibitor, wherein the CXCR4 inhibitor is burixafor; and (3) if the subject does not have a cell containing said CXCR4-ADRB2 heteromer, then administering to the cancer subject a single inhibitor of either the burixafor or the ADRB2 inhibitor.

In another aspect, provided herein are methods for treating cancer in a subject having a cell containing a CXCR4-ADRB2 heteromer, wherein enhanced downstream signaling results from the CXCR4-ADRB2 heteromer, the method including: (1) determining whether the subject has the cell containing the CXCR4-ADRB2 heteromer by: obtaining or having obtained a biological sample from the subject; and performing or having performed an assay on the biological sample to determine if: (i) the subject's cell contains said CXCR4-ADRB2 heteromer; or (ii) a combination of a CXCR4 inhibitor and an ADRB2 inhibitor: alters heteromer-specific properties or function of said CXCR4-ADRB2 heteromer in the subject's derived cell(s); alters heteromer-specific properties of the subject's derived cell(s) containing said CXCR4-ADRB2 heteromer; or decreases cancer progression in the subject having cells containing said CXCR4-ADRB2 heteromer; and (2) if the subject has a cell containing said CXCR4-ADRB2 heteromer, then administering to the cancer subject a combination of a CXCR4 inhibitor and an ADRB2 inhibitor, wherein the CXCR4 inhibitor is burixafor; and (3) if the subject does not have a cell containing said CXCR4-ADRB2 heteromer, then administering to the cancer subject a single inhibitor of either the burixafor or the ADRB2 inhibitor; wherein: (a) progression of the cancer in the subject having said cell containing the CXCR4-ADRB2 heteromer is decreased in the range of 5-100% more upon administering to the cancer subject the combination of the burixafor and the ADRB2 inhibitor, relative to administering the single inhibitor of either the burixafor or the ADRB2 inhibitor; (b) efficacy of the burixafor is increased in the range of 5-2000% when administered in combination with the ADRB2 inhibitor to the subject having said cell containing the CXCR4-ADRB2 heteromer, relative to efficacy of the burixafor when administered as a single inhibitor; and/or (c) efficacy of the ADRB2 inhibitor is increased in the range of 5-2000% when administered in combination with the burixafor to the subject having said cell containing the CXCR4-ADRB2 heteromer, relative to efficacy of the ADRB2 inhibitor when administered as a single inhibitor.

In another aspect, provided herein are methods for treating cancer in a subject having a cell containing a CXCR4-ADRB2 heteromer, wherein enhanced downstream signaling results from the CXCR4-ADRB2 heteromer, the method including: (1) determining whether the subject's cell contains the CXCR4-ADRB2 heteromer by: obtaining or having obtained a biological sample from the subject and performing or having performed an assay on the biological sample to determine if said CXCR4-ADRB2 heteromer is present in the subject's cell; wherein the assay performed on the biological sample is or comprises one or more of the following: a co-internalization assay, a colocalization assay, in situ hybridization, immunohistochemistry, immunoelectron microscopy, a proximity-based assay, a co-immunoprecipitation assay, enzyme-linked immunosorbent assay (ELISA), flow cytometry, RNAseq, RT-qPCR, microarray, or a fluorescent animal assay; and (2) if the subject's cell contains said CXCR4-ADRB2 heteromer, then administering to the cancer subject a combination of a CXCR4 inhibitor and an ADRB2 inhibitor, wherein the CXCR4 inhibitor is burixafor; and (3) if the subject's cell does not contain said CXCR4-ADRB2 heteromer, then administering to the cancer subject a single inhibitor of either the burixafor or the ADRB2 inhibitor.

In another aspect, provided herein are methods for treating cancer in a subject having a cell containing a CXCR4-ADRB2 heteromer, wherein enhanced downstream signaling results from the CXCR4-ADRB2 heteromer, the method including: (1) determining whether the subject's cell contains the CXCR4-ADRB2 heteromer by: obtaining or having obtained a biological sample from the subject and performing or having performed an assay on the biological sample to determine if said CXCR4-ADRB2 heteromer is present in the subject's cell; wherein the assay performed on the biological sample is or comprises one or more of the following: a co-internalization assay, a colocalization assay, in situ hybridization, immunohistochemistry, immunoelectron microscopy, a proximity-based assay, a co-immunoprecipitation assay, enzyme-linked immunosorbent assay (ELISA), flow cytometry, RNAseq, RT-qPCR, microarray, or a fluorescent animal assay; and (2) if the subject's cell contains said CXCR4-ADRB2 heteromer, then administering to the cancer subject a combination of a CXCR4 inhibitor and an ADRB2 inhibitor, wherein the CXCR4 inhibitor is burixafor; and (3) if the subject's cell does not contain said CXCR4-ADRB2 heteromer, then administering to the cancer subject a single inhibitor of either the burixafor or the ADRB2 inhibitor; wherein: (a) progression of the cancer in the subject having said cell containing the CXCR4-ADRB2 heteromer is decreased in the range of 5-100% more upon administering to the cancer subject the combination of the burixafor and the ADRB2 inhibitor, relative to administering the single inhibitor of either the burixafor or the ADRB2 inhibitor; (b) efficacy of the burixafor is increased in the range of 5-2000% when administered in combination with the ADRB2 inhibitor to the subject having said cell containing the CXCR4-ADRB2 heteromer, relative to efficacy of the burixafor when administered as a single inhibitor; and/or (c) efficacy of the ADRB2 inhibitor is increased in the range of 5-2000% when administered in combination with the burixafor to the subject having said cell containing the CXCR4-ADRB2 heteromer, relative to efficacy of the ADRB2 inhibitor when administered as a single inhibitor.

In another aspect, provided herein are pharmaceutical kits for use in treating cancer in a subject having a cell containing a CXCR4-ADRB2 heteromer, the pharmaceutical kit including: (a) a CXCR4 inhibitor, wherein the CXCR4 inhibitor is burixafor; and (b) an ADRB2 inhibitor; wherein enhanced downstream signaling results from the CXCR4-ADRB2 heteromer.

In another aspect, provided herein are pharmaceutical compositions for use in treating cancer in a subject having a cell containing a CXCR4-ADRB2 heteromer, the pharmaceutical composition including: (a) a CXCR4 inhibitor, wherein the CXCR4 inhibitor is burixafor; (b) an ADRB2 inhibitor; and (c) a pharmaceutically acceptable carrier; wherein enhanced downstream signaling results from the CXCR4-ADRB2 heteromer.

In another aspect, provided herein are pharmaceutical compositions including: (a) a CXCR4 inhibitor, wherein the CXCR4 inhibitor is burixafor; (b) an ADRB2 inhibitor; and (c) a pharmaceutically acceptable carrier.

### BRIEF DESCRIPTION OF THE DRAWINGS

FIG. 1: Shows schematic drawing of bimolecular fluorescence complementation (BiFC) assay. GPCR A is fused with N-terminal fragment of yellow fluorescent protein (YFP) Venus (VN) and GPCR B is fused with C-terminal fragment of Venus (VC). When GPCR A and B form heteromer, the complementary VN and VC are close enough to form functional Venus.

FIGS. 2A-2D: Show identification of CXCR4 interacting with ADRB2 using BiFC assay. Representative images that showed negative BiFC signal; CXCR4-VN and HA-VC (FIG. 2A), and CXCR4-VN and GCGR-VC (FIG. 2C). Representative images of CXCR4 and GPCRx that showed positive BiFC signal: CXCR4-VN and CXCR4-VC (FIG. 2B), and CXCR4-VN and ADRB2-VC (FIG. 2D).

FIGS. 3A-3B: Show principle of GPCR co-internalization study. Cells co-expressing CXCR4-GFP and GPCRx are treated with GPCRx specific agonist. (FIG. 3A) CXCR4 and GPCRx does not physically interact with each other. GPCRx agonist induces internalization of GPCRx, but not CXCR4-GFP. (FIG. 3B) CXCR4 and GPCRx physically interact and forms heteromer. GPCRx agonist induces internalization of GPCRx, and CXCR4-GFP is co-internalized with GPCRx.

FIG. 4A-4B: Show co-internalization of CXCR4-EGFP upon stimulation of GPCRx with its corresponding agonist (control: CXCR4-GFP (FIG. 4A)). U-2 OS cells were co-transduced with adenoviruses encoding CXCR4-EGFP and GPCRx-VC, and the following GPCRx partner was examined to form heteromer with CXCR4-EGFP and to induce co-internalization of the CXCR4-EGFP: GPCRx represents ADRB2 (FIG. 4B).

FIGS. 5A-5D: Show enhancement of the calcium response in cells co-expressing CXCR4 and ADRB2 upon co-stimulation with their respective selective agonists. MDA-MB-231 human breast cancer cells were transduced with adenoviruses encoding CXCR4 and HA-VC (FIG. 5A), ADRB2 and HA-VC (FIG. 5B), or CXCR4 and ADRB2 (FIG. 5C). Adenoviruses encoding HA-VC were used to adjust the total amount of adenoviruses transduced. Cells were allowed to express GPCR for 2 days, incubated with Cal-520 AM for 2 hr, and were treated with 15 nM of CXCL12, 100 nM of salmeterol (ADRB2-selective agonist), or CXCL12 and salmeterol together. Calcium mobilization was measured using FlexStation 3 Multi-Mode Microplate Reader. The results were normalized for base-line activity. Data represent three independent experiments (mean±SEM). (FIG. 5D) Calcium mobilization was quantified by calculating the area-under-the-curve (AUC) of each graph in A-C. Data were normalized to CXCL12-stimulated calcium response in cells expressing CXCR4 alone. Sum represents the calculated additive effect of the responses obtained after stimulation of CXCL12 and salmeterol individually in cells co-expressing CXCR4 and ADRB2, to allow the visualization of the potentiation. ***P<0.001; Student's t test.

FIG. 6: Shows co-administration with both antagonists efficiently suppressed the enhanced calcium response when cells expressing CXCR4 and ADRB2, and containing the CXCR4-ADRB2 heteromer, were simultaneously stimulated with CXCL12 and ADRB2 agonist salmeterol. MDA-MB-231 cells were co-transduced with adenoviruses encoding CXCR4 and ADRB2. Cells were incubated with Cal-520 AM for 2 hr, ADRB2 antagonist or vehicle for 30 min, and stimulated with indicated amounts of CXCL12, ADRB2 agonist salmeterol, or CXCL12 and ADRB2 agonist salmeterol. Calcium mobilization was quantified as described in FIG. 5D. Data represent three independent experiments (mean±SEM). *P<0.05, **P<0.01, ***P<0.001; Student's t test.

FIG. 7: Shows principle of internalization inhibition study. Cells co-expressing CXCR4-GFP and GPCRx are treated with CXCL12 (SDF-1) and/or GPCRx specific antagonist. Scenario A: CXCR4 and GPCRx forms heteromer. CXCR4 agonist, CXCL12 (SDF-1) induced internalization of CXCR4-GFP alone or CXCR4-GFP with GPCRx. Scenario B: GPCRx antagonist does not induce internalization of CXCR4-GFP. Scenario C: Internalization of CXCL12 (SDF-1) stimulated CXCR4-GFP is inhibited by GPCRx specific antagonist.

FIG. 8: Shows inhibition of internalization by ADRB2 antagonist for CXCR4-ADRB2 heteromers. CXCR4-GFP expressing U-2 OS cells were transduced with adenovirus encoding ADRB2. Treatment by CXCL12, a CXCR4 agonist, induced CXCR4-ADRB2 internalization. But Carvedilol, an ADRB2 antagonist, did not induce internalization. Co-treatment with CXCL12 and Carvedilol partially inhibited CXCL12 induced CXCR4-ADRB2 internalization.

FIG. 9: Shows the effect of ADRB2 antagonist on the survival of patient derived cells (PDCs) from cancer patients. Effect of ADRB2 antagonist (Carvedilol) on the survival of PDC. Carvedilol induced significantly decreased survival of cells (IC50=11.69 μM).

FIGS. 10A-10C: Show CXCR4-ADRB2 heterodimer detection by PLA and RT-qPCR in U-2 OS cells over-expressing CXCR4 and ADRB2. CXCR4-GFP expressing U-2 OS cells were transduced with adenovirus encoding ADRB2 at different MOIs for 2 days. Then CXCR4-ADRB2 co-expressing U-2 OS cells were performed PLA. FIG. 10A: Image of CXCR4-ADRB2 heteromer detection by PLA. FIG. 10B: Increase of the PLA signal proportionate to the expression level of ADRB2 in a dose dependent manner. FIG. 10C: Data from RT-qPCR showing endogenous ADRB2 expression level of U-2 OS cells.

FIGS. 11A-11B: Show CXCR4-ADRB2 heteromer detection in PDC by PLA. GBM originated PDCs (sample IDs: 986T, 948T, 783T, 777T, 352T1, 352T2, 578T, 559T, 464T, 448T, 096T) were plated at chamber slide and CXCR4-ADRB2 heteromer were detected by PLA with CXCR4 and ADRB2 specific antibodies. FIG. 11A: Image of CXCR4-ADRB2 heteromer detection. Nuclei were visualized with DAPI staining and CXCR4-ADRB2 heteromers were shown as small dots. FIG. 11B: Ratio of CXCR4-ADRB2 heteromer in PDC.

FIGS. 12A-12B: Show results from CXCR4-GPCRx heteromer detection in PDX. GBM originated PDXs (sample IDs; 777T, 783T, 948T, 559T) were detected CXCR4-ADRB2 heteromer by PLA with CXCR4 and ADRB2 specific antibodies. FIG. 12A: Image of CXCR4-ADRB2 heteromer detection. Nuclei were visualized with DAPI staining and CXCR4-ADRB2 heteromers were shown as small dots. FIG. 12B: Ratio of CXCR4-ADRB2 heteromer in PDX.

FIG. 13: Shows the enhancement of the calcium response in cells co-expressing CXCR4 and ADRB2 upon co-stimulation with an ADRB2 agonist. MDA-MB-231 cells were transduced with adenoviruses encoding CXCR4 and ADRB2. Cells were cultured for 3 days, stained with Cal-520 AM, and were treated with either CXCL12 (30 nM) alone, increasing doses of salmeterol alone, or increasing doses of salmeterol in combination with 30 nM of CXCL12. Calcium mobilization was measured using FlexStation 3. Sum represents the calculated additive value of the responses evoked by 30 nM of CXCL12 alone (open square) and ADRB2 ligand alone at indicated doses (filled circle). Sum graph was depicted as a broken line with inverted triangles. Statistically significant differences between the sum (inverted triangle) and co-treatment (filled square) at each point were determined by Student's t test. *P<0.05; **P<0.01; ***P<0.001; Data represent mean±SD (n=3).

FIG. 14: Shows efficient suppression of the enhanced calcium response by co-treatment of anti-CXCR4 antibody and ADRB2 antagonist when cells expressing CXCR4-ADRB2 heteromer were simultaneously stimulated with CXCL12 and ADRB2 agonist. MDA-MB-231 cells were co-transduced with adenoviruses encoding CXCR4 and ADRB2. Cells were treated with ADRB2 antagonist (Carvedilol), anti-CXCR4 antibody (12G5), or vehicle of indicated concentration and incubated with Cal 6 for 2 hours. Cells were subsequently stimulated with indicated amounts of CXCL12, ADRB2 agonist (Salmeterol), or CXCL12 and ADRB2 agonist.

FIGS. 15A-15B: FIG. 15A: Shows images of three mice that were transplanted with a parental cell A549, A549-CXCR4 overexpressing CXCR4 stably and A549-CXCR4-ADRB2 stably overexpressing CXCR4-ADRB2 heteromer, respectively, at 28 days after transplantation;

FIG. 15B: Shows graph comparing tumor growth between transplanted cells from FIG. 15A; tumor growth was monitored every third or fourth day by measuring the length (L) and width (W) of the tumor and calculating tumor volume in the basis of the following formula: Volume=0.5 LW². The results are presented as the mean±standard deviation of 3 individuals.

FIGS. 16A-16B: Show ERK activation by stimulation of CXCL12 and/or Salmeterol in CXCR4-ADRB2 heteromer expressing MDA-MB-231 cells. FIG. 16A shows Western blot analysis of phospho-ERK1/2 Thr202/Tyr204 and total ERK1/2 in MDA-MB-231 cells, MDA-MB-231-CXCR4 and MDA-MB-231-CXCR4-ADRB2 treated with CXCL12 (10 nM) and/or Salmeterol (10 nM), for 20 minutes. FIG. 16B shows densitometric analysis (iBright Analysis Software) of phospho-ERK1/2 Thr202/Tyr204 protein expression relative to total ERK protein levels.

FIGS. 17A-17B: Show ERK activation by stimulation of CXCL12 and/or Salmeterol in CXCR4-ADRB2 heteromer expressing A549 cells. FIG. 17A shows Western blot analysis of phospho-ERK1/2 Thr202/Tyr204 and total ERK1/2 in A549, A549-CXCR4, and A549-CXCR4-ADRB2 cells treated with CXCL12 (10 nM) and/or Salmeterol (10 nM), for 10 minutes. FIG. 17B shows Densitometric analysis (iBright Analysis Software) of phospho-ERK1/2 Thr202/Tyr204 protein expression relative to total ERK protein levels.

FIGS. 18A-18E: Show the effect of CXCR4 antagonists on CXCR4-CXCL12 mediated proliferation increase. A549-double negative (RLuc-Luc2P) and A549-CXCR4-ADRB2 cells overexpressing CXCR4 and ADRB2 were plated into 96-well plates and stimulated for 72 hr with CXCL12 and/or indicated drugs (FIG. 18A: AMD3100 (10 μM), FIG. 18B: LY2510924 (10 μM), FIG. 18C: AMD070 (1 μM), FIG. 18D: TG-0054 (10 μM), and FIG. 18E: BKT-140 (10 μM)) in serum free condition. Fluorescence from three replicate wells were measured and data are represented as mean ratio of fluorescence (Drug treatment/Vehicle)±SEM.

FIGS. 19A-19C: Show the correlation between CXCR4-ADRB2 heteromer expression and tumor growth: FIG. 19A shows detection of CXCR4-ADRB2 heteromer by PLA in A549 parent cell, CXCR4 homomer expressing A549-CXCR4 cell line, and CXCR4-ADRB2 heteromer expressing A549-CXCR4-ADRB2 cell line; FIG. 19B shows quantification of CXCR4-ADRB2 heteromer by PLA in A549, A549-CXCR4, and A549-CXCR4-ADRB2; and FIG. 19C shows comparison of tumor growth between A549 parent cell bearing mice and A549-CXCR4-ADRB2 cell bearing mice.

FIGS. 20A-20C: Show the correlation between CXCR4-ADRB2 heteromer expression and tumor growth. FIG. 20A shows detection of CXCR4-ADRB2 heteromer by PLA in MDA-MB-231 parent cell, CXCR4 homomer expressing MDA-MB-231-CXCR4 cell line, and CXCR4-ADRB2 heteromer expressing MDA-MB-231-CXCR4-ADRB2 cell line; FIG. 20B shows quantification of CXCR4-ADRB2 heteromer by PLA in MDA-MB-231, MDA-MB-231-CXCR4, and MDA-MB-231-CXCR4-ADRB2; and FIG. 20C shows comparison of tumor growth among MDA-MB-231 parent cell, MDA-MB-231-CXCR4, or MDA-MB-231-CXCR4-ADRB2 cell bearing mice.

FIGS. 21A-21D: Show the effect of CXCR4 inhibitor alone on tumor growth in CXCR4-ADRB2 heteromer expressing A549 xenografted mouse. Mice transplanted with the A549 cell line expressing the CXCR4-ADRB2 heteromer were subjected to various kinds of CXCR4 inhibitors, AMD3100 (FIG. 21A), LY2510924 (FIG. 21B), AMD070 (FIG. 21C), or TG-0054 (FIG. 21D) in a dose dependent manner to compare the tumor size.

FIGS. 22A-22D: Show the relative growth of tumor when CXCR4-ADRB2 heteromer expressing cells were transplanted into mice and treated with a CXCR4 inhibitor and ADRB2 inhibitor carvedilol, alone or in combination. FIGS. 22A-22C: Shows the relative growth of tumor when CXCR4-ADRB2 heteromer expressing MDA-MB-231 cells were transplanted orthotopically into mice and treated with CXCR4 inhibitor (AMD3100 (FIG. 22A), LY2510924 (FIG. 22B), or AMD70 (FIG. 22C)) and ADRB2 inhibitor carvedilol, alone or in combination. The results are presented as the mean±standard deviation of 5 individuals. FIG. 22D: Shows the relative growth of tumor when CXCR4-ADRB2 heteromer expressing A549 cells were transplanted into mice (an A549 xenograft model) and treated with AMD3100 (CXCR4 inhibitor) and carvedilol (ADRB2 inhibitor), alone or in combination. Tumor growth was monitored every third or fourth day by measuring the length (L) and width (W) of the tumor and calculating tumor volume in the basis of the following formula: Volume=0.5 LW². The results are presented as the mean±standard deviation of 10 individuals.

FIG. 23A: Show a ligand-based TR-FRET signal observed on A549 cells transiently infected with Ad-CXCR4 and Ad-ADRB2 at indicated MOIs and labeled with TZ14011-tb and Propranolol-g2 in the absence or presence of Propranolol (1 M) as a competitor.

FIG. 23B: Show an antibody-based TR-FRET signal observed on U2OS cells transiently infected with Ad-CXCR4 and Ad-ADRB2 at indicated MOIs. Followed by incubation with rabbit anti-CXCR4 antibody and mouse anti-ADRB2 antibody, goat anti-rabbit IgG labeled with terbium cryptate and goat anti-mouse IgG labeled with Alexa Fluor 647 were used for TR-FRET.

FIGS. 24A-24C: Show CXCR4-ADRB2 heteromer detection by PLA and quantification of CXCR4 or ADRB2 via RNA expression level by RT-qPCR in solid tumor cancer cell lines: A549 (lung cancer), U2OS (osteosarcoma), and MDA-MB-231 (breast carcinoma). FIG. 24A shows RNA expression levels of CXCR4 and ADRB2 by RT-qPCR; FIG. 24B shows images of CXCR4 and ADRB2 heteromer detected by PLA; and FIG. 24C shows quantification of CXCR4 and ADRB2 heteromer by PLA.

FIGS. 25A-25C: Show CXCR4-ADRB2 heteromer detection by PLA and quantification of CXCR4 or ADRB2 via RNA expression level by RT-qPCR in blood cancer cell lines: HL60 (leukemia), U937 (leukemia), and RPMI8226 (Myeloma). FIG. 25A shows RNA expression levels of CXCR4 and ADRB2 by RT-qPCR; FIG. 25B shows images of CXCR4 and ADRB2 heteromer detected by PLA; and FIG. 25C shows quantification of CXCR4 and ADRB2 heteromer by PLA.

FIGS. 26A-26B: Shows enhancement of calcium response in cells expressing CXCR4-ADRB2 heteromer when co-stimulated with CXCL12 and an ADRB2 agonist (formoterol). FIG. 26A shows enhancement of calcium response in U937 cells expressing CXCR4-ADRB2 heteromer upon co-stimulation with CXCL12 and ADRB2 agonist formoterol; FIG. 26B shows enhancement of calcium response in HL-60 cells expressing CXCR4-ADRB2 heteromer upon co-stimulation with CXCL12 and ADRB2 agonist formoterol.

### ABBREVIATIONS

Unless indicated otherwise, the following includes abbreviations for terms disclosed herein: acute myeloid leukemia (AML), adenovirus high-throughput system (AdHTS), Adrenoceptor Beta 2 (ADRB2), bimolecular fluorescence complementation (BiFC), Bioluminescence Resonance Energy Transfer (BRET), bovine serum albumin (BSA), Cancer stem cells (CSCs), chronic lymphocytic leukemia (CLL), chronic myeloid leukemia (CML), chronic obstructive pulmonary disease (COPD), C-terminal fragment of Venus (VC), C-X-C Motif Chemokine ligand 12 (CXCL12), CXC receptor 4 (CXCR4), enzyme-linked immunosorbent assay (ELISA), formalin-fixed paraffin-embedded (FFPE), fluorescence resonance energy transfer (FRET), G protein-coupled receptor (GPCR), glioblastoma multiforme (GBM), Glucagon receptor (GCGR), GPCR heteromer identification technology (GPCR-HIT), Granulocyte-colony stimulating factor (G-CSF), hematopoietic stem cells (HSCs), hepatocellular carcinoma (HCC), human immunodeficiency virus (HIV), International Union of Basic and Clinical Pharmacology Committee on Receptor Nomenclature and Drug Classification (NC-IUPHAR), Multiple myeloma (MM), multiplicity of infection (MOI), Myelodysplastic Syndromes (MDS), non-Hodgkin lymphoma (NHL), non-small-cell lung cancer (NSCLC), N-terminal fragments of Venus (VN), patient derived cell (PDC), Patient-Derived Xenograft (PDX), positron emission tomography (PET), Computed Tomography (CT), proximity ligation assay (PLA), reverse transcription-quantitative polymerase chain reaction (sometimes referred to as RT-qPCR, or qRT-PCR, or qPCR, or realtime PCR), Single-photon emission computed tomography (SPECT), small lymphocytic lymphoma (SLL), small-cell lung cancer (SCLC), Stromal cell-derived factor 1 (SDF-1), systemic lupus erythematosus (SLE), Threshold cycles (Ct), time-resolved FRET (TR-FRET), tumor microenvironment (TME), vascular smooth muscle cells (VSMC), WHIM syndrome (Warts, Hypogammaglobulinemia, Infections, and Myelokathexis), green fluorescence protein (GFP), and yellow fluorescence protein (YFP).

### DETAILED DESCRIPTION OF THE INVENTION

As used herein, the articles “a”, “an”, and “the”, refer to one or to more than one of the grammatical object of the article. By way of example, a sample refers to one sample or two or more samples.

As used herein, the term “subject” refers to a mammal. A subject can be a human or a non-human mammal such as a dog, cat, bovid, equine, mouse, rat, rabbit, or transgenic species thereof. The subject can be a patient, or a cancer patient.

As used herein, the term “sample” refers to a material or mixture of materials containing one or more components of interest. A sample from a subject refers to a sample obtained from the subject, including samples of biological tissue or fluid origin, obtained, reached, or collected in vivo or in situ. A sample can be obtained from a region of a subject containing precancerous or cancer cells or tissues. Such samples can be, but are not limited to, organs, tissues, fractions and cells isolated from a mammal. In certain embodiments, the sample may be a biological sample, such as a biological fluid sample or a biological tissue sample. A sample also can be a tissue biopsy. In certain embodiments, the biological sample includes, but is not limited to, lymph node, a blood sample, a plasma sample, whole blood, partially purified blood, serum, bone marrow, cell lysate, a cell culture, a cell line, a tissue, oral tissue, gastrointestinal tissue, an organ, an organelle, a biological fluid, a urine sample, a skin sample, a saliva sample, a cerebral fluid sample, or an eye fluid sample.

As used herein, the terms “treat”, “treating”, and “treatment”, when used in reference to a cancer patient, refer to an action that reduces the severity of the cancer, or retards or slows the progression of the cancer, including (a) inhibiting the cancer growth, or arresting development of the cancer, and (b) causing regression of the cancer, or delaying or minimizing one or more symptoms associated with the presence of the cancer.

As used herein, the term “administer”, “administering”, or “administration”, refers to the act of delivering, or causing to be delivered, a compound, a combination of compounds, or a pharmaceutical composition comprising the same, to the body of a subject by a method described herein or otherwise known in the art. Administering a compound, a combination of compounds, or a pharmaceutical composition comprising the same, includes prescribing a compound, a combination of compounds, or a pharmaceutical composition comprising the same, to be delivered into the body of a patient. Exemplary forms of administration include oral dosage forms, such as tablets, capsules, syrups, suspensions; injectable dosage forms, such as intravenous (IV), intramuscular (IM), or intraperitoneal (IP); subcutaneous (SC), intracranial (IC); transdermal dosage forms, including creams, jellies, powders, or patches; buccal dosage forms; inhalation powders, sprays, suspensions, and rectal suppositories.

As used herein, the phrase “therapeutic agent” refers to any agent that can be used in the treatment, amelioration, prevention, or management of a cancer and/or a symptom related thereto. In certain embodiments, a therapeutic agent refers to an inhibitor of CXCR4-ADRB2 heteromer of the invention. A therapeutic agent can be an agent which is well known to be useful for, or has been or is currently being used for the treatment, amelioration, prevention, or management of a cancer and/or a symptom related thereto.

As used herein, the phrase “effective amount” as used herein refers to an amount sufficient to effect beneficial or desired results. An effective amount can be administered in one or more administrations, applications or dosages. Such delivery is dependent on a number of variables including the time period for which the individual dosage unit is to be used, the bioavailability of the agent, the route of administration, etc.

As used herein, the term “therapeutically effective amount” of a compound (e.g., a therapeutic agent, such as an inhibitor, an antagonist, or any other therapeutic agent provided herein) or a combination of compounds when used in connection with a disease or disorder refers to an amount sufficient to provide a therapeutic benefit in the treatment or management of the disease or disorder or to delay or minimize one or more symptoms associated with the disease or disorder. A therapeutically effective amount of a compound means an amount of the compound that when used alone or in combination with other therapies, would provide a therapeutic benefit in the treatment or management of the disease or disorder. The term encompasses an amount that improves overall therapy, reduces or avoids symptoms, or enhances the therapeutic efficacy of another therapeutic agent. The term also refers to the amount of a compound that sufficiently elicits the biological or medical response of a biological molecule (e.g., a protein, enzyme, RNA, or DNA), cell, tissue, system, animal, or human, which is being sought by a researcher, veterinarian, medical doctor, or clinician.

As used herein, the term “heteromer” refers to macromolecular complex composed of a CXCR4 unit (or sometimes referred to as a CXCR4 protomer when identified in the context of a CXCR4-containing heteromer) and a ADRB2 unit (or sometimes referred to as a ADRB2 protomer when identified in the context of a ADRB2-containing heteromer) with biochemical properties that are demonstrably different from the biochemical properties of the CXCR4 monomer or the ADRB2 monomer, respectively, or demonstrably different from the biochemical properties of both the CXCR4 monomer and the ADRB2 monomer, respectively. Heteromerization can be evaluated by in situ hybridization, immunohistochemistry, RNAseq, Reverse transcription-quantitative PCR (sometimes referred to as RT-qPCR, or qRT-PCR, or qPCR, or realtime PCR), expression levels of each monomer or protomer of the identified heteromer (e.g., expression levels of CXCR4 and ADRB2 as associated with a CXCR4-ADRB2 heteromer), microarray, proximity ligation assay (PLA), time-resolved FRET (TR-FRET), whole-body Single-photon emission computed tomography (SPECT) or Positron Emission Tomography/Computed Tomography (PET/CT).

As used herein, the phrases “intracellular Ca2+ assay”, “calcium mobilization assay”, or their variants, refer to cell-based assay to measure the calcium flux associated with GPCR activation or inhibition, such as CXCR4 activation or inhibition and/or ADRB2 activation or inhibition. The method utilizes a calcium sensitive fluorescent dye that is taken up into the cytoplasm of most cells. The dye binds the calcium released from intracellular store and its fluorescence increases. The change in the fluorescence intensity is directly correlated to the amount of intracellular calcium that is released into cytoplasm in response to ligand activation of the receptor of interest. In certain embodiments, the GPCR is CXCR4. In certain embodiments, the GPCR is ADRB2. In certain embodiments, the cell-based assay measures the calcium flux associated with a CXCR4-ADRB2 heteromer activation or inhibition.

As used herein, the phrase “proximity-based assay”, refers to biophysical and biochemical techniques that are able to monitor proximity and/or binding of two protein molecules in vitro (in cell lysates) and in live cells, such as the proximity and/or binding of a CXCR4 protein (monomer or unit) and an ADRB2 protein (monomer or unit), including bioluminescence resonance energy transfer (BRET), fluorescence resonance energy transfer (FRET), bimolecular fluorescence complementation (BiFC), Proximity ligation assay (PLA), cysteine crosslinking, and co-immunoprecipitation (Ferre, S., et al., (2009) Building a new conceptual framework for receptor heteromers, Nat Chem Biol 5, 131-134; Gomes, I., et al., (2016) G Protein-Coupled Receptor Heteromers, Annu Rev Pharmacol Toxicol 56, 403-425).

Alternative methods for detecting CXCR4-ADRB2 heteromer formation include, but are not limited to: immunostaing (Bushlin, I., et al., (2012) Dimerization with cannabinoid receptors allosterically modulates delta opioid receptor activity during neuropathic pain, PLoS One 7, e49789; Decaillot, F. M., et al., (2008) Cell surface targeting of mu-delta opioid receptor heterodimers by RTP4, Proc Natl Acad Sci USA 105, 16045-16050); immunoelectron microscopy (Fernandez-Duenas, V., et al., (2015) Untangling dopamine-adenosine receptor-receptor assembly in experimental parkinsonism in rats, Dis Model Mech 8, 57-63); BRET (Pfleger, K. D., et al., (2006) Illuminating insights into protein-protein interactions using bioluminescence resonance energy transfer (BRET), Nat Methods 3, 165-174); Time-resolved FRET assays (Fernandez-Duenas, V., et al., 2015); In Situ Hybridization (He, S. Q., et al., (2011) Facilitation of mu-opioid receptor activity by preventing delta-opioid receptor-mediated codegradation, Neuron 69, 120-131); FRET (Lohse, M. J., et al., (2012) Fluorescence/bioluminescence resonance energy transfer techniques to study G-protein-coupled receptor activation and signaling, Pharmacol Rev 64, 299-336); β-arrestin recruitment assay using GPCR heteromer identification technology (GPCR-HIT, Dimerix Bioscience) (Mustafa, S., et al., (2011) G protein-coupled receptor heteromer identification technology: identification and profiling of GPCR heteromers, J Lab Autom 16, 285-291) using BRET, FRET, BiFC, Bimolecular Luminescence Complementation, enzyme fragmentation assay, and Tango Tango GPCR assay system (Thermo Fisher Scientific) (Mustafa, S., et al., (2010) Uncovering GPCR heteromer-biased ligands, Drug Discov Today Technol 7, e1-e94); PRESTO-Tango system (Kroeze, W. K., et al., (2015) PRESTO-Tango as an open-source resource for interrogation of the druggable human GPCRome, Nat Struct Mol Biol 22, 362-369); regulated secretion/aggregation technology (ARIAD Pharmaceuticals) (Hansen, J. L., et al., (2009) Lack of evidence for AT1R/B2R heterodimerization in COS-7, HEK293, and NIH3T3 cells: how common is the AT1R/B2R heterodimer? J Biol Chem 284, 1831-1839); Receptor Selection and Amplification Technology (ACADIA Pharmaceuticals) (Hansen, J. L., et al., 2009); DimerScreen (Cara Therapeutics) (Mustafa, S., 2010); Dimer/interacting protein translocation assay (Patobios) (Mustafa, S., 2010); Co-immunoprecipitation (Abd Alla, J., et al., (2009) Calreticulin enhances B2 bradykinin receptor maturation and heterodimerization, Biochem Biophys Res Commun 387, 186-190); GPCR internalization assays using surface enzyme-linked immunosorbent assay (ELISA) (Decaillot, F. M., et al., 2008) or Flow Cytometry (Law, P. Y., et al., (2005) Heterodimerization of mu- and delta-opioid receptors occurs at the cell surface only and requires receptor-G protein interactions, J Biol Chem 280, 11152-11164); Whole Cell Phosphorylation Assays (Pfeiffer, M., et al., (2002) Heterodimerization of somatostatin and opioid receptors cross-modulates phosphorylation, internalization, and desensitization, J Biol Chem 277, 19762-19772); and Proximity-ligation assay (PLA) (Frederick, A. L., et al., (2015) Evidence against dopamine D1/D2 receptor heteromers, Mol Psychiatry 20, 1373-1385).

Alternative methods for detecting changes in pharmacological properties, signaling properties, and/or trafficking properties, in cells expressing both CXCR4 and ADRB2 include, but are not limited to: Radioligand Binding Assays (Bushlin, I., et al., 2012; Pfeiffer, M., et al., 2002); Cell Surface Biotinylation and Immunoblotting (He, S. Q., et al., 2011); immunostaing (Bushlin, I., et al., 2012; Decaillot, F. M., et al., 2008); immunoelectron microscopy (Fernandez-Duenas, V., et al., 2015); [35S]GTP-γS Binding assays (Bushlin, I., et al., 2012); Calcuim imaging or assays using dyes such as Fura 2-acetomethoxy ester (Molecular Probes), Fluo-4 NW calcium dye (Thermo Fisher Scientific), or FLIPR5 dye (Molecular Devices); cAMP assays using radioimmunoassay kit (Amersham Biosciences); AlphaScreen (PerkinElmer Life Sciences); Parameter Cyclic AMP Assay (R&D Systems); femto cAMP kit (Cisbio); cAMP Direct Immunoassay Kit (Calbiochem) or GloSensor cAMP assay (Promega); GTPase assay (Pello, O. M., et al., (2008) Ligand stabilization of CXCR4/delta-opioid receptor heterodimers reveals a mechanism for immune response regulation, Eur J Immunol 38, 537-549); PKA activation (Stefan, E., et al., (2007) Quantification of dynamic protein complexes using Renilla luciferase fragment complementation applied to protein kinase A activities in vivo, Proc Natl Acad Sci USA 104, 16916-16921); ERK1/2 and/or Akt/PKB Phosphorylation Assays (Callen, L., et al., (2012) Cannabinoid receptors CB1 and CB2 form functional heteromers in brain, J Biol Chem 287, 20851-20865); reporter assays such as cAMP response element (CRE); serum response element (SRE); serum response factor response element (SRF-RE); and Secreted alkaline phosphatase Assay (Decaillot, F. M., et al., 2011); Measurement of Inositol 1-Phosphate Production Using TR-FRET or [3H]myo-Inositol (Mustafa, S., et al., (2012) Identification and profiling of novel alpha1A-adrenoceptor-CXC chemokine receptor 2 heteromer, J Biol Chem 287, 12952-12965); RT-qPCR for measuring downstream target gene expression (Mustafa, S., et al., 2012); and Adenylyl Cyclase Activity (George, S. R., et al., (2000) Oligomerization of mu- and delta-opioid receptors. Generation of novel functional properties. J Biol Chem 275, 26128-26135); next generation sequencing (NGS); and any other assay that can detect a change in receptor function as a result of receptor heterodimerization.

As used herein, the term “ADRB2” refers to Adrenoceptor Beta 2 (also referred to as beta-2 adrenergic receptor or β2 adrenoreceptor), a cell membrane-spanning beta-adrenergic receptor that interacts with epinephrine, a hormone and neurotransmitter (ligand synonym, adrenaline) whose signaling, via a downstream L-type calcium channel interaction, mediates physiologic responses such as smooth muscle relaxation and bronchodilation (Gregorio, G. G., et al., 2017). ADRB2 is also identified by unique exemplary database identifiers (IDs) and alternate names as shown in Table 1. ADRB2 functions in muscular system such as smooth muscle relaxation, motor nerve terminals, glycogenolysis and in circulatory system such as heart muscle contraction, cardiac output increase. In the normal eye, beta-2 stimulation by salbutamol increases intraocular pressure via net. In digestive system, the ADRB2 induces glycogenolysis and gluconeogenesis in liver and insulin secretion from pancreas (Fitzpatrick, D., et al., (2004) “Table 20:2” (Mass: Sunderland)). Activation of ADRB2 can stimulate signaling pathways that promote tumor growth and metastasis (Antoni, M. H., et al., (2006) Nat Rev Cancer 6: 240-248; Thaker, P. H., et al., (2006) Nat Med 12: 939-944), including the Ras-mediated Raf proto-oncogene serine/threonine-protein kinase (Raf)/dual specificity mitogen-activated protein kinase kinase (MEK)/extracellular signal-regulated kinase (ERK), phosphoinositide 3-kinase (PI3K)/RAC serine/threonine-protein kinase (Akt) and cAMP/protein kinase A/mitogen-activated protein kinase pathways that promote cellular proliferation and invasion, and suppress apoptosis in cancer cells, which can enhance tumor growth and facilitate metastasis (Qu\hat{ó}c Lu'o'ng, K. V., et al., (2012) Cancer Manag Res 4: 431-445).

As used herein, the term “CXCR4” refers to C-X-C Motif Chemokine Receptor 4, also identified by unique exemplary database identifiers (IDs) and alternate names as shown in Table 1 (Chatterjee, S., et al., 2014; Debnath, B., et al., 2013; Domanska, U. M., et al., 2013; Guo, F., et al., (2016) CXCL12/CXCR4: a symbiotic bridge linking cancer cells and their stromal neighbors in oncogenic communication networks, Oncogene 35, 816-826; Peled, A., et al., (2012) Development of novel CXCR4-based therapeutics, Expert Opin Investig Drugs 21, 341-353; Roccaro, A. M., et al., (2014) SDF-1 inhibition targets the bone marrow niche for cancer therapy, Cell Rep 9, 118-128; Walenkamp, A. M. E., et al., (2017) CXCR4 Ligands: The Next Big Hit? J Nucl Med 58, 77S-82S). The binding of CXCL12 to CXCR4 initiates divergent signaling pathways downstream of ligand binding, which can result in a variety of responses such as chemotaxis, cell survival and/or proliferation, increase in intracellular calcium, and gene transcription. Chemotaxis has been shown to be mediated via MAPK either through PKC, or through Gui, which can signal through Erk/1/2 (Mellado, M., et al., (2001) Annu Rev Immunol; 19:397-421; Bendall, L. J., et al., (2005) Cancer Res; 65:3290-8). The pair of chemokine CXCL12 and chemokine receptor CXCR4 play important roles in many stages of tumorigenesis. CXCR4 is overexpressed in a variety of human cancers, and this overexpression is correlated with increased risk for recurrence and poor overall survival in multiple cancer subjects including breast, lung, kidney, colon, ovarian, and brain cancers, as well as lymphoma and leukemia (Balkwill, F., (2004) Nat Rev Cancer; 4:540-50. 1-5; Orimo, A., et al., (2005) Cell; 121:335-48; Domanska, U. M., et al., 2013). The critical roles of CXCR4 in cancer and other diseases have triggered the development of selective CXCR4 inhibitors for clinical use.

TABLE 1

| Gene name | Full name | Other names | Exemplary IDs |
|---|---|---|---|
| CXCR4 | C-X-C Motif | Leukocyte-Derived Seven | GCID: GC02M136114 |
| | Chemokine | Transmembrane Domain Receptor; | HGNC: 2561 |
| | Receptor 4 | Lipopolysaccharide-Associated | Entrez Gene: 7852 |
| | | Protein 3; Stromal Cell-Derived | Ensembl: ENSG00000121966 |
| | | Factor 1 Receptor; Chemokine (C- | OMIM: 162643 |
| | | X-C Motif) Receptor 4; LPS- | UniProtKB: P61073 |
| | | Associated Protein 3 Seven | |
| | | Transmembrane Helix Receptor; C- | |
| | | X-C Chemokine Receptor Type 4; | |
| | | Neuropeptide Y Receptor Y3; | |
| | | Neuropeptide Y3 Receptor; | |
| | | Chemokine Receptor; Seven- | |
| | | Transmembrane-Segment Receptor, | |
| | | Spleen; Chemokine (C-X-C Motif), | |
| | | Receptor 4 (Fusin); SDF-1 | |
| | | Receptor; CD184 Antigen; Fusin; | |
| | | LAP-3; LESTR; NPYRL; FB22; | |
| | | HM89; LCR1; D2S201E; | |
| | | HSY3RR; NPYY3R; | |
| | | CXC-R4; CXCR-4; CD184; | |
| | | NPY3R; WHIMS; LAP3; NPYR; | |
| | | WHIM | |
| ADRB2 | Adrenoceptor | Adrenergic, Beta-2-, Receptor, | GCID: GC05P148825 |
| | Beta 2 | Surface; Beta-2 Adrenoreceptor; | HGNC: 286 |
| | | Beta-2 Adrenoceptor; ADRB2R; | Entrez Gene: 154 |
| | | B2AR; Adrenoceptor Beta 2, | Ensembl: ENSG00000169252 |
| | | Surface; Adrenoceptor Beta 2 | OMIM: 109690 |
| | | Surface; Beta-2 Adrenergic | UniProtKB: P07550 |
| | | Receptor; Catecholamine Receptor; | |
| | | BETA2AR; ADRBR; BAR | |

*GCID: Genecards identification
HGNC: HUGO Gene Nomenclature Committee

As used herein, the term “CXCL12” (or Stroma Derived Factor 1 (“SDF-1”)) refers to a strong chemotactic agent for lymphocytes. During embryogenesis, CXCL12 directs the migration of hematopoietic cells from fetal liver to bone, and in adulthood, CXCL12 plays an important role in angiogenesis by recruiting endothelial progenitor cells through a CXCR4-dependent mechanism. CXCL12 is also expressed within the splenic red pulp and lymph node medullary cords (Pitt, et al., (2015) Cancer Cell, 27:755-768; and Zhao, et al., (2011) Proc. Natl. Acad. Sci. USA 108:337-342). An exemplary amino acid sequence and a corresponding encoding nucleic acid sequence of human CXCL12 may be found at GENBANK accession Nos.: NP_954637.1 and NM 199168.3, respectively.

Salmeterol is a long-acting 02 adrenergic receptor agonist (LABA) and has an aryl alkyl group with a chain length of 11 atoms from the amine. This bulkiness is believed to make the compound more lipophilic and selective for β2 adrenergic receptors. Salmeterol, first marketed and manufactured by Glaxo (now GlaxoSmithKline, GSK) in the 1980s, was released as Serevent® in 1990. The product is marketed by GSK under the Allen & Hanburys brand in the UK. Salmeterol used is the maintenance and prevention of asthma symptoms and the maintenance of chronic obstructive pulmonary disease (COPD) symptoms (2010 Global initiative for chronic obstructive disease). Symptoms of bronchospasm include shortness of breath, wheezing, coughing and chest tightness. Salmeterol is also used to prevent breathing difficulties during exercise (exercise-induced bronchoconstriction).

As used herein, the term “ADRB2 inhibitor” refers to a molecule that inhibits or suppresses the function of an ADRB2 monomer, or an ADRB2 unit or protomer of a CXCR4-ADRB2 heteromer. Non-limiting examples of an ADRB2 inhibitor that may be used in the methods of treatment, methods for suppressing, pharmaceutical composition and/or pharmaceutical kit, and methods and uses of the same, provided herein include, but are not limited to, an ADRB2 antagonist, an ADRB2 inverse agonist, an ADRB2 positive allosteric modulator, an ADRB2 negative allosteric modulator, an ADRB2-specific antibody or its antigen binding portions, including single-domain antibody-like scaffolds, a bivalent ligand which has a pharmacophore selective for ADRB2 joined by a spacer arm to a pharmacophore selective for CXCR4, a bispecific antibody against ADRB2 and CXCR4, a radiolabeled ADRB2 ligand linked with a CXCR4 ligand, and small molecule ligands that inhibit CXCR4-ADRB2 heteromer-selective signaling. In certain embodiments, the ADRB2 inhibitor inhibits or suppresses the function of the ADRB2 monomer. In certain embodiments, the ADRB2 inhibitor inhibits or suppresses the function of the ADRB2 unit or protomer of a CXCR4-ADRB2 heteromer. In certain embodiments, the ADRB2 inhibitor inhibits or suppresses the function of the CXCR4-ADRB2 heteromer. In certain embodiments, the ADRB2 inhibitor inhibits or suppresses the enhanced response upon stimulation with an ADRB2 agonist of an ADRB2 monomer. In certain embodiments, the ADRB2 inhibitor inhibits or suppresses the enhanced response upon stimulation with an ADRB2 agonist of an ADRB2 unit of a CXCR4-ADRB2 heteromer. In certain embodiments, the ADRB2 inhibitor inhibits or suppresses the enhanced response upon co-stimulation with an ADRB2 agonist and a CXCR4 agonist of an ADRB2 unit of a CXCR4-ADRB2 heteromer. In certain embodiments, the ADRB2 inhibitor inhibits or suppresses the enhanced response upon co-stimulation with an ADRB2 agonist and a CXCR4 agonist of the CXCR4-ADRB2 heteromer. In certain embodiments, the enhanced response is an enhanced Ca2+ response. In certain embodiments, the enhanced response is an enhanced cancer progression in the subject having cell(s) containing said CXCR4-ADRB2 heteromer. In certain embodiments, the enhanced cancer progression is an enhanced cell proliferation in the subject having cell(s) containing said CXCR4-ADRB2 heteromer. In certain embodiments, the enhanced cancer progression is an enhanced cell migration in the subject having cell(s) containing said CXCR4-ADRB2 heteromer. In certain embodiments, the enhanced cancer progression is an enhanced metastasis in the subject having cell(s) containing said CXCR4-ADRB2 heteromer. In certain embodiments, the enhanced cancer progression is an enhanced tumor growth in the subject having cell(s) containing said CXCR4-ADRB2 heteromer. In certain embodiments, the enhanced cancer progression is an enhanced angiogenesis in the subject having cell(s) containing said CXCR4-ADRB2 heteromer. In certain embodiments, the cell(s) are cancer cell(s). In certain embodiments, the cell(s) are derived from a subject. In certain embodiments, the cell(s) are from a biological sample obtained from a subject. Certain examples of ADRB2 inhibitors are listed in Table 2.

As used herein, the term “CXCR4 inhibitor” refers to a molecule that inhibits or suppresses the function of a CXCR4 monomer, or a CXCR4 unit or protomer of a CXCR4-ADRB2 heteromer. Non-limiting examples of a CXCR4 inhibitor that may be used in the methods of treatment, methods for suppressing, pharmaceutical composition and/or pharmaceutical kit, and methods and uses of the same, provided herein include, but are not limited to, a CXCR4 antagonist, a CXCR4 inverse agonist, a CXCR4 positive allosteric modulator, a CXCR4 negative allosteric modulator, a CXCR4-specific antibody or its antigen binding portions, including single-domain antibody-like scaffolds, a bivalent ligand which has a pharmacophore selective for CXCR4 joined by a spacer arm to a pharmacophore selective for ADRB2, a bispecific antibody against a CXCR4 and ADRB2, a radiolabeled a CXCR4 ligand linked with an ADRB2 ligand, and small molecule ligands that inhibit CXCR4-ADRB2 heteromer-selective signaling. In certain embodiments, the CXCR4 inhibitor inhibits or suppresses the function of the CXCR4 monomer. In certain embodiments, the CXCR4 inhibitor inhibits or suppresses the function of the CXCR4 unit or protomer of a CXCR4-ADRB2 heteromer. In certain embodiments, the CXCR4 inhibitor inhibits or suppresses the function of the CXCR4-ADRB2 heteromer. In certain embodiments, the CXCR4 inhibitor inhibits or suppresses the enhanced response upon stimulation with a CXCR4 agonist of a CXCR4 monomer. In certain embodiments, the CXCR4 inhibitor inhibits or suppresses the enhanced response upon stimulation with a CXCR4 agonist of a CXCR4 unit of a CXCR4-ADRB2 heteromer. In certain embodiments, the CXCR4 inhibitor inhibits or suppresses the enhanced response upon co-stimulation with a CXCR4 agonist and an ADRB2 agonist of a CXCR4 unit of a CXCR4-ADRB2 heteromer. In certain embodiments, the CXCR4 inhibitor inhibits or suppresses the enhanced response upon co-stimulation with a CXCR4 agonist and an ADRB2 agonist of the CXCR4-ADRB2 heteromer. In certain embodiments, the enhanced response is an enhanced Ca2+ response. In certain embodiments, the enhanced response is an enhanced cancer progression in the subject having cell(s) containing said CXCR4-ADRB2 heteromer. In certain embodiments, the enhanced cancer progression is an enhanced cell proliferation in the subject having cell(s) containing said CXCR4-ADRB2 heteromer. In certain embodiments, the enhanced cancer progression is an enhanced cell migration in the subject having cell(s) containing said CXCR4-ADRB2 heteromer. In certain embodiments, the enhanced cancer progression is an enhanced metastasis in the subject having cell(s) containing said CXCR4-ADRB2 heteromer. In certain embodiments, the enhanced cancer progression is an enhanced tumor growth in the subject having cell(s) containing said CXCR4-ADRB2 heteromer. In certain embodiments, the enhanced cancer progression is an enhanced angiogenesis in the subject having cell(s) containing said CXCR4-ADRB2 heteromer. In certain embodiments, the cell(s) are cancer cell(s). In certain embodiments, the cell(s) are derived from a subject. In certain embodiments, the cell(s) are from a biological sample obtained from a subject. Certain examples of CXCR4 inhibitors are listed in Table 2.

As used herein, the term “antagonist” refers to a type of receptor ligand or drug that blocks or dampens a biological response by binding to and blocking a receptor, also called blockers. Antagonists have affinity for their cognate receptors and their binding disrupts the interaction and inhibit the function of an agonist or inverse agonist at the cognate receptors. For example, an ADRB2 antagonist may be an ADRB2 ligand or ADRB2 drug that blocks or dampens a biological response by binding to and/or blocking an ADRB2 receptor. In certain embodiments, the ADRB2 antagonist may disrupt the interaction of an ADRB2 agonist or an ADRB2 inverse agonist with ADRB2 (e.g., ADRB2 monomer, or ADRB2 protomer or unit of a CXCR4-ADRB2 heteromer) and/or inhibit the function of an ADRB2 agonist or an ADRB2 inverse agonist with ADRB2 (e.g., ADRB2 monomer, or ADRB2 protomer or unit of a CXCR4-ADRB2 heteromer). In certain embodiments, the ADRB2 antagonist blocks, inhibits or suppresses the function of the ADRB2 monomer. In certain embodiments, the ADRB2 antagonist blocks, inhibits or suppresses the function of the ADRB2 unit or protomer of a CXCR4-ADRB2 heteromer. In certain embodiments, the ADRB2 antagonist blocks, inhibits or suppresses the function of the CXCR4-ADRB2 heteromer. In certain embodiments, the ADRB2 antagonist blocks, inhibits or suppresses the enhanced response upon stimulation with an ADRB2 agonist of an ADRB2 monomer. In certain embodiments, the ADRB2 antagonist blocks, inhibits or suppresses the enhanced response upon stimulation with an ADRB2 agonist of an ADRB2 unit of a CXCR4-ADRB2 heteromer. In certain embodiments, the ADRB2 antagonist blocks, inhibits or suppresses the enhanced response upon co-stimulation with an ADRB2 agonist and a CXCR4 agonist of an ADRB2 unit of a CXCR4-ADRB2 heteromer. In certain embodiments, the ADRB2 antagonist blocks, inhibits or suppresses the enhanced response upon co-stimulation with an ADRB2 agonist and a CXCR4 agonist of the CXCR4-ADRB2 heteromer. For example, a CXCR4 antagonist may be a CXCR4 ligand or CXCR4 drug that blocks or dampens a biological response by binding to and/or blocking a CXCR4 receptor. In certain embodiments, the CXCR4 antagonist may disrupt the interaction of a CXCR4 agonist or a CXCR4 inverse agonist with CXCR4 (e.g., CXCR4 monomer, or CXCR4 protomer or unit of a CXCR4-ADRB2 heteromer) and/or inhibit the function of a CXCR4 agonist or a CXCR4 inverse agonist with CXCR4 (e.g., CXCR4 monomer, or CXCR4 protomer or unit of a CXCR4-ADRB2 heteromer). In certain embodiments, the CXCR4 antagonist blocks, inhibits or suppresses the function of the CXCR4 monomer. In certain embodiments, the CXCR4 antagonist blocks, inhibits or suppresses the function of the CXCR4 unit or protomer of a CXCR4-ADRB2 heteromer. In certain embodiments, the CXCR4 antagonist blocks, inhibits or suppresses the function of the CXCR4-ADRB2 heteromer. In certain embodiments, the CXCR4 antagonist blocks, inhibits or suppresses the enhanced response upon stimulation with an CXCR4 agonist of an CXCR4 monomer. In certain embodiments, the CXCR4 antagonist blocks, inhibits or suppresses the enhanced response upon stimulation with an CXCR4 agonist of an CXCR4 unit of a CXCR4-ADRB2 heteromer. In certain embodiments, the CXCR4 antagonist blocks, inhibits or suppresses the enhanced response upon co-stimulation with an ADRB2 agonist and a CXCR4 agonist of an CXCR4 unit of a CXCR4-ADRB2 heteromer. In certain embodiments, the CXCR4 antagonist blocks, inhibits or suppresses the enhanced response upon co-stimulation with an ADRB2 agonist and a CXCR4 agonist of the CXCR4-ADRB2 heteromer. In certain embodiments, the enhanced response is an enhanced Ca2+ response. In certain embodiments, the enhanced response is an enhanced cancer progression in the subject having cell(s) containing said CXCR4-ADRB2 heteromer. In certain embodiments, the enhanced cancer progression is an enhanced cell proliferation in the subject having cell(s) containing said CXCR4-ADRB2 heteromer. In certain embodiments, the enhanced cancer progression is an enhanced cell migration in the subject having cell(s) containing said CXCR4-ADRB2 heteromer. In certain embodiments, the enhanced cancer progression is an enhanced metastasis in the subject having cell(s) containing said CXCR4-ADRB2 heteromer. In certain embodiments, the enhanced cancer progression is an enhanced tumor growth in the subject having cell(s) containing said CXCR4-ADRB2 heteromer. In certain embodiments, the enhanced cancer progression is an enhanced angiogenesis in the subject having cell(s) containing said CXCR4-ADRB2 heteromer. In certain embodiments, the cell(s) are cancer cell(s). In certain embodiments, the cell(s) are derived from a subject. In certain embodiments, the cell(s) are from a biological sample obtained from a subject. Certain examples of CXCR4 antagonists and ADRB2 antagonists are listed in Table 2.

TABLE 2

| | | Exemplary |
|---|---|---|
| | | Antibodies/ |
| Gene | | nanobodies/ |
| name | Exemplary Antagonists/Inverse agonists | i-bodies/others |
| CXCR4 | ALX40-4C, AMD070 (AMD11070, X4P-001), AMD3100 (plerixafor), | AD-114, AD-114- |
| | AMD3465, ATI 2341, BKT140 (BL-8040; TF14016; 4F-Benzoyl-TN14003), | 6H, AD-114-Im7- |
| | CTCE-9908, CX549, D-[Lys3] GHRP-6, FC122, FC131, GMI-1359, | FH, AD-114- |
| | GSK812397, GST-NT21MP, isothiourea-1a, isothiourea-1t (IT1t), KRH-1636, | PA600-6H, ALX- |
| | KRH-3955, LY2510924, MSX-122, N-[¹¹C]Methyl-AMD3465, POL6326, | 0651, LY2624587, |
| | SDF-1 1-9[P2G] dimer, SDF1 P2G, T134, T140, T22, TC 14012, TG-0054 | PF-06747143, |
| | (Burixafor), USL311, viral macrophage inflammatory protein-II (vMIP-II), | ulocuplumab |
| | WZ811, [⁶⁴Cu]-AMD3100, [⁶⁴Cu]-AMD3465, [⁶⁸Ga]pentixafor, | (MDX1338/BMS- |
| | [⁹⁰Y]pentixather, [^{99m}Tc]O₂-AMD3100, [¹⁷⁷Lu]pentixather, and 508MCl | 936564), 12G5, |
| | (Compound 26). | 238D2, and 238D4 |
| ADRB2 | Alprenolol, atenolol, betaxolol, bupranolol, butoxamine, carazolol, carvedilol, | |
| | CGP 12177, cicloprolol, ICI 118551, ICYP, labetalol, levobetaxolol, | |
| | levobunolol, LK 204-545, metoprolol, nadolol, NIHP, NIP, propafenone, | |
| | propranolol, sotalol, SR59230A, and timolol. | |

As used herein, the phrases “protein-protein interaction inhibitor”, “PPI inhibitor”, or their variants, refer to any molecules that can interfere with protein-protein interactions. Protein-protein interaction, unlike enzyme-substrate interaction involving well-defined binding pockets, is a transient interaction or association between proteins over relatively large areas and is often driven by electrostatic interactions, hydrophobic interactions, hydrogen bonds, and/or Van der Waals forces. PPI inhibitors may include, but not limited to, membrane-permeable peptides or lipid fused to a peptide sequence that disrupts the GPCR heteromeric interface, for example, transmembrane helix, intracellular loop, or C-terminal tail of the CXCR4 and or ADRB2 unit. The PPI inhibitor of the CXCR4-ADRB2 heteromer, for example, may be a membrane-permeable peptide or cell-penetrating peptide (CPP) conjugated with peptide that targets the CXCR4-ADRB2 heteromeric interface(s), or may be a cell-penetrating lipidated peptide targeting the CXCR4-ADRB2 heteromeric interface(s).

For example, the membrane-permeable peptide or cell-penetrating peptide includes: HIV-1 TAT peptides, such as TAT₄₈₋₆₀ and TAT₄₉₋₅₇; Penetratins, such as pAntp (43-58); Polyarginines (Rn such as R5 to R12); Diatos peptide vector 1047 (DPV1047, Vectocell®); MPG (HIV gp41 fused to the nuclear localization signal (NLS) of the SV40 large T antigen); Pep-1 (tryptophan-rich cluster fused to the NLS of SV40 large T antigen); pVEC peptide (vascular endothelial cadherin); p14 alternative reading frame (ARF) protein-based ARF (1-22); N-terminus of the unprocessed bovine prion protein BPrPr (1-28); Model amphipathic peptide (MAP); Transportans; Azurin-derived p28 peptide; amphipathic β-sheet peptides, such as VT5; proline-rich CPPs, such as Bac 7 (Baci-24); hydrophobic CPPs, such as C105Y derived from al-Antitrypsin; PFVYLI derived from synthetic C105Y; Pep-7 peptide (CHL8 peptide phage clone); and modified hydrophobic CPPs, such as stapled peptides and prenylated peptides (Guidotti, G., et al., (2017) Cell-Penetrating Peptides: From Basic Research to Clinics, Trends Pharmacol Sci 38, 406-424; Kristensen, M., et al., (2016) Applications and Challenges for Use of Cell-Penetrating Peptides as Delivery Vectors for Peptide and Protein Cargos, Int J Mol Sci 17). The membrane-permeable peptide or cell-penetrating peptide can further include, for example, TAT-derived cell-penetrating peptides, signal sequence-based (e.g., NLS) cell-penetrating peptides, hydrophobic membrane translocating sequence (MTS) peptides, and arginine-rich molecular transporters. The cell-penetrating lapidated peptide includes, for example, pepducins, such as ICL1/2/3, C-tail-short palmitoylated peptides (Covic, L., et al., (2002) Activation and inhibition of G protein-coupled receptors by cell-penetrating membrane-tethered peptides, Proc Natl Acad Sci USA, 99, 643-648; and O'Callaghan, K., et al., (2012) Turning receptors on and off with intracellular pepducins: new insights into G-protein-coupled receptor drug development. J Biol Chem 287, 12787-12796).

The peptide(s) that target the CXCR4-ADRB2 heteromeric interface may be, for example, a transmembrane domain of CXCR4, transmembrane domain of ADRB2, intracellular loop of CXCR4, intracellular loop of ADRB2, C-terminal domain of CXCR4, or C-terminal domain of ADRB2., extracellular loop of CXCR4, extracellular loop of ADRB2, N-terminal region of CXCR4, or N-terminal region of ADRB2.

As used herein, the terms “express”, “expression”, or “expressing”, and the like, when used in connection with a gene, refer to the process by which the information carried by the gene becomes manifest as the phenotype, including transcription of the gene to a messenger RNA (mRNA), the subsequent translation of the mRNA molecule to a polypeptide chain and its assembly into the ultimate protein. In certain embodiments, a disease, such as cancer, may be characterized in terms of expression of a particular gene, such as in terms of expression of the ultimate protein from the particular gene. For example, a cancer may be characterized as a CXCR4-expressing cancer, an ADRB2-expressing cancer, or a CXCR4-expressing and ADRB2-expressing cancer.

As used herein, the phrase “expression level”, when used in connection with a gene, refers to the amount or accumulation of the expression product of the gene, such as, for example, the amount of a RNA product of the gene (the RNA level of the gene) or the amount of a protein product of the gene (the protein level of the gene). If the gene has more than one allele, the expression level of a gene refers to the total amount of accumulation of the expression product of all existing alleles for this gene, unless otherwise specified. For example, a cancer may be associated with an expression level (amount) of a particular product of the gene, such as a particular RNA product of the gene or a particular protein product of the gene. For example, a cancer may be associated with an expression level (amount) of a particular product of the gene, such as a particular RNA product of the gene or a particular protein product of the gene. For example, a cancer may have a particular expression level of the CXCR4 gene. For example, a cancer may have a particular expression level of the ADRB2 gene. For example, a cancer may have a particular expression level of the CXCR4 gene and a particular expression level of the ADRB2 gene. For example, a cancer may have a particular expression level of CXCR4 protein. For example, a cancer may have a particular expression level of ADRB2 protein. For example, a cancer may have a particular expression level of CXCR4 protein and a particular expression level of ADRB2 protein.

As used herein, the term “reference”, when used in connection with a quantifiable value, refers to a predetermined value that one can use to determine the significance of the value as measured in a sample.

As used herein, the phrase “reference expression level” refers to a predetermined expression level of a gene that one can use to determine the significance of the expression level of the gene in a cell or in a sample. A reference expression level of a gene can be the expression level of the gene in a reference cell determined by a person of ordinary skill in the art. For example, the reference expression level of a CXCR4 gene can be its average expression level in cells, such as T cells or cancer cells. Accordingly, one can determine that the expression level of the CXCR4 gene in a cell (or sample of cells), if higher than the average expression level of the gene in cells, such as T cells or cancer cells, indicates that the cell (or sample of cells) is a CXCR4-expressing cell (or a CXCR4-expressing sample of cells). For example, the reference expression level of an ADRB2 gene can be its average expression level in cells, such as T cells or cancer cells. Accordingly, one can determine that the expression level of the ADRB2 gene in a cell (or sample of cells), if higher than the average expression level of the gene in cells, such as T cells or cancer cells, indicates that the cell is an ADRB2-expressing cell (or an ADRB2-expressing sample of cells). A reference expression level of a gene can also be a cut-off value determined by a person of ordinary skill in the art through statistical analysis of the expression levels of the gene in various sample cell populations. For example, a cancer may have an expression level of a CXCR4 gene in a sample higher than a reference level of the CXCR4 gene. For example, a cancer may have an expression level of CXCR4 protein in a sample higher than a reference level of CXCR4 protein. For example, a cancer may have an expression level of an ADRB2 gene in a sample higher than a reference level of the ADRB2 gene. For example, a cancer may have an expression level of ADRB2 protein in a sample higher than a reference level of ADRB2 protein. For example, a cancer may have an expression level of a CXCR4 gene and an expression level of an ADRB2 gene in a sample higher than a reference level of the CXCR4 gene and a reference level of the ADRB2 gene, respectively. For example, a cancer may have an expression level of CXCR4 protein and an expression level of ADRB2 protein in a sample higher than a reference level of CXCR4 protein and a reference level of ADRB2 protein, respectively. In certain embodiments, for example, by analyzing the expression levels of a gene in sample cell populations having at least 50%, at least 60%, at least 70%, at least 80%, at least 90% cells known to express that gene, a person of ordinary skill in the art can determine a cut-off value as the reference expression level of the gene, which can be used to indicate the percentages of cells expressing the gene in a cell population with unknown constitution.

As used herein, the terms “selecting” and “selected”, in reference to a subject (e.g., a cancer subject, such as a cancer patient), are used to mean that a particular subject is specifically chosen from a larger group of subjects on the basis of (due to) the particular subject having a predetermined criteria or a set of predetermined criteria, e.g., the subject having a CXCLR4 expression level greater than a reference level, the subject having an ADRB2 expression level greater than a reference level, or the subject having a CXCR4 expression level and an ADRB2 expression level greater than respective reference levels. Similarly, “selectively treating a subject” refers to providing treatment to a subject (e.g., a cancer subject, such as a cancer patient) that is specifically chosen from a larger group of subjects on the basis of (due to) the particular subject having a predetermined criteria or a set of predetermined criteria, e.g., the subject having a CXCR4 expression level greater than a reference level, the subject having an ADRB2 expression level greater than a reference level, or the subject having a CXCR4 expression level and an ADRB2 expression level greater than respective reference levels. Similarly, “selectively administering” refers to administering a drug to a subject (e.g., a cancer subject, such as a cancer patient) that is specifically chosen from a larger group of subjects on the basis of (due to) the particular subject having a predetermined criteria or a set of predetermined criteria, e.g., the subject having a CXCR4 expression level greater than a reference level, the subject having an ADRB2 expression level greater than a reference level, or the subject having a CXCR4 expression level and an ADRB2 expression level greater than respective reference levels. By selecting, selectively treating and selectively administering, it is meant that a subject is delivered a personalized therapy for a disease or disorder, e.g., cancer, based on the subject's biology, rather than being delivered a standard treatment regimen based solely on having the disease or disorder (e.g., cancer).

Traditionally GPCRs were believed to function as monomers that interact with hetero-trimeric G proteins upon ligand binding, and drugs were developed based on monomeric or homomeric GPCRs (Milligan, G. (2008) A day in the life of a G protein-coupled receptor: the contribution to function of G protein-coupled receptor dimerization, Br J Pharmacol 153 Suppl 1, S216-229). This view has changed drastically by the discoveries that GPCRs can form heteromers, and heteromerization is obligatory for some GPCRs. GPCR heteromerization is known to alter GPCR maturation and cell surface delivery, ligand binding affinity, signaling intensity and pathways, as well as receptor desensitization and recycling (Terrillon, S., et al., (2004) Roles of G-protein-coupled receptor dimerization, EMBO Rep 5, 30-34; Ferre, S., et al., (2009); Rozenfeld, R., et al., (2010) Receptor heteromerization and drug discovery, Trends Pharmacol Sci 31, 124-130; Gomes, I., et al., (2016); Farran, B. (2017) An update on the physiological and therapeutic relevance of GPCR oligomers, Pharmacol Res 117, 303-327). Different GPCR heteromers display distinct functional and pharmacological properties, and GPCR heteromerization can vary depending on cell types, tissues, and diseases or pathological conditions (Terrillon, S., et al., 2004; Ferre, S., et al., 2009; Rozenfeld, R., et al., 2010; Gomes, I., et al., 2016; Farran, B., 2017). Now GPCR heteromerization is regarded as a general phenomenon, and deciphering GPCR heteromerization opens new avenues for understanding receptor function, physiology, roles in diseases and pathological conditions. Accordingly, identification of GPCR heteromers and their functional properties offers new opportunity for developing new pharmaceuticals or finding new use of old drugs with fewer side effects, higher efficacy, and increased tissue selectivity (Ferre, S., et al., 2009; Rozenfeld, R., et al., 2010; Farran, B., 2017).

The identification of bona fide GPCR heteromer requires intensive and critical evaluation. To distinguish GPCR heteromers from simple association of GPCRs, researchers in this field and the International Union of Basic and Clinical Pharmacology Committee on Receptor Nomenclature and Drug Classification (NC-IUPHAR) have declared GPCR heteromer as “macromolecular complex composed of at least two (functional) receptor units [protomers] with biochemical properties that are demonstrably different from those of its individual components” and these heteromers exist in native tissue (Ferre, S., et al., 2009; Gomes, I., et al., 2016; Pin, J. P., et al., (2007) International Union of Basic and Clinical Pharmacology. LXVII. Recommendations for the recognition and nomenclature of G protein-coupled receptor heteromultimers, Pharmacol Rev 59, 5-13). They proposed three criteria to demonstrate GPCR heteromers: (1) Heteromers should exhibit appropriate co-localization and interaction to enable allosterism using co-immunoprecipitation, in situ hybridization, or proximity-based techniques including proximity ligation assays in cells/tissues that express both receptors and not in cells/tissues that lack one of the receptors; (2) Heteromers should exhibit distinct properties such as changes in signaling, ligand binding, and/or trafficking, only in cells/tissues expressing both receptors but not in cells/tissues that lack one of the receptors; and (3) Heteromer-selective reagents should alter heteromer-specific properties. Heteromer-selective reagents include heteromer-selective antibodies, membrane-permeable peptides, and bivalent/bifunctional ligands (Gomes, I., et al., 2016; Pin, J. P., et al., 2007). Although many GPCR heteromers have been identified in vitro using recombinant receptors expressed in heterologous cells, only a few have demonstrated novel properties, and very few have shown evidence for GPCR heteromerization in native tissue due to technical problems (Gomes, I., et al., 2016). NC-IUPHAR announced that one should provide evidence that satisfies at least two of the above three criteria for approval of new GPCR heteromers (Pin, J. P., et al., 2007).

As disclosed herein, to establish whether criterion 1 of 3 is satisfied (relating to whether heteromer components colocalize and physically interact, either directly or via intermediate proteins acting as conduits for allosterism) in determining the presence/existence of a CXCR4-ADRB2 heteromer, one or more of the following methods may be utilized to including, but not limited to: co-internalization assays; co-localization assays (determining co-localization of the receptor portomers within a cellular compartment) such as in situ hybridization, immunohistochemistry, or immunoelectron microscopy; proximity-based assays, such as proximity-based biophysical techniques, including resonance energy transfer (RET), bioluminescence RET (BRET), fluorescence RET (FRET), time-resolved fluorescence RET (TR-FRET), antibody-aided FRET, ligand-aided FRET, bimolecular fluorescence complementation (BiFC), expression levels of CXCR4 and ADRB2, and proximity ligation assays (PLA); co-immunoprecipitation assays; or fluorescent animals. For example, BiFC, co-internalization assay, expression levels of CXCR4 and ADRB2, or PLA, were utilized to evaluate whether a CXCR4-ADRB2 heteromer satisfied criterion 1 of 3.

As disclosed herein, to establish whether criterion 2 of 3 is satisfied (relating to whether a CXCR4-ADRB2 heteromer exhibits properties distinct from those of the individual protomers), such as an enhanced downstream signaling, for example an enhanced calcium mobilization (such as determined by a calcium mobilization assay), a two-tiered approach was utilized on those CXCR4-ADRB2 heteromers that satisfied criterion 1 of 3 discussed above: (1) determine the presence/absence of an enhanced downstream signaling, for example an enhanced calcium mobilization (synergism) in the individual protomer context—comparing calcium mobilization in cells co-expressing HA-VC and one of the protomers (either CXCR4 or ADRB2) upon (a) co-stimulation with CXCL12 and the ADRB2 agonist, relative to (b) stimulation with either CXCL12 alone or the ADRB2 agonist alone; and (2) determine the presence/absence of an enhanced downstream signaling, for example an enhanced calcium mobilization (synergism) in the CXCR4-ADRB2 heteromer context—comparing calcium mobilization in cells co-expressing CXCR4 and ADRB2 upon (a) co-stimulation with CXCL12 and the ADRB2 agonist, relative to (b) the sum of stimulation with either CXCL12 alone or the ADRB2 agonist alone. As disclosed herein, to satisfy criterion 2 of 3 and be considered a CXCR4-ADRB2 heteromer that results in an enhanced downstream signaling, for example an enhanced calcium mobilization, there must be (1) an absence of an enhanced downstream signaling, for example an enhanced calcium mobilization in either protomer context (i.e., the CXCR4 and HA-VC context or the ADRB2 and HA-VC context), and (2) a presence of an enhanced downstream signaling, for example an enhanced calcium mobilization in the CXCR4-ADRB2 heteromer context. In both the protomer context and the CXCR4-ADRB2 heteromer context, the concentration of CXCL12 utilized to stimulate the cells (either as a single agent or in combination with the ADRB2 agonist) and the concentration of the ADRB2 agonist (either an endogenous agonist or a known selective agonist for the ADRB2) utilized to stimulate the cells (either as a single agent or in combination with CXCL12) are independently at a concentration of 100× the EC50 concentration or lower. For example, the concentration of CXCL12 utilized to stimulate the cells (either as a single agent or in combination with the ADRB2 agonist) was at a concentration of 15 nM (which is approximately the EC50 concentration against CXCR4).

As disclosed herein, to establish whether criterion 3 of 3 is satisfied (relating to whether heteromer-selective reagents should alter heteromer-specific properties) in determining the presence/existence of a CXCR4-ADRB2 heteromer, subject derived cells, having satisfied criterion 1 of 3 and 2 of 3, are effected in the presence of an antagonist (a CXCR4 antagonist, a ADRB2 antagonist, or a CXCR4-ADRB2 heteromer antagonist), such as effecting cell proliferation of the subject derived cells containing a CXCR4-ADRB2 heteromer.

In some embodiments, the method for treating, method for suppressing, pharmaceutical composition, or pharmaceutical kit, as disclosed herein, include or rely upon establishing that an association of CXCR4 and ADRB2 in a cell satisfies at least two of the following criteria or characteristics to be considered a CXCR4-ADRB2 heteromer, comprising: 1) the CXCR4-ADRB2 heteromer components in a cell colocalize and physically interact, either directly or via intermediate proteins acting as conduits for allosterism, as determined via one or more of the following: a co-internalization assay, a colocalization assay, in situ hybridization, immunohistochemistry, immunoelectron microscopy, expression levels of CXCR4 and ADRB2, a proximity-based assay, a co-immunoprecipitation assay, or a fluorescent animal assay; 2) an enhanced amount of calcium mobilization, such that: a) either CXCR4 or ADRB2 in an individual protomer context in a cell, upon co-stimulation with CXCL12 and an ADRB2 agonist, results in a calcium mobilization amount that is equal to or less than the sum of calcium mobilization amounts resulting from single agonist stimulation with either the CXCL12 or the ADRB2 agonist; and b) the CXCR4-ADRB2 heteromer exhibits an enhanced calcium mobilization upon co-stimulation with the CXCL12 and the ADRB2 agonist relative to the sum of calcium mobilization amounts resulting from single agonist stimulation with either the CXCL12 or the ADRB2 agonist; as determined via a calcium mobilization assay; or 3) a CXCR4-ADRB2 heteromer-selective reagent: i) alters heteromer-specific properties of the CXCR4-ADRB2 heteromer in a subject derived cell; ii) alters heteromer-specific function of the CXCR4-ADRB2 heteromer in a subject derived cell; iii) alters heteromer-specific properties of a subject derived cell containing the CXCR4-ADRB2 heteromer; or iv) decreases cancer progression in the subject having cells containing said CXCR4-ADRB2 heteromer (such as a decrease in cancer progression of the subject's derived cell(s) containing said CXCR4-ADRB2 heteromer, a decrease in cell proliferation of the subject's derived cell(s) containing said CXCR4-ADRB2 heteromer, a decrease in cell migration of the subject's derived cell(s) containing said CXCR4-ADRB2 heteromer, a decrease in metastasis of the subject's derived cell(s) containing said CXCR4-ADRB2 heteromer, and/or a decrease in angiogenesis of the subject's derived cell(s) containing said CXCR4-ADRB2 heteromer). In some embodiments, the CXCR4-ADRB2 heteromer-selective reagent alters heteromer-specific properties of the CXCR4-ADRB2 heteromer in a subject derived cell, as determined by at least one of the following methods: PLA, radioligand binding assays, [35S]GTP-γS Binding assays, Calcuim assay, cAMP assay, GTPase assay, PKA activation, ERK1/2 and/or Akt/PKB Phosphorylation assays, Src and STAT3 phosphorylation assays, CRE-reporter assay, NFAT-RE-reporter assay, SRE-reporter assay, SRF-RE reporter assay, Secreted alkaline phosphatase Assay, Inositol 1-Phosphate Production assay, Adenylyl Cyclase Activity assay, analysis of target gene expression by RT-PCR, RT-qPCR, RNAseq, next generation sequencing (NGS), or microarray. In some embodiments, the CXCR4-ADRB2 heteromer-selective reagent alters heteromer-specific function of the CXCR4-ADRB2 heteromer in a subject derived cell, as determined by at least one of the following methods: PLA, radioligand binding assays, [35S]GTP-γS Binding assays, Calcuim assay, cAMP assay, GTPase assay, PKA activation, ERK1/2 and/or Akt/PKB Phosphorylation assays, Src and STAT3 phosphorylation assays, CRE-reporter assay, NFAT-RE-reporter assay, SRE-reporter assay, SRF-RE reporter assay, NF-kB-RE reporter assay, Secreted alkaline phosphatase Assay, Inositol 1-Phosphate Production assay, Adenylyl Cyclase Activity assay, analysis of target gene expression by RT-PCR, RT-qPCR, RNAseq, or microarray. In some embodiments, the CXCR4-ADRB2 heteromer-selective reagent alters heteromer-specific properties of a subject derived cell containing the CXCR4-ADRB2 heteromer, as determined by at least one of the following methods: assays on proliferation, migration, invasion, and drug resistance (survival) of cancer cells, modulation of immune cell function, angiogenesis, vasculogenesis, metastasis, drug resistance, tissue microarray (TMA), and cancer cell-tumor microenvironment (TME) interaction. For example, in some embodiments, the method for treating, method for suppressing, pharmaceutical composition, or pharmaceutical kit, as disclosed herein, include or rely upon establishing that an association of CXCR4 and ADRB2 in a cell satisfies at least two of the following criteria or characteristics to be considered a CXCR4-ADRB2 heteromer, comprising: 1) the CXCR4-ADRB2 heteromer components in a cell colocalize and physically interact, either directly or via intermediate proteins acting as conduits for allosterism, as determined via one or more of the following: a co-internalization assay, bimolecular fluorescence complementation (BiFC), expression levels of CXCR4 and ADRB2, or a proximity ligation assay (PLA); 2) an enhanced amount of calcium mobilization, such that: a) either CXCR4 or ADRB2 in an individual protomer context in a cell, upon co-stimulation with CXCL12 and an ADRB2 agonist, results in a calcium mobilization amount that is equal to or less than the sum of calcium mobilization amounts resulting from single agonist stimulation with either the CXCL12 or the ADRB2 agonist; and b) the CXCR4-ADRB2 heteromer exhibits an enhanced calcium mobilization upon co-stimulation with the CXCL12 and the ADRB2 agonist relative to the sum of calcium mobilization amounts resulting from single agonist stimulation with either the CXCL12 or the ADRB2 agonist; as determined via a calcium mobilization assay; or 3) a CXCR4-ADRB2 heteromer-selective reagent: i) alters heteromer-specific properties of the CXCR4-ADRB2 heteromer in a subject derived cell; ii) alters heteromer-specific function of the CXCR4-ADRB2 heteromer in a subject derived cell; iii) alters heteromer-specific properties of a subject derived cell containing the CXCR4-ADRB2 heteromer; or iv) decreases cancer progression in the subject having cells containing said CXCR4-ADRB2 heteromer (such as a decrease in cancer progression of the subject's derived cell(s) containing said CXCR4-ADRB2 heteromer, a decrease in cell proliferation of the subject's derived cell(s) containing said CXCR4-ADRB2 heteromer, a decrease in cell migration of the subject's derived cell(s) containing said CXCR4-ADRB2 heteromer, a decrease in metastasis of the subject's derived cell(s) containing said CXCR4-ADRB2 heteromer, and/or a decrease in angiogenesis of the subject's derived cell(s) containing said CXCR4-ADRB2 heteromer). In some embodiments, the method for treating, method for suppressing, pharmaceutical composition, or pharmaceutical kit, as disclosed herein, include or rely upon establishing that an association of CXCR4 and ADRB2 in a cell satisfies criteria 1 and 2 to be considered a CXCR4-ADRB2 heteromer. In some embodiments, the method for treating, method for suppressing, pharmaceutical composition, or pharmaceutical kit, as disclosed herein, include or rely upon establishing that an association of CXCR4 and ADRB2 in a cell satisfies criteria 1 and 3 to be considered a CXCR4-ADRB2 heteromer. In some embodiments, the method for treating, method for suppressing, pharmaceutical composition, or pharmaceutical kit, as disclosed herein, include or rely upon establishing that an association of CXCR4 and ADRB2 in a cell satisfies criteria 2 and 3 to be considered a CXCR4-ADRB2 heteromer. In some embodiments, the method for treating, method for suppressing, pharmaceutical composition, or pharmaceutical kit, as disclosed herein, include or rely upon establishing that an association of CXCR4 and ADRB2 in a cell satisfies criteria 1, 2, and 3, to be considered a CXCR4-ADRB2 heteromer.

As disclosed herein, the CXCR4-ADRB2 heteromer satisfying at least two of the three criteria may have, cause, or produce an enhanced downstream signaling. The enhanced downstream signaling may be the result from the CXCR4-ADRB2 heteromer, such as from agonism of the CXCR4-ADRB2 heteromer, from CXCR4 agonism of the CXCR4-ADRB2 heteromer, from ADRB2 agonism of the CXCR4-ADRB2 heteromer, and/or from CXCR4 agonism and ADRB2 agonism of the CXCR4-ADRB2 heteromer. In some embodiments, the enhanced downstream signaling may be downstream of the CXCR4, the ADRB2, or the CXCR4-ADRB2 heteromer. In some embodiments, the enhanced downstream signaling may be from the CXCR4-ADRB2 heteromer, relative to downstream signaling from a CXCR4 protomer or an ADRB2 protomer in their respective individual protomer context. In some embodiments, the enhanced downstream signaling may be from the CXCR4-ADRB2 heteromer, relative to downstream signaling from a CXCR4 protomer in an individual protomer context. In some embodiments, the enhanced downstream signaling may be from the CXCR4-ADRB2 heteromer, relative to downstream signaling from an ADRB2 protomer in an individual protomer context. In some embodiments, the enhanced downstream signaling may be from the CXCR4-ADRB2 heteromer, relative to downstream signaling from a CXCR4 protomer and an ADRB2 protomer in their respective individual protomer context. The enhanced downstream signaling from said CXCR4-ADRB2 heteromer, may in some embodiments, be suppressed in the cancer subject, such as suppressed in the subject's cancer cells. In some embodiments, the enhanced downstream signaling from said CXCR4-ADRB2 heteromer, may be an enhanced amount of calcium mobilization (or synergistic amount of calcium mobilization), which may be determined by an intracellular Ca2+ assay, such as a calcium mobilization assay.

As disclosed herein, the CXCR4-ADRB2 heteromer satisfying at least two of the three criteria may have, cause, or produce an enhanced downstream signaling, wherein the enhanced downstream signaling is an enhanced amount of calcium mobilization. The enhanced amount of calcium mobilization from the CXCR4-ADRB2 heteromer may be a calcium mobilization amount that, upon co-stimulation with the CXCL12 and the ADRB2 agonist, is at least 10% greater, than the sum of calcium mobilization amounts resulting from single agonist stimulation of said cells with either the CXCL12 or the ADRB2 agonist, as determined via a calcium mobilization assay. In some embodiments, the enhanced amount of calcium mobilization from the CXCR4-ADRB2 heteromer may be a synergistic amount of calcium mobilization that, upon co-stimulation with the CXCL12 and the ADRB2 agonist, is at least 10% greater, than the sum of calcium mobilization amounts resulting from single agonist stimulation of said cells with either the CXCL12 or the ADRB2 agonist, as determined via a calcium mobilization assay. For example, the enhanced amount of calcium mobilization (or synergistic amount of calcium mobilization) from the CXCR4-ADRB2 heteromer may be a calcium mobilization amount that, upon co-stimulation with the CXCL12 and the ADRB2 agonist, is at least 20% greater, at least 30% greater, at least 40% greater, at least 50% greater, at least 75% greater, at least 90% greater, at least 100% greater, at least 150% greater, or at least 200% greater, than the sum of calcium mobilization amounts resulting from single agonist stimulation of said cells with either the CXCL12 or the ADRB2 agonist, as determined via a calcium mobilization assay. In some embodiments, the enhanced amount of calcium mobilization (or synergistic amount of calcium mobilization) from the CXCR4-ADRB2 heteromer may be a calcium mobilization amount that, upon co-stimulation with the CXCL12 and the ADRB2 agonist, may be between 10-100% greater than the sum of calcium mobilization amounts resulting from single agonist stimulation of said cells with either the CXCL12 or the ADRB2 agonist, as determined via a calcium mobilization assay, for example, may be a calcium mobilization amount that, upon co-stimulation with the CXCL12 and the ADRB2 agonist, may be between 25-100% greater, 50-100% greater, 75-100% greater, or 100-200% greater, than the sum of calcium mobilization amounts resulting from single agonist stimulation of said cells with either the CXCL12 or the ADRB2 agonist, as determined via a calcium mobilization assay.

In some embodiments, according to the method for treating, method for suppressing, pharmaceutical composition, or pharmaceutical kit, as disclosed herein, the CXCR4-ADRB2 heteromer satisfies at least two of the three criteria, thereby having, causing, or producing, an enhanced downstream signaling, wherein the enhanced downstream signaling is an enhanced amount of calcium mobilization, such that: a) either the CXCR4 or the ADRB2 in an individual protomer context in the cell upon co-stimulation with CXCL12 and an ADRB2 agonist results in a calcium mobilization amount that is equal to or less than the sum of calcium mobilization amounts resulting from single agonist stimulation with either the CXCL12 or the ADRB2 agonist; and b) the CXCR4-ADRB2 heteromer exhibits an enhanced calcium mobilization upon co-stimulation with the CXCL12 and the ADRB2 agonist relative to the sum of calcium mobilization amounts resulting from single agonist stimulation with either the CXCL12 or the ADRB2 agonist; as determined via a calcium mobilization assay. For example, in some embodiments, the enhanced downstream signaling from the CXCR4-ADRB2 heteromer is an enhanced amount of calcium mobilization, such that: i) the calcium mobilization from the protomer CXCR4 or ADRB2, in the individual protomer context in the cell, is non-synergistic, as determined via calcium mobilization assay; and ii) the calcium mobilization from the CXCR4-ADRB2 heteromer in the cell is synergistic, as determined via a calcium mobilization assay. In some embodiments, the individual protomer context may be: a) the individual protomer CXCR4 in the cell, in the absence of the individual protomer ADRB2; or b) the individual protomer ADRB2 in the cell, in the absence of the individual protomer CXCR4; upon co-stimulation with CXCL12 and an ADRB2 agonist results in a calcium mobilization amount that is equal to or less than the sum of calcium mobilization amounts resulting from single agonist stimulation with either the CXCL12 or the ADRB2 agonist, as determined via a calcium mobilization assay. In some embodiments, the individual protomer context may be, independently: a) the individual protomer CXCR4 in the cell, in the absence of the individual protomer ADRB2; and b) the individual protomer ADRB2 in the cell, in the absence of the individual protomer CXCR4; upon co-stimulation with CXCL12 and an ADRB2 agonist results in a calcium mobilization amount that is equal to or less than the sum of calcium mobilization amounts resulting from single agonist stimulation with either the CXCL12 or the ADRB2 agonist, as determined via a calcium mobilization assay. For example, in some embodiments, the CXCR4-ADRB2 heteromer, upon co-stimulation with the CXCL12 and the ADRB2 agonist, may result in a calcium mobilization amount that is greater than the sum of calcium mobilization amounts resulting from single agonist stimulation of said cells with either the CXCL12 or the ADRB2 agonist, as determined via a calcium mobilization assay.

Provided herein are methods for suppressing enhanced downstream signaling resulting from a CXCR4-ADRB2 heteromer in a cell of a subject suffering from cancer, the method comprising administering to the subject: (a) a CXCR4 inhibitor, wherein the CXCR4 inhibitor is burixafor; and (b) an ADRB2 inhibitor; wherein: (i) the enhanced downstream signaling results from the CXCR4-ADRB2 heteromer; and (ii) the administered combination of inhibitors suppresses the enhanced downstream signaling from said CXCR4-ADRB2 heteromer in the cancer subject. In specific embodiments, the CXCR4 inhibitor is burixafor and the ADRB2 inhibitor is carvedilol.

Provided herein are methods for treating cancer in a subject having a cell containing a CXCR4-ADRB2 heteromer, the method comprising administering to the subject: (a) a CXCR4 inhibitor, wherein the CXCR4 inhibitor is burixafor; and (b) an ADRB2 inhibitor; wherein: (i) enhanced downstream signaling results from the CXCR4-ADRB2 heteromer; and (ii) the administered combination of inhibitors suppresses the enhanced downstream signaling from said CXCR4-ADRB2 heteromer in the cancer subject. In specific embodiments, the CXCR4 inhibitor is burixafor and the ADRB2 inhibitor is carvedilol.

Provided herein are methods for treating cancer in a subject having a cell containing a CXCR4-ADRB2 heteromer, the method comprising: (a) determining whether the subject's cell contains a CXCR4-ADRB2 heteromer, wherein enhanced downstream signaling results from the CXCR4-ADRB2 heteromer; and (b) if the subject's cell contains said CXCR4-ADRB2 heteromer, then administering to the cancer subject: (i) a CXCR4 inhibitor, wherein the CXCR4 inhibitor is burixafor; and (ii) an ADRB2 inhibitor. In specific embodiments, the CXCR4 inhibitor is burixafor and the ADRB2 inhibitor is carvedilol.

Provided herein are methods for treating cancer in a subject having a cell containing a CXCR4-ADRB2 heteromer, wherein enhanced downstream signaling results from the CXCR4-ADRB2 heteromer, the method comprising: (1) determining whether the subject has the cell containing the CXCR4-ADRB2 heteromer by: obtaining or having obtained a biological sample from the subject; and performing or having performed an assay on the biological sample to determine if: (i) the subject's cell contains said CXCR4-ADRB2 heteromer; or (ii) a combination of a CXCR4 inhibitor and an ADRB2 inhibitor: alters heteromer-specific properties or function of said CXCR4-ADRB2 heteromer in the subject's derived cell(s); alters heteromer-specific properties of the subject's derived cell(s) containing said CXCR4-ADRB2 heteromer; or decreases cancer progression in the subject having cells containing said CXCR4-ADRB2 heteromer; and (2) if the subject has a cell containing said CXCR4-ADRB2 heteromer, then administering to the cancer subject a combination of a CXCR4 inhibitor and an ADRB2 inhibitor, wherein the CXCR4 inhibitor is burixafor; and (3) if the subject does not have a cell containing said CXCR4-ADRB2 heteromer, then administering to the cancer subject a single inhibitor of either the burixafor or the ADRB2 inhibitor. In specific embodiments, the CXCR4 inhibitor is burixafor and the ADRB2 inhibitor is carvedilol.

Provided herein are methods for treating cancer in a subject having a cell containing a CXCR4-ADRB2 heteromer, wherein enhanced downstream signaling results from the CXCR4-ADRB2 heteromer, the method comprising: (1) determining whether the subject has the cell containing the CXCR4-ADRB2 heteromer by: obtaining or having obtained a biological sample from the subject; and performing or having performed an assay on the biological sample to determine if: (i) the subject's cell contains said CXCR4-ADRB2 heteromer; or (ii) a combination of a CXCR4 inhibitor and an ADRB2 inhibitor: alters heteromer-specific properties or function of said CXCR4-ADRB2 heteromer in the subject's derived cell(s); alters heteromer-specific properties of the subject's derived cell(s) containing said CXCR4-ADRB2 heteromer; or decreases cancer progression in the subject having cells containing said CXCR4-ADRB2 heteromer; and (2) if the subject has a cell containing said CXCR4-ADRB2 heteromer, then administering to the cancer subject a combination of a CXCR4 inhibitor and an ADRB2 inhibitor, wherein the CXCR4 inhibitor is burixafor; and (3) if the subject does not have a cell containing said CXCR4-ADRB2 heteromer, then administering to the cancer subject a single inhibitor of either the burixafor or the ADRB2 inhibitor; wherein: (a) progression of the cancer in the subject having said cell containing the CXCR4-ADRB2 heteromer is decreased in the range of 5-100% more upon administering to the cancer subject the combination of the burixafor and the ADRB2 inhibitor, relative to administering the single inhibitor of either the burixafor or the ADRB2 inhibitor; (b) efficacy of the burixafor is increased in the range of 5-2000% when administered in combination with the ADRB2 inhibitor to the subject having said cell containing the CXCR4-ADRB2 heteromer, relative to efficacy of the burixafor when administered as a single inhibitor; and/or (c) efficacy of the ADRB2 inhibitor is increased in the range of 5-2000% when administered in combination with the burixafor to the subject having said cell containing the CXCR4-ADRB2 heteromer, relative to efficacy of the ADRB2 inhibitor when administered as a single inhibitor. In specific embodiments, the CXCR4 inhibitor is burixafor and the ADRB2 inhibitor is carvedilol.

Provided herein are methods for treating cancer in a subject having a cell containing a CXCR4-ADRB2 heteromer, wherein enhanced downstream signaling results from the CXCR4-ADRB2 heteromer, the method comprising: (1) determining whether the subject's cell contains the CXCR4-ADRB2 heteromer by: obtaining or having obtained a biological sample from the subject and performing or having performed an assay on the biological sample to determine if said CXCR4-ADRB2 heteromer is present in the subject's cell; wherein the assay performed on the biological sample is or comprises one or more of the following: a co-internalization assay, a colocalization assay, in situ hybridization, immunohistochemistry, immunoelectron microscopy, a proximity-based assay, a co-immunoprecipitation assay, enzyme-linked immunosorbent assay (ELISA), flow cytometry, RNAseq, RT-qPCR, microarray, or a fluorescent animal assay; and (2) if the subject's cell contains said CXCR4-ADRB2 heteromer, then administering to the cancer subject a combination of a CXCR4 inhibitor and an ADRB2 inhibitor, wherein the CXCR4 inhibitor is burixafor; and (3) if the subject's cell does not contain said CXCR4-ADRB2 heteromer, then administering to the cancer subject a single inhibitor of either the burixafor or the ADRB2 inhibitor. In specific embodiments, the CXCR4 inhibitor is burixafor and the ADRB2 inhibitor is carvedilol.

Provided herein are methods for treating cancer in a subject having a cell containing a CXCR4-ADRB2 heteromer, wherein enhanced downstream signaling results from the CXCR4-ADRB2 heteromer, the method comprising: (1) determining whether the subject's cell contains the CXCR4-ADRB2 heteromer by: obtaining or having obtained a biological sample from the subject and performing or having performed an assay on the biological sample to determine if said CXCR4-ADRB2 heteromer is present in the subject's cell; wherein the assay performed on the biological sample is or comprises one or more of the following: a co-internalization assay, a colocalization assay, in situ hybridization, immunohistochemistry, immunoelectron microscopy, a proximity-based assay, a co-immunoprecipitation assay, enzyme-linked immunosorbent assay (ELISA), flow cytometry, RNAseq, RT-qPCR, microarray, or a fluorescent animal assay; and (2) if the subject's cell contains said CXCR4-ADRB2 heteromer, then administering to the cancer subject a combination of a CXCR4 inhibitor and an ADRB2 inhibitor, wherein the CXCR4 inhibitor is burixafor; and (3) if the subject's cell does not contain said CXCR4-ADRB2 heteromer, then administering to the cancer subject a single inhibitor of either the burixafor or the ADRB2 inhibitor; wherein: (a) progression of the cancer in the subject having said cell containing the CXCR4-ADRB2 heteromer is decreased in the range of 5-100% more upon administering to the cancer subject the combination of the burixafor and the ADRB2 inhibitor, relative to administering the single inhibitor of either the burixafor or the ADRB2 inhibitor; (b) efficacy of the burixafor is increased in the range of 5-2000% when administered in combination with the ADRB2 inhibitor to the subject having said cell containing the CXCR4-ADRB2 heteromer, relative to efficacy of the burixafor when administered as a single inhibitor; and/or (c) efficacy of the ADRB2 inhibitor is increased in the range of 5-2000% when administered in combination with the burixafor to the subject having said cell containing the CXCR4-ADRB2 heteromer, relative to efficacy of the ADRB2 inhibitor when administered as a single inhibitor. In specific embodiments, the CXCR4 inhibitor is burixafor and the ADRB2 inhibitor is carvedilol.

Provided herein are pharmaceutical kits for use in treating cancer in a subject having a cell containing a CXCR4-ADRB2 heteromer, the pharmaceutical kit comprising: (a) a CXCR4 inhibitor, wherein the CXCR4 inhibitor is burixafor; and (b) an ADRB2 inhibitor; wherein enhanced downstream signaling results from the CXCR4-ADRB2 heteromer. In specific embodiments, the CXCR4 inhibitor is burixafor and the ADRB2 inhibitor is carvedilol.

Provided herein are pharmaceutical compositions for use in treating cancer in a subject having a cell containing a CXCR4-ADRB2 heteromer, the pharmaceutical composition comprising: (a) a CXCR4 inhibitor, wherein the CXCR4 inhibitor is burixafor; (b) an ADRB2 inhibitor; and (c) a pharmaceutically acceptable carrier; wherein enhanced downstream signaling results from the CXCR4-ADRB2 heteromer. In specific embodiments, the CXCR4 inhibitor is burixafor and the ADRB2 inhibitor is carvedilol.

Provided herein are pharmaceutical compositions comprising: (a) a CXCR4 inhibitor, wherein the CXCR4 inhibitor is burixafor; (b) an ADRB2 inhibitor; and (c) a pharmaceutically acceptable carrier. In specific embodiments, the CXCR4 inhibitor is burixafor and the ADRB2 inhibitor is carvedilol.

Provided herein are methods for suppressing enhanced downstream signaling resulting from a CXCR4-ADRB2 heteromer in a cell, the method comprising administering to the cell: (a) a CXCR4 inhibitor, wherein the CXCR4 inhibitor is burixafor; and (b) an ADRB2 inhibitor; wherein: (i) the enhanced downstream signaling results from the CXCR4-ADRB2 heteromer; and (ii) the contacting with the burixafor and the ADRB2 inhibitor suppresses the enhanced downstream signaling from said CXCR4-ADRB2 heteromer in the cell. In certain embodiments, the method further includes determining whether the cell contains the CXCR4-ADRB2 heteromer. In specific embodiments, the cell is a subject's cell. In specific embodiments, the subject's cell is a cancer cell. In specific embodiments, the cell is a cancer cell. In specific embodiments, the CXCR4 inhibitor is burixafor and the ADRB2 inhibitor is carvedilol.

Provided herein are pharmaceutical kits for use in suppressing enhanced downstream signaling resulting from a CXCR4-ADRB2 heteromer in a cell, the pharmaceutical kit comprising: (a) a CXCR4 inhibitor, wherein the CXCR4 inhibitor is burixafor; and (b) an ADRB2 inhibitor; wherein enhanced downstream signaling results from the CXCR4-ADRB2 heteromer. In specific embodiments, the cell is a subject's cell. In specific embodiments, the subject's cell is a cancer cell. In specific embodiments, the cell is a cancer cell. In specific embodiments, the CXCR4 inhibitor is burixafor and the ADRB2 inhibitor is carvedilol.

Provided herein are pharmaceutical compositions for use suppressing enhanced downstream signaling resulting from a CXCR4-ADRB2 heteromer in a cell, the pharmaceutical composition comprising: (a) a CXCR4 inhibitor, wherein the CXCR4 inhibitor is burixafor; (b) an ADRB2 inhibitor; and (c) a pharmaceutically acceptable carrier; wherein enhanced downstream signaling results from the CXCR4-ADRB2 heteromer. In specific embodiments, the cell is a subject's cell. In specific embodiments, the subject's cell is a cancer cell. In specific embodiments, the cell is a cancer cell. In specific embodiments, the CXCR4 inhibitor is burixafor and the ADRB2 inhibitor is carvedilol.

In some embodiments, the method for suppressing, the method for treating, the pharmaceutical kit for use, or the pharmaceutical composition for use, provided herein further include detecting the presence of the CXCR4-ADRB2 heteromer in the cancer subject. In some embodiments, the methods and uses provided herein further include identifying the CXCR4-ADRB2 heteromer in the cancer subject. In some embodiments, the methods and uses provided herein further include obtaining a biological sample from the subject. In some embodiments, the methods and uses provided herein further include performing an assay on the biological sample obtained from said subject. In some embodiments, the methods and uses provided herein further include: (i) obtaining or having obtained a biological sample from the cancer subject; (ii) conducting or having conducted a diagnostic assay to determine presence, identity, or presence and identity, of a CXCR4-ADRB2 heteromer in the obtained biological sample from the cancer subject; and (iii) selecting the ADRB2 inhibitor to administer in combination with burixafor to suppress the enhanced downstream signaling resulting from the CXCR4-ADRB2 heteromer. In some embodiments, the methods and uses provided herein further include determining whether the subject's cell contains the CXCR4-ADRB2 heteromer, comprising performing an assay on a biological sample obtained from the subject. In some embodiments, the methods and uses provided herein further include determining whether the subject's cell contains the CXCR4-ADRB2 heteromer, comprising obtaining a biological sample from the subject, and performing an assay on the biological sample obtained from said subject. In some embodiments, the methods and uses provided herein include wherein the biological sample obtained from said subject contains the CXCR4-ADRB2 heteromer. In some embodiments, the subject's biological sample is a biological fluid sample. In some embodiments, the biological fluid sample is a blood sample, a plasma sample, a saliva sample, a cerebral fluid sample, an eye fluid sample, or a urine sample. In some embodiments, the methods and uses provided herein further include wherein a liquid biopsy is performed on the biological fluid sample. In some embodiments, the subject's biological sample is a biological tissue sample. In some embodiments, the biological tissue sample is an organ tissue sample, a bone tissue sample, or a tumor tissue sample. In some embodiments, the methods and uses provided herein further include wherein a tissue sample assay is performed on the biological tissue sample. In some embodiments, the methods and uses provided herein include wherein the subject's cell contains the CXCR4-ADRB2 heteromer. In some embodiments, the cell is a cancer cell. In some embodiments, the subject's cell is a cancer cell. In some embodiments, the subject is a patient.

In some embodiments, the method for suppressing, the method for treating, the pharmaceutical kit for use, or the pharmaceutical composition for use, provided herein further include wherein the CXCR4-ADRB2 heteromer has two or more of the following characteristics: (1) the CXCR4-ADRB2 heteromer components colocalize and physically interact in a cell, either directly or via intermediate proteins acting as conduits for allosterism; (2) an enhanced downstream signaling results from the CXCR4-ADRB2 heteromer; and/or (3) a combination of burixafor and an ADRB2 inhibitor: (i) alters heteromer-specific properties of the CXCR4-ADRB2 heteromer in the subject's derived cell(s); (ii) alters heteromer-specific function of the CXCR4-ADRB2 heteromer in the subject's derived cell(s); (iii) alters heteromer-specific properties of the subject's derived cell(s) containing the CXCR4-ADRB2 heteromer; and/or (iv) decreases cancer progression in the subject having cells containing said CXCR4-ADRB2 heteromer.

In some embodiments, the method for suppressing, the method for treating, the pharmaceutical kit for use, or the pharmaceutical composition for use, provided herein include wherein the CXCR4-ADRB2 heteromer is characterized as having the following characteristic: the CXCR4-ADRB2 heteromer components in a cell colocalize and physically interact, either directly or via intermediate proteins acting as conduits for allosterism. In specific embodiments, the methods and uses provided herein further include performing an assay to identify or determine the colocalization and physical interaction of the CXCR4 and ADRB2 components of the CXCR4-ADRB2 heteromer in the cell, either directly or via intermediate proteins acting as conduits for allosterism. In specific embodiments, the assay determining the colocalization and physically interaction of the CXCR4-ADRB2 heteromer components in the cell, includes one or more of the following: a co-internalization assay, a colocalization assay, in situ hybridization, immunohistochemistry, immunoelectron microscopy, a proximity-based assay, a co-immunoprecipitation assay, enzyme-linked immunosorbent assay (ELISA), flow cytometry, RNAseq, RT-PCR, RT-qPCR, expression level of CXCR4, expression level of ADRB2, expression level of CXCR4 and ADRB2, microarray, or a fluorescent animal assay. In specific embodiments, the assay is a co-internalization assay. In specific embodiments, the assay is a colocalization assay. In specific embodiments, the assay is an in situ hybridization assay. In specific embodiments, the assay is a co-immunoprecipitation assay. In specific embodiments, the assay is a proximity-based assay. In specific embodiments, the proximity-based assay is, or comprises, resonance energy transfer (RET), bioluminescence RET (BRET), fluorescence RET (FRET), time-resolved fluorescence RET (TR-FRET), antibody-based FRET, ligand-based FRET, bimolecular fluorescence complementation (BiFC), or a proximity ligation assay (PLA). In specific embodiments, the TR-FRET is a ligand-based TR-FRET. In specific embodiments, the TR-FRET is an antibody-based TR-FRET. In specific embodiments, the assay is bimolecular fluorescence complementation (BiFC). In specific embodiments, the assay is a proximity ligation assay (PLA). In specific embodiments, the assay is an enzyme-linked immunosorbent assay (ELISA). In specific embodiments, the assay is a flow cytometry assay. In specific embodiments, the assay determines the expression level of CXCR4, the expression level of ADRB2, and/or the expression level of CXCR4 and ADRB2. In specific embodiments, the assay determines the expression level of CXCR4, the expression level of ADRB2, and/or the expression level of CXCR4 and ADRB2, via RNAseq. In specific embodiments, the assay determines the expression level of CXCR4, the expression level of ADRB2, and/or the expression level of CXCR4 and ADRB2, via RT-PCR. In specific embodiments, the assay determines the expression level of CXCR4, the expression level of ADRB2, and/or the expression level of CXCR4 and ADRB2, via RT-qPCR. In specific embodiments, the assay is a microarray assay. In specific embodiments, the assay is a fluorescent animal assay. In specific embodiments, the assay determines the presence of the CXCR4-ADRB2 heteromer in the biological sample obtained from said subject. In specific embodiments, the assay determines the presence of the CXCR4-ADRB2 heteromer in the subject. In specific embodiments, the assay determines the presence of the CXCR4-ADRB2 heteromer in the subject's cell.

In some embodiments, the method for suppressing, the method for treating, the pharmaceutical kit for use, or the pharmaceutical composition for use, provided herein include wherein the CXCR4-ADRB2 heteromer is characterized as having the following characteristic: enhanced downstream signaling results from the CXCR4-ADRB2 heteromer. In specific embodiments, the CXCR4-ADRB2 heteromer produces the enhanced downstream signaling. In specific embodiments, the enhanced downstream signaling results from the presence of the CXCR4-ADRB2 heteromer in said subject's cell. In specific embodiments, the CXCR4-ADRB2 heteromer produces the enhanced downstream signaling in the subject's cell. In specific embodiments, the enhanced downstream signaling results from agonism of the CXCR4-ADRB2 heteromer. In specific embodiments, the enhanced downstream signaling results from CXCR4 agonism of the CXCR4-ADRB2 heteromer. In specific embodiments, the enhanced downstream signaling results from ADRB2 agonism of the CXCR4-ADRB2 heteromer. In specific embodiments, the enhanced downstream signaling results from CXCR4 agonism and ADRB2 agonism of the CXCR4-ADRB2 heteromer. In specific embodiments, the enhanced downstream signaling is downstream of the CXCR4, the ADRB2, or the CXCR4-ADRB2 heteromer. In specific embodiments, the enhanced downstream signaling is downstream of the CXCR4. In specific embodiments, the enhanced downstream signaling is downstream of the ADRB2. In specific embodiments, the enhanced downstream signaling is downstream of the CXCR4-ADRB2 heteromer. In specific embodiments, the enhanced downstream signaling from the CXCR4-ADRB2 heteromer is relative to downstream signaling from a CXCR4 protomer or an ADRB2 protomer in their respective individual protomer context. In specific embodiments, the enhanced downstream signaling from the CXCR4-ADRB2 heteromer is relative to downstream signaling from a CXCR4 protomer in an individual protomer context. In specific embodiments, the enhanced downstream signaling from the CXCR4-ADRB2 heteromer is relative to downstream signaling from an ADRB2 protomer in an individual protomer context. In specific embodiments, the enhanced downstream signaling from the CXCR4-ADRB2 heteromer is relative to downstream signaling from a CXCR4 protomer and an ADRB2 protomer in their respective individual protomer context. In specific embodiments, the enhanced downstream signaling is an enhanced amount of calcium mobilization. In specific embodiments, the enhanced response is an enhanced cancer progression in the subject having cell(s) containing said CXCR4-ADRB2 heteromer. In specific embodiments, the enhanced cancer progression is an enhanced cell proliferation in the subject having cell(s) containing said CXCR4-ADRB2 heteromer. In specific embodiments, the enhanced cancer progression is an enhanced cell migration in the subject having cell(s) containing said CXCR4-ADRB2 heteromer. In specific embodiments, the enhanced cancer progression is an enhanced metastasis in the subject having cell(s) containing said CXCR4-ADRB2 heteromer. In specific embodiments, the enhanced cancer progression is an enhanced tumor growth in the subject having cell(s) containing said CXCR4-ADRB2 heteromer. In specific embodiments, the enhanced cancer progression is an enhanced angiogenesis in the subject having cell(s) containing said CXCR4-ADRB2 heteromer. In specific embodiments, the cell(s) are cancer cell(s). In specific embodiments, the cell(s) are subject's derived cell(s). In specific embodiments, the cell(s) are from a biological sample obtained from a subject.

In some embodiments, the method for suppressing, the method for treating, the pharmaceutical kit for use, or the pharmaceutical composition for use, provided herein include wherein the CXCR4-ADRB2 heteromer is characterized as having the following characteristic: enhanced downstream signaling results from the CXCR4-ADRB2 heteromer; wherein the enhanced amount of calcium mobilization is determined by an intracellular Ca2+ assay. In specific embodiments, the intracellular Ca2+ assay is a calcium mobilization assay. In specific embodiments, the calcium mobilization assay determines the CXCR4-ADRB2 heteromer produces the enhanced downstream signaling. In specific embodiments, the calcium mobilization assay determines the enhanced downstream signaling results from the presence of the CXCR4-ADRB2 heteromer. In specific embodiments, the CXCR4-ADRB2 heteromer exhibits the enhanced amount of calcium mobilization, such that: (a) either the CXCR4 or the ADRB2 in an individual protomer context in the cell upon co-stimulation with CXCL12 and an ADRB2 agonist results in a calcium mobilization amount that is equal to or less than the sum of calcium mobilization amounts resulting from single agonist stimulation with either the CXCL12 or the ADRB2 agonist; and (b) the CXCR4-ADRB2 heteromer exhibits an enhanced calcium mobilization upon co-stimulation with the CXCL12 and the ADRB2 agonist relative to the sum of calcium mobilization amounts resulting from single agonist stimulation with either the CXCL12 or the ADRB2 agonist; as determined via a calcium mobilization assay. In specific embodiments, the calcium mobilization from the protomer CXCR4 or ADRB2, in the individual protomer context in the cell, is non-synergistic, as determined via calcium mobilization assay; and the calcium mobilization from the CXCR4-ADRB2 heteromer in the cell is synergistic, as determined via a calcium mobilization assay. In specific embodiments, wherein in the individual protomer context: (a) the individual protomer CXCR4 in the cell, in the absence of the individual protomer ADRB2; or (b) the individual protomer ADRB2 in the cell, in the absence of the individual protomer CXCR4; upon co-stimulation with CXCL12 and an ADRB2 agonist results in a calcium mobilization amount that is equal to or less than the sum of calcium mobilization amounts resulting from single agonist stimulation with either the CXCL12 or the ADRB2 agonist, as determined via a calcium mobilization assay. In specific embodiments, wherein in the individual protomer context, independently: (a) the individual protomer CXCR4 in the cell, in the absence of the individual protomer ADRB2; and (b) the individual protomer ADRB2 in the cell, in the absence of the individual protomer CXCR4; upon co-stimulation with CXCL12 and an ADRB2 agonist results in a calcium mobilization amount that is equal to or less than the sum of calcium mobilization amounts resulting from single agonist stimulation with either the CXCL12 or the ADRB2 agonist, as determined via a calcium mobilization assay. In specific embodiments, upon co-stimulation with the CXCL12 and the ADRB2 agonist of the CXCR4-ADRB2 heteromer results in a calcium mobilization amount that is greater than the sum of calcium mobilization amounts resulting from single agonist stimulation of said cells with either the CXCL12 or the ADRB2 agonist, as determined via a calcium mobilization assay. In specific embodiments, the calcium mobilization amount resulting from the co-stimulation of the CXCR4-ADRB2 heteromer is an enhanced amount of calcium mobilization, relative to the sum of calcium mobilizations resulting from single agonist stimulation of said CXCR4-ADRB2 heteromer, as determined via a calcium mobilization assay. In specific embodiments, the enhanced amount of calcium mobilization resulting from the CXCR4-ADRB2 heteromer is a calcium mobilization amount that, upon co-stimulation with CXCL12 and ADRB2 agonist, is greater than the sum of calcium mobilization amounts resulting from single agonist stimulation of said cells with either the CXCL12 or the ADRB2 agonist, as determined via a calcium mobilization assay. In specific embodiments, the enhanced amount of calcium mobilization resulting from the CXCR4-ADRB2 heteromer is a calcium mobilization amount that, upon co-stimulation with the CXCL12 and the ADRB2 agonist, is at least 10% greater, at least 20% greater, at least 30% greater, at least 40% greater, at least 50% greater, at least 75% greater, or at least 90% greater, than the sum of calcium mobilization amounts resulting from single agonist stimulation of said cells with either the CXCL12 or the ADRB2 agonist, as determined via a calcium mobilization assay. In specific embodiments, the enhanced amount of calcium mobilization resulting from the CXCR4-ADRB2 heteromer is a calcium mobilization amount that, upon co-stimulation with the CXCL12 and the ADRB2 agonist, is at least 100% greater than the sum of calcium mobilization amounts resulting from single agonist stimulation of said cells with either the CXCL12 or the ADRB2 agonist, as determined via a calcium mobilization assay. In specific embodiments, the enhanced amount of calcium mobilization is a synergistic amount of calcium mobilization. In specific embodiments, the synergistic amount of calcium mobilization from the cells containing the CXCR4-ADRB2 heteromer is a calcium mobilization amount that, upon co-stimulation with the CXCL12 and the ADRB2 agonist, is at least 10% greater, at least 20% greater, at least 30% greater, at least 40% greater, at least 50% greater, at least 75% greater, or at least 90% greater, than the sum of calcium mobilization amounts resulting from single agonist stimulation of said cells with either the CXCL12 or the ADRB2 agonist, as determined via a calcium mobilization assay.

In some embodiments, the method for suppressing, the method for treating, the pharmaceutical kit for use, or the pharmaceutical composition for use, provided herein include wherein the CXCR4-ADRB2 heteromer is characterized as having the following characteristic: an enhanced response (or enhanced downstream signaling) resulting from stimulation of said CXCR4-ADRB2 heteromer, such as co-stimulation of said CXCR4-ADRB2 heteromer. In certain embodiments, the enhanced response (or enhanced downstream signaling) is an enhanced amount of calcium mobilization. In certain embodiments, the calcium mobilization is determined by an intracellular Ca2+ assay. In specific embodiments, the intracellular Ca2+ assay is a calcium mobilization assay. In certain embodiments, the enhanced response is an enhanced cancer progression in the subject having cell(s) containing said CXCR4-ADRB2 heteromer. In certain embodiments, the enhanced cancer progression is an enhanced cell proliferation in the subject having cell(s) containing said CXCR4-ADRB2 heteromer. In certain embodiments, the enhanced cancer progression is an enhanced cell migration in the subject having cell(s) containing said CXCR4-ADRB2 heteromer. In certain embodiments, the enhanced cancer progression is an enhanced metastasis in the subject having cell(s) containing said CXCR4-ADRB2 heteromer. In certain embodiments, the enhanced cancer progression is an enhanced tumor growth in the subject having cell(s) containing said CXCR4-ADRB2 heteromer. In certain embodiments, the enhanced cancer progression is an enhanced angiogenesis in the subject having cell(s) containing said CXCR4-ADRB2 heteromer. In certain embodiments, the cell(s) are cancer cell(s). In certain embodiments, the cell(s) are derived from a subject. In certain embodiments, the cell(s) are from a biological sample obtained from a subject.

In some embodiments, the method for suppressing, the method for treating, the pharmaceutical kit for use, or the pharmaceutical composition for use, provided herein include wherein the CXCR4-ADRB2 heteromer is characterized as having one or more of the characteristics selected from the group consisting of: (1) a combination of burixafor and an ADRB2 inhibitor alters heteromer-specific properties of the CXCR4-ADRB2 heteromer in the subject's derived cell(s); (2) a combination of burixafor and an ADRB2 inhibitor alters heteromer-specific function of the CXCR4-ADRB2 heteromer in the subject's derived cell(s); (3) a combination of burixafor and an ADRB2 inhibitor alters heteromer-specific properties of the subject's derived cell(s) containing the CXCR4-ADRB2 heteromer; and (4) a combination of burixafor and an ADRB2 inhibitor decreases cancer progression of the subject derived cell(s) containing the CXCR4-ADRB2 heteromer. In specific embodiments, the CXCR4-ADRB2 heteromer is characterized as follows: a combination of burixafor and an ADRB2 inhibitor alters heteromer-specific properties of the CXCR4-ADRB2 heteromer in the subject's derived cell(s). In specific embodiments, the CXCR4-ADRB2 heteromer is characterized as follows: a combination of burixafor and an ADRB2 inhibitor alters heteromer-specific function of the CXCR4-ADRB2 heteromer in the subject's derived cell(s). In specific embodiments, the CXCR4-ADRB2 heteromer is characterized as follows: a combination of burixafor and an ADRB2 inhibitor alters heteromer-specific properties of the subject's derived cell(s) containing the CXCR4-ADRB2 heteromer. In specific embodiments, the CXCR4-ADRB2 heteromer is characterized as follows: a combination of burixafor and an ADRB2 inhibitor decreases cancer progression of the subject derived cell(s) containing the CXCR4-ADRB2 heteromer. In specific embodiments, the decreased cancer progression comprises a decrease in cancer progression of the subject's derived cell(s) containing said CXCR4-ADRB2 heteromer. In specific embodiments, the decreased cancer progression comprises a decrease in cell proliferation of the subject's derived cell(s) containing said CXCR4-ADRB2 heteromer. In specific embodiments, the decreased cancer progression comprises a decrease in cell migration of the subject's derived cell(s) containing said CXCR4-ADRB2 heteromer. In specific embodiments, the decreased cancer progression comprises a decrease in metastasis of the subject's derived cell(s) containing said CXCR4-ADRB2 heteromer. In specific embodiments, the decreased cancer progression comprises a decrease in angiogenesis of the subject's derived cell(s) containing said CXCR4-ADRB2 heteromer. In specific embodiments, the CXCR4 inhibitor is burixafor and the ADRB2 inhibitor is carvedilol.

In some embodiments, the method for suppressing, the method for treating, the pharmaceutical kit for use, or the pharmaceutical composition for use, provided herein include wherein the CXCR4-ADRB2 heteromer is characterized as having the following characteristic: a greater amount of downstream ERK signaling results from co-stimulation of the CXCR4-ADRB2 heteromer with a CXCR4 agonist and an ADRB2 agonist, relative to the amount of downstream ERK signaling resulting from mono-stimulation of said CXCR4-ADRB2 heteromer with either the CXCR4 agonist or the ADRB2 agonist. In some embodiments, the amount of downstream ERK signaling resulting from co-stimulation with the CXCR4 agonist and the ADRB2 agonist is greater than the amount resulting from mono-stimulation with the CXCR4 agonist. In some embodiments, the amount of downstream ERK signaling resulting from co-stimulation with the CXCR4 agonist and the ADRB2 agonist is at least 5% greater than the amount resulting from mono-stimulation with the CXCR4 agonist. In some embodiments, the amount of downstream ERK signaling resulting from co-stimulation with the CXCR4 agonist and the ADRB2 agonist is at least 10%, at least 25%, at least 50%, at least 75%, or at least 90%, greater than the amount resulting from mono-stimulation with the CXCR4 agonist. In some embodiments, the amount of downstream ERK signaling resulting from co-stimulation with the CXCR4 agonist and the ADRB2 agonist in the range of between 5-15%, 10-25%, 20-50%, 40-75%, or 60-100%, greater than the amount resulting from mono-stimulation with the CXCR4 agonist. In some embodiments, the amount of downstream ERK signaling resulting from co-stimulation with the CXCR4 agonist and the ADRB2 agonist is greater than the amount resulting from mono-stimulation with the ADRB2 agonist. In some embodiments, the amount of downstream ERK signaling resulting from co-stimulation with the CXCR4 agonist and the ADRB2 agonist is at least 5% greater than the amount resulting from mono-stimulation with the ADRB2 agonist. In some embodiments, the amount of downstream ERK signaling resulting from co-stimulation with the CXCR4 agonist and the ADRB2 agonist is at least 10%, at least 25%, at least 50%, at least 75%, or at least 90%, greater than the amount resulting from mono-stimulation with the ADRB2 agonist. In some embodiments, the amount of downstream ERK signaling resulting from co-stimulation with the CXCR4 agonist and the ADRB2 agonist in the range of between 5-15%, 10-25%, 20-50%, 40-75%, or 60-100%, greater than the amount resulting from mono-stimulation with the ADRB2 agonist. In specific embodiments, the CXCR4 agonist is CXCL12 and the ADRB2 agonist is salmeterol.

In some embodiments, the method for suppressing, the method for treating, the pharmaceutical kit for use, or the pharmaceutical composition for use, provided herein include wherein the administered combination of the burixafor and the ADRB2 inhibitor: (i) alters heteromer-specific properties of the CXCR4-ADRB2 heteromer in the subject's derived cell(s); (ii) alters heteromer-specific function of the CXCR4-ADRB2 heteromer in the subject's derived cell(s); (iii) alters heteromer-specific properties of the subject's derived cell(s) containing the CXCR4-ADRB2 heteromer; or (iv) decreases cancer progression in the subject having cells containing said CXCR4-ADRB2 heteromer. In specific embodiments, the administered combination of the burixafor and the ADRB2 inhibitor alters heteromer-specific properties of the CXCR4-ADRB2 heteromer in the subject's derived cell(s). In specific embodiments, the administered combination of the burixafor and the ADRB2 inhibitor alters heteromer-specific function of the CXCR4-ADRB2 heteromer in the subject's derived cell(s). In specific embodiments, the administered combination of the burixafor and the ADRB2 inhibitor alters heteromer-specific properties of the subject's derived cell(s) containing the CXCR4-ADRB2 heteromer. In specific embodiments, the administered combination of the burixafor and the ADRB2 inhibitor decreases cancer progression in the subject having cells containing said CXCR4-ADRB2 heteromer. In specific embodiments, the decreased cancer progression comprises a decrease in cancer progression of the subject's derived cell(s) containing said CXCR4-ADRB2 heteromer. In specific embodiments, the decreased cancer progression comprises a decrease in cell proliferation of the subject's derived cell(s) containing said CXCR4-ADRB2 heteromer. In specific embodiments, the decreased cancer progression comprises a decrease in cell migration of the subject's derived cell(s) containing said CXCR4-ADRB2 heteromer. In specific embodiments, the decreased cancer progression comprises a decrease in metastasis of the subject's derived cell(s) containing said CXCR4-ADRB2 heteromer. In specific embodiments, the decreased cancer progression comprises a decrease in angiogenesis of the subject's derived cell(s) containing said CXCR4-ADRB2 heteromer. In specific embodiments, the CXCR4 inhibitor is burixafor and the ADRB2 inhibitor is carvedilol.

In some embodiments, the method for suppressing, the method for treating, the pharmaceutical kit for use, or the pharmaceutical composition for use, provided herein include wherein the administered combination of the burixafor and the ADRB2 inhibitor suppresses the enhanced downstream signaling from said CXCR4-ADRB2 heteromer, for example, from said CXCR4-ADRB2 heteromer in the cancer subject, such as from said CXCR4-ADRB2 heteromer in the cell, or from said CXCR4-ADRB2 heteromer in the cell of the cancer subject. In specific embodiments, the CXCR4-ADRB2 heteromer components comprise individual protomers of CXCR4 and ADRB2. In some embodiments, the cell containing either the CXCR4 or the ADRB2 in an individual protomer context comprises: (i) the individual protomer CXCR4 in the presence or absence of the individual protomer ADRB2; or (ii) the individual protomer ADRB2 in the presence or absence of the individual protomer CXCR4; respectively. In some embodiments, the cell containing the CXCR4 in an individual protomer context comprises the individual protomer CXCR4 in the presence or absence of the individual protomer ADRB2. In specific embodiments, the cell containing the CXCR4 in an individual protomer context comprises said individual protomer CXCR4 in the absence of the individual protomer ADRB2. In specific embodiments, the cell containing the CXCR4 in an individual protomer context comprises said individual protomer CXCR4 in the presence of the individual protomer ADRB2. In some embodiments, the cell containing the ADRB2 in an individual protomer context comprises the individual protomer ADRB2 in the presence or absence of the individual protomer CXCR4. In specific embodiments, the cell containing the ADRB2 in an individual protomer context comprises said individual protomer ADRB2 in the absence of the individual protomer CXCR4. In specific embodiments, the cell containing the ADRB2 in an individual protomer context comprises said individual protomer ADRB2 in the presence of the individual protomer CXCR4. In specific embodiments, the CXCR4 inhibitor is burixafor and the ADRB2 inhibitor is carvedilol.

In some embodiments, the progression of the cancer is decreased synergistically upon administration of the combination of inhibitors, relative to administering the CXCR4 inhibitor or ADRB2 inhibitor as the single inhibitor to said subject. In some embodiments, the decreased in the progression of the cancer upon administration of the combination of inhibitors is greater than the sum of the decreased levels of progression achieved by administering the CXCR4 inhibitor or ADRB2 inhibitor as the single inhibitor to said subject. In some embodiments, according to the method for treating, method for suppressing, pharmaceutical kit, or pharmaceutical composition, provided herein, the progression of the cancer in the subject having said cancer cell containing the CXCR4-ADRB2 heteromer is decreased in the range of 5-100% more upon administration of the combination of inhibitors, relative to administering the CXCR4 inhibitor or ADRB2 inhibitor as the single inhibitor to said subject, such as decreased in the range of 5-100% more, 10-100% more, 20-100% more, 30-100% more, 40-100% more, 50-100% more, 60-100% more, 75-100% more, 5-75% more, 5-50% more, or 5-25% more, upon administration of the combination of inhibitors, relative to administering the CXCR4 inhibitor or ADRB2 inhibitor as the single inhibitor to said subject. In specific embodiments, the CXCR4 inhibitor is burixafor. In specific embodiments, the ADRB2 inhibitor is carvedilol. In specific embodiments, the progression of the cancer is determined by percentage change in tumor size. In specific embodiments, the progression of the cancer is determined by percentage change in tumor size over a period of 1 month, 2 months, 3 months, 6 months, 1 year, or 2 years.

In some embodiments, according to the method for treating, method for suppressing, pharmaceutical kit, or pharmaceutical composition, provided herein, the efficacy of a CXCR4 inhibitor is increased in the range of 5-5000% when administered in combination with the ADRB2 inhibitor to the subject having said cancer cell containing the CXCR4-ADRB2 heteromer, relative to efficacy of the CXCR4 inhibitor when administered as a single inhibitor, such as increased in the range of 5-4500%, 5-4000%, 5-3500%, 5-3000%, 5-2500%, 5-2000%, 5-1750%, 5-1500%, 5-1250%, 5-1000%, 5-900%, 5-800%, 5-700%, 5-500%, 5-400%, 5-250%, 5-200%, 5-100%, 5-75%, 5-50%, 5-40%, 5-30%, 5-25%, 1000-3000%, 2000-4000%, 3000-5000%, 3500-4500%, 4000-5000%, 100-2000%, 200-2000%, 300-2000%, 500-2000%, 750-2000%, 1000-2000%, 1250-2000%, 1500-2000%, 5-1500%, 25-1500%, 50-1500%, 75-1500%, 100-1500%, 200-1500%, 300-1500%, 500-1500%, 750-1500%, 1000-1500%, or 1250-1500%, when administered in combination with the ADRB2 inhibitor to the subject having said cancer cell containing the CXCR4-ADRB2 heteromer, relative to efficacy of the CXCR4 inhibitor when administered as a single inhibitor. In specific embodiments, the CXCR4 inhibitor is burixafor. In specific embodiments, the ADRB2 inhibitor is carvedilol. In specific embodiments, the increase in efficacy of the CXCR4 inhibitor against the CXCR4-ADRB2 heteromer in the subject's cells (such as in suppressing the enhanced downstream signaling from the CXCR4-ADRB2 heteromer) is determined by the change in IC50 value when the CXCR4 inhibitor is administered in combination with the ADRB2 inhibitor relative to the IC50 value when the CXCR4 inhibitor is administered as a single inhibitor. In specific embodiments, the determining of an IC50 value of a CXCR4 inhibitor according to the assays disclosed herein may utilize a concentration of the ADRB2 inhibitor in the range of between 1-10 μM, such as at a concentration of 1, 2, 3, 4, 5, 6, 7, 8, 9, or 10 μM. In specific embodiments, the determining of an IC50 value of a CXCR4 inhibitor according to the assays disclosed herein may utilize the ADRB2 inhibitor at its IC90 concentration against ADRB2, such as a concentration of the ADRB2 inhibitor in the range of between 1-1,000 nM, for example, between 1-500 nM, between 100-750 nM, or between 600-1,000 nM, such as at a concentration of 10, 25, 50, 100, 250, 500, 600, 700, 800, 900, or 1,000 nM.

In some embodiments, according to the method for treating, method for suppressing, pharmaceutical kit, or pharmaceutical composition, provided herein, the efficacy of a ADRB2 inhibitor is increased in the range of 5-5000% when administered in combination with the CXCR4 inhibitor to the subject having said cancer cell containing the CXCR4-ADRB2 heteromer, relative to efficacy of the ADRB2 inhibitor when administered as a single inhibitor, such as increased in the range of 5-4500%, 5-4000%, 5-3500%, 5-3000%, 5-2500%, 5-2000%, 5-1750%, 5-1500%, 5-1250%, 5-1000%, 5-900%, 5-800%, 5-700%, 5-500%, 5-400%, 5-250%, 5-200%, 5-100%, 5-75%, 5-50%, 5-40%, 5-30%, 5-25%, 1000-3000%, 2000-4000%, 3000-5000%, 3500-4500%, 4000-5000%, 100-2000%, 200-2000%, 300-2000%, 500-2000%, 750-2000%, 1000-2000%, 1250-2000%, 1500-2000%, 5-1500%, 25-1500%, 50-1500%, 75-1500%, 100-1500%, 200-1500%, 300-1500%, 500-1500%, 750-1500%, 1000-1500%, or 1250-1500%, when administered in combination with the CXCR4 inhibitor to the subject having said cancer cell containing the CXCR4-ADRB2 heteromer, relative to efficacy of the ADRB2 inhibitor when administered as a single inhibitor. In specific embodiments, the CXCR4 inhibitor is burixafor. In specific embodiments, the ADRB2 inhibitor is carvedilol. In specific embodiments, the increase in efficacy of the ADRB2 inhibitor against the CXCR4-ADRB2 heteromer in the subject's cells (such as in suppressing the enhanced downstream signaling from the CXCR4-ADRB2 heteromer) is determined by the change in IC50 value when the ADRB2 inhibitor is administered in combination with the CXCR4 inhibitor relative to the IC50 value when the ADRB2 inhibitor is administered as a single inhibitor. In specific embodiments, the determining of an IC50 value of a ADRB2 inhibitor according to the assays disclosed herein may utilize a concentration of the CXCR4 inhibitor in the range of between 1-10 μM, such as at a concentration of 1, 2, 3, 4, 5, 6, 7, 8, 9, or 10 μM. In specific embodiments, the determining of an IC50 value of a ADRB2 inhibitor according to the assays disclosed herein may utilize the CXCR4 inhibitor at its IC90 concentration against CXCR4, such as a concentration of the CXCR4 inhibitor in the range of between 1-1,000 nM, for example, between 1-500 nM, between 100-750 nM, or between 600-1,000 nM, such as at a concentration of 10, 25, 50, 100, 250, 500, 600, 700, 800, 900, or 1,000 nM.

In some embodiments, the method for treating, method for suppressing, pharmaceutical kit for use, or pharmaceutical composition for use, provided herein, include administering a CXCR4 inhibitor and an ADRB2 inhibitor, wherein the method or use suppresses enhanced downstream signaling resulting from the CXCR4-ADRB2 heteromer in the cancer subject in the range of between 5-2000 fold, relative to single inhibitor administration, such as suppresses the enhanced downstream signaling from said CXCR4-ADRB2 heteromer in the cancer subject in the range of between 5-1750 fold, 5-1500 fold, 5-1250 fold, 5-1000 fold, 5-900 fold, 5-800 fold, 5-700 fold, 5-500 fold, 5-400 fold, 5-250 fold, 5-200 fold, 5-100 fold, 5-75 fold, 5-50 fold, 5-40 fold, 5-30 fold, 5-25 fold, 100-2000 fold, 200-2000 fold, 300-2000 fold, 500-2000 fold, 750-2000 fold, 1000-2000 fold, 1250-2000 fold, 1500-2000 fold, 5-1500 fold, 25-1500 fold, 50-1500 fold, 75-1500 fold, 100-1500 fold, 200-1500 fold, 300-1500 fold, 500-1500 fold, 750-1500 fold, 1000-1500 fold, or 1250-1500 fold, relative to single inhibitor administration. In specific embodiments, the CXCR4 inhibitor is burixafor. In specific embodiments, the ADRB2 inhibitor is carvedilol.

In some embodiments, the method for treating, method for suppressing, pharmaceutical kit for use, or pharmaceutical composition for use, provided herein, include administering a CXCR4 inhibitor and an ADRB2 inhibitor, wherein the method or use suppresses enhanced downstream signaling resulting from the CXCR4-ADRB2 heteromer in the cancer subject in the range of between 5-2000 fold, relative to suppression of downstream signaling from either a CXCR4 protomer or an ADRB2 protomer in their respective individual protomer context, such as suppresses the enhanced downstream signaling from said CXCR4-ADRB2 heteromer in the cancer subject in the range of between 5-1750 fold, 5-1500 fold, 5-1250 fold, 5-1000 fold, 5-900 fold, 5-800 fold, 5-700 fold, 5-500 fold, 5-400 fold, 5-250 fold, 5-200 fold, 5-100 fold, 5-75 fold, 5-50 fold, 5-40 fold, 5-30 fold, 5-25 fold, 100-2000 fold, 200-2000 fold, 300-2000 fold, 500-2000 fold, 750-2000 fold, 1000-2000 fold, 1250-2000 fold, 1500-2000 fold, 5-1500 fold, 25-1500 fold, 50-1500 fold, 75-1500 fold, 100-1500 fold, 200-1500 fold, 300-1500 fold, 500-1500 fold, 750-1500 fold, 1000-1500 fold, or 1250-1500 fold, relative to suppression of downstream signaling from either a CXCR4 protomer or an ADRB2 protomer in their respective individual protomer context. In specific embodiments, the CXCR4 inhibitor is burixafor. In specific embodiments, the ADRB2 inhibitor is carvedilol.

Initial testing and evaluation of an inhibitor, or combination of inhibitors, regarding whether effective, or therapeutically effective, in suppressing an enhanced downstream signaling from a CXCR4-ADRB2 heteromer, according to the methods disclosed herein, and/or in determining an IC50 value according to the assays disclosed herein, may utilize a concentration of the inhibitor in the range of between 1-10 μM (or each of the inhibitors of the combination at concentrations in the range of between 1-10 μM), such as at a concentration of 1, 2, 3, 4, 5, 6, 7, 8, 9, or 10 μM. If suppression of the signal by the inhibitor is not appreciable enough for a determinable measurement, then a greater concentration of the inhibitor may be used to better evaluate a determinable measurement, such as an IC50 value. If suppression of the signal by the inhibitor is very strong, then a lower concentration of the inhibitor may be used to better evaluate a determinable measurement, such as an IC50 value.

In some embodiments, the methods provided herein are for the treatment, amelioration, or prevention of a disease in a subject in need thereof. In some embodiments, the methods provided here may include administering a therapeutically effective amount of a pharmaceutical composition provided herein to the subject. For example, the pharmaceutical composition provided herein may include a CXCR4 inhibitor, an ADRB2 inhibitor, or a combination of a CXCR4 inhibitor and an ADRB2 inhibitor; and a pharmaceutically acceptable carrier. Diseases that may be treated or prevented using the methods of the invention include, but are not limited to, cancer, tumor, metastasis, and/or angiogenesis. For example, in some embodiments, the methods provided herein are useful for treating cancers or related symptoms wherein the cells of cancer, tumor, and/or microenvironment expresses the CXCR4-ADRB2 heteromer. In some embodiments, the cancer is a hematological cancer or a solid tumor. In some embodiments, the cancer is a relapsed or refractory cancer. In some embodiments, the cancer is a hematological cancer. In some embodiments, the hematological cancer is selected from the group consisting of: a lymphoma, a leukemia, a myeloma, and a multiple myeloma. In some embodiments, the hematological cancer is selected from the group consisting of: multiple myeloma, acute myeloid leukemia, acute monocytic leukemia, diffuse large B-cell lymphoma, B-cell acute lymphoblastic leukemia, Hodgkins lymphoma, acute promyelocytic leukemia, chronic eosinophilic leukemia, and Burkitt lymphoma. In some embodiments, non-limiting examples of cancers or tumors that can be treated, ameliorated, or prevented using the methods of the invention include tumors of the gastrointestinal tract, for example, breast cancer, lung cancer, small cell carcinoma of the lung, hepatocellular carcinoma, brain cancer, kidney cancer, pancreatic cancer or pancreatic adenocarcinoma, ovarian cancer, prostate cancer, melanoma, lymphoma, leukemia, multiple myeloma, renal cell carcinoma, soft tissue sarcoma, gastrointestinal cancer, stomach cancer, colon cancer, colorectal cancer, colorectal adenocarcinoma, bladder adenocarcinoma, esophageal cancer, and adenocarcinoma of the stomach, esophagus, throat, and urogenital tract. In some embodiments, the lymphoma is a B cell lymphoma, a T-cell lymphoma, or a NK cell lymphoma. In some embodiments, the lymphoma is a relapsed or refractory lymphoma. In some embodiments, the lymphoma is selected from the group consisting of: Hodgkin's lymphoma, non-Hodgkin's lymphoma, cutaneous B-cell lymphoma, activated B-cell lymphoma, Diffuse Large B-Cell Lymphoma (DLBCL), Burkitt lymphoma, mantle cell lymphoma (MCL), follicular lymphoma (FL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphoma (PTCL), cutaneous T-Cell lymphoma (CTCL), mantle zone lymphoma, and low grade follicular lymphoma. In some embodiments, the leukemia is: (a) an acute leukemia selected from the group consisting of: acute lymphocytic leukemia (ALL), T-cell acute lymphocytic leukemia (T-ALL), B-cell acute lymphoblastic leukemia, acute monocytic leukemia, acute promyelocytic leukemia, acute myelocytic leukemia (AML), acute myeloid leukemia, acute myelogenous leukemia and myeloblasts, promyeiocytic, myelomonocytic, monocytic, and erythroleukemia; (b) a chronic leukemia selected from the group consisting of: chronic myelocytic (granulocytic) leukemia, chronic myelogenous leukemia (chronic myeloid leukemia; CML), and chronic lymphocytic leukemia (CLL); or (c) chronic myelomonocytic leukemia (CMML), chronic eosinophilic leukemia, juvenile myelomonocytic leukemia (JMML), polycythemia vera, natural killer cell leukemia (NK leukemia), or hairy cell leukemia. In some embodiments, the cancer is a solid tumor. In some embodiments, the solid tumor is a benign tumor or a cancer. In some embodiments, the solid tumor is a carcinoma or a sarcoma. In some embodiments, the solid tumor is selected from the group consisting of: pancreatic adenocarcinoma, pancreatic ductal adenocarcinoma, renal cell carcinoma, breast adenocarcinoma, breast carcinoma, breast ductal adenocarcinoma, ovarian serous adenocarcinoma, ovarian clear cell adenocarcinoma, ovarian mucinous cystadenocarcinoma, uterine carcinosarcoma, endometrium adenocarcinoma, endometrial stromal sarcoma, endometrial carcinoma, gastric tubular adenocarcinoma, gastric adenosquamous carcinoma, multiple endocrine neoplasia, melanoma, thyroid gland carcinoma, prostate carcinoma, hepatocellular carcinoma, intrahepatic cholangiocarcinoma, lung adenocarcinoma, small cell lung carcinoma, adenosquamous lung carcinoma, malignant epithelioid mesothelioma, glioblastoma, medulloblastoma, astrocytoma, alveolar rhabdomyosarcoma, and osteosarcoma. In some embodiments, the solid tumor is selected from the group consisting of: fibrosarcoma, myxosarcoma, liposarcoma, chondrosarcoma, osteosarcoma, synovioma, mesothelioma, malignant epithelioid mesothelioma, Ewing's tumor, leiomyosarcoma, rhabdomyosarcoma, stomach cancer, colorectal cancer, esophageal cancer, colon carcinoma, lymphoid malignancy, pancreatic cancer, pancreatic adenocarcinoma, pancreatic ductal adenocarcinoma, breast cancer, breast adenocarcinoma, breast ductal adenocarcinoma, lung cancer, small cell lung carcinoma, lung adenocarcinoma, adenosquamous lung carcinoma, alveolar rhabdomyosarcoma, ovarian cancer, ovarian clear cell adenocarcinoma, ovarian mucinous cystadenocarcinoma, ovarian serous adenocarcinoma, prostate cancer, hepatocellular carcinoma, soft tissue sarcomas, squamous cell carcinoma, basal cell carcinoma, adenocarcinoma, sweat gland carcinoma, medullary thyroid carcinoma, papillary thyroid carcinoma, thyroid gland carcinoma, pheochromocytomas sebaceous gland carcinoma, papillary carcinoma, papillary adenocarcinomas, medullary carcinoma, bronchogenic carcinoma, gastrointestinal cancer, gastric tubular adenocarcinoma, gastric adenosquamous carcinoma, kidney cancer, intrahepatic cholangiocarcinoma, renal cell carcinoma, hepatoma, bile duct carcinoma, choriocarcinoma, Wilms' tumor, uterine carcinosarcoma, endometrium adenocarcinoma, endometrial stromal sarcoma, endometrial carcinoma, cervical cancer, testicular tumor, seminoma, bladder carcinoma, melanoma, multiple myeloma, multiple endocrine neoplasia, CNS tumor, glioblastoma, astrocytoma, CNS lymphoma, germinoma, meduloblastoma, Schwannoma craniopharyogioma, ependymoma, pineaioma, hemangioblastoma, acoustic neuroma, oligodendroglioma, menangioma, neuroblastoma, retinoblastoma, and brain metastases. In some embodiments, the solid tumor is selected from the group consisting of: breast cancer, lung cancer, and hepatocellular carcinoma. In some embodiments, the solid tumor is breast cancer. In some embodiments, the solid tumor is lung cancer. In some embodiments, the solid tumor is hepatocellular carcinoma. In some embodiments, the method for treating cancer is a method for suppressing enhanced downstream signaling resulting from the CXCR4-ADRB2 heteromer. In some embodiments, the method for suppressing enhanced downstream signaling resulting from the CXCR4-ADRB2 heteromer is a method for treating cancer. In some embodiments, the cancer is a CXCR4-expressing cancer. In some embodiments, the cancer is an ADRB2-expressing cancer. In some embodiments, the cancer is a CXCR4-expressing cancer and an ADRB2-expressing cancer. In some embodiments, the CXCR4 expression level in the subject is greater than a reference level. In some embodiments, the ADRB2 expression level in the subject is greater than a reference level. In some embodiments, the CXCR4 expression level and the ADRB2 expression level in the subject are greater than respective reference levels. In some embodiments, the CXCR4 expression level in the cell is greater than a reference level. In some embodiments, the ADRB2 expression level in the cell is greater than a reference level. In some embodiments, the CXCR4 expression level and the ADRB2 expression level in the cell are greater than respective reference levels.

In some embodiments, the method for suppressing, the method for treating, the pharmaceutical kit for use, or the pharmaceutical composition for use, provided herein further include determining the expression level of the CXCR4 gene in the cell, in the subject, or in the sample obtained from the subject, wherein the cell, the subject, or the sample obtained from the subject is determined to have a CXCR4-expressing cancer if the expression level is greater than a reference level of the CXCR4 gene. In specific embodiments, the methods or uses provided herein determine the expression level of the CXCR4 gene in the cell. In specific embodiments, the methods or uses provided herein determine the expression level of the CXCR4 gene in the subject. In specific embodiments, the methods or uses provided herein determine the expression level of the CXCR4 gene in the sample obtained from the subject. In specific embodiments, the cancer is a CXCR4-expressing cancer. In specific embodiments, the CXCR4 gene expression level in the cell is greater than a reference level. In specific embodiments, the CXCR4 gene expression level in the subject is greater than a reference level. In specific embodiments, the CXCR4 gene expression level in the sample obtained from the subject is greater than a reference level. In specific embodiments, wherein the CXCR4 gene expression level is greater than the reference level, the methods or uses provided herein include administering a CXCR4 inhibitor and an ADRB2 inhibitor. In specific embodiments, the CXCR4 inhibitor is burixafor. In specific embodiments, the ADRB2 inhibitor is carvedilol.

In some embodiments, the method for suppressing, the method for treating, the pharmaceutical kit for use, or the pharmaceutical composition for use, provided herein further include determining the expression level of the ADRB2 gene in the cell, in the subject, or in the sample obtained from the subject, wherein the cell, the subject, or the sample obtained from the subject is determined to have an ADRB2-expressing cancer if the expression level is greater than a reference level of the ADRB2 gene. In specific embodiments, the methods or uses provided herein determine the expression level of the ADRB2 gene in the cell. In specific embodiments, the methods or uses provided herein determine the expression level of the ADRB2 gene in the subject. In specific embodiments, the methods or uses provided herein determine the expression level of the ADRB2 gene in the sample obtained from the subject. In specific embodiments, the cancer is an ADRB2-expressing cancer. In specific embodiments, the ADRB2 gene expression level in the cell is greater than a reference level. In specific embodiments, the ADRB2 gene expression level in the subject is greater than a reference level. In specific embodiments, the ADRB2 gene expression level in the sample obtained from the subject is greater than a reference level. In specific embodiments, wherein the ADRB2 gene expression level is greater than the reference level, the methods or uses provided herein include administering a CXCR4 inhibitor and an ADRB2 inhibitor. In specific embodiments, the CXCR4 inhibitor is burixafor. In specific embodiments, the ADRB2 inhibitor is carvedilol.

In some embodiments, the method for suppressing, the method for treating, the pharmaceutical kit for use, or the pharmaceutical composition for use, provided herein further include determining the expression level of the CXCR4 gene and the ADRB2 gene in the cell, in the subject, or in the sample obtained from the subject, wherein the cell, the subject, or the sample obtained from the subject is determined to have a CXCR4-expressing and ADRB2-expressing cancer if the expression levels of the CXCR4 gene and the ADRB2 gene are greater than respective reference levels of the CXCR4 gene and the ADRB2 gene. In specific embodiments, the methods or uses provided herein determine the expression level of the CXCR4 gene and the ADRB2 gene in the cell. In specific embodiments, the methods or uses provided herein determine the expression level of the CXCR4 gene and the ADRB2 gene in the subject. In specific embodiments, the methods or uses provided herein determine the expression level of the CXCR4 gene and the ADRB2 gene in the sample obtained from the subject. In specific embodiments, the cancer is a CXCR4-expressing and ADRB2-expressing cancer. In specific embodiments, the CXCR4 gene and the ADRB2 gene expression levels in the cell are greater than the respective reference levels. In specific embodiments, the CXCR4 gene and the ADRB2 gene expression levels in the subject are greater than the respective reference levels. In specific embodiments, the CXCR4 gene and the ADRB2 gene expression levels in the sample obtained from the subject are greater than the respective reference levels. In specific embodiments, wherein the CXCR4 gene and the ADRB2 gene expression levels are greater than the respective reference levels, the methods or uses provided herein include administering a CXCR4 inhibitor and an ADRB2 inhibitor. In specific embodiments, the CXCR4 inhibitor is burixafor. In specific embodiments, the ADRB2 inhibitor is carvedilol.

In some embodiments, the method for suppressing, the method for treating, the pharmaceutical kit for use, or the pharmaceutical composition for use, provided herein further include determining the expression level of the CXCR4 protein in the cell, in the subject, or in the sample obtained from the subject, wherein the cell, the subject, or the sample obtained from the subject is determined to have a CXCR4-expressing cancer if the expression level is greater than a reference level of the CXCR4 protein. In specific embodiments, the methods or uses provided herein determine the expression level of the CXCR4 protein in the cell. In specific embodiments, the methods or uses provided herein determine the expression level of the CXCR4 protein in the subject. In specific embodiments, the methods or uses provided herein determine the expression level of the CXCR4 protein in the sample obtained from the subject. In specific embodiments, the cancer is a CXCR4-expressing cancer. In specific embodiments, the CXCR4 protein expression level in the cell is greater than a reference level. In specific embodiments, the CXCR4 protein expression level in the subject is greater than a reference level. In specific embodiments, the CXCR4 protein expression level in the sample obtained from the subject is greater than a reference level. In specific embodiments, wherein the CXCR4 protein expression level is greater than the reference level, the methods or uses provided herein include administering a CXCR4 inhibitor and an ADRB2 inhibitor. In specific embodiments, the CXCR4 inhibitor is burixafor. In specific embodiments, the ADRB2 inhibitor is carvedilol.

In some embodiments, the method for suppressing, the method for treating, the pharmaceutical kit for use, or the pharmaceutical composition for use, provided herein further include determining the expression level of the ADRB2 protein in the cell, in the subject, or in the sample obtained from the subject, wherein the cell, the subject, or the sample obtained from the subject is determined to have an ADRB2-expressing cancer if the expression level is greater than a reference level of the ADRB2 protein. In specific embodiments, the methods or uses provided herein determine the expression level of the ADRB2 protein in the cell. In specific embodiments, the methods or uses provided herein determine the expression level of the ADRB2 protein in the subject. In specific embodiments, the methods or uses provided herein determine the expression level of the ADRB2 protein in the sample obtained from the subject. In specific embodiments, the cancer is an ADRB2-expressing cancer. In specific embodiments, the ADRB2 protein expression level in the cell is greater than a reference level. In specific embodiments, the ADRB2 protein expression level in the subject is greater than a reference level. In specific embodiments, the ADRB2 protein expression level in the sample obtained from the subject is greater than a reference level. In specific embodiments, wherein the ADRB2 protein expression level is greater than the reference level, the methods or uses provided herein include administering a CXCR4 inhibitor and an ADRB2 inhibitor. In specific embodiments, the CXCR4 inhibitor is burixafor. In specific embodiments, the ADRB2 inhibitor is carvedilol.

In some embodiments, the method for suppressing, the method for treating, the pharmaceutical kit for use, or the pharmaceutical composition for use, provided herein further include determining the expression level of the CXCR4 protein and the ADRB2 protein in the cell, in the subject, or in the sample obtained from the subject, wherein the cell, the subject, or the sample obtained from the subject is determined to have a CXCR4-expressing and ADRB2-expressing cancer if the expression levels of the CXCR4 protein and the ADRB2 protein are greater than respective reference levels of the CXCR4 protein and the ADRB2 protein. In specific embodiments, the methods or uses provided herein determine the expression level of the CXCR4 protein and the ADRB2 protein in the cell. In specific embodiments, the methods or uses provided herein determine the expression level of the CXCR4 protein and the ADRB2 protein in the subject. In specific embodiments, the methods or uses provided herein determine the expression level of the CXCR4 protein and the ADRB2 protein in the sample obtained from the subject. In specific embodiments, the cancer is a CXCR4-expressing and ADRB2-expressing cancer. In specific embodiments, the CXCR4 protein and the ADRB2 protein expression levels in the cell are greater than the respective reference levels. In specific embodiments, the CXCR4 protein and the ADRB2 protein expression levels in the subject are greater than the respective reference levels. In specific embodiments, the CXCR4 protein and the ADRB2 protein expression levels in the sample obtained from the subject are greater than the respective reference levels. In specific embodiments, wherein the CXCR4 protein and the ADRB2 protein expression levels are greater than the respective reference levels, the methods or uses provided herein include administering a CXCR4 inhibitor and an ADRB2 inhibitor. In specific embodiments, the CXCR4 inhibitor is burixafor. In specific embodiments, the ADRB2 inhibitor is carvedilol.

In some embodiments, a pharmaceutical composition (sometimes referred to herein as “pharmaceutical formulations”) provided herein includes a combination of a CXCR4 inhibitor and an ADRB2 inhibitor, and a pharmaceutically acceptable carrier. In some embodiments, a pharmaceutical composition provided herein includes a CXCR4 inhibitor and a pharmaceutically acceptable carrier. In some embodiments, a pharmaceutical composition provided herein includes an ADRB2 inhibitor, and a pharmaceutically acceptable carrier. A pharmaceutically acceptable carrier that can be used in the pharmaceutical compositions of the invention include any of the standard pharmaceutical carriers known in the art, such as physiologically acceptable carriers, excipients or stabilizers, for example, phosphate buffered saline solution, water and emulsions such as an oil and water emulsion, and various types of wetting agents. These pharmaceutical compositions can be prepared in liquid unit dose forms or any other dosing form that is sufficient for delivery of the CXCR4 inhibitor, the ADRB2 inhibitor, or the combination of the CXCR4 inhibitor and the ADRB2 inhibitor, to the target area of the subject in need of treatment. For example, the pharmaceutical compositions can be prepared in any manner appropriate for the chosen mode of administration, e.g., intravascular, intramuscular, subcutaneous, intradermal, intrathecal, etc. Other optional components, e.g., pharmaceutical grade stabilizers, buffers, preservatives, excipients and the like can be readily selected by one of skill in the art. The preparation of a pharmaceutically composition, having due regard to pH, isotonicity, stability and the like, is within the level of skill in the art.

In some embodiments, a pharmaceutical composition provided herein, which may be utilized in the method for suppressing, the method for treating, the pharmaceutical kit for use, or the pharmaceutical composition for use provided herein, includes: (a) a CXCR4 inhibitor, wherein the CXCR4 inhibitor is burixafor; (b) an ADRB2 inhibitor; and (c) a pharmaceutically acceptable carrier. In some embodiments, the pharmaceutical composition provided herein, or the methods of uses provided herein utilizing the same, includes wherein the ADRB2 inhibitor is an antagonist of ADRB2, an inverse agonist of ADRB2, a partial antagonist of ADRB2, an allosteric modulator of ADRB2, an antibody of ADRB2, an antibody fragment of ADRB2, a ligand of ADRB2, or an antibody-drug conjugate. In specific embodiments, the pharmaceutical composition provided herein, or the methods of uses provided herein utilizing the same, includes wherein the ADRB2 inhibitor is an antagonist ADRB2. In specific embodiments, the pharmaceutical composition provided herein, or the methods of uses provided herein utilizing the same, includes wherein the ADRB2 inhibitor is an inverse agonist ADRB2. In specific embodiments, the pharmaceutical composition provided herein, or the methods of uses provided herein utilizing the same, includes wherein the ADRB2 inhibitor is a partial antagonist ADRB2. In specific embodiments, the pharmaceutical composition provided herein, or the methods of uses provided herein utilizing the same, includes wherein the ADRB2 inhibitor is an allosteric modulator of ADRB2. In specific embodiments, the pharmaceutical composition provided herein, or the methods of uses provided herein utilizing the same, includes wherein the ADRB2 inhibitor is an antibody of ADRB2. In specific embodiments, the pharmaceutical composition provided herein, or the methods of uses provided herein utilizing the same, includes wherein the ADRB2 inhibitor is an antibody fragment of ADRB2. In specific embodiments, the pharmaceutical composition provided herein, or the methods of uses provided herein utilizing the same, includes wherein the ADRB2 inhibitor is a ligand of ADRB2. In specific embodiments, the pharmaceutical composition provided herein, or the methods of uses provided herein utilizing the same, includes wherein the ADRB2 inhibitor is an antibody-drug conjugate. In specific embodiments, the pharmaceutical composition provided herein, or the methods of uses provided herein utilizing the same, includes wherein the ADRB2 inhibitor is selected from the group consisting of: Alprenolol, atenolol, betaxolol, bupranolol, butoxamine, carazolol, carvedilol, CGP 12177, cicloprolol, ICI 118551, ICYP, labetalol, levobetaxolol, levobunolol, LK 204-545, metoprolol, nadolol, NIHP, NIP, propafenone, propranolol, sotalol, SR59230A, and timolol. In specific embodiments, the pharmaceutical composition provided herein, or the methods of uses provided herein utilizing the same, includes wherein the ADRB2 inhibitor is carvedilol. In specific embodiments, the pharmaceutical composition provided herein, or the methods of uses provided herein utilizing the same, includes wherein a therapeutically effective amount of the burixafor is administered to the subject. In specific embodiments, the pharmaceutical composition provided herein, or the methods of uses provided herein utilizing the same, includes wherein a sub-therapeutically effective amount of the burixafor is administered to the subject. In specific embodiments, the pharmaceutical composition provided herein, or the methods of uses provided herein utilizing the same, includes wherein a therapeutically effective amount of the ADRB2 inhibitor is administered to the subject. In specific embodiments, the pharmaceutical composition provided herein, or the methods of uses provided herein utilizing the same, includes wherein a sub-therapeutically effective amount of the ADRB2 inhibitor is administered to the subject.

In some embodiments, the method for suppressing, the method for treating, the pharmaceutical kit for use, or the pharmaceutical composition for use, provided herein include wherein the burixafor is administered as a pharmaceutical composition comprising a pharmaceutically acceptable carrier. In some embodiments, the methods or uses provided herein include wherein the ADRB2 inhibitor is administered as a pharmaceutical composition comprising a pharmaceutically acceptable carrier. In some embodiments, the methods or uses provided herein include wherein the combination of the burixafor and the ADRB2 inhibitor is administered sequentially, concurrently, or simultaneously. In some embodiments, the methods or uses provided herein include wherein the combination of the burixafor and the ADRB2 inhibitor is administered as a pharmaceutical composition further comprising a pharmaceutically acceptable carrier. In some embodiments, the methods or uses provided herein include wherein the burixafor and the ADRB2 inhibitor are administered as a combination of pharmaceutical compositions. In some embodiments, the methods or uses provided herein include wherein the combination of pharmaceutical compositions comprises: (a) a pharmaceutical composition comprising the burixafor and a pharmaceutically acceptable carrier; and (b) a pharmaceutical composition comprising the ADRB2 inhibitor and a pharmaceutically acceptable carrier. In some embodiments, the methods or uses provided herein include wherein the combination of the pharmaceutical compositions is administered sequentially, concurrently, or simultaneously.

Pharmaceutical formulations containing a CXCR4 inhibitor and an ADRB2 inhibitor, pharmaceutical formulations containing a CXCR4 inhibitor, and pharmaceutical formulations containing an ADRB2 inhibitor, provided herein can be prepared for storage by mixing the inhibitors having the desired degree of purity with optional physiologically acceptable carriers, excipients or stabilizers (Remington's Pharmaceutical Sciences (1990) Mack Publishing Co., Easton, Pa.), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; low molecular weight polypeptides of less than about 10 residues; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes, e.g., Zn-protein complexes; and/or non-ionic surfactants such as TWEEN™, PLURONICS™, or polyethylene glycol (PEG).

It is understood that modifications which do not substantially affect the activity of the various embodiments of this invention are also provided within the definition of the invention provided herein. Accordingly, the following examples are intended to illustrate but not limit the invention disclosed herein.

### EXAMPLES

### Example 1. Evaluation of CXCR4-GPCRx Heteromer Formation by BiFC Assay

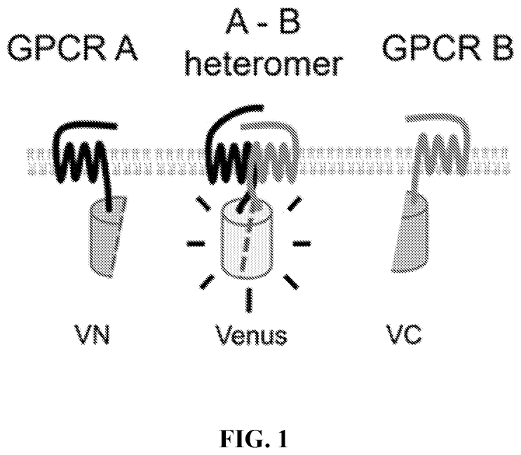

To identify novel CXCR4-GPCRx heteromers, recombinant adenoviruses were prepared encoding 143 GPCRs fused with N-terminal fragments of yellow fluorescent protein Venus (VN) and 147 GPCRs fused with C-terminal fragment of Venus (VC) (See, e.g., Song, Y. B., et al., (2014) Monitoring G protein-coupled receptor activation using an adenovirus-based beta-arrestin bimolecular fluorescence complementation assay, Anal Biochem 449, 32-41; South Korean Patent No. KR 101029972 B1, issued Apr. 12, 2011; Y. B., (2012) “Global analysis of GPCR dimerization using AdBiFC assay,” A Thesis Submitted in Partial Fulfillment of the Requirements for the Degree of Doctor of Philosophy, School of Biological Sciences, Seoul National University, 126 pages). CXCR4-GPCR heteromers were identified using bimolecular fluorescence complementation (BiFC) assay (FIG. 1), in which two complementary VN and VC fragments of Venus reconstitute a fluorescent signal only when both fragments are close enough through interaction between two different proteins to which they are fused (Hu, C. D., et al., (2002) Visualization of interactions among bZIP and Rel family proteins in living cells using bimolecular fluorescence complementation, Mol Cell 9, 789-798).

U-2 OS cells were plated in 96-well plates, were co-transduced with 30 MOI each of adenoviruses encoding CXCR4-VN and GPCRx-VC or CXCR4-VC and GPCRx-VN, and were allowed to express GPCRs for 2 days. After staining the cells with Hoechst 33342, BiFC and nuclear images were obtained from three fields per well using IN Cell Analyzer 1000. Images of about 200 cells from each well were analyzed with Multi-target analysis software in IN Cell Developer ToolBox (GE Healthcare, Waukesha, Wis.). Cell boundary was marked based on Hoechst signal, and fluorescence intensity per cell was measured. Cells with fluorescence intensities higher than background level were considered as BiFC positive cells. Dead cells that showed extremely high intensities were excluded from the cell count. Positive cells were determined, and positive cell count ratio (“BiFC score”) was calculated as (positive cells/total cells)×100.

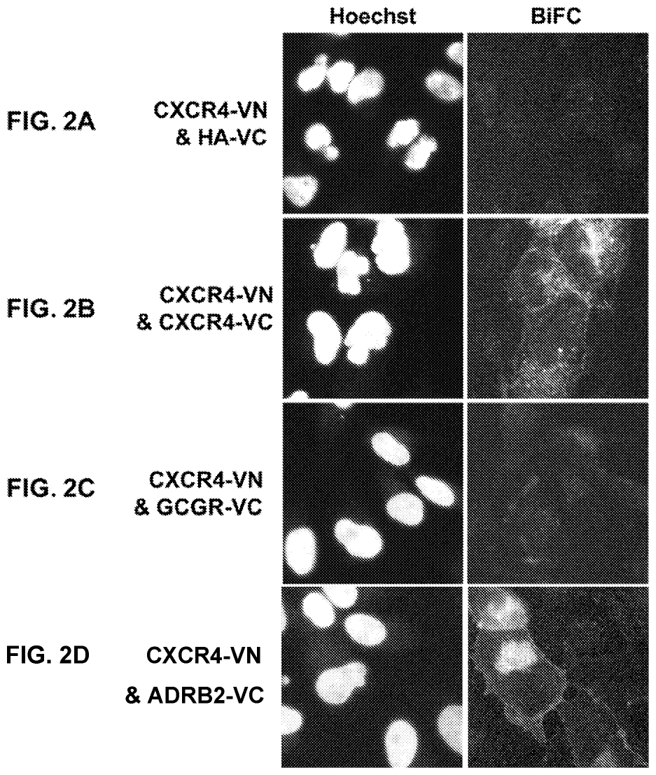

When CXCR4-VN was co-expressed with HA-VC (FIG. 2A) or GCGR-VC, a GPCR encoding glucagon receptor (FIG. 2C), no yellow fluorescence protein (YFP) signal (BiFC signal) was observed. In contrast, when CXCR4-VN was co-expressed with CXCR4-VC (FIG. 2B), BiFC signal was observed in the plasma membrane and in the cytoplasm. A strong BiFC signal was observed in the plasma membrane and in the cytoplasm when CXCR4-VN was co-transduced with ADRB2-VC (FIG. 2D). Cells that showed BiFC fluorescence signal higher than background level was counted as BiFC positive cells, and BiFC score was calculated.

Protein-protein interaction can be affected by fusion tags such as fluorescence protein fragments in BiFC or Renilla luciferase in BRET through interfering with the expression, folding, or localization of the partner protein. Partner protein can also impair the expression or folding of the fusion tags, and affect proximity-based assay result. Thus, the absence of signal between two proteins in specific combination does not necessarily imply that the proteins do not interact, but simply that the attached donor and acceptor molecules are in particular conformation that does not allow interaction to occur (Eidne, K. A., et al., (2002) Applications of novel resonance energy transfer techniques to study dynamic hormone receptor interactions in living cells, Trends Endocrinol Metab 13, 415-421; Kerppola, T. K., (2006) Design and implementation of bimolecular fluorescence complementation (BiFC) assays for the visualization of protein interactions in living cells, Nat Protoc 1, 1278-1286).

Therefore, CXCR4 and GPCRx that gave BiFC signal in either CXCR4-VN and GPCRx-VC or CXCR4-VC and GPCRx-VN combination was considered as interacting proteins. The BiFC score of CXCR4-VN and CXCR4-VC pair, a well-known homomer, was 9.9. Thus, CXCR4-GPCRx pairs that gave BiFC score equal or higher than 10 were selected as candidates for CXCR-GPCRx heteromer. The CXCR4-GPCRx pair of CXCR4-ADRB2 gave a BiFC score of 24, and was therefore considered as a CXCR-GPCRx heteromer candidate. Evaluation of additional GPCRs with respect to BiFC analysis was reported in International Application PCT/KR2018/016166, filed Dec. 18, 2018.

### Example 2. Evaluation of CXCR4-GPCRx Heteromer Formation by Co-Internalization Assay

Some of the GPCR heteromers are known to exhibit, as a result of the heteromerization, altered trafficking properties such as maturation of the partner GPCR (GABA(B) receptor) (White, 1998), agonist-mediated internalization of the partner GPCR from the cell surface (DOR-GRPR, A2A-D2R) (Hillion, J., et al., (2002) Coaggregation, cointernalization, and codesensitization of adenosine A2A receptors and dopamine D2 receptors, J Biol Chem 277, 18091-18097; Liu, X. Y., et al., (2011) Unidirectional cross-activation of GRPR by MOR1D uncouples itch and analgesia induced by opioids, Cell 147, 447-458; Torvinen, M., et al., (2005) Trafficking of adenosine A2A and dopamine D2 receptors, J Mol Neurosci 25, 191-200), and changes in the localization of the partner GPCR from an intracellular compartment to the cell surface (DOR-CB1) (Rozenfeld, R., et al., (2012) Receptor heteromerization expands the repertoire of cannabinoid signaling in rodent neurons, PLoS One 7, e29239).

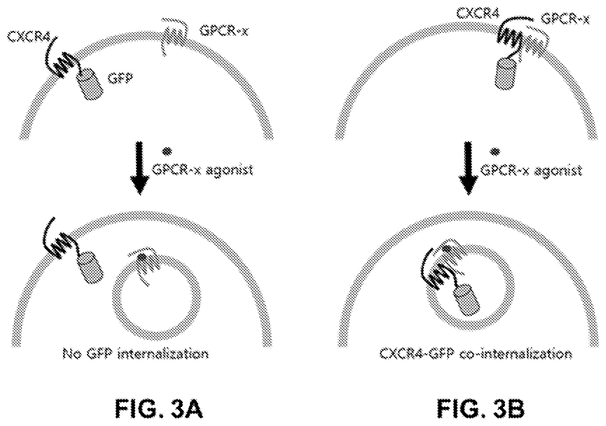

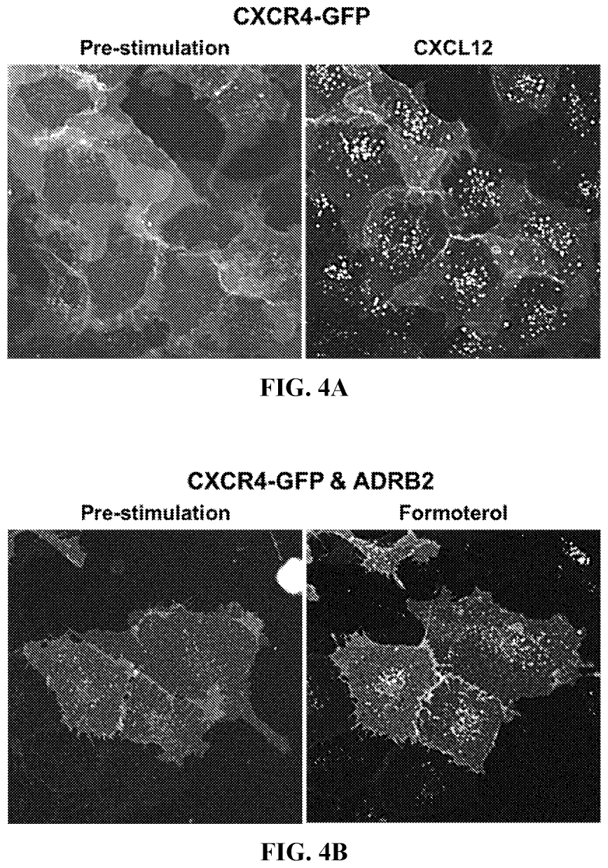

Co-internalization of pairs of co-expressed GPCRs in response to agonists selective for only one of the pair has been used to confirm GPCR heteromerization (Milligan, G., 2008). To examine if GPCRx modulates the trafficking of CXCR4 when co-expressed and forms CXCR4-GPCRx heteromer and also to confirm the physical interaction between CXCR4 and GPCRx identified using BiFC assay, cells were co-transduced with adenoviruses encoding CXCR4-GFP and GPCRx and GFP images were obtained before and 30 min after stimulation with GPCRx agonist. Loss of GFP expression on the cell surface or appearance of GFP granules inside the cells were considered as CXCR4-GFP co-internalization with GPCRx (FIGS. 3A-3B).

In FIG. 4A-4B, cells were stimulated with GPCRx agonists specific for CXCR4 (FIG. 4A) and ADRB2 (FIG. 4B), with 10 nM of CXCL12 (FIG. 4A) and 100 nM of formoterol (FIG. 4B), respectively. Images were obtained before and 30 min after agonist stimulation, and analyzed using IN Cell Analyzer 2000. The selection of the concentration of these GPCRx agonists was initially set at the EC50 concentration (or alternatively, the Ki or Kd concentration) for the respective specific GPCRx, and if the resulting signal was too intense, then the concertation was lowered below the EC50, and if the resulting signal was too weak, then the concertation was increased to no more than 10,000× the EC50 concentration.

Stimulation of cells expressing CXCR4-GFP with CXCL12 resulted in re-location of GFP from the plasma membrane to distinct intracellular granules, showing internalization of surface CXCR4-GFP into the cytoplasm (FIG. 4A). Regarding the CXCR4-ADRB2 heteromer identified by BiFC assay, the ADRB2 induced internalization of CXCR4 as revealed by increased intracellular GFP granules and reduced GFP signal in the plasma membrane in cells co-expressing CXCR4-GFP and ADRB2 (FIG. 4B), confirming heteromer co-internalization. Evaluation of additional GPCRs with respect to induced internalization of CXCR4 was reported in International Application PCT/KR2018/016166, filed Dec. 18, 2018.

### Example 3. Evaluation by Ca2+ Mobilization Assay of Enhanced CXCR4 Downstream Signaling Upon CXCR4-GPCRx Heteromer Formation and Inhibition of the Enhanced Signaling

To further examine if a CXCR4-GPCRx heteromer exhibited properties distinct from those of the individual protomers (in cells lacking one of the receptors), calcium signaling in the presence of either or both agonists in cells expressing either GPCR or both GPCRs together was evaluated. In this example, as noted in Example 2, the selection of the concentration of these GPCRx agonists was initially set at the EC50 concentration (or alternatively, the Ki or Kd concentration) for the respective specific GPCRx, and if the resulting Ca2+ signal was too intense, then the concentration was lowered below the EC50, and if the resulting Ca2+ signal was too weak, then the concertation was increased to no more than 100× the EC50 concentration.

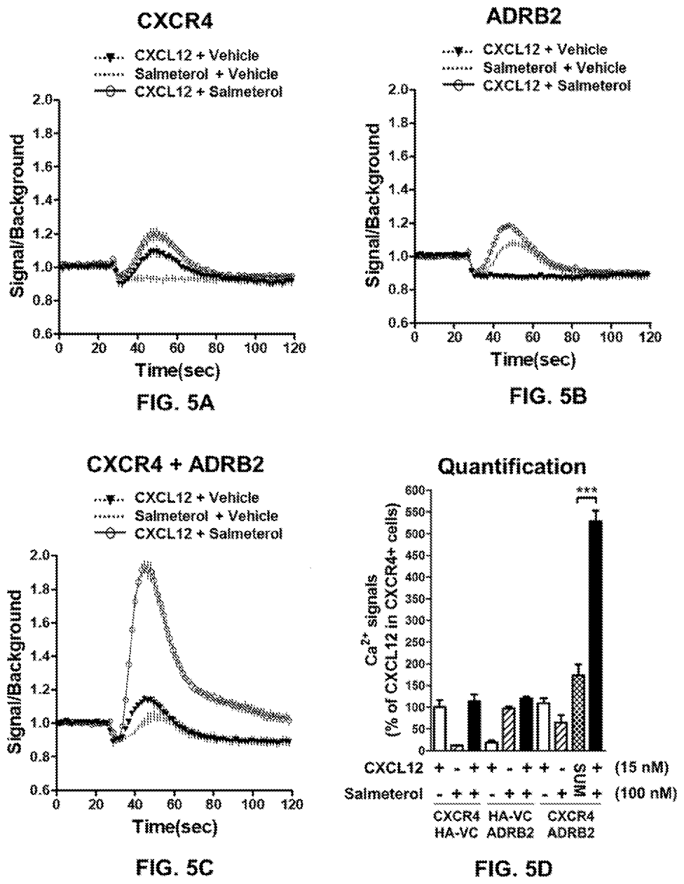

When MDA-MB-231 human breast cancer cells were transduced with adenoviruses encoding CXCR4, stimulation with CXCL12 evoked intracellular calcium mobilization (FIG. 5A). Stimulation of the cells with salmeterol, an ADRB2-selective agonist, did not induce calcium response, demonstrating the calcium response evoked by CXCL12 was mediated by CXCR4. Co-stimulation of the cells with CXCL12 and salmeterol induced similar calcium response compared to the one induced by CXCL12 alone. In cells overexpressing ADRB2 alone, CXCL12 alone did not induced calcium response while salmeterol alone induced calcium response, showing that salmeterol induces calcium response through ADRB2 (FIG. 5B). Co-stimulation with both agonists induced similar calcium response to the one stimulated by salmeterol alone.

In cells overexpressing both CXCR4 and ADRB2, stimulation with each agonist induced calcium responses similar to the ones shown in cells expressing CXCR4 or ADRB2 alone (FIG. 5C vs 5A and 5B). In contrast, co-stimulation with both agonists together significantly increased the calcium response compared to the ones evoked by individual agonists (FIGS. 5C & 5D). The enhanced calcium signaling was observed only in cells expressing both CXCR4 and ADRB2, but not in cells expressing either CXCR4 or ADRB2 alone. These results demonstrate that the CXCR4-ADRB2 heteromer exhibits properties distinct from those of its respective individual GPCR protomers.

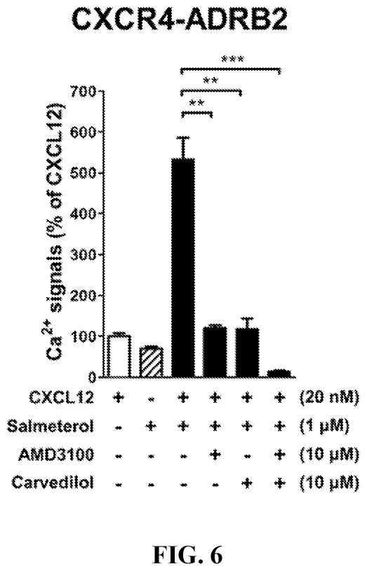

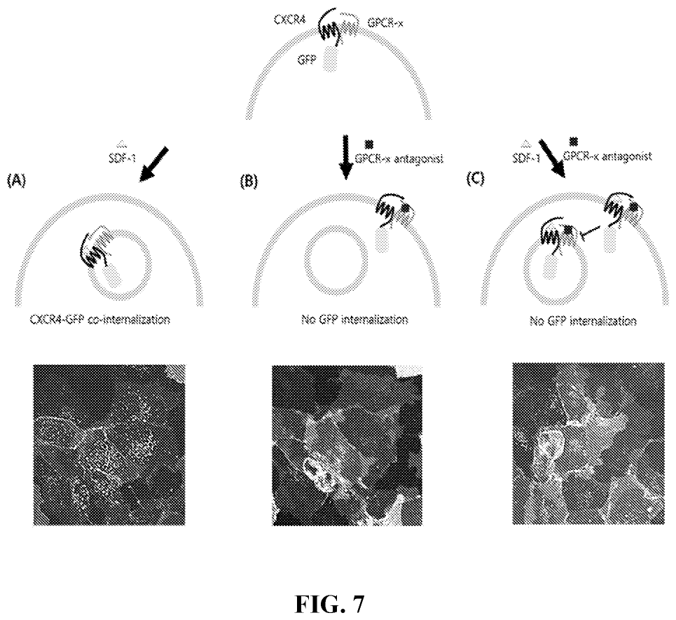

It was further examined if the enhanced calcium responses in cells co-expressing CXCR4 and GPCRx upon co-stimulation with CXCL12 and GPCRx ligands are inhibited by GPCRx antagonists. Specifically, as shown in FIG. 6, in cells co-expressing CXCR4 and ADRB2, administration of ADRB2 antagonist carvedilol (10 μM) suppressed the enhanced calcium signaling significantly, and co-administration of both a CXCR4 antagonist (AMD3100; 10 μM) with ADRB2 antagonist carvedilol (10 μM) resulted in greater suppression of the calcium signaling. These results demonstrate that an CXCR4 antagonist, an ADRB2 antagonist, or a combination of an CXCR4 antagonist with an ADRB2 antagonist, may be used as therapeutics against CXCR4-ADRB2 heteromers. Evaluation of additional GPCRs with respect to calcium signalling in the presence of one or both respective agonists, and in the presence of one or both respective antagonists, was reported in International Application PCT/KR2018/016166, filed Dec. 18, 2018.

The co-administration results also suggest that small doses of antagonists targeting each respective protomer of a heteromer may provide a novel therapeutic tool to efficiently suppress CXCR4-GPCRx heteromer response while avoiding side effects associated with high doses of the individual respective antagonists.

### Example 4. Inhibition of Internalization by GPCRx Antagonists

To further study if co-internalization of CXCR4 heterodimer was blocked by partner GPCRx antagonists, internalization inhibition assay was performed. As shown in FIG. 4B (wherein GPCRx is ADRB2), CXCR4-GFP expressing U-2 OS cells were co-internalized by partner GPCRx specific agonist when CXCR4 and GPCRx were simultaneously transduced to cells (control: CXCR4-GFP (FIG. 4A)). If CXCR4 formed heterodimer with GPCRx and co-internalized by partner GPCRx, it can be blocked by GPCRx specific antagonist.

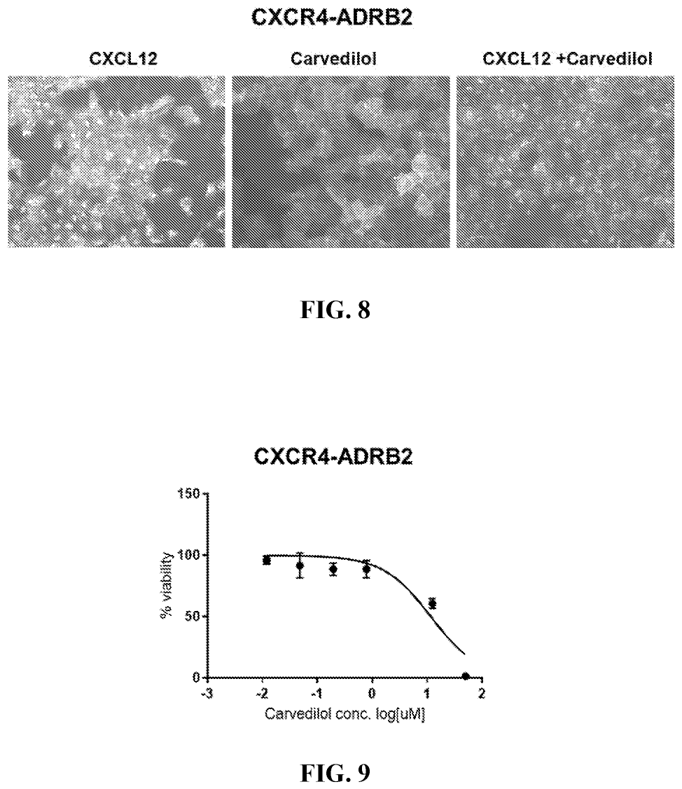

U-2 OS cells stably expressing CXCR4-GFP were transduced with Adenoviruses encoding GPCRx (ADRB2). After 2 days, images were obtained before and 20 minutes after stimulating cells with CXCR4-specific agonist, CXCL12 (20 nM), and/or GPCRx-specific antagonist (10 μM). Internalization of CXCR4-GFP was observed as GFP granules using IN Cell Analyzer 2500. Loss of GFP expression on the cell surface or appearance of GFP granules inside the cells were considered as CXCR4-GFP co-internalization. Here, the GPCRx evaluated was ADRB2. Stimulation with the CXCR4 agonist, CXCL12, induced internalization of CXCR4-GFP with ADRB2 (FIG. 8, left panel). Administration of an ADRB2 antagonist, carvedilol, had no effect on the internalization of the heteromer (FIG. 8, middle panel). Internalization of CXCL12 stimulated CXCR4-GFP with ADRB2 were inhibited by ADRB2 antagonist (FIG. 8, right panel). The evaluation of additional GPCRs with respect to inhibition of internalization by GPCRx antagonists was reported in International Application PCT/KR2018/016166, filed Dec. 18, 2018.

These data suggest that, by inhibition of internalization of CXCR4 heteromer, abnormal downstream signal in CXCR4-ADRB2 heteromer overexpressed cells, such as cancers, can be blocked for therapeutics purposes.

### Example 5. Evaluation by Cell Proliferation Assay of Phenotypic Effects of Inhibitor of CXCR4-GPCRx Heteromer Signaling on Tumor Growth

To develop a CXCR4-GPCRx heteromer-based therapeutic agent, the effects of co-administration of CXCR4 antagonist and GPCRx antagonist on cell proliferation was evaluated, particularly with respect to ADRB2.

Single cell suspensions of resected and dissociated glioblastoma tissues from patients were prepared (provided by Samsung Seoul hospital in Seoul, Korea). These cells were cultured under conditions optimal for propagation and non-differentiation of normal neuronal stem cell. Media were composed serum free Neurobasal media supplemented with basic FGF and EGF.

The effects of GPCRx antagonist on the survival of patient derived cells (PDCs) were assessed using ATPlite (PerkinElmer, Cat. No. 6016739 reagent). ATPlite is an Adenosine Triphospate monitoring system based on firefly luciferase. This luminescence assay is the alternative to colorimetric, fluorometric and radioisotopic assays for the quantitative evaluation of proliferation and cytotoxicity of cultured mammalian cells. Cells were seeded in 384-well plate at 500 cells/well in 40 μL culture media. After overnight growth, the cells were cultured for 7 days in the presence of several dose of GPCRx antagonist or DMSO alone. After a 7-day incubation, 15 μL ATPlite was added into the each well and the plate were shaken for 5 minutes in an orbital shaker at 700 rpm. The luminescent signal was detected within 30 minutes at PerkinElmer TopCount detection instrument. The cell viability was calculated using the equation: Cell viability (%)=(OD of antagonist treatment/OD of DMSO only treatment)×100%.

When Carvedilol, ADRB2 specific antagonist, was administered to PDC expressing CXCR4 and ADRB2, the growth of cells was inhibited significantly (IC50=11.69 μM, FIG. 9). The evaluation of additional GPCRs with respect to cell proliferation was reported in International Application PCT/KR2018/016166, filed Dec. 18, 2018.

These results suggest that CXCR4 heteromer induced abnormal cell proliferation can be blocked by ADRB2 antagonist in CXCR4-ADRB2 heteromer expressing cells, indicating that inhibition of cancer cell growth using ADRB2 antagonist in CXCR4-ADRB2 heteromer bearing patients can overcome the limitations of mono-therapy administering CXCR4 inhibitors alone as cancer therapeutics.

### Example 6. Evaluation of CXCR4-GPCRx Heteromer Formation in Patient Derived Cells (PDC) Using Proximity Ligation Assay (PLA)

To investigate the existence of GPCR complexes in native tissues, various approaches such as atomic force microscopy (Fotiadis, D., et al., (2006) Structure of the rhodopsin dimer: a working model for G-protein-coupled receptors, Curr Opin Struct Biol 16, 252-259), co-immunoprecipitation (Gomes, I., et al., (2004) A role for heterodimerization of mu and delta opiate receptors in enhancing morphine analgesia, Proc Natl Acad Sci USA, 101(14):5135-5139) and binding or functional assays (Wreggett, K. A., et al., (1995) Cooperativity manifest in the binding properties of purified cardiac muscarinic receptors, J Biol Chem 270, 22488-22499) have been used. The most convenient methods to monitor interactions are based on resonance energy transfer performed with labeled proteins. The labeling can be performed by selective probes such as antibodies or fluorescent ligands (Roess, D. A., et al., (2000) Luteinizing hormone receptors are self-associated in the plasma membrane, Endocrinology 141, 4518-4523; Patel, R. C., et al., (2002) Ligand binding to somatostatin receptors induces receptor-specific oligomer formation in live cells, Proc Natl Acad Sci USA 99, 3294-3299).

Bazin et al. employed a time-resolved fluorescence resonance energy transfer (TR-FRET)-based approach that offers a much higher signal-to-noise ratio (Bazin, H., et al., (2002) Time resolved amplification of cryptate emission: a versatile technology to trace biomolecular interactions, J Biotechnol 82, 233-250). FRET is based on the transfer of energy between two fluorophores, a donor and an acceptor, when in close proximity. Molecular interactions between biomolecules can be assessed by coupling each partner with a fluorescent label and by detecting the level of energy transfer. Introducing a time delay of approximately 50 to 150 μseconds between the system excitation and fluorescence measurement allows the signal to be cleared of all non-specific short-lived emissions.

Proximity ligation assay (PLA) is a technology that extends the capabilities of traditional immunoassays to include direct detection of proteins, protein interactions and modifications with high specificity and sensitivity (Gullberg, M., et al., (2004) Cytokine detection by antibody-based proximity ligation, Proc Natl Acad Sci USA 101, 8420-8424). Two primary antibodies raised in different species recognize the target antigen on the proteins of interest. Secondary antibodies directed against the constant regions of the different primary antibodies, called PLA probes, bind to the primary antibodies. Each of the PLA probes has a unique short DNA strand attached to it. If the PLA probes are in close proximity (that is, if the two original proteins of interest are in close proximity, or part of a protein complex, as shown in the figures), the DNA strands can participate in rolling circle DNA synthesis when appropriate substrates and enzymes are added. The DNA synthesis reaction results in several-hundred fold amplification of the DNA circle. Next, fluorescent-labeled complementary oligonucleotide probes are added, and they bind to the amplified DNA. The resulting high concentration of fluorescence is easily visible as a distinct bright spot when viewed with a fluorescence microscope (Gustafsdottir, S. M., et al., (2005) Proximity ligation assays for sensitive and specific protein analyses, Anal Biochem 345, 2-9).

CXCR4 overexpressing cell line, U20S-CXCR4, was infected with ADRB2 expressing adenovirus, Ad-ADRB2 at the dose of 0, 2.5, 10, 40 MOIs for 2 days. PLA was performed as described previously (Brueggemann, L. I., et al., (2014) Differential protein kinase C-dependent modulation of Kv7.4 and Kv7.5 subunits of vascular Kv7 channels, J Biol Chem 289, 2099-2111; Tripathi, A., et al., (2014) CXC chemokine receptor 4 signaling upon co-activation with stromal cell-derived factor-lalpha and ubiquitin, Cytokine 65, 121-125). To perform PLA, infected cells were fixed with 4% paraformaldehyde (PFA) on sixteen-well tissue culture slides. Slides were blocked with blocking solution provided by Duolink and incubated with mouse anti-CXCR4 (1:200, Santacruz, Sc-53534), Rabbit anti-ADRB2 (1:200, Thermoscientific, PA5-33333), Rabbit anti-CHRM1 (1:200, Ls bio, Ls-C313301) at 37° C. for 1 h in a humidifying chamber. Slides were then washed and incubated (1 h at 37° C.) with secondary anti-rabbit and anti-mouse antibodies conjugated with plus and minus Duolink II PLA probes. Slides were washed again and then incubated with ligation-ligase solution (30 min at 37° C.) followed by incubation with amplification-polymerase solution (2 h at 37° C.). Slides were then mounted with minimal volume of Duolink II mounting medium with 4′,6-diamidino-2phenylindole (DAPI) for 15-30 min, and PLA signals [Duolink In Situ Detection Reagents Green (λ excitation/emission 495/527 nm) or Red (λ excitation/emission 575/623 nm)] were identified as fluorescent spots under a IN Cell analyzer 2500.

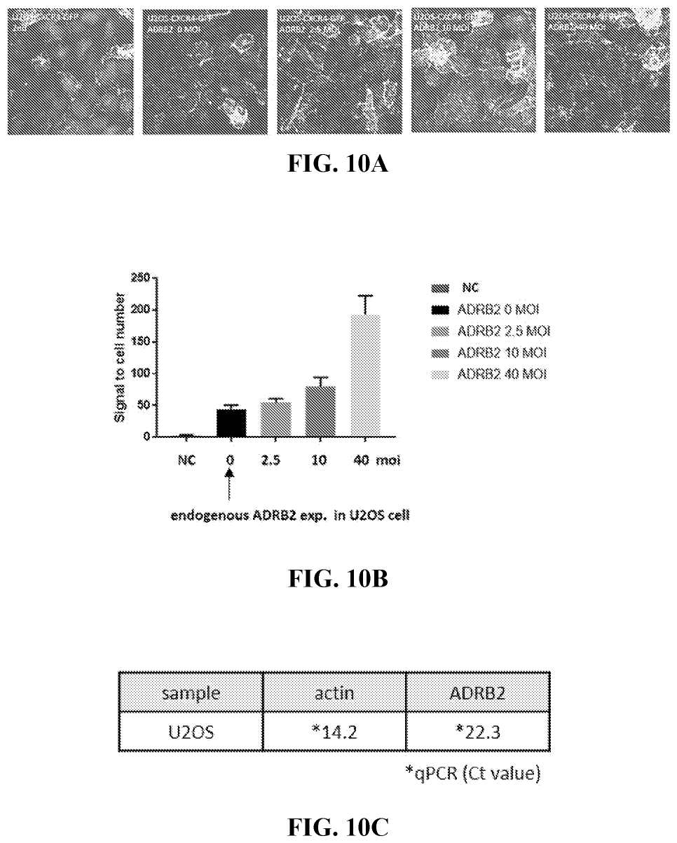

As shown in FIGS. 10A-10B, the PLA signal increases in a dose dependent manner as the expression level of ADRB2. In FIG. 10A, images of PLA signal from U20S cells expressing CXCR4-ADRB2 heteromer over a series of MOIs (multiplicity of infection). In FIG. 10B, the red signal spots were counted and calculated by normalization against negative control. The PLA signal increased proportionate to the expression level of ADRB2 in a dose dependent manner. Analysis by qRT-PCR was performed with ADRB2 specific primers to investigate endogenous ADRB2 expression. As shown in FIG. 10C, endogenous ADRB2 expression level of U20S cell was quite high, indicating that the PLA signal is detected even in the section without virus infection (at 0 MOI for ADRB2).

Traditionally Glioblastoma (GBM) is the most common and lethal primary brain tumor. Preclinical cancer biology has largely relied on the use of human cancer cell lines in vitro and the xenograft process of established these cell lines. However, the process of establishing conventional cell lines results in irreversible loss of important biological properties and, as a result, the xenograft tumor models do not maintain genomic and phenotypic characteristics present in the original tumor.

Patient derived cells (PDC) derived directly from glioblastoma harbor extensive similarities to normal neural stem cells and recapitulate the genotype, gene expression patterns, and in vivo biology of human glioblastomas.

To perform PLA with PDC samples, the patient derived cells were plated and fixed with 4% PFA on sixteen-well tissue culture slides. Slides were blocked with blocking solution provided by Duolink and incubated with mouse anti-CXCR4 (1:200, Santa Cruz, Sc-53534), rabbit anti-ADRB2 (1:200, Thermo Scientific, PA5-33333), rabbit anti-CHRM1 (1:200, Lsbio, Ls-C313301) at 37° C. for 1 h in a humidifying chamber. Slides were then washed and incubated (1 h at 37° C.) with secondary anti-rabbit and anti-mouse antibodies conjugated with plus and minus Duolink II PLA probes. Slides were washed again and then incubated with ligation-ligase solution (30 min at 37° C.) followed by incubation with amplification-polymerase solution (2 h at 37° C.). Slides were then mounted with minimal volume of Duolink II mounting medium with 4′,6′-diamidino-2-phenylindole (DAPI) for 15-30 min, and PLA signals [Duolink In Situ Detection Reagents Green (λ excitation/emission 495/527 nm) or Red (λ excitation/emission 575/623 nm)] were identified as fluorescent spots under the IN Cell analyzer 2500.

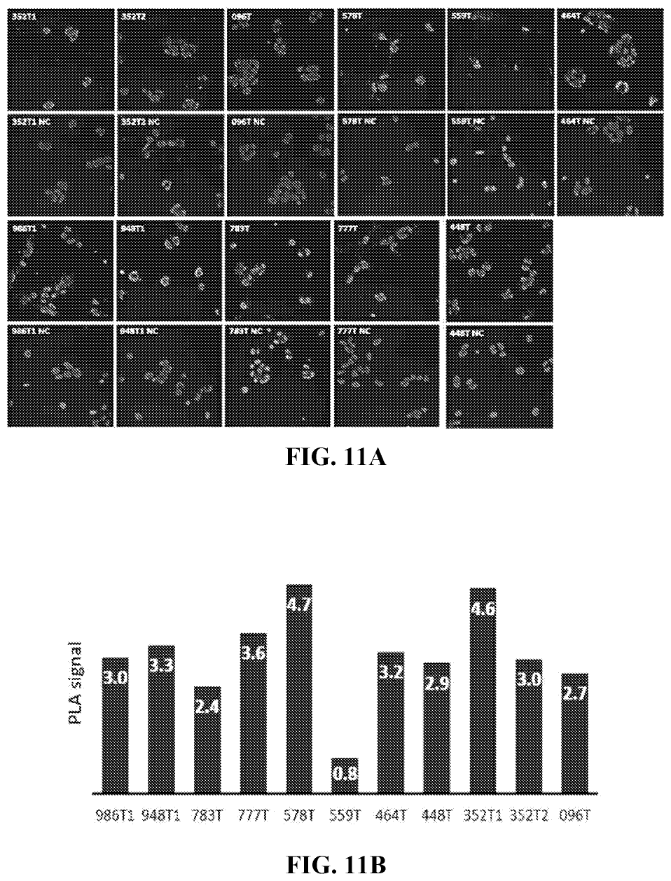

As shown in FIGS. 11A-11B, PLA ratio refers to the CXCR4-ADRB2 heteromer, and the frequency of the heteromer formation varies depending on the patient. PLA ratio was calculated as: number of fluorescent spots in PDC sample/number of fluorescent spots in negative control. Negative control (NC) represents background fluorescence signal, indicated by the number of spots when only the secondary antibody conjugated with plus and minus Duolink II PLA probes is treated without primary antibody (mouse anti-CXCR4, rabbit anti-ADRB2) treatment in PLA processing. These data demonstrate quantitative analysis of CXCR4-ADRB2 heterodimer in cancer patient samples. The evaluation of additional GPCRs with respect to heteromer formation in PDC was reported in International Application PCT/KR2018/016166, filed Dec. 18, 2018.

### Example 7. Evaluation of CXCR4-GPCRx Heteromer Formation In Vivo Using PDX Model

To perform PLA with Patient-Derived Xenograft (PDX) samples, the glioblastoma patient derived FFPE samples were used (provided by Samsung Seoul hospital in Seoul, Korea). After FFPE sample were de-paraffinized and performed heat induced antigen retrieval for 15 minutes at 100° C. Slides were blocked with blocking solution provided by Duolink and incubated with rabbit anti-CXCR4 (1:200, Thermoscientific, PA3305), mouse anti-ADRB2 (1:200, Santacruz, Sc-271322), at 37° C. for 1 h in a humidifying chamber. The other process was same as described above (PLA with PDC).

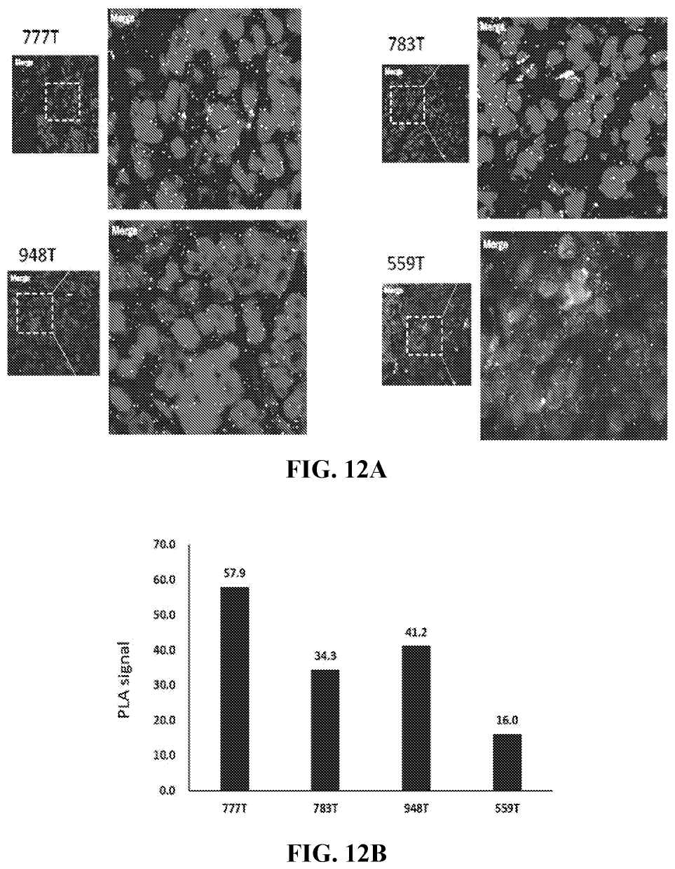

In the FIG. 12A, nuclei were visualized with DAPI staining, and CXCR4-ADRB4 heteromers were stained with PLA as small dots. As shown in FIG. 12B, the PLA ratio is different according to the patient and based on this result, indicating that it is possible to perform personalized medicine by the companion diagnostics.

### Example 8. Evaluation by Ca2+ Mobilization Assay of Enhanced CXCR4 Downstream Signaling Upon CXCR4-GPCRx Heteromer Formation

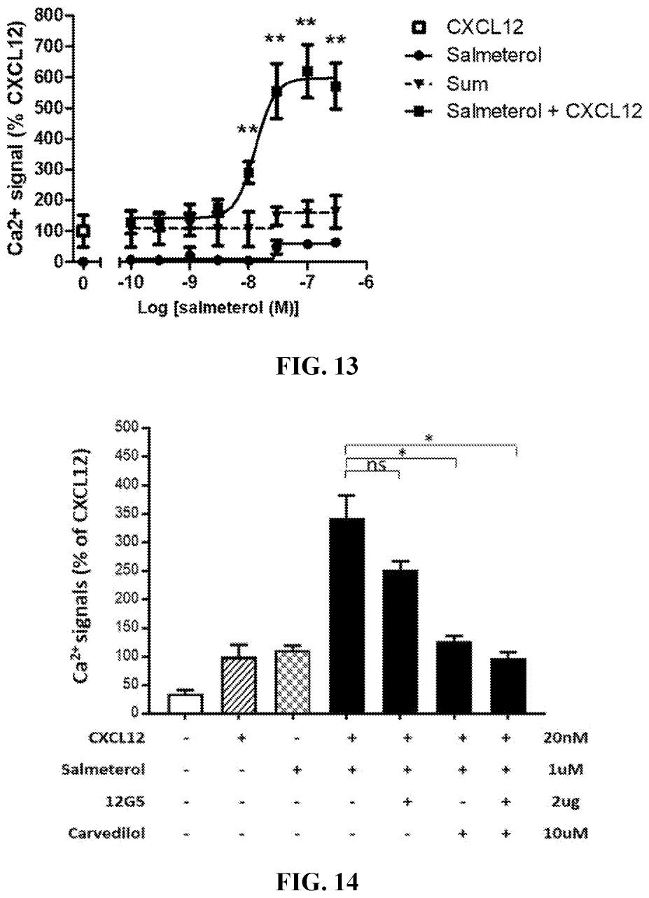

MDA-MB-231 cells were transduced with adenoviruses encoding CXCR4 and ADRB2 (FIG. 13). Cells were cultured for 3 days, stained with Cal-520 AM, and were treated with either CXCL12 (30 nM) alone, increasing doses of salmeterol alone or salmeterol in combination with 30 nM of CXCL12. Calcium mobilization was measured using FlexStation 3. In MDA-MB-231 cells overexpressing both CXCR4 and ADRB2, stimulation with salmeterol alone did not elicit calcium mobilization dose-dependently (FIG. 13). But when the cells were co-stimulated with salmeterol in the presence of CXCL12, calcium signaling was greatly enhanced in a broad range of salmeterol concentrations, such as concentrations in the range of between 10 nM to 300 nM.

### Example 9. Evaluation by Ca2+ Mobilization Assay of Enhanced CXCR4 Downstream Signaling Upon CXCR4-GPCRx Heteromer Formation and Inhibition of the Enhanced Signaling

In MDA-MB-231 cells overexpressing both CXCR4 and ADRB2, the co-stimulation with CXCL12, a CXCR4 agonist, and salmeterol, an ADRB2-selective agonist, significantly increased the calcium response, relative to the calcium responses evoked by mono-stimulation with the respective individual agonists (FIG. 14). These results demonstrate that the CXCR4-ADRB2 heteromer exhibits properties distinct from those of the individual GPCRs CXCR4 and ADRB2.

It was also examined if enhanced calcium responses in cells co-expressing CXCR4 and ADRB2 upon co-stimulation with CXCL12 and ADRB2 ligands are inhibited by anti-CXCR4 antibody as a CXCR4 antagonist. In cells co-expressing CXCR4 and ADRB2, administration of 2 μg of anti-CXCR4 antibody, 12G5, suppressed the enhanced calcium signaling as shown in FIG. 14. And co-administration of both antagonists (Carvedilol, ADRB2 antagonist and 12G5, CXCR4 antagonist together) resulted in a more significant suppression of the calcium signaling. These results demonstrate that an anti-CXCR4 antibody and an ADRB2 antagonist may be used as an efficient therapeutic (or combo-therapy) against CXCR4-ADRB2 heteromer mediated diseases.

In particular, utilizing the calcium mobilization assay, MDA-MB-231 human breast cancer cells were seeded at 20,000 cells per well in a black clear bottom 96-well plate (Corning Costar, #3340) in 100 μL of RPMI 1640 supplemented with 10% FBS. The next day, the cells were co-transduced with 10 MOI of CXCR4 and 30 MOI of GPCRx. After 2 days, cells were treated with ADRB2 antagonist, Carvedilol (Tocris), anti-CXCR4 antibody, 12G5 (Thermo Scientific, 35-8800) with indicated amounts and incubated with Cal 6 (FLIPR® Calcium 6 Assay Kit by Molecular Devices, Cat. R8191) for 2 hr. The cells were then stimulated with indicated amounts of CXCL12, ADRB2 agonist, or CXCL12 and ADRB2 agonist. Calcium mobilizations were measured using FlexStation 3 Multi-Mode Microplate Reader. The results were normalized for base-line activity. Calcium mobilizations were quantified by calculating the area-under-the-curve (AUC) of each graph. Data were normalized to CXCL12-stimulated calcium response in cells expressing CXCR4 alone. Data represent three independent experiments (mean±SEM). *P<0.05; Student's t test.

### Example 10. Effect of CXCR4-ADRB2 Heteromer on Tumor Growth

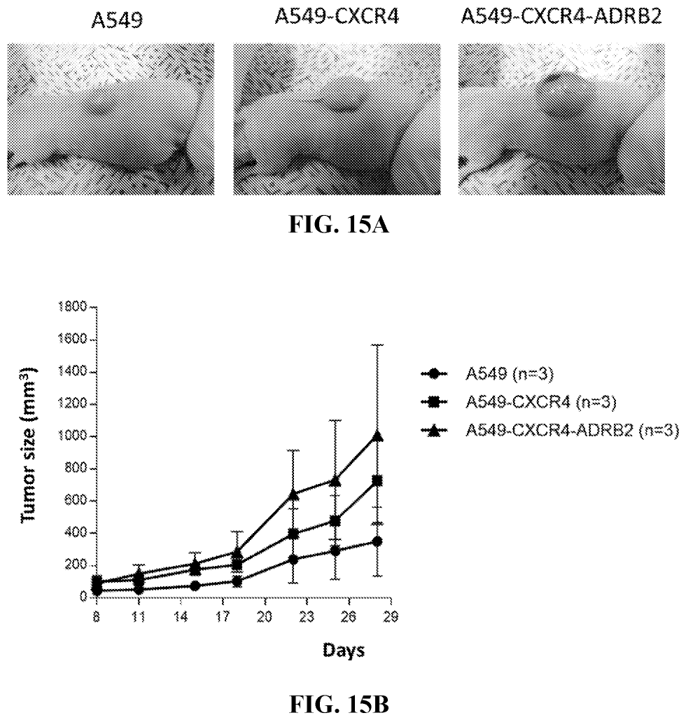

To investigate the effect of CXCR4-ADRB2 heteromer on tumor growth, cell lines stably overexpressing CXCR4 alone (“A549-CXCR4 cells”) or both CXCR4 and ADRB2, such that cells contain CXCR4-ADRB2 heteromer (“A549-CXCR4-ADRB2 cells”), were prepared in A549 lung cancer cell, and the same amount of cells (1×10⁷ cell/head) were injected subcutaneously in nude mice to compare tumor growth rates. As shown in FIGS. 15A-15B, the tumor size at 28 days after transplantation was 351.4±214.7 mm³ for A549, 726.9±259.6 mm³ for A549-CXCR4 cells, and 1012.2±556.1 mm³ for A549-CXCR4-ADRB2 cells. The tumor growth rate of mice transplanted with the A549-CXCR4 cells (overexpressing CXCR4) was faster than that of mice bearing only parental A549 cells, and the fastest tumor growth was observed in mice transplanted with the A549-CXCR4-ADRB2 cells (overexpressing both CXCR4 and ADRB2). These results suggest that the formation of CXCR4-ADRB2 heteromer synergistically increases the Ca²⁺ response and thus promotes tumor growth.

Images of three mice, which were transplanted with either parental cell A549, A549-CXCR4 cells (stably overexpressing CXCR4), or A549-CXCR4-ADRB2 cells (stably overexpressing both CXCR4 and ADRB2), at 28 days after transplantation, are shown in FIG. 15A. These images show that the tumor size of the mouse bearing the A549-CXCR4-ADRB2 cells (stably overexpressing both CXCR4 and ADRB2) was the largest (accelerated the most) among them. The tumor growth rates of these three different mice over time are graphically illustrated in FIG. 15B. Tumor growth was monitored every third or fourth day by measuring the length (L) and width (W) of the tumor and calculating tumor volume on the basis of the following formula: Volume=0.5 LW². The mice bearing the A549-CXCR4 cells showed relatively fast tumor growth as compared to the mice bearing parental cell A549, and the tumor growth was the fastest in mice transplanted with the A549-CXCR4-ADRB2 cells (stably overexpressing both CXCR4 and ADRB2).

### Example 11. Ca2+ Mobilization Inhibition of the Enhanced CXCR4 Downstream Signaling Upon CXCR4-ADRB2 Heteromer Formation—Comparison of Single Inhibitor Treatment to Combination Inhibitor Treatment

The degree of inhibition (measured as IC50 values for Ca²⁺ response) was compared for burixafor (a CXCR4 inhibitor, also referred to as TG-0054), evaluated as single treatment in MDA-MB-231 cells expressing CXCR4 alone (monomer or individual protomer context; “MDA-MB-231-CXCR4 cells”), as single treatment in MDA-MB-231 cell lines containing CXCR4-ADRB2 heteromer that overexpress both CXCR4 and ADRB2 (“MDA-MB-231-CXCR4-ADRB2 cells”), and as co-treatment with an ADRB2 inhibitor (Carvedilol; 10 μM) in MDA-MB-231-CXCR4-ADRB2 cells.

MDA-MB-231 cells were transduced with adenoviruses encoding CXCR4 only, or CXCR4 and ADRB2. The cells were cultured for 2 days and were treated with burixafor (CXCR4 inhibitor) alone or co-treated with burixafor and an ADRB2 inhibitor (Carvedilol; 10 μM). The cells were then stained with Cal-6 for 2 hours and stimulated with CXCR4 agonist (CXCL12; 20 nM) and ADRB2 agonist (Salmeterol; 1 μM). Calcium mobilization was measured using FlexStation3, the results of which are shown in Table 3, showing IC50 of Ca2+ response in: (1) MDA-MB-231-CXCR4 cells treated with burixafor only (2^{nd} column); (2) MDA-MB-231-CXCR4-ADRB2 cells treated simultaneously with burixafor and ADRB2 inhibitor Carvedilol (3^{rd} column); and (3) MDA-MB-231-CXCR4-ADRB2 cells treated only with burixafor (4^{th} column).

TABLE 3

| | IC₅₀ [nM] | | |
|---|---|---|---|
| CXCR4 | | CXCR4 + ADRB2 | CXCR4 + ADRB2 |
| inhibitor | CXCR4 | (+Carvedilol 10 uM) | (−Carvedilol 10 uM) |
| TG-0054 | 65.78 ± 1.34 | 0.02 ± 0.013 | 90.54 ± 31.27 |

As shown in Table 3, the IC50 value of Ca²⁺ response in the MDA-MB-231-CXCR4-ADRB2 cells containing the CXCR4-ADRB2 heteromer decreased by 4500 times or more when treated in combination with an ADRB2 inhibitor, Carvedilol (3^{rd} column), relative to single treatment with the CXCR4 inhibitor, TG-0054 alone (4^{th} column). This result suggests that co-treating with burixafor and ADRB2 inhibitor more effectively inhibits increased Ca²⁺ response than single treatment with burixafor alone in MDA-MB-231-CXCR4-ADRB2 cells containing the CXCR4-ADRB2 heteromer.

To determine the possibility that CXCR4-ADRB2 heteromer formation induces conformation change and/or changes the binding affinity for the CXCR4 inhibitor, the IC50 values of Ca²⁺ response was compared between single treatment with burixafor (TG-0054) in MDA-MB-231-CXCR4 cells expressing CXCR4 alone and MDA-MB-231-CXCR4-ADRB2 cells containing the CXCR4-ADRB2 heteromer. The result showed that single treatment with a CXCR4 inhibitor altered the IC50 value only by about 1.4 fold in MDA-MB-231-CXCR4-ADRB2 cells (4^{th} column), relative to MDA-MB-231-CXCR4 cells (2^{nd} column). This result suggests that co-treatment with CXCR4 inhibitor, e.g., burixafor (TG-0054), and ADRB2 inhibitor can increase, to a dramatic extent for certain CXCR4 inhibitors, the therapeutic efficacy towards CXCR4-ADRB2 heteromer containing subjects and/or subject cells/tissues, compared with single treatment with CXCR4 inhibitor.

As shown in Table 3, co-treatment with burixafor and carvedilol results in decreased IC50 value of Ca²⁺ response in the cells containing the CXCR4-ADRB2 heteromer. Since the over-dose of the ADRB2 antagonist may affect the activity of the counterpart CXCR4, the change in IC50 value of CXCR4 antagonists was measured by the combination of Carvedilol at its IC90 value concentration of 650 nM in combination with a series of CXCR4 antagonists. The data is provided in Table 4.

TABLE 4

| | IC₅₀ [nM] | | |
|---|---|---|---|
| CXCR4 | | CXCR4 + ADRB2 | CXCR4 + ADRB2 |
| inhibitor | CXCR4 | (+Carvedilol 600 nM) | (−Carvedilol 600 nM) |
| AMD3100 | 54.41 ± 4.80 | 2.12 ± 0.86 | 22.45 ± 8.77 |
| Ulocuplumab | 0.41 ± 0.01 | 0.03 ± 0.02 | 0.88 ± 0.34 |
| BKT140 | 578.9 ± 204.1 | 1.94 ± 0.77 | 88.66 ± 59.03 |
| TG-0054 | 65.78 ± 1.34 | 1.49 ± 0.07 | 90.54 ± 31.27 |

As shown in Table 4, the IC50 value of a CXCR4 inhibitor, in terms of Ca²⁺ response in the CXCR4-ADRB2 heteromer context, decreased when administered in combination with an ADRB2 inhibitor (3^{rd} column), relative to single administration with the CXCR4 inhibitor alone (4^{th} column). For instance, The IC 50 value of Ca²⁺ response in the CXCR4-ADRB2 heteromer context decreased about 10.5 fold (from 22.45 nM to 2.12 nM), about 29.3 fold (from 0.88 nM to 0.03 nM), about 45.7 fold (from 88.66 nM to 1.94 nM), about 60.7 fold (from 90.54 nM to 1.49 nM) upon co-administration of Carvedilol (600 nM, its IC90 concentration value against ADRB2 in the monomer context) with AMD3100, Ulocuplumab, BKT140, TG-0054, respectively. This result suggests that co-administration with low dose of CXCR4 inhibitor and ADRB2 inhibitor can increase the therapeutic efficacy towards CXCR4-ADRB2 heteromer containing subjects compared with single administration with CXCR4 inhibitor.

### Example 12. Stimulation with CXCL12 and/or Salmeterol Induces Activation of ERK Signaling in Cell Lines Containing CXCR4-ADRB2 Heteromer

ERK1/2 regulate chemotaxis or survival in different cell types (Riol-Blanco, L., et al., (2005) The chemokine receptor CCR7 activates in dendritic cells two signaling modules that independently regulate chemotaxis and migratory speed, J. Immunol. 174(7), 4070-4080; Klemke, R. L., et al., (1997) Regulation of cell motility by mitogen-activated protein kinase, J. Cell Biol. 137(2), 481-492; Copp, J., et al., (2009) ORC-specific phosphorylation of mammalian target of rapamycin (mTOR): phospho-Ser2481 is a marker for intact mTOR signaling complex 2, Cancer Res. 69(5), 1821-1827). ERK1/2 activity was evaluated upon stimulation of CXCR4 or CXCR4-ADRB2 heteromer in cells with CXCL12 (CXCR4 agonist) and/or salmeterol (ADRB2 agonist). Specifically, CXCL12 stimulation of MDA-MB-231 cells expressing Rluc-luc2P (“MDA-MB-231 parent cells”), MDA-MB-231 cells expressing CXCR4 (“MDA-MB-231-CXCR4 cells”), and MDA-MB-231 cells containing CXCR4-ADRB2 heteromer (“MDA-MB-231-CXCR4-ADRB2 cells”), induced activation of ERK1/2 after 10 minutes, and reached the highest level of activity after 60 minutes. Salmeterol stimulation of the MDA-MB-231 parent cells, MDA-MB-231-CXCR4 cells, and MDA-MB-231-CXCR4-ADRB2 cells, also induced ERK activation after 20 minutes and decayed to basal levels after 60 minutes (data not shown).

To determine the synergistic effect of CXCR4 and ADRB2 signaling, the levels of ERK1/2 activation were measured after CXCL12 and salmeterol stimulation. MDA-MB-231 cells and A549 cells, expressing Rluc-luc2P or CXCR4, or containing CXCR4-ADRB2 heteromer, were seeded in 6 well plates at a density of 5×10⁵ cells per well. After 16 hours of serum starvation, the respective cells were treated with CXCL12 10 nM and/or salmeterol 10 nM for 20 minutes in MDA-MB-231 parent cells, MDA-MB-231-CXCR4 cells, and MDA-MB-231-CXCR4-ADRB2 cells and for 10 minutes in A549 cells expressing Rluc-luc2P (“A549 parent cells”), A549 cells expressing CXCR4 alone (monomer or individual protomer context; “A549-CXCR4 cells”), and A549 cell lines containing CXCR4-ADRB2 heteromer that stably overexpress both CXCR4 and ADRB2 (“A549-CXCR4-ADRB2 cells”). Thereafter, each of the sets of cells were harvested and performed western blotting analysis.

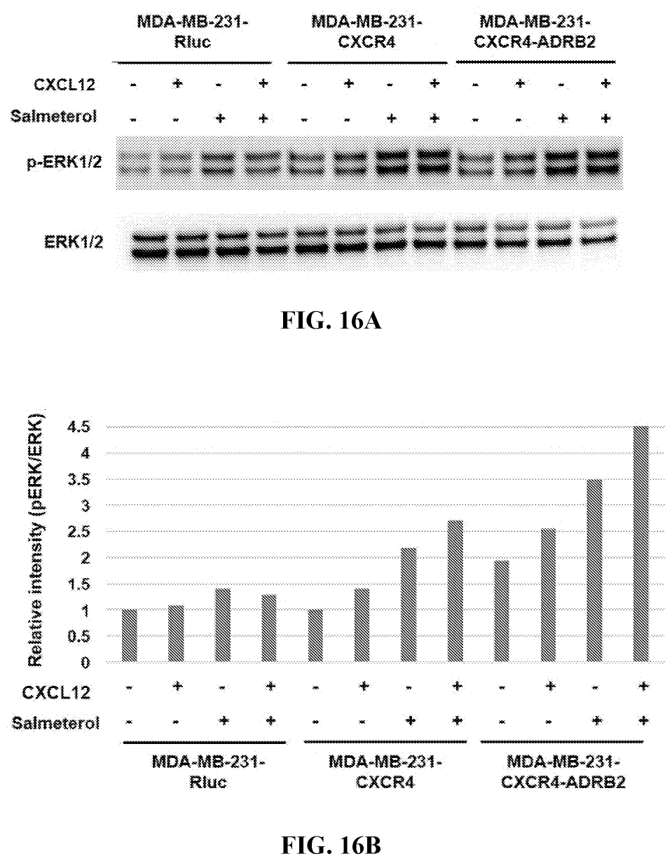

In stimulations with CXCL12 or salmeterol, alone, the level of ERK1/2 activation in MDA-MB-231-CXCR4 cells were higher than MDA-MB-231 parent cells. ERK1/2 phosphorylation was significantly increased in MDA-MB-231-CXCR4-ADRB2 cells compared to MDA-MB-231-CXCR4 cells. When the MDA-MB-231 parent cells were stimulated with CXCL12 and salmeterol at the same time, ERK1/2 phosphorylation was induced at a similar level to that of the single agonist stimulation. In the MDA-MB-231-CXCR4 cells, ERK1/2 phosphorylation was increased by the sum of each agonist alone, while in the MDA-MB-231-CXCR4-ADRB2 cells, ERK1/2 activation was synergistically increased (FIGS. 16A-16B).

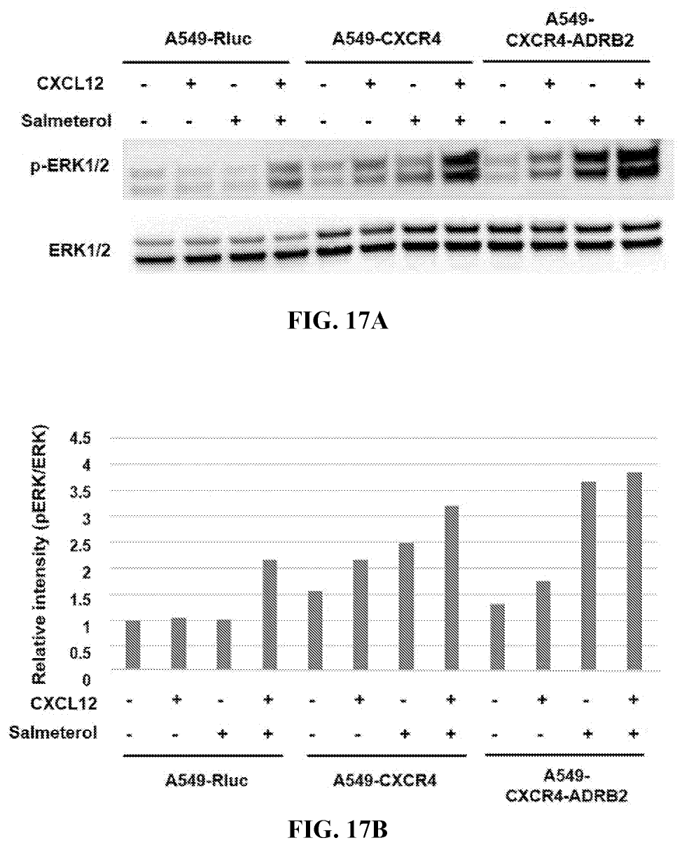

ERK1/2 activation was also investigated in A549 parent cells, A549-CXCR4 cells, and A549-CXCR4-ADRB2 cells. ERK1/2 phosphorylation was detected in A549-CXCR4 cells or A549-CXCR4-ADRB2 cells upon stimulation with CXCL12 or salmeterol, alone. In A549 parent cells, ERK1/2 phosphorylation was not induced by stimulation with CXCL12 or salmeterol, alone, but was significantly increased upon co-stimulation with CXCL12 and salmeterol. In A549-CXCR4 cells, ERK1/2 phosphorylation was induced by each agonist alone, and increased by the sum of each stimulation when co-stimulated with CXCL12 and salmeterol. In A549-CXCR4-ADRB2 cells, ERK1/2 phosphorylation was induced much more when stimulated with salmeterol alone than with CXCL12 alone, and was significantly activated when co-stimulated with CXCL12 and salmeterol (FIGS. 17A-17B).

These results suggest that the expression of CXCR4 and ADRB2 in cancer cells, and particularly when cancer cells contain CXCR4-ADRB2 heteromer, may result in significantly inducing downstream ERK activation and may have a considerable effect on cancer cell proliferation and chemotaxis.

### Example 13. The Effect of CXCR4 Antagonists on CXCR4/CXCL12 Mediated Proliferation Increase in A549 Cell Lines Containing CXCR4-ADRB2 Heteromer

To investigate the impact of the presence of a CXCR4-ADRB2 heteromer in cancer cells with respect to cancer cell growth, the growth of A549 cells containing CXCR4-ADRB2 heteromer that stably overexpress both CXCR4 and ADRB2 (“A549-CXCR4-ADRB2 cells”) was compared with that of A549 cells expressing Rluc-luc2P (A549-double negative cells; “A549 parent cells”) as a control, and depending on the presence of CXCL12 agonist.

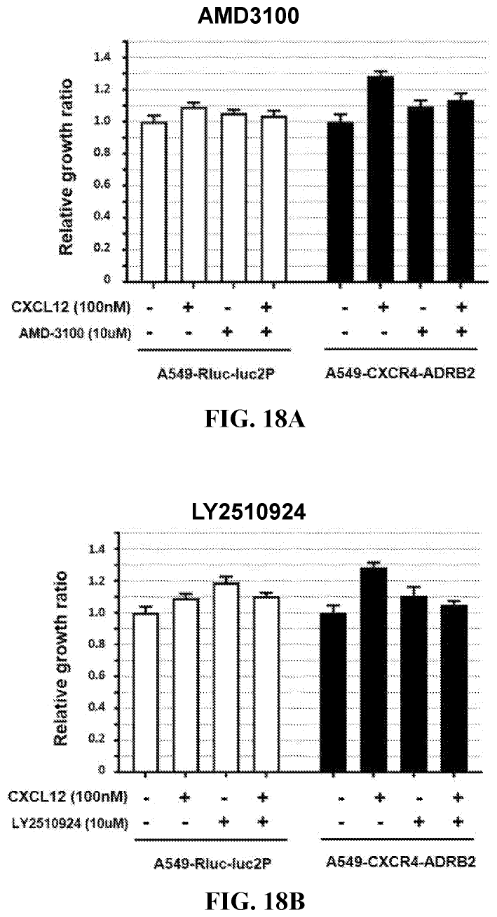

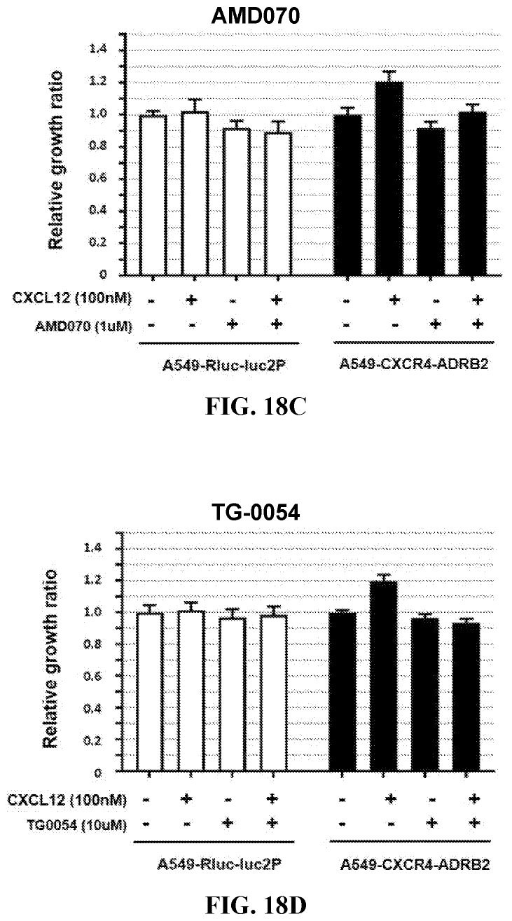

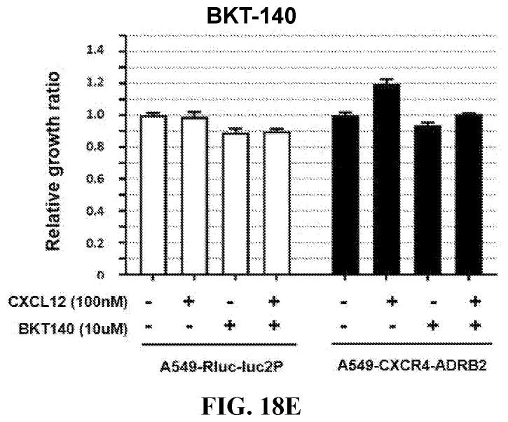

A549 parent cells or A549-CXCR4-ADRB2 cells were plated at a density of 1×10⁴ cells/well in 96-well plates and incubated in culture medium for 24 hr to allow adherence. After washing, Serum free medium±CXCL12, with or without indicated CXCR4 inhibitors (FIG. 18A: AMD3100 (10 μM), FIG. 18B: LY2510924 (10 μM), FIG. 18C: AMD070 (1 μM), FIG. 18D: TG-0054 (10 μM), and FIG. 18E: BKT-140 (10 μM)) in serum free condition were added and the cells were incubated for 72 hr (96 hr post seeding). At the end of incubation, cell proliferation was assessed by using Prestoblue Cell Viability Reagent (Thermo Fisher Scientific) according to the manufacture's instruction.

FIGS. 18A-18E show cell proliferation rate was observed to increase by about 20% in the presence of CXCL12 agonist, and this increase was blocked by each of the CXCR4 antagonists tested. Under normal cell growth conditions (10% serum), no difference in the cell proliferation rates was observed between A549 parent cells and A549-CXCR4-ADRB2 cells. However, in serum free conditions, A549-CXCR4-ADRB2 cells (not the A549 parent cells) exhibited a cell proliferation increase in the presence of CXCL12 in a dose dependent manner (data not shown). The maximum increase of about 20% was achieved in the presence of CXCL12 at 100 nM, for 72 hr. CXCL12 mediated cell proliferation increase was diminished as serum concentration increased (data not shown). To confirm that the increase of cell proliferation rate is mediated by CXCR4/CXCL12 specific signaling, several CXCR4 specific antagonists, AMD3100, LY210924, AMD070, TG-0054, and BKT-140, were evaluated. Each of the CXCR4 antagonists tested blocked the CXCL12 effect with respect to cell proliferation. The CXCR4/CXCL12 signaling axis increases the cell proliferation rate by about 20% in serum free condition, which is consistent to previous reports that CXCR4/CXCL12 induces proliferation increase only in suboptimal condition (Balkwill, Fran (2004) Nature Reviews Cancer, Cancer and the Chemokine Network, 4:540-550).

Inhibitors targeting CXCR4 may inhibit, for example, only as much as is increased by the presence of CXCL12, and the use of higher concentrations of CXCR4 inhibitors may cause cytotoxicity (data not shown). These results may explain the observations of low efficacy of tumor growth inhibition and adverse effects associated with the use of CXCR4 inhibitors as sole active agents in the clinic for cancer treatments. In subjects containing CXCR4-ADRB2 heteromer, while treatment with CXCR4 inhibitor alone may not effectively inhibit tumor growth, the co-administering of a CXCR4 inhibitor and ADRB2 inhibitor may effectively inhibit tumor growth.

### Example 14. Correlation Between CXCR4-ADRB2 Heteromer Amount and Tumor Growth

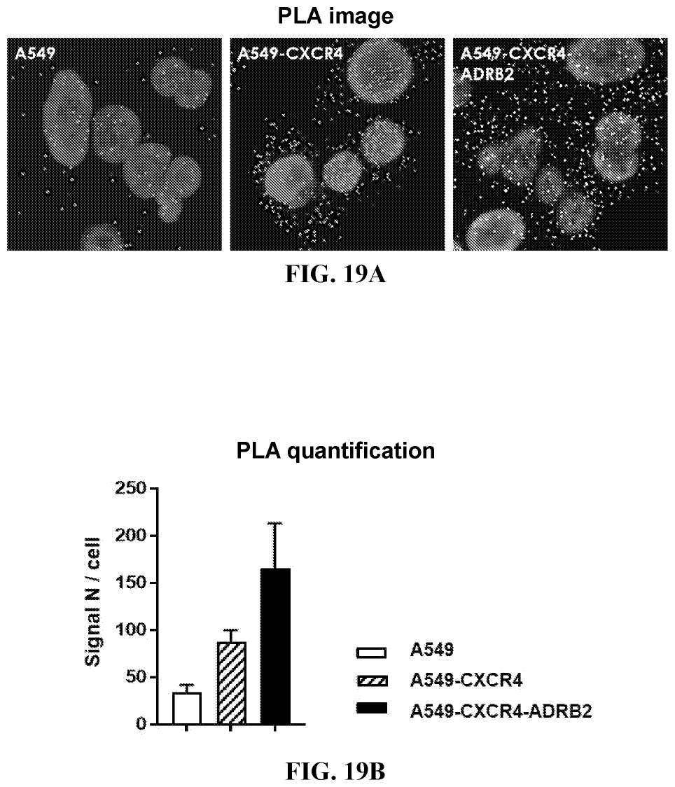

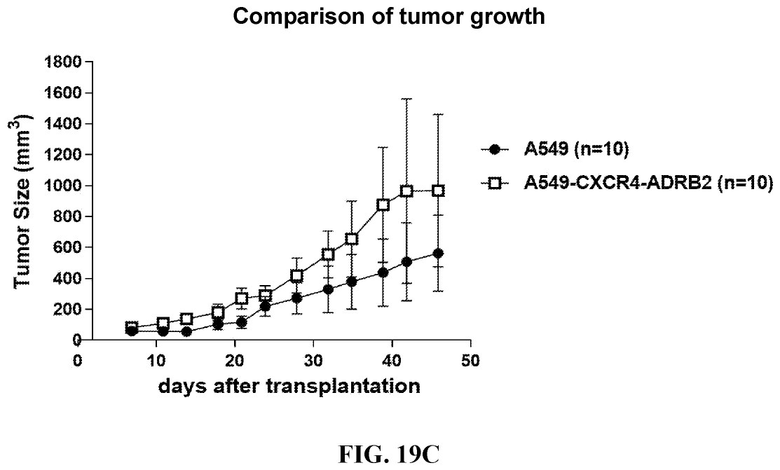

To investigate the correlation between the amount of CXCR4-ADRB2 heteromer and tumor growth, A549 cell lines stably overexpressing CXCR4 homomer (“A549-CXCR4 cells”) and A549 cell lines containing CXCR4-ADRB2 heteromer that stably overexpress both CXCR4 and ADRB2 were prepared (“A549-CXCR4-ADRB2 cells”). The amount of CXCR4-ADRB2 heteromer was compared via PLA in the engineered cell lines. FIGS. 19A-19B show that, as determined by PLA, there are about 30, about 80, and more than 150 CXCR4-ADRB2 heteromers that were detected in A549 parent cells, A549-CXCR4 cells, and A549-CXCR4-ADRB2 cells, respectively.

To determine whether containing the CXCR4-ADRB2 heteromer, as verified by PLA at the cellular level, increases tumor growth in a dose dependent manner, A549 parent cells and A549-CXCR4-ADRB2 cells were prepared and the same amount of cells (1×10⁷ cell/head) were injected subcutaneously in nude mice to compare tumor growth rates. Tumor growth was monitored every third or fourth day by measuring the length (L) and width (W) of the tumor and calculating tumor volume on the basis of the following formula: Volume=0.5 LW². As shown in FIG. 19C, the tumor size at 44 days after transplantation was 562.8±245.9 mm³ in A549 xenograft mouse (having A549 parent cells) and 967.2±493.0 mm³ on A549-CXCR4-CXCR4-ADRB2 xenograft mouse (having A549-CXCR4-ADRB2 cells). The tumor growth rate of mice transplanted with the A549-CXCR4-ADRB2 cells was faster than that of mice transplanted with A549 parent cells. These results suggest that the formation of CXCR4-ADRB2 heteromer synergistically increases the Ca²⁺ response and thus promotes tumor growth.

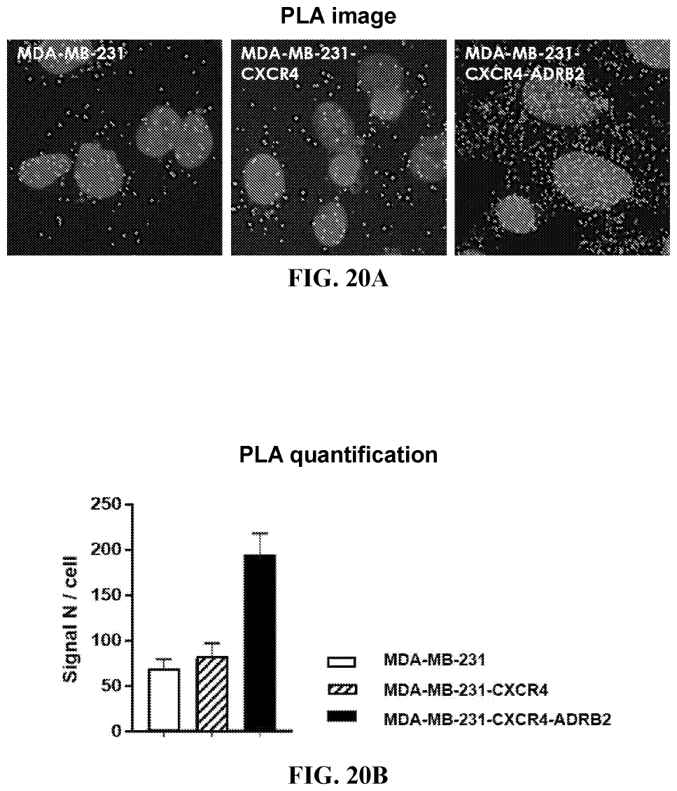

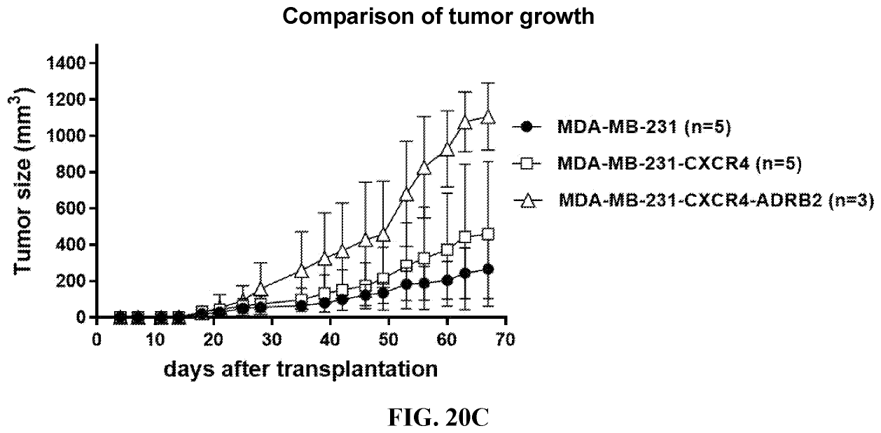

The correlation between the (presence and) amount of CXCR4-ADRB2 heteromer and tumor proliferation was confirmed in the A549 lung cancer cell line and in the MDA-MB-231 breast cancer cell line. In the same manner as in A549 cell line context, the MDA-MB-231-CXCR4 cell line overexpressing CXCR4 homomer (“MDA-MB-231-CXCR4 cells”) and MDA-MB-231 cell line containing CXCR4-ADRB2 heteromer by overexpressing both CXCR4 and ADRB2 (“MDA-MB-231-CXCR4-ADRB2 cells”) were prepared and then the amount of CXCR4-ADRB2 heteromer expression was compared through PLA. As shown in FIGS. 20A-20B, there are about 65, about 80, and more than 180, CXCR4-ADRB2 heteromers detected in the MDA-MB-231 parent cells, the MDA-MB-231-CXCR4 cells, and the MDA-MB-231-CXCR4-ADRB2 cells, respectively. To determine whether the (presence and) amount of the CXCR4-ADRB2 heteromer, as verified by PLA at the cellular level, increases tumor growth in a dose dependent manner, MDA-MB-231 parent cells, MDA-MB-231-CXCR4 cells, and MDA-MB-231-CXCR4-ADRB2 cells were prepared and the same amount of cells (5×10⁶ cell/head) were injected orthotopically at mammary fat pad in nude mice to compare tumor growth rates. Shown in FIG. 20C, the tumor size at 67 days after transplantation was 265.8±161.8 mm³ in MDA-MB-231 xenograft mouse (having MDA-MB-231 parent cells), 459.2±399.6 mm³ on MDA-MB-231-CXCR4 xenograft (having MDA-MB-231-CXCR4 cells), and 1107.0±184.8 mm³ on MDA-MB-231-CXCR4-ADRB2 xenograft mouse (having MDA-MB-231-CXCR4-ADRB2 cells). Tumor size was observed in the order of MDA-MB-231-CXCR4-ADRB2 xenograft mouse, MDA-MB-231-CXCR4 xenograft mouse, and MDA-MB-231 xenograft mouse, which indicates that the size of the tumor is increased depending on the amount of CXCR4-ADRB2 heteromer present. These results suggest that as the amount of the CXCR4-ADRB2 heteromer increases, the tumor becomes more malignant. Accordingly, the cancer having the CXCR4-ADRB2 heteromer may be effectively treated through the administration of an inhibitor, or combination of inhibitors, such as a CXCR4 inhibitor and an ADRB2 inhibitor, targeting the CXCR4-ADRB2 heteromer.

### Example 15. The Effect of CXCR4 Inhibitor Alone on Tumor Growth in A549 Xenografted Mouse Containing CXCR4-ADRB2 Heteromer

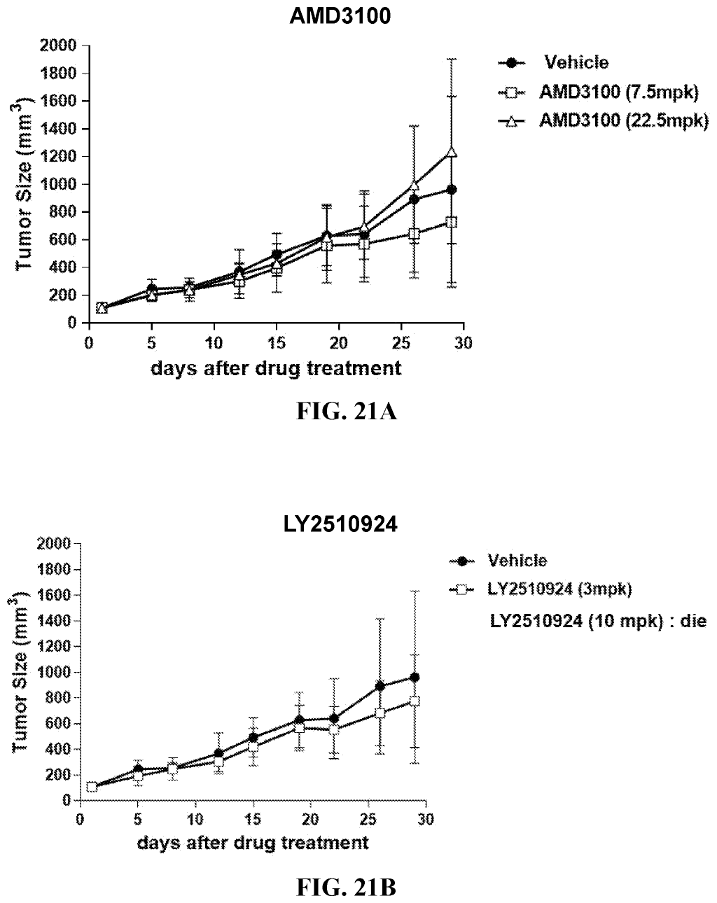

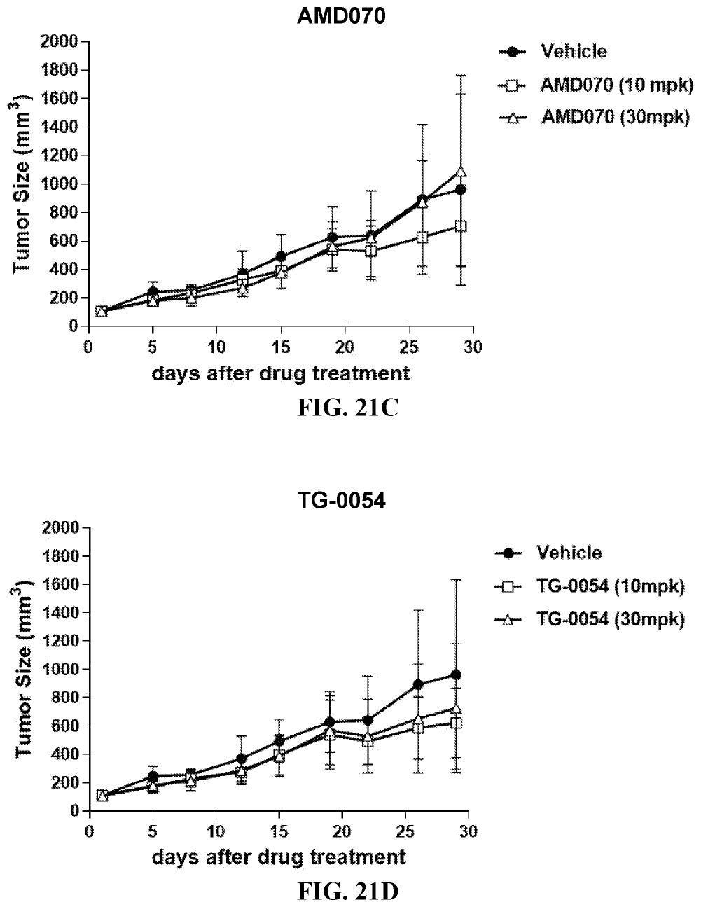

The antitumor effect in mice transplanted with A549 cells containing CXCR4-ADRB2 by stably overexpressing both CXCR4 and ADRB2 (“A549-CXCR4-ADRB2 cells”) was evaluated using CXCR4 inhibitors currently under development or on the market. A549-CXCR4-ADRB2 cells (1×10⁷ cell/head) were injected subcutaneously in nude mice. Upon tumor sizes reaching an average size of about 50-100 mm³, the mice (n=10/test group) were treated with a CXCR4 inhibitor: AMD3100 (FIG. 21A), LY2510924 (FIG. 21B), AMD070 (FIG. 21C), or TG-0054 (FIG. 21D). The CXCR4 inhibitors were administered once a day for 4 weeks.

As shown in FIGS. 21A-21D, most of the CXCR4 inhibitors tested showed limited inhibition of tumor growth. In addition, a dose-dependent tumor decrease was not observed, and the inhibitory effect was insignificant. Moreover, despite the administration of more than the usual dose, tumor growth inhibition was limited. Treatment with LY2510924 at a high dose of 10 mpk resulted in the death of 3 out of 10 mice tested in only 2 doses.

As noted in Example 11, and Tables 3-4, in cells containing the CXCR4-ADRB2 heteromer (having increased Ca 2+ signal), co-treating with a CXCR4 antagonist and an ADRB2 inhibitor is more effective that single treatment with CXCR4 antagonist alone. As noted in the cell growth assay (Example 13, and FIGS. 18A-18E), the CXCR4 inhibitor decreased the increased cell proliferation resulting from CXCL12 stimulation. Increasing the amount of CXCR4 inhibitor resulted in cytotoxicity. These results suggest that co-administration of a combination of a CXCR4 inhibitor with an ADRB2 inhibitor may be more effective than administration of a CXCR4 inhibitor alone in inhibiting the function of CXCR4-ADRB2 heteromers and in inhibiting tumor growth.

### Example 16. The Effect of CXCR4 Inhibitor and ADRB2 Inhibitor Alone or in Combination on Tumor Growth in CXCR4-ADRB2 Heteromer Containing MDA-MB-231 Cell Orthotopic Xenografted Mouse (or A549 Cell Xenograft Mouse)

CXCR4-ADRB2 heteromer containing MDA-MB-231 cell orthotopic xenografted mouse:

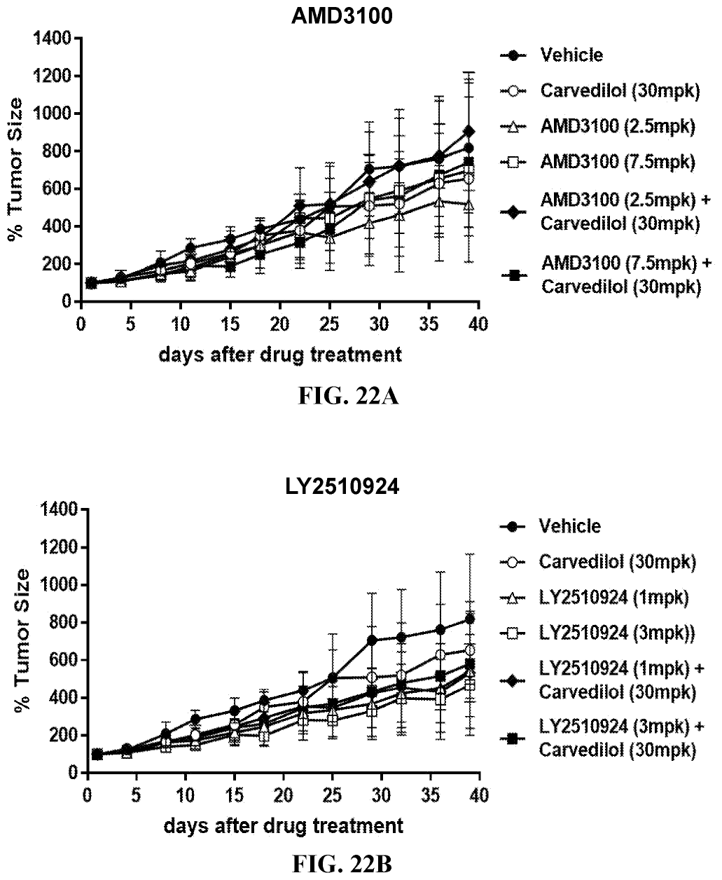

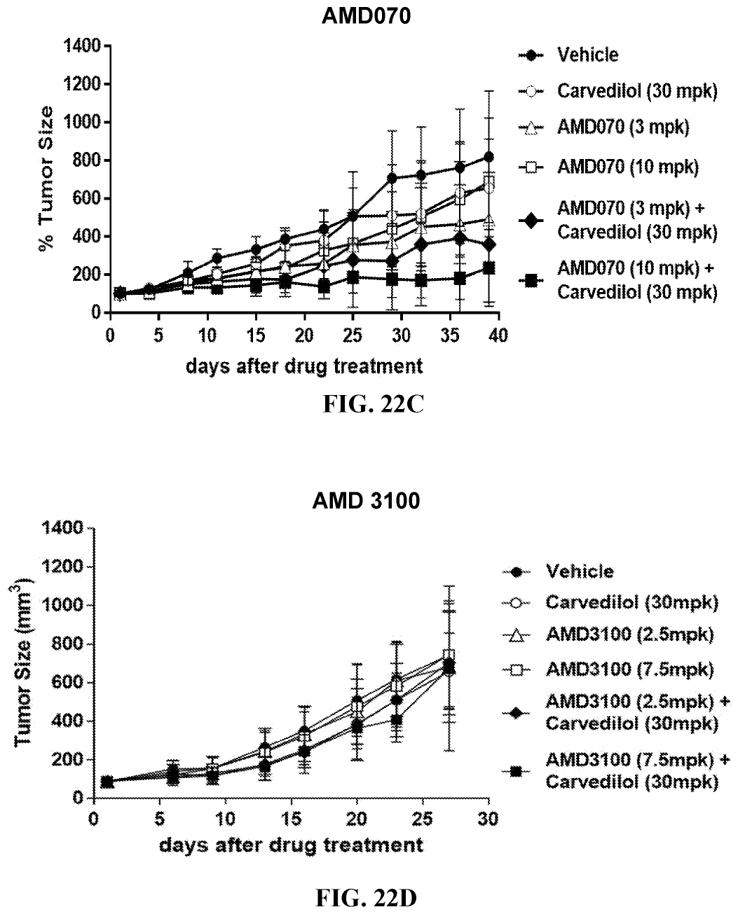

In mice transplanted with MDA-MB-231 cells containing the CXCR4-ADRB2 heteromer by stably overexpressing both CXCR4 and ADRB2 (“MDA-MB-231-CXCR4-ADRB2 cells”), the administration of the CXCR4 inhibitor alone was not an effective antitumor treatment. As shown in FIGS. 22A-22C, inhibition of increased tumor growth due to the presence of the CXCR4-ADRB2 heteromer (antitumor effect) was evaluated by comparing the administering of a CXCR4 inhibitor (AMD3100 (FIG. 22A), LY2510924 (FIG. 22B), AMD070 (FIG. 22C)) and ADRB2 inhibitor (Carvedilol), alone or in combination. Balb/c-nu/nu female mice were orthotopically transplanted with MDA-MB-231-CXCR4-ADRB2 cells (1×10⁶ cell/head). AMD3100 (2.5 mg/kg, 7.5 mg/kg), LY2510924 (1 mg/kg, 3 mg/kg), AMD070 (3 mg/kg, 10 mg/kg) and/or Carvedilol (30 mg/kg) was administered once a day for 4 weeks (28 times in total). AMD3100, LY2510924, and AMD070 were administered subcutaneously, and Carvedilol were administered orally. The size of the tumors was calculated by converting the tumor size to 100 on the first day of drug administration (% tumor growth). As shown in FIG. 22C, the administration of the CXCR4 inhibitor AMD070 alone or the ADRB2 inhibitor Carvedilol alone showed a limited suppression of tumor growth. However, the co-administration of AMD070 with Carvedilol effectively inhibited the growth of the tumor, compared with the respective single administrations, and as the amount of CXCR4 inhibitor AMD070 increased, the growth of the tumor was also dose-dependently decreased when the combination (including carvedilol) was administered. These results suggest that inhibiting the increased size of the tumor due to CXCR4-ADRB2 heteromer formation (antitumor effect) may be provided through co-administration of a CXCR4 inhibitor and an ADRB2 inhibitor.

CXCR4-ADRB2 Heteromer Containing A549 Cell Xenografted Mouse:

To investigate the anti-tumor effect of CXCR4-ADRB2 heteromer inhibitors on tumor growth, A549 cells containing the CXCR4-ADRB2 heteromer by stably overexpressing both CXCR4 and ADRB2 (“A549-CXCR4-ADRB2 cells”), (1×10⁷ cell/head) were injected subcutaneously in nude mice. When tumor sizes were reached an average of 50-100 mm³, CXCR4 inhibitor (AMD3100), ADRB2 inhibitor (carvedilol), alone or in combination, were administered.

FIG. 22D is a graph comparing tumor growth rates for the in vivo antitumor effect of CXCR4 inhibitor AMD3100 (2.5 mg/kg, 7.5 mg/kg) and/or ADRB2 inhibitor Carvedilol (30 mg/kg), administered once a day for 4 weeks (28 times in total) via single administrations or co-administrations; AMD3100 was administered subcutaneously and Carvedilol was administered orally. The tumor growth was monitored every third or fourth day by measuring the length (L) and width (W) of the tumor and calculating tumor volume based on the following formula: Volume=0.5 LW².

### Examples 17A-17B. CXCR4-ADRB2 Heteromer Detection by Ligand Based TR-FRET

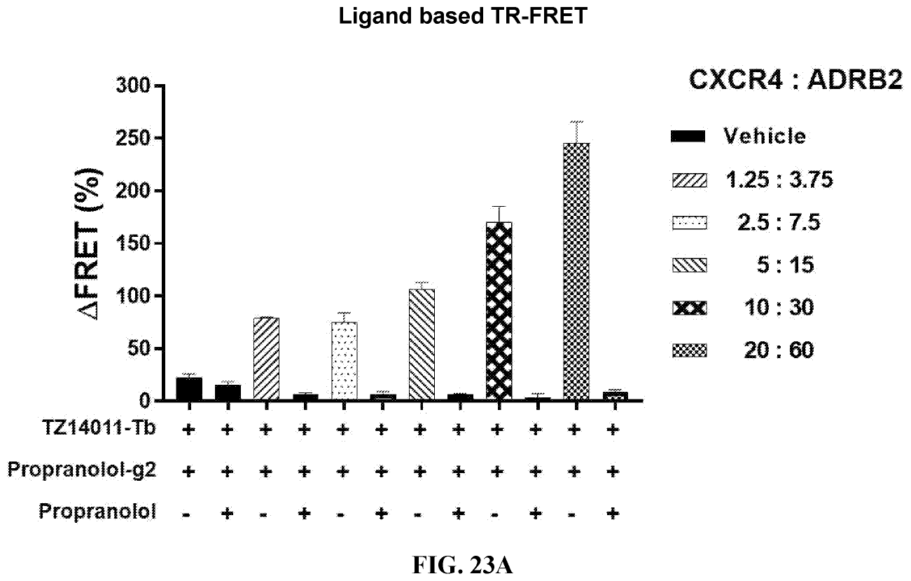

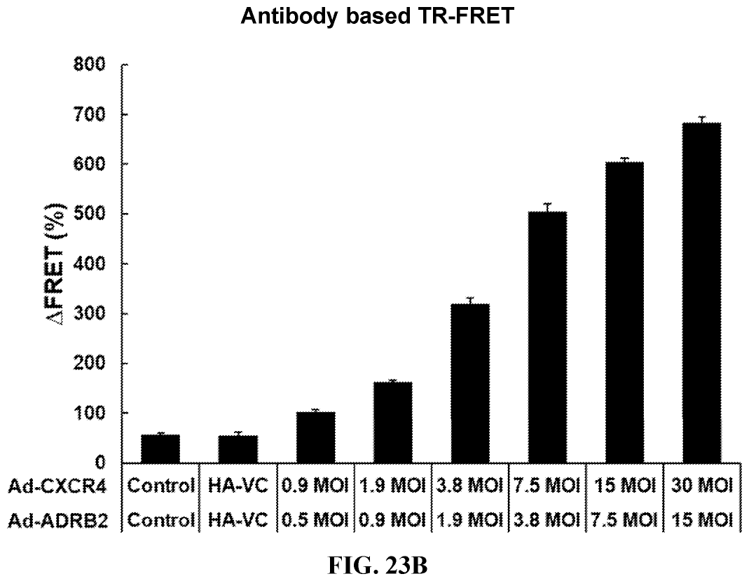

Detection of a CXCR4-ADRB2 heteromer was evaluated using Time-resolved fluorescence energy transfer (TR-FRET). TR-FRET combines the low background aspect of time-resolved fluorometry (TRF) with the homogeneous assay format of FRET. The resulting assay provides an increase in flexibility, reliability and sensitivity FRET involves two fluorophores, a donor and an acceptor. Excitation of the donor by an energy source produces an energy transfer to the acceptor if the two are within a given proximity to each other. The acceptor in turn emits light at its characteristic wavelength. A549 cells were seeded in 96-well plates at a density of 20,000 cells per well. CXCR4 and ADRB2 were transiently over-expressed in A549 cells by adenovirus infection followed by 48 h incubation at 37° C., 5% C02. CXCR4 expressing adenovirus was treated at MOI of 0, 0.1, 0.5, 1.25, 2.5, 5, 10 and 20, and ADRB2 expressing adenovirus was treated at three times higher MOI. Adenoviruses encoding HA-VC were used to adjust the total amount of adenoviruses transduced. To characterize the CXCR4-ADRB2 heteromers, a TR-FRET assay with labeled ligand was performed with fluorescently labeled Propranolol and terbium labeled TZ14011. FIG. 23A shows that the FRET signal is increased depending on the amount of CXCR4 and ADRB2 expression (which impacts the amount of CXCR4-ADRB2 heteromer that forms), and when the unlabeled propranolol is treated as a competitor to the ADRB2 antagonist propranolol-g2, the FRET signal disappears, indicating that it is a CXCR4-ADRB2 specific signal. These results indicate that CXCR4-ADRB2 heteromer can be quantitatively detected by the ligand-based TR-FRET method.

In addition to using a ligand-fluorescence complex in which fluorescence is conjugated to a GPCR-specific ligand, an antibody-fluorescent complex in which fluorescence is conjugated to a GPCR-specific antibody or secondary antibody may be used for TR-FRET.

U2OS cells were seeded in 96-well plates at a density of 20,000 cells per well. After the overnight incubation in the 37° C. humidified incubator, both CXCR4 and ADRB2 were transiently overexpressed using adenoviral system (0.9-30 MOI of CXCR4-carrying adenovirus and 0.5-15 MOI of ADRB2-carrying adenovirus). To analyze a dynamic range and a limit of detection of antibody-based TR-FRET for CXCR4 and ADRB2, wide range of multiplicity of infection (MOI) of CXCR4- and ADRB2-carrying adenovirus (Ad-CXCR4 and Ad-ADRB2, respectively) was adopted. The cells were washed and fixed with 4% paraformaldehyde at room temperature for 10 minutes following the 48 h infection of each adenovirus. After washing the cells twice, to permeabilize the cells, DPBS containing 0.1% Triton X-100 was treated at room temperature for 10 minutes. The cells were then blocked at room temperature for 30 minutes, followed by incubation with both rabbit anti-CXCR4 antibody and mouse anti-ADRB2 antibody at room temperature for 3 hours. After washing the cells with DPBS for 4 times, goat anti-rabbit IgG labeled with terbium cryptate and goat anti-mouse IgG labeled with Alexa Fluor 647 were treated at room temperature for 1 hour. The cells were then washed with DPBS for 4 times, followed by analysis using Varioskan LUX Multimode Microplate Reader.

The performance of antibody-based TR-FRET showed the highest signal in the ratio of 2 Ad-CXCR4:1 Ad-ADRB2 (FIG. 23B). Accordingly, Ad-CXCR4 and Ad-ADRB2 were serially diluted in half from 30 MOI and 15 MOI, respectively. HA-VC-carrying adenovirus was used as a negative control. The FRET efficiency was successfully detected in 0.9 MOI of Ad-CXCR4 and 0.5 MOI of Ad-ADRB2, and a dose-dependency was also observed. These results suggest that antibody-based TR-FRET may be a promising tool for GPCR heteromer diagnostics.

### Examples 18A-18C. CXCR4-ADRB2 Heteromer Detection by PLA and Quantification of CXCR4 or ADRB2 RNA Expression Level by RT-qPCR in Blood Cancer and Solid Tumor Cell Lines

The relationship between the amount of RNA produced by individual GPCRs, CXCR4 and ADRB2, and the amount of CXCR4-ADRB2 heteromers detected in blood cancer and solid tumor cell lines was examined. CXCR4 is overexpressed in a variety of human cancers, for example, hematological cancers such as myeloma and leukemia, and solid tumors, such as lung cancer and breast carcinoma. And this overexpression is correlated with increased risk for recurrence and poor overall survival.

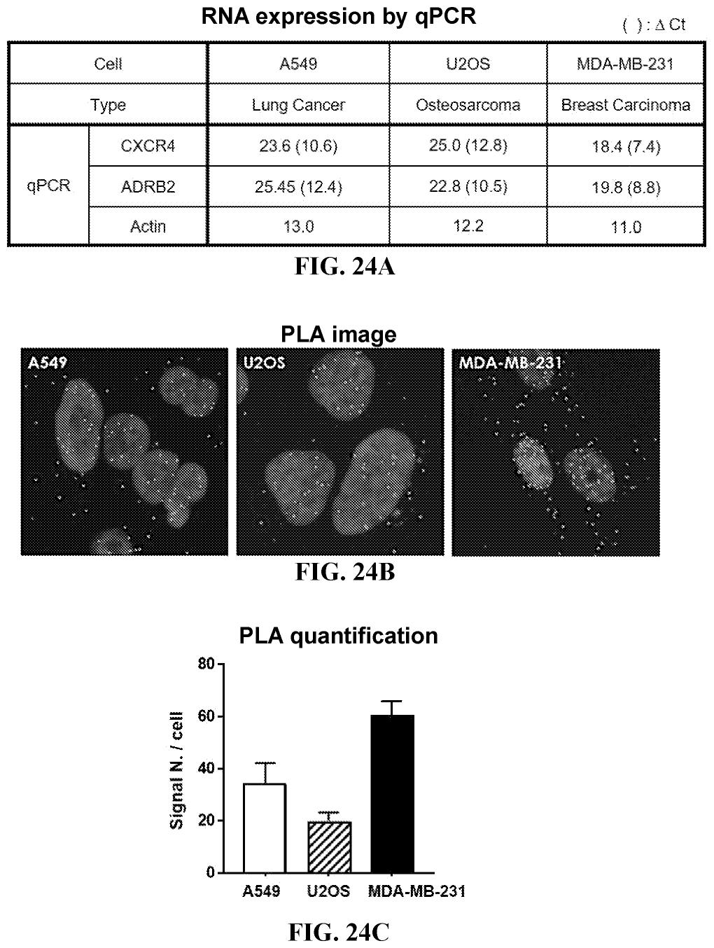

The expression of CXCR4 and ADRB2 RNA was observed in all three solid tumor cell lines tested by qPCR: A549 (lung cancer), U2OS (osteosarcoma), and MDA-MB-231 (breast carcinoma) (FIG. 24A). To evaluate the endogenous expression level of CXCR4-ADRB2 heteromers in the A549, U2OS, and MDA-MB-231 cells, PLA was utilized using two different primary antibodies for CXCR4 and ADRB2. Resulting rolling circle amplification (RCA) products were shown as red fluorescence signal, widely stained throughout the cell surface, covering the nucleus (stained with DAPI) and the cytosol (FIG. 24B). Mean PLA signal for each cell was displayed as RCA products divided by cell number. In average, each MDA-MB-231 cell had the value of about 60 RCA products followed by A549 cells having about 35 RCA products, and U2OS cells having about 20 RCA products (FIG. 24C). The RNA expression level of CXCR4 and ADRB2 was highest in MDA-MB-231 cell line compared to that of A549 and U2OS cells (delta Ct of CXCR4/ADRB2: 7.4/8.8, 10.6/12.4, 12.8/10.5), and the PLA value was also highest in MDA-MB-231 compared to A549 or U2OS cell. It can be seen that there is a significant correlation between RNA expression and PLA values. These results indicate that patients with CXCR4-ADRB2 heteromer can be screened by analysis of PLA and RNA expression levels.

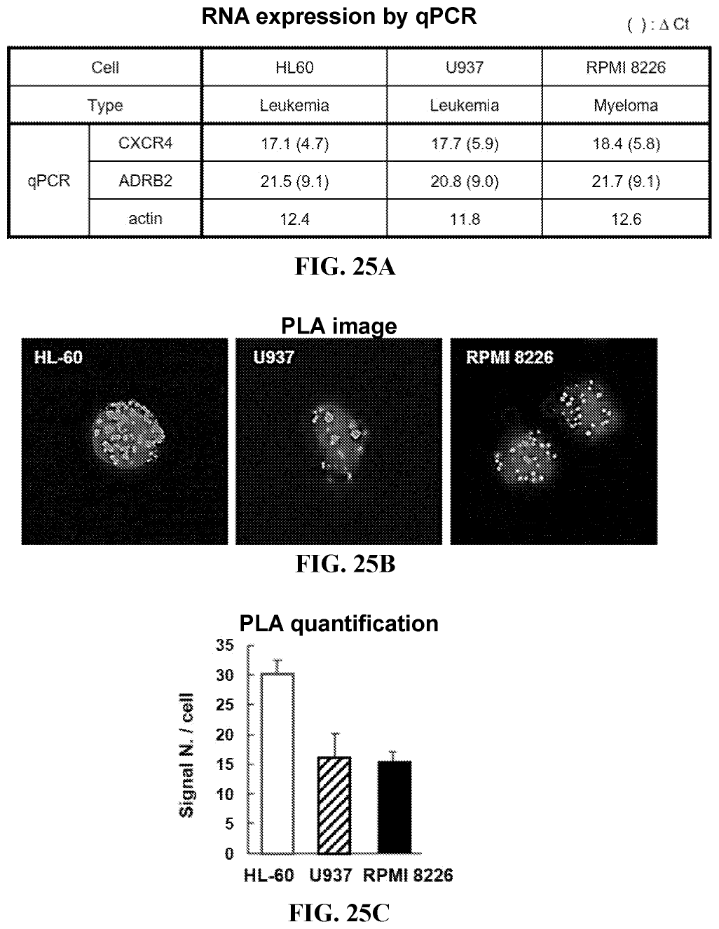

The expression of CXCR4 and ADRB2 RNA was observed in all three blood cancer cell lines tested by qPCR: HL60 (leukemia), U937 (leukemia), and RPMI 8226 (myeloma) (FIG. 25A). To evaluate the endogenous expression level of CXCR4-ADRB2 heteromers in the HL-60, U937 and RPMI 8226 cells, PLA was utilized using two different primary antibodies for CXCR4 and ADRB2. Resulting rolling circle amplification (RCA) products were shown as red fluorescence signal stained throughout the cell surface (FIG. 25B). Mean PLA signal for each cell was displayed as RCA products divided by cell number. In average, each HL-60 cell had the value of 30 RCA products followed by both U937 and RPMI 8226 cells, which were around half of the value of HL-60 (FIG. 25C). Comparing the RNA expression levels of CXCR4 and ADRB2 by quantitative PCR, ADRB2 expression was similar in the three cells (delta Ct: 9), but the expression level of CXCR4 in HL60 cells was twice that of the other cell lines U937 and RPMI 8226 (delta Ct values: 4.7, 5.9, 5.8). These differences show a similar pattern in the PLA values, indicating a significant correlation between RNA expression level and PLA values. These results show that the PLA signal of suspension cells is able to be successfully detected, showing that HL-60 cells have twice as many CXCR4-ADRB2 heteromers as U937 and RPMI 8226 cells.

The diagnosis of CXCR4 heteromer is essential for screening subjects who express CXCR4 heteromers and supplying selected drugs according to the type of partner GPCR. The PLA, which has already been set up in our institute as a method to diagnose CXCR4 heteromer, is a method of diagnosing the tissue extracted from solid cancer subjects using the antibody in the formalin fixed paraffin embedded (FFPE) sample. These results provide a possibility to detect CXCR4 heteromers in liquid biopsies such as blood cancer subject samples, urine, and sediment.

### Example 19. Evaluation by Ca2+ Mobilization Assay of Enhanced CXCR4 Downstream Signaling in Native Cells Endogenously Expressing CXCR4 and ADRB2 Protomers

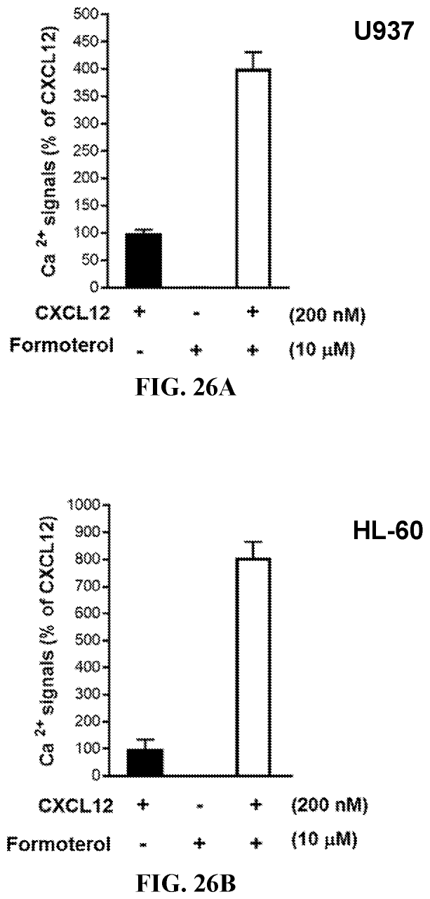

The properties of a CXCR4-GPCRx heteromer, such as a CXCR4-ADRB2 heteromer were evaluated via the calcium mobilization assay in U937 cells (FIG. 26A) and HL-60 cells (FIG. 26B), each endogenously expressing both CXCR4 and ADRB2, by measuring the resulting calcium signaling upon stimulation or co-stimulation with one or both of their respective agonists. Calcium mobilization was measured using FlexStation3.

In both U937 cells (FIG. 26A) and HL-60 cells (FIG. 26B), stimulation with the CXCR4 agonist CXCL12 alone (at 200 nM) evoked an intercellular calcium mobilization, while stimulation with the ADRB2 agonist formoterol alone (at 10 μM) did not induce a calcium response. However, co-stimulation with both agonists (CXCL12 and formoterol, at 200 nM and 10 μM, respectively) produced a significantly increased calcium response in both U937 cells (FIG. 26A) and HL-60 cells (FIG. 26B), relative to the response evoked by mono-agonist stimulation (about 4 fold increased calcium response in U937 cells, relative to mono-stimulation with CXCL12, and about 8 fold increased calcium response in HL-60 cells, relative to mono-stimulation with CXCL12).

### Example 20. Ca2+ Mobilization Inhibition of Enhanced CXCR4 Downstream Signaling Upon CXCR4-ADRB2 Heteromer Formation in U937 and HL-60 Cell Lines—Comparison of Single Inhibitor Treatment to Combination Inhibitor Treatment

The efficiency of CXCR4 inhibitors in suppressing the increased calcium response of CXCR4-ADRB2 heteromers in both U937 cells and HL-60 cells (Example 19) was measured in the presence or absence of carvedilol, a representative ADRB2 inhibitor. As discussed in Example 19, the increased calcium response of the CXCR4-ADRB2 heteromers were induced by agonists CXCL12 (200 nM) and formoterol (10 μM), and the CXCR4 inhibitors (in the presence or absence of the ADRB2 inhibitor carvedilol) were added 2 hours before treating the cells with the agonists. Calcium mobilization was measured using FlexStation3, and the resulting IC50 of Ca2+ response were calculated using GraphPad Prism software. The data is shown in Table 5.

TABLE 5

| | CXCR4 inhibitor IC₅₀ [nM] | | | |
|---|---|---|---|---|
| CXCR4 | U937 | | HL60 | |
| inhibitor | +Carvedilol 10 uM | −Carvedilol 10 uM | +Carvedilol 10 uM | −Carvedilol 10 uM |
| AMD3100 | 0.52 ± 0.06 | 3.34 ± 2.45 | 0.34 ± 0.31 | 1.50 ± 1.13 |
| Ulocuplumab | 0.26 ± 0.21 | 1.25 ± 0.44 | 0.006 ± 0.003 | 0.02 ± 0.003 |
| AMD070 | 4.08 ± 2.75 | 4.83 ± 0.24 | 0.28 ± 0.16 | 3.56 ± 3.17 |
| TG-0054 | 0.35 ± 0.23 | 24 ± 12.6 | 0.32 ± 0.11 | 4 ± 1.98 |

As shown in Table 5, in U937 cells, CXCR4 inhibitors AMD3100, ulocuplumab and TG-0054 suppressed the CXCR4-ADRB2 signaling more efficiently in the presence of 10 μM of carvedilol as shown with the reduced IC₅₀ values of the respective CXCR4 inhibitors. In particular, the IC50 value of TG-0054 was reduced significantly in the presence of carvedilol. The IC50 value of Ca2+ response decreased about 6.4 fold (from 3.34 nM to 0.52 nM), about 4.8 fold (from 1.25 nM to 0.26 nM) and about 69 fold (from 24 nM to 0.35 nM) upon co-treatment of carvedilol with AMD3100, ulocuplumab, or TG-0054, respectively.

As shown in Table 5, in HL-60 cells, each of the CXCR4 inhibitors suppressed the CXCR4-ADRB2 signaling more efficiently in the presence of 10 μM of Carvedilol. The IC50 value of Ca2+ response decreased about 4.4 fold (from 1.50 nM to 0.34 nM), about 3.3 fold (from 0.02 nM to 0.006 nM), about 12.7 fold (from 3.56 nM to 0.28 nM) and about 12.5 fold (from 4 nM to 0.32 nM), upon co-treatment of carvedilol with AMD3100, ulocuplumab, AMD070 and TG-0054, respectively.

These results suggest that co-treatment with a CXCR4 inhibitor and an ADRB2 inhibitor together inhibits increased Ca2+ response more effectively than single treatment with CXCR4 inhibitor alone in cells containing the CXCR4-ADRB2 heteromer.

### Example 21. Ca2+ Mobilization Inhibition of Enhanced CXCR4 Downstream Signaling Upon CXCR4-ADRB2 Heteromer Formation in NIDA-IB-231 Cell Line—Comparison of Single Inhibitor Treatment to Combination Inhibitor Treatment

The efficiency of ADRB2 inhibitors in suppressing the calcium response of ADRB2 was measured in MDA-MB-231 cells transduced with adenovirus encoding ADRB2 only, and the signaling was measured upon stimulating the cells with salmeterol (1 μM). The data is shown in Table 6. In addition, the efficiency of ADRB2 inhibitors in suppressing the increased calcium response (enhanced signaling) of CXCR4-ADRB2 heteromers in MDA-MB-231 cells co-transduced with adenoviruses encoding CXCR4 and ADRB2 was measured in the presence or absence of AMD3100, a representative CXCR4 inhibitor. Ca2+ flux was measured upon co-stimulating the MDA-MB-231 cells with CXCL12 (20 nM) and salmeterol (1 μM), and the ADRB2 inhibitors (in the presence or absence of the CXCR4 inhibitor AMD3100) were added 2 hours before treating the cells with the agonists. Calcium mobilization was measured using FlexStation3, and the resulting IC50 values of calcium response were calculated using GraphPad Prism software. The data is shown in Table 6.

TABLE 6

CXCR4 + ADRB2

| ADRB2 | ADRB2 | +AMD3100 | −AMD3100 |
|---|---|---|---|
| inhibitor | only | (650 nM) | (650 nM) |
| Bupranolol | 0.82 ± 0.72 | 0.10 ± 0.05 | 3.12 ± 2.27 |
| Labetalol | 24.81 ± 24.87 | 0.54 ± 0.6 | 57.98 ± 49.24 |

As shown in Table 6, in MDA-MB-231 cells, single treatment of bupranolol inhibited ADRB2 signaling with 0.82 nM as the IC50 value, and single treatment of labetalol inhibited ADRB2 signaling with 24.81 nM as the IC50 value. ADRB2 inhibitors, bupranolol or labetalol, suppressed the CXCR4-ADRB2 signaling more efficiently with greater potency in the presence of 650 nM (IC90) of AMD3100 as shown with the reduced IC50 values, relative to the IC50 values of ADRB2 inhibitors in the absence of AMD3100. The value of IC50 of bupranolol decreased by about 33 times in combination with AMD3100 (from 3.12 nM to 0.10 nM), relative to a single treatment, while the IC50 of labetalol decreased by about 107 times in combination with AMD3100 (from 57.98 nM to 0.54 nM), relative to a single treatment. These results suggest that co-treatment with a CXCR4 inhibitor and an ADRB2 inhibitor together inhibits increased Ca2+ response more effectively than single treatment with ADRB2 inhibitor alone in cells containing the CXCR4-ADRB2 heteromer.

### Example 22. Ca2+ Mobilization Inhibition of Enhanced CXCR4 Downstream Signaling Upon CXCR4-ADRB2 Heteromer Formation in NIDA-IB-231 Cell Line—Comparison of TG-0054 Single Treatment to Combination Inhibitor Treatment

The efficiency of TG-0054, a CXCR4 inhibitor, in suppressing the response of CXCR4 was measured in MDA-MB-231 cells transduced with adenovirus encoding CXCR4 only, and the signaling was measured upon stimulating the cells with CXCL12 (20 nM). In MDA-MB-231 cells (expressing CXCR4), TG-0054 was measured to have an IC50 value (nM) of 65.78±1.34. Next, the efficiency of TG-0054 in suppressing the enhanced signaling of CXCR4-ADRB2 heteromer was measured in MDA-MB-231 cells co-transduced with adenoviruses encoding CXCR4 and ADRB2 in the presence or absence of ADRB2 inhibitors (at 10 μM). The data is shown in Table 7. Ca2+ flux was measured upon co-stimulating the MDA-MB-231 cells with CXCL12 (20 nM) and salmeterol (1 μM), and the CXCR4 inhibitor TG-0054 (in the presence or absence of the ADRB2 inhibitors) were added 2 hours before treating the cells with the agonists. Calcium mobilization was measured using FlexStation3, and the resulting IC50 values of calcium response were calculated using GraphPad Prism software. The data is shown in Table 7.

TABLE 7

| TG-0054 + ADRB2 inhibitor | TG-0054 IC₅₀ [nM] |
|---|---|
| −(TG-0054 only) | 92.89 ± 31.27 |
| Carvedilol | 0.02 ± 0.013 |
| Labetalol | 0.026 ± 0.014 |
| Alprenolol | 9.19 ± 5.94 |
| Carazolol | 6.98 ± 0.05 |
| Propafenone | 6.53 ± 1.67 |
| Timolol | 11.02 ± 8.06 |

As noted above, in MDA-MB-231 cells, single treatment of TG-0054 inhibited CXCR4 signaling with 65.78 nM as the IC50 value in CXCR4 only transduced MDA-MB-231. As shown in Table 7, TG-0054 suppressed the CXCR4-ADRB2 signaling more efficiently with great potency in the presence of ADRB2 inhibitors, as shown with the reduced IC50 values relative to single treatment in the absence of an ADRB2 inhibitor. The IC50 value of TG-0054 decreased by about 4645 times in combination with carvedilol (from 92.89 nM to 0.02 nM), decreased by about 3573 times in combination with labetalol (from 92.89 nM to 0.026 nM), and decreased by about 10 times or more when treated in combination with any one of ADRB2 inhibitors alprenolol, carazolol, propafenone, or timolol. These results suggest that co-treating with TG-0054 and an ADRB2 inhibitor together more effectively inhibits increased Ca2+ response than single treatment with TG-0054 alone in cells containing the CXCR4-ADRB2 heteromer.

### Exemplary Embodiments

Embodiment A1. A method for suppressing enhanced downstream signaling resulting from a CXCR4-ADRB2 heteromer in a cell of a subject suffering from cancer, the method comprising administering to the subject:
a) a CXCR4 inhibitor, wherein the CXCR4 inhibitor is burixafor; and
b) an ADRB2 inhibitor;
   wherein:
i) the enhanced downstream signaling results from the CXCR4-ADRB2 heteromer; and
ii) the administered burixafor and ADRB2 inhibitor suppresses the enhanced downstream signaling from said CXCR4-ADRB2 heteromer in the cancer subject.

Embodiment A2. A method for treating cancer in a subject having a cell containing a CXCR4-ADRB2 heteromer, the method comprising administering to the subject:
a) a CXCR4 inhibitor, wherein the CXCR4 inhibitor is burixafor; and
b) an ADRB2 inhibitor;
   wherein:
i) enhanced downstream signaling results from the CXCR4-ADRB2 heteromer; and
ii) the administered burixafor and ADRB2 inhibitor suppresses the enhanced downstream signaling from said CXCR4-ADRB2 heteromer in the cancer subject.

Embodiment A3. A method for treating cancer in a subject having a cell containing a CXCR4-ADRB2 heteromer, the method comprising:
a) determining whether the subject's cell contains a CXCR4-ADRB2 heteromer, wherein enhanced downstream signaling results from the CXCR4-ADRB2 heteromer; and
b) if the subject's cell contains said CXCR4-ADRB2 heteromer, then administering to the cancer subject:
   i) a CXCR4 inhibitor, wherein the CXCR4 inhibitor is burixafor; and
   ii) an ADRB2 inhibitor.

Embodiment A4. A method for treating cancer in a subject having a cell containing a CXCR4-ADRB2 heteromer, wherein enhanced downstream signaling results from the CXCR4-ADRB2 heteromer, the method comprising:
1) determining whether the subject has the cell containing the CXCR4-ADRB2 heteromer by: obtaining or having obtained a biological sample from the subject; and performing or having performed an assay on the biological sample to determine if:
   i) the subject's cell contains said CXCR4-ADRB2 heteromer; or
   ii) a combination of a CXCR4 inhibitor and an ADRB2 inhibitor: alters heteromer-specific properties or function of said CXCR4-ADRB2 heteromer in the subject's derived cell(s); alters heteromer-specific properties of the subject's derived cell(s) containing said CXCR4-ADRB2 heteromer; or decreases cancer progression in the subject having cells containing said CXCR4-ADRB2 heteromer; and
2) if the subject has a cell containing said CXCR4-ADRB2 heteromer, then administering to the cancer subject a combination of a CXCR4 inhibitor and an ADRB2 inhibitor, wherein the CXCR4 inhibitor is burixafor; and
3) if the subject does not have a cell containing said CXCR4-ADRB2 heteromer, then administering to the cancer subject a single inhibitor of either the burixafor or the ADRB2 inhibitor.

Embodiment A5. A method for treating cancer in a subject having a cell containing a CXCR4-ADRB2 heteromer, wherein enhanced downstream signaling results from the CXCR4-ADRB2 heteromer, the method comprising:
1) determining whether the subject has the cell containing the CXCR4-ADRB2 heteromer by: obtaining or having obtained a biological sample from the subject; and performing or having performed an assay on the biological sample to determine if:
   i) the subject's cell contains said CXCR4-ADRB2 heteromer; or
   ii) a combination of a CXCR4 inhibitor and an ADRB2 inhibitor: alters heteromer-specific properties or function of said CXCR4-ADRB2 heteromer in the subject's derived cell(s); alters heteromer-specific properties of the subject's derived cell(s) containing said CXCR4-ADRB2 heteromer; or decreases cancer progression in the subject having cells containing said CXCR4-ADRB2 heteromer; and
2) if the subject has a cell containing said CXCR4-ADRB2 heteromer, then administering to the cancer subject a combination of a CXCR4 inhibitor and an ADRB2 inhibitor, wherein the CXCR4 inhibitor is burixafor; and
3) if the subject does not have a cell containing said CXCR4-ADRB2 heteromer, then administering to the cancer subject a single inhibitor of either the burixafor or the ADRB2 inhibitor;
   wherein:
a) progression of the cancer in the subject having said cell containing the CXCR4-ADRB2 heteromer is decreased in the range of 5-100% more upon administering to the cancer subject the combination of the burixafor and the ADRB2 inhibitor, relative to administering the single inhibitor of either the burixafor or the ADRB2 inhibitor;
b) efficacy of the burixafor is increased in the range of 5-2000% when administered in combination with the ADRB2 inhibitor to the subject having said cell containing the CXCR4-ADRB2 heteromer, relative to efficacy of the burixafor when administered as a single inhibitor; and/or
c) efficacy of the ADRB2 inhibitor is increased in the range of 5-2000% when administered in combination with the burixafor to the subject having said cell containing the CXCR4-ADRB2 heteromer, relative to efficacy of the ADRB2 inhibitor when administered as a single inhibitor.

Embodiment A6. A method for treating cancer in a subject having a cell containing a CXCR4-ADRB2 heteromer, wherein enhanced downstream signaling results from the CXCR4-ADRB2 heteromer, the method comprising:
1) determining whether the subject's cell contains the CXCR4-ADRB2 heteromer by: obtaining or having obtained a biological sample from the subject and performing or having performed an assay on the biological sample to determine if said CXCR4-ADRB2 heteromer is present in the subject's cell; wherein the assay performed on the biological sample is or comprises one or more of the following: a co-internalization assay, a colocalization assay, in situ hybridization, immunohistochemistry, immunoelectron microscopy, a proximity-based assay, a co-immunoprecipitation assay, enzyme-linked immunosorbent assay (ELISA), flow cytometry, RNAseq, RT-qPCR, expression level of CXCR4, expression level of ADRB2, expression level of CXCR4 and ADRB2, microarray, or a fluorescent animal assay; and
2) if the subject's cell contains said CXCR4-ADRB2 heteromer, then administering to the cancer subject a combination of a CXCR4 inhibitor and an ADRB2 inhibitor, wherein the CXCR4 inhibitor is burixafor; and
3) if the subject's cell does not contain said CXCR4-ADRB2 heteromer, then administering to the cancer subject a single inhibitor of either the burixafor or the ADRB2 inhibitor.

Embodiment A7. A method for treating cancer in a subject having a cell containing a CXCR4-ADRB2 heteromer, wherein enhanced downstream signaling results from the CXCR4-ADRB2 heteromer, the method comprising:
1) determining whether the subject's cell contains the CXCR4-ADRB2 heteromer by: obtaining or having obtained a biological sample from the subject and performing or having performed an assay on the biological sample to determine if said CXCR4-ADRB2 heteromer is present in the subject's cell; wherein the assay performed on the biological sample is or comprises one or more of the following: a co-internalization assay, a colocalization assay, in situ hybridization, immunohistochemistry, immunoelectron microscopy, a proximity-based assay, a co-immunoprecipitation assay, enzyme-linked immunosorbent assay (ELISA), flow cytometry, RNAseq, RT-qPCR, expression level of CXCR4, expression level of ADRB2, expression level of CXCR4 and ADRB2, microarray, or a fluorescent animal assay; and
2) if the subject's cell contains said CXCR4-ADRB2 heteromer, then administering to the cancer subject a combination of a CXCR4 inhibitor and an ADRB2 inhibitor, wherein the CXCR4 inhibitor is burixafor; and
3) if the subject's cell does not contain said CXCR4-ADRB2 heteromer, then administering to the cancer subject a single inhibitor of either the burixafor or the ADRB2 inhibitor:
   wherein:
a) progression of the cancer in the subject having said cell containing the CXCR4-ADRB2 heteromer is decreased in the range of 5-100% more upon administering to the cancer subject the combination of the burixafor and the ADRB2 inhibitor, relative to administering the single inhibitor of either the burixafor or the ADRB2 inhibitor;
b) efficacy of the burixafor is increased in the range of 5-2000% when administered in combination with the ADRB2 inhibitor to the subject having said cell containing the CXCR4-ADRB2 heteromer, relative to efficacy of the burixafor when administered as a single inhibitor; and/or
c) efficacy of the ADRB2 inhibitor is increased in the range of 5-2000% when administered in combination with the burixafor to the subject having said cell containing the CXCR4-ADRB2 heteromer, relative to efficacy of the ADRB2 inhibitor when administered as a single inhibitor.

Embodiment A8. A pharmaceutical kit for use in treating cancer in a subject having a cell containing a CXCR4-ADRB2 heteromer, the pharmaceutical kit comprising:
a) a CXCR4 inhibitor, wherein the CXCR4 inhibitor is burixafor; and
b) an ADRB2 inhibitor;
   wherein enhanced downstream signaling results from the CXCR4-ADRB2 heteromer.

Embodiment A9. A pharmaceutical composition for use in treating cancer in a subject having a cell containing a CXCR4-ADRB2 heteromer, the pharmaceutical composition comprising:
a) a CXCR4 inhibitor, wherein the CXCR4 inhibitor is burixafor;
b) an ADRB2 inhibitor; and
c) a pharmaceutically acceptable carrier;
   wherein enhanced downstream signaling results from the CXCR4-ADRB2 heteromer.

Embodiment A10. A method for suppressing enhanced downstream signaling resulting from a CXCR4-ADRB2 heteromer in a cell, the method comprising administering to the cell:
a) a CXCR4 inhibitor, wherein the CXCR4 inhibitor is burixafor; and
b) an ADRB2 inhibitor;
   wherein:
i) the enhanced downstream signaling results from the CXCR4-ADRB2 heteromer; and
ii) the contacting with the burixafor and the ADRB2 inhibitor suppresses the enhanced downstream signaling from said CXCR4-ADRB2 heteromer in the cell.

Embodiment A11. The method for suppressing of embodiment A10, wherein the method further comprises determining whether the cell contains the CXCR4-ADRB2 heteromer.

Embodiment A12. A pharmaceutical kit for use in suppressing enhanced downstream signaling resulting from a CXCR4-ADRB2 heteromer in a cell, the pharmaceutical kit comprising:
a) a CXCR4 inhibitor, wherein the CXCR4 inhibitor is burixafor; and
b) an ADRB2 inhibitor;
   wherein enhanced downstream signaling results from the CXCR4-ADRB2 heteromer.

Embodiment A13. A pharmaceutical composition for use suppressing enhanced downstream signaling resulting from a CXCR4-ADRB2 heteromer in a cell, the pharmaceutical composition comprising:
a) a CXCR4 inhibitor, wherein the CXCR4 inhibitor is burixafor;
b) an ADRB2 inhibitor; and
c) a pharmaceutically acceptable carrier;
   wherein enhanced downstream signaling results from the CXCR4-ADRB2 heteromer.

Embodiment A14. The method for suppressing of any one of embodiments A10-A13, wherein the cell is a subject's cell.

Embodiment A15. The method for suppressing of embodiment A14, wherein the subject's cell is a cancer cell.

Embodiment A16. The method for suppressing of any one of embodiments A1-A13, wherein the cell is a cancer cell.

Embodiment A17. The method for suppressing, the method for treating, the pharmaceutical kit for use, or the pharmaceutical composition for use, of any one of embodiments A1-A9, wherein the method further comprises detecting the presence of the CXCR4-ADRB2 heteromer in the cancer subject.

Embodiment A18. The method for suppressing, the method for treating, the pharmaceutical kit for use, or the pharmaceutical composition for use, of any one of embodiments A1-A9 or A17, wherein the method further comprises identifying the CXCR4-ADRB2 heteromer in the cancer subject.

Embodiment A19. The method for suppressing, the method for treating, the pharmaceutical kit for use, or the pharmaceutical composition for use, of any one of embodiments A1-A9 or A17-A18, wherein the method further comprises obtaining a biological sample from the subject.

Embodiment A20. The method for suppressing, the method for treating, the pharmaceutical kit for use, or the pharmaceutical composition for use, of any one of embodiments A1-A9 or A17-A19, wherein the method further comprises performing an assay on the biological sample obtained from said subject.

Embodiment A21. The method for suppressing, the method for treating, the pharmaceutical kit for use, or the pharmaceutical composition for use, of any one of embodiments A1-A9 or A17-A20, wherein the method further comprises:
i) obtaining or having obtained a biological sample from the cancer subject;
ii) conducting or having conducted a diagnostic assay to determine presence, identity, or presence and identity, of a CXCR4-ADRB2 heteromer in the obtained biological sample from the cancer subject; and
iii) selecting the ADRB2 inhibitor to administer in combination with burixafor to suppress the enhanced downstream signaling resulting from the CXCR4-ADRB2 heteromer.

Embodiment A22. The method for suppressing, the method for treating, the pharmaceutical kit for use, or the pharmaceutical composition for use, of any one of embodiments A1-A9 or A17-A21, wherein the method further comprises determining whether the subject's cell contains the CXCR4-ADRB2 heteromer, comprising performing an assay on a biological sample obtained from the subject.

Embodiment A23. The method for suppressing, the method for treating, the pharmaceutical kit for use, or the pharmaceutical composition for use, of any one of embodiments A1-A9 or A17-A22, wherein the method further comprises determining whether the subject's cell contains the CXCR4-ADRB2 heteromer, comprising obtaining a biological sample from the subject, and performing an assay on the biological sample obtained from said subject.

Embodiment A24. The method for suppressing, the method for treating, the pharmaceutical kit for use, or the pharmaceutical composition for use, of any one of embodiments A1-A9 or A17-A23, wherein the biological sample obtained from said subject contains the CXCR4-ADRB2 heteromer.

Embodiment A25. The method for suppressing, the method for treating, the pharmaceutical kit for use, or the pharmaceutical composition for use, of any one of embodiments A1-A9 or A14-A24, wherein the subject's cell contains the CXCR4-ADRB2 heteromer.

Embodiment A26. The method for suppressing, the method for treating, the pharmaceutical kit for use, or the pharmaceutical composition for use, of any one of embodiments A1-A25, wherein the CXCR4-ADRB2 heteromer has two or more of the following characteristics:
1) the CXCR4-ADRB2 heteromer components colocalize and physically interact in the cell, either directly or via intermediate proteins acting as conduits for allosterism;
2) an enhanced downstream signaling results from the CXCR4-ADRB2 heteromer; and/or
3) a combination of burixafor and an ADRB2 inhibitor:
   i) alters heteromer-specific properties of the CXCR4-ADRB2 heteromer in the cell;
   ii) alters heteromer-specific function of the CXCR4-ADRB2 heteromer in the cell; and/or
   iii) alters heteromer-specific properties of the cell containing the CXCR4-ADRB2 heteromer.

Embodiment A27. The method for suppressing, the method for treating, the pharmaceutical kit for use, or the pharmaceutical composition for use, of any one of embodiments A1-A9 or A14-A25, wherein the CXCR4-ADRB2 heteromer has two or more of the following characteristics:
1) the CXCR4-ADRB2 heteromer components colocalize and physically interact in the cell, either directly or via intermediate proteins acting as conduits for allosterism;
2) an enhanced downstream signaling results from the CXCR4-ADRB2 heteromer; and/or
3) a combination of burixafor and an ADRB2 inhibitor:
   i) alters heteromer-specific properties of the CXCR4-ADRB2 heteromer in the subject's derived cell(s);
   ii) alters heteromer-specific function of the CXCR4-ADRB2 heteromer in the subject's derived cell(s);
   iii) alters heteromer-specific properties of the subject's derived cell(s) containing the CXCR4-ADRB2 heteromer; and/or
   iv) decreases cancer progression in the subject having cells containing said CXCR4-ADRB2 heteromer.

Embodiment A28. The method for suppressing, the method for treating, the pharmaceutical kit for use, or the pharmaceutical composition for use, of any one of embodiments A1-A27, wherein the CXCR4-ADRB2 heteromer is characterized as having the following characteristic: the CXCR4-ADRB2 heteromer components in the cell colocalize and physically interact, either directly or via intermediate proteins acting as conduits for allosterism.

Embodiment A29. The method for suppressing, the method for treating, the pharmaceutical kit for use, or the pharmaceutical composition for use, of any one of embodiments A1-A28, wherein the CXCR4-ADRB2 heteromer components in the cell colocalize and physically interact, either directly or via intermediate proteins acting as conduits for allosterism, is determined by one or more of the following: a co-internalization assay, a colocalization assay, in situ hybridization, immunohistochemistry, immunoelectron microscopy, a proximity-based assay, a co-immunoprecipitation assay, enzyme-linked immunosorbent assay (ELISA), flow cytometry, RNAseq, RT-qPCR, expression level of CXCR4, expression level of ADRB2, expression level of CXCR4 and ADRB2, microarray, or a fluorescent animal assay.

Embodiment A30. The method for suppressing, the method for treating, the pharmaceutical kit for use, or the pharmaceutical composition for use, of any one of embodiments A1-A29, wherein the proximity-based assay is, or comprises, resonance energy transfer (RET), bioluminescence RET (BRET), fluorescence RET (FRET), time-resolved fluorescence RET (TR-FRET), antibody-based FRET, ligand-based FRET, bimolecular fluorescence complementation (BiFC), or a proximity ligation assay (PLA).

Embodiment A31. The method for suppressing, the method for treating, the pharmaceutical kit for use, or the pharmaceutical composition for use, of embodiment A30, wherein the TR-FRET is a ligand-based TR-FRET.

Embodiment A32. The method for suppressing, the method for treating, the pharmaceutical kit for use, or the pharmaceutical composition for use, of embodiment A30, wherein the TR-FRET is an antibody-based TR-FRET.

Embodiment A33. The method for suppressing, the method for treating, the pharmaceutical kit for use, or the pharmaceutical composition for use, of any one of embodiments A1-A30, wherein the CXCR4-ADRB2 heteromer components in the cell colocalize and physically interact, either directly or via intermediate proteins acting as conduits for allosterism, is determined by one or more of the following: a co-internalization assay, bimolecular fluorescence complementation (BiFC), RT-PCR, RT-qPCR, expression level of CXCR4, expression level of ADRB2, expression level of CXCR4 and ADRB2, or a proximity ligation assay (PLA).

Embodiment A34. The method for suppressing, the method for treating, the pharmaceutical kit for use, or the pharmaceutical composition for use, of embodiment A33, wherein the CXCR4-ADRB2 heteromer components in the cell colocalize and physically interact, either directly or via intermediate proteins acting as conduits for allosterism, is determined by a co-internalization assay.

Embodiment A35. The method for suppressing, the method for treating, the pharmaceutical kit for use, or the pharmaceutical composition for use, of embodiment A33, wherein the CXCR4-ADRB2 heteromer components in the cell colocalize and physically interact, either directly or via intermediate proteins acting as conduits for allosterism, is determined by bimolecular fluorescence complementation (BiFC).

Embodiment A36. The method for suppressing, the method for treating, the pharmaceutical kit for use, or the pharmaceutical composition for use, of embodiment A33, wherein the CXCR4-ADRB2 heteromer components in the cell colocalize and physically interact, either directly or via intermediate proteins acting as conduits for allosterism, is determined by a proximity ligation assay (PLA).

Embodiment A37. The method for suppressing, the method for treating, the pharmaceutical kit for use, or the pharmaceutical composition for use, of any one of embodiments A1-A9 or A17-A36, wherein the assay determines the presence of the CXCR4-ADRB2 heteromer in the biological sample obtained from said subject.

Embodiment A38. The method for suppressing, the method for treating, the pharmaceutical kit for use, or the pharmaceutical composition for use, of any one of embodiments A1-A37, wherein the assay determines colocalization and interaction of the CXCR4 and ADRB2 components of the CXCR4-ADRB2 heteromer.

Embodiment A39. The method for suppressing, the method for treating, the pharmaceutical kit for use, or the pharmaceutical composition for use, of any one of embodiments A1-A9 or A14-A38, wherein the assay determines the presence of the CXCR4-ADRB2 heteromer in the subject's cell.

Embodiment A40. The method for suppressing, the method for treating, the pharmaceutical kit for use, or the pharmaceutical composition for use, of any one of embodiments A1-A9 or A17-A39, wherein the assay determines the presence of the CXCR4-ADRB2 heteromer in the biological sample obtained from said subject.

Embodiment A41. The method for suppressing, the method for treating, the pharmaceutical kit for use, or the pharmaceutical composition for use, of any one of embodiments A1-A9 or A17-A40, wherein the assay determines the presence of the CXCR4-ADRB2 heteromer in the subject.

Embodiment A42. The method for suppressing, the method for treating, the pharmaceutical kit for use, or the pharmaceutical composition for use, of any one of embodiments A1-A41, wherein the CXCR4-ADRB2 heteromer is characterized as having the following characteristic: enhanced downstream signaling results from the CXCR4-ADRB2 heteromer.

Embodiment A43. The method for suppressing, the method for treating, the pharmaceutical kit for use, or the pharmaceutical composition for use, of any one of embodiments A1-A9 or A14-A42, wherein the enhanced downstream signaling results from the CXCR4-ADRB2 heteromer in said subject's cell.

Embodiment A44. The method for suppressing, the method for treating, the pharmaceutical kit for use, or the pharmaceutical composition for use, of any one of embodiments A1-A9 or A14-A43, wherein the enhanced downstream signaling results from the presence of the CXCR4-ADRB2 heteromer in said subject's cell.

Embodiment A45. The method for suppressing, the method for treating, the pharmaceutical kit for use, or the pharmaceutical composition for use, of any one of embodiments A1-A44, wherein the CXCR4-ADRB2 heteromer produces the enhanced downstream signaling.

Embodiment A46. The method for suppressing, the method for treating, the pharmaceutical kit for use, or the pharmaceutical composition for use, of any one of embodiments A1-A9 or A14-A45, wherein the CXCR4-ADRB2 heteromer produces the enhanced downstream signaling in the subject's cell.

Embodiment A47. The method for suppressing, the method for treating, the pharmaceutical kit for use, or the pharmaceutical composition for use, of any one of embodiments A1-A46, wherein the enhanced downstream signaling results from agonism of the CXCR4-ADRB2 heteromer.

Embodiment A48. The method for suppressing, the method for treating, the pharmaceutical kit for use, or the pharmaceutical composition for use, of any one of embodiments A1-A47, wherein the enhanced downstream signaling results from CXCR4 agonism of the CXCR4-ADRB2 heteromer.

Embodiment A49. The method for suppressing, the method for treating, the pharmaceutical kit for use, or the pharmaceutical composition for use, of any one of embodiments A1-A47, wherein the enhanced downstream signaling results from ADRB2 agonism of the CXCR4-ADRB2 heteromer.

Embodiment A50. The method for suppressing, the method for treating, the pharmaceutical kit for use, or the pharmaceutical composition for use, of any one of embodiments A1-A47, wherein the enhanced downstream signaling results from CXCR4 agonism and ADRB2 agonism of the CXCR4-ADRB2 heteromer.

Embodiment A51. The method for suppressing, the method for treating, the pharmaceutical kit for use, or the pharmaceutical composition for use, of any one of embodiments A1-A50, wherein the enhanced downstream signaling is downstream of the CXCR4, the ADRB2, or the CXCR4-ADRB2 heteromer.

Embodiment A52. The method for suppressing, the method for treating, the pharmaceutical kit for use, or the pharmaceutical composition for use, of embodiment A51, wherein the enhanced downstream signaling is downstream of the CXCR4.

Embodiment A53. The method for suppressing, the method for treating, the pharmaceutical kit for use, or the pharmaceutical composition for use, of embodiment A51, wherein the enhanced downstream signaling is downstream of the ADRB2.

Embodiment A54. The method for suppressing, the method for treating, the pharmaceutical kit for use, or the pharmaceutical composition for use, of embodiment A51, wherein the enhanced downstream signaling is downstream of the CXCR4-ADRB2 heteromer.

Embodiment A55. The method for suppressing, the method for treating, the pharmaceutical kit for use, or the pharmaceutical composition for use, of any one of embodiments A1-A50, wherein the enhanced downstream signaling from the CXCR4-ADRB2 heteromer is relative to downstream signaling from a CXCR4 protomer or an ADRB2 protomer in their respective individual protomer context.

Embodiment A56. The method for suppressing, the method for treating, the pharmaceutical kit for use, or the pharmaceutical composition for use, of embodiment A55, wherein the enhanced downstream signaling from the CXCR4-ADRB2 heteromer is relative to downstream signaling from a CXCR4 protomer in an individual protomer context.

Embodiment A57. The method for suppressing, the method for treating, the pharmaceutical kit for use, or the pharmaceutical composition for use, of embodiment A55, wherein the enhanced downstream signaling from the CXCR4-ADRB2 heteromer is relative to downstream signaling from an ADRB2 protomer in an individual protomer context.

Embodiment A58. The method for suppressing, the method for treating, the pharmaceutical kit for use, or the pharmaceutical composition for use, of embodiment A55, wherein the enhanced downstream signaling from the CXCR4-ADRB2 heteromer is relative to downstream signaling from a CXCR4 protomer and an ADRB2 protomer in their respective individual protomer context.

Embodiment A59. The method for suppressing, the method for treating, the pharmaceutical kit for use, or the pharmaceutical composition for use, of any one of embodiments A1-A58, wherein the enhanced downstream signaling is an enhanced amount of calcium mobilization.

Embodiment A60. The method for suppressing, the method for treating, the pharmaceutical kit for use, or the pharmaceutical composition for use, of embodiment A59, wherein the enhanced amount of calcium mobilization is determined by an intracellular Ca2+ assay.

Embodiment A61. The method for suppressing, the method for treating, the pharmaceutical kit for use, or the pharmaceutical composition for use, of any one of embodiments A1-A60, wherein the enhanced downstream signaling from the CXCR4-ADRB2 heteromer is determined by an intracellular Ca2+ assay.

Embodiment A62. The method for suppressing, the method for treating, the pharmaceutical kit for use, or the pharmaceutical composition for use, of embodiment A61, wherein the intracellular Ca2+ assay is a calcium mobilization assay.

Embodiment A63. The method for suppressing, the method for treating, the pharmaceutical kit for use, or the pharmaceutical composition for use, of embodiment A62, wherein the calcium mobilization assay determines the CXCR4-ADRB2 heteromer produces the enhanced downstream signaling.

Embodiment A64. The method for suppressing, the method for treating, the pharmaceutical kit for use, or the pharmaceutical composition for use, of embodiment A62 or A63, wherein the calcium mobilization assay determines the enhanced downstream signaling results from the presence of the CXCR4-ADRB2 heteromer.

Embodiment A65. The method for suppressing, the method for treating, the pharmaceutical kit for use, or the pharmaceutical composition for use, of any one of embodiments A1-A64, wherein the CXCR4-ADRB2 heteromer exhibits the enhanced amount of calcium mobilization, such that:
a) either the CXCR4 or the ADRB2 in an individual protomer context in the cell upon co-stimulation with CXCL12 and an ADRB2 agonist results in a calcium mobilization amount that is equal to or less than the sum of calcium mobilization amounts resulting from single agonist stimulation with either the CXCL12 or the ADRB2 agonist; and
b) the CXCR4-ADRB2 heteromer exhibits an enhanced calcium mobilization upon co-stimulation with the CXCL12 and the ADRB2 agonist relative to the sum of calcium mobilization amounts resulting from single agonist stimulation with either the CXCL12 or the ADRB2 agonist;
   as determined via a calcium mobilization assay.

Embodiment A66. The method for suppressing, the method for treating, the pharmaceutical kit for use, or the pharmaceutical composition for use, of any one of embodiments A1-A65, wherein:
i) the calcium mobilization from the protomer CXCR4 or ADRB2, in the individual protomer context in the cell, is non-synergistic, as determined via calcium mobilization assay; and
ii) the calcium mobilization from the CXCR4-ADRB2 heteromer in the cell is synergistic, as determined via a calcium mobilization assay.

Embodiment A67. The method for suppressing, the method for treating, the pharmaceutical kit for use, or the pharmaceutical composition for use, of any one of embodiments A1-A66, wherein in the individual protomer context:
a) the individual protomer CXCR4 in the cell, in the absence of the individual protomer ADRB2; or
b) the individual protomer ADRB2 in the cell, in the absence of the individual protomer CXCR4;
   upon co-stimulation with CXCL12 and an ADRB2 agonist results in a calcium mobilization amount that is equal to or less than the sum of calcium mobilization amounts resulting from single agonist stimulation with either the CXCL12 or the ADRB2 agonist, as determined via a calcium mobilization assay.

Embodiment A68. The method for suppressing, the method for treating, the pharmaceutical kit for use, or the pharmaceutical composition for use, of any one of embodiments A1-A67, wherein in the individual protomer context, independently:
a) the individual protomer CXCR4 in the cell, in the absence of the individual protomer ADRB2; and
b) the individual protomer ADRB2 in the cell, in the absence of the individual protomer CXCR4;
   upon co-stimulation with CXCL12 and an ADRB2 agonist results in a calcium mobilization amount that is equal to or less than the sum of calcium mobilization amounts resulting from single agonist stimulation with either the CXCL12 or the ADRB2 agonist, as determined via a calcium mobilization assay.

Embodiment A69. The method for suppressing, the method for treating, the pharmaceutical kit for use, or the pharmaceutical composition for use, of any one of embodiments A1-A68, wherein the CXCR4-ADRB2 heteromer upon co-stimulation with the CXCL12 and the ADRB2 agonist results in a calcium mobilization amount that is greater than the sum of calcium mobilization amounts resulting from single agonist stimulation of said cells with either the CXCL12 or the ADRB2 agonist, as determined via a calcium mobilization assay.

Embodiment A70. The method for suppressing, the method for treating, the pharmaceutical kit for use, or the pharmaceutical composition for use, of any one of embodiments A1-A69, wherein the calcium mobilization amount resulting from the co-stimulation of the CXCR4-ADRB2 heteromer is an enhanced amount of calcium mobilization, relative to the sum of calcium mobilizations resulting from single agonist stimulation of said CXCR4-ADRB2 heteromer, as determined via a calcium mobilization assay.

Embodiment A71. The method for suppressing, the method for treating, the pharmaceutical kit for use, or the pharmaceutical composition for use, of any one of embodiments A1-A70, wherein the enhanced amount of calcium mobilization resulting from the CXCR4-ADRB2 heteromer is a calcium mobilization amount that, upon co-stimulation with CXCL12 and ADRB2 agonist, is greater than the sum of calcium mobilization amounts resulting from single agonist stimulation of said cells with either the CXCL12 or the ADRB2 agonist, as determined via a calcium mobilization assay.

Embodiment A72. The method for suppressing, the method for treating, the pharmaceutical kit for use, or the pharmaceutical composition for use, of embodiment A71, wherein the enhanced amount of calcium mobilization resulting from the CXCR4-ADRB2 heteromer is a calcium mobilization amount that, upon co-stimulation with the CXCL12 and the ADRB2 agonist, is at least 10% greater, at least 20% greater, at least 30% greater, at least 40% greater, at least 50% greater, at least 75% greater, or at least 90% greater, than the sum of calcium mobilization amounts resulting from single agonist stimulation of said cells with either the CXCL12 or the ADRB2 agonist, as determined via a calcium mobilization assay.

Embodiment A73. The method for suppressing, the method for treating, the pharmaceutical kit for use, or the pharmaceutical composition for use, of embodiment A71 or A72, wherein the enhanced amount of calcium mobilization resulting from the CXCR4-ADRB2 heteromer is a calcium mobilization amount that, upon co-stimulation with the CXCL12 and the ADRB2 agonist, is at least 100% greater than the sum of calcium mobilization amounts resulting from single agonist stimulation of said cells with either the CXCL12 or the ADRB2 agonist, as determined via a calcium mobilization assay.

Embodiment A74. The method for suppressing, the method for treating, the pharmaceutical kit for use, or the pharmaceutical composition for use, of any one of embodiments A71-A73, wherein the enhanced amount of calcium mobilization is a synergistic amount of calcium mobilization.

Embodiment A75. The method for suppressing, the method for treating, the pharmaceutical kit for use, or the pharmaceutical composition for use, of embodiment A74, wherein the synergistic amount of calcium mobilization from the cells containing the CXCR4-ADRB2 heteromer is a calcium mobilization amount that, upon co-stimulation with the CXCL12 and the ADRB2 agonist, is at least 10% greater, at least 20% greater, at least 30% greater, at least 40% greater, at least 50% greater, at least 75% greater, or at least 90% greater, than the sum of calcium mobilization amounts resulting from single agonist stimulation of said cells with either the CXCL12 or the ADRB2 agonist, as determined via a calcium mobilization assay.

Embodiment A76. The method for suppressing, the method for treating, the pharmaceutical kit for use, or the pharmaceutical composition for use, of any one of embodiments A1-A75, wherein the enhanced amount of calcium mobilization is characterized as follows:
a) either the CXCR4 or the ADRB2 in an individual protomer context in a cell, upon co-stimulation with CXCL12 and an ADRB2 agonist, results in a calcium mobilization amount that is equal to or less than the sum of calcium mobilization amounts resulting from single agonist stimulation with either the CXCL12 or the ADRB2 agonist; and
b) the CXCR4-ADRB2 heteromer exhibits an enhanced calcium mobilization upon co-stimulation with the CXCL12 and the ADRB2 agonist relative to the sum of calcium mobilization amounts resulting from single agonist stimulation with either the CXCL12 or the ADRB2 agonist;
   as determined via a calcium mobilization assay.

Embodiment A77. The method for suppressing, the method for treating, the pharmaceutical kit for use, or the pharmaceutical composition for use, of any one of embodiments A1-A76, wherein the CXCR4-ADRB2 heteromer is characterized as having the following characteristic: a greater amount of downstream ERK signaling results from co-stimulation of the CXCR4-ADRB2 heteromer with a CXCR4 agonist and an ADRB2 agonist, relative to the amount of downstream ERK signaling resulting from mono-stimulation of said CXCR4-ADRB2 heteromer with either the CXCR4 agonist or the ADRB2 agonist.

Embodiment A78. The method for suppressing, the method for treating, the pharmaceutical kit for use, or the pharmaceutical composition for use, of embodiment A77, wherein the amount of downstream ERK signaling resulting from co-stimulation with the CXCR4 agonist and the ADRB2 agonist is greater than the amount resulting from mono-stimulation with the CXCR4 agonist.

Embodiment A79. The method for suppressing, the method for treating, the pharmaceutical kit for use, or the pharmaceutical composition for use, of embodiment A78, wherein the amount of downstream ERK signaling resulting from co-stimulation with the CXCR4 agonist and the ADRB2 agonist is at least 5% greater than the amount resulting from mono-stimulation with the CXCR4 agonist.

Embodiment A80. The method for suppressing, the method for treating, the pharmaceutical kit for use, or the pharmaceutical composition for use, of embodiment A78, wherein the amount of downstream ERK signaling resulting from co-stimulation with the CXCR4 agonist and the ADRB2 agonist is at least 10%, at least 25%, at least 50%, at least 75%, or at least 90%, greater than the amount resulting from mono-stimulation with the CXCR4 agonist.

Embodiment A81. The method for suppressing, the method for treating, the pharmaceutical kit for use, or the pharmaceutical composition for use, of any one of embodiments A78-A80, wherein the amount of downstream ERK signaling resulting from co-stimulation with the CXCR4 agonist and the ADRB2 agonist in the range of between 5-15%, 10-25%, 20-50%, 40-75%, or 60-100%, greater than the amount resulting from mono-stimulation with the CXCR4 agonist.

Embodiment A82. The method for suppressing, the method for treating, the pharmaceutical kit for use, or the pharmaceutical composition for use, of embodiment A77, wherein the amount of downstream ERK signaling resulting from co-stimulation with the CXCR4 agonist and the ADRB2 agonist is greater than the amount resulting from mono-stimulation with the ADRB2 agonist.

Embodiment A83. The method for suppressing, the method for treating, the pharmaceutical kit for use, or the pharmaceutical composition for use, of embodiment A82, wherein the amount of downstream ERK signaling resulting from co-stimulation with the CXCR4 agonist and the ADRB2 agonist is at least 5% greater than the amount resulting from mono-stimulation with the ADRB2 agonist.

Embodiment A84. The method for suppressing, the method for treating, the pharmaceutical kit for use, or the pharmaceutical composition for use, of embodiment A82, wherein the amount of downstream ERK signaling resulting from co-stimulation with the CXCR4 agonist and the ADRB2 agonist is at least 10%, at least 25%, at least 50%, at least 75%, or at least 90%, greater than the amount resulting from mono-stimulation with the ADRB2 agonist.

Embodiment A85. The method for suppressing, the method for treating, the pharmaceutical kit for use, or the pharmaceutical composition for use, of any one of embodiments A82-A84, wherein the amount of downstream ERK signaling resulting from co-stimulation with the CXCR4 agonist and the ADRB2 agonist in the range of between 5-15%, 10-25%, 20-50%, 40-75%, or 60-100%, greater than the amount resulting from mono-stimulation with the ADRB2 agonist.

Embodiment A86. The method for suppressing, the method for treating, the pharmaceutical kit for use, or the pharmaceutical composition for use, of any one of embodiments A1-A9 or A14-A85, wherein the CXCR4-ADRB2 heteromer is characterized as having the following characteristic: a combination of burixafor and an ADRB2 inhibitor alters heteromer-specific properties of the CXCR4-ADRB2 heteromer in the subject's derived cell(s).

Embodiment A87. The method for suppressing, the method for treating, the pharmaceutical kit for use, or the pharmaceutical composition for use, of any one of embodiments A1-A9 or A14-A86, wherein the CXCR4-ADRB2 heteromer is characterized as having the following characteristic: a combination of burixafor and an ADRB2 inhibitor alters heteromer-specific function of the CXCR4-ADRB2 heteromer in the subject's derived cell(s).

Embodiment A88. The method for suppressing, the method for treating, the pharmaceutical kit for use, or the pharmaceutical composition for use, of any one of embodiments A1-A9 or A14-A87, wherein the CXCR4-ADRB2 heteromer is characterized as having the following characteristic: a combination of burixafor and an ADRB2 inhibitor alters heteromer-specific properties of the subject's derived cell(s) containing the CXCR4-ADRB2 heteromer.

Embodiment A89. The method for suppressing, the method for treating, the pharmaceutical kit for use, or the pharmaceutical composition for use, of any one of embodiments A1-A9 or A14-A88, wherein the CXCR4-ADRB2 heteromer is characterized as having the following characteristic: a combination of burixafor and an ADRB2 inhibitor decreases cancer progression of the subject derived cell(s) containing the CXCR4-ADRB2 heteromer.

Embodiment A90. The method for suppressing, the method for treating, the pharmaceutical kit for use, or the pharmaceutical composition for use, of any one of embodiments A1-A89, wherein the administered combination of the burixafor and the ADRB2 inhibitor suppresses the enhanced downstream signaling from said CXCR4-ADRB2 heteromer.

Embodiment A91. The method for suppressing, the method for treating, the pharmaceutical kit for use, or the pharmaceutical composition for use, of any one of embodiments A1-A90, wherein the administered combination of the burixafor and the ADRB2 inhibitor suppresses the enhanced downstream signaling from said CXCR4-ADRB2 heteromer in the cell.

Embodiment A92. The method for suppressing, the method for treating, the pharmaceutical kit for use, or the pharmaceutical composition for use, of any one of embodiments A1-A9 or A17-A91, wherein the administered combination of the burixafor and the ADRB2 inhibitor suppresses the enhanced downstream signaling from said CXCR4-ADRB2 heteromer in the cancer subject.

Embodiment A93. The method for suppressing, the method for treating, the pharmaceutical kit for use, or the pharmaceutical composition for use, of any one of embodiments A1-A9 or A17-A92, wherein the administered combination of the burixafor and the ADRB2 inhibitor suppresses the enhanced downstream signaling from said CXCR4-ADRB2 heteromer in the cell of the cancer subject.

Embodiment A94. The method for suppressing, the method for treating, the pharmaceutical kit for use, or the pharmaceutical composition for use, of any one of embodiments A1-A93, wherein the CXCR4-ADRB2 heteromer components comprise individual protomers of CXCR4 and ADRB2.

Embodiment A95. The method for suppressing, the method for treating, the pharmaceutical kit for use, or the pharmaceutical composition for use, of any one of embodiments A1-A94, wherein the cell containing the CXCR4 in an individual protomer context comprises the individual protomer CXCR4 in the presence or absence of the individual protomer ADRB2.

Embodiment A96. The method for suppressing, the method for treating, the pharmaceutical kit for use, or the pharmaceutical composition for use, of embodiment A95, wherein the cell containing the CXCR4 in an individual protomer context comprises said individual protomer CXCR4 in the absence of the individual protomer ADRB2.

Embodiment A97. The method for suppressing, the method for treating, the pharmaceutical kit for use, or the pharmaceutical composition for use, of embodiment A95, wherein the cell containing the CXCR4 in an individual protomer context comprises said individual protomer CXCR4 in the presence of the individual protomer ADRB2.

Embodiment A98. The method for suppressing, the method for treating, the pharmaceutical kit for use, or the pharmaceutical composition for use, of any one of embodiments A1-A94, wherein the cell containing the ADRB2 in an individual protomer context comprises the individual protomer ADRB2 in the presence or absence of the individual protomer CXCR4.

Embodiment A99. The method for suppressing, the method for treating, the pharmaceutical kit for use, or the pharmaceutical composition for use, of embodiment A98, wherein the cell containing the ADRB2 in an individual protomer context comprises said individual protomer ADRB2 in the absence of the individual protomer CXCR4.

Embodiment A100. The method for suppressing, the method for treating, the pharmaceutical kit for use, or the pharmaceutical composition for use, of embodiment A98, wherein the cell containing the ADRB2 in an individual protomer context comprises said individual protomer ADRB2 in the presence of the individual protomer CXCR4.

Embodiment A101. The method for suppressing, the method for treating, the pharmaceutical kit for use, or the pharmaceutical composition for use, of any one of embodiments A1-A100, wherein the administered combination of burixafor and an ADRB2 inhibitor:
i) alters heteromer-specific properties of the CXCR4-ADRB2 heteromer in the cell;
ii) alters heteromer-specific function of the CXCR4-ADRB2 heteromer in the cell; and/or
iii) alters heteromer-specific properties of the cell containing the CXCR4-ADRB2 heteromer.

Embodiment A102. The method for suppressing, the method for treating, the pharmaceutical kit for use, or the pharmaceutical composition for use, of any one of embodiments A1-A9 or A14-A101, wherein the administered combination of the burixafor and the ADRB2 inhibitor:
i) alters heteromer-specific properties of the CXCR4-ADRB2 heteromer in the subject's derived cell(s);
ii) alters heteromer-specific function of the CXCR4-ADRB2 heteromer in the subject's derived cell(s);
iii) alters heteromer-specific properties of the subject's derived cell(s) containing the CXCR4-ADRB2 heteromer; or
iv) decreases cancer progression in the subject having cells containing said CXCR4-ADRB2 heteromer.

Embodiment A103. The method for suppressing, the method for treating, the pharmaceutical kit for use, or the pharmaceutical composition for use, of embodiment A102, wherein the administered combination of the burixafor and the ADRB2 inhibitor alters heteromer-specific properties of the CXCR4-ADRB2 heteromer in the subject's derived cell(s).

Embodiment A104. The method for suppressing, the method for treating, the pharmaceutical kit for use, or the pharmaceutical composition for use, of embodiment A102 or 103, wherein the administered combination of the burixafor and the ADRB2 inhibitor alters heteromer-specific function of the CXCR4-ADRB2 heteromer in the subject's derived cell(s).

Embodiment A105. The method for suppressing, the method for treating, the pharmaceutical kit for use, or the pharmaceutical composition for use, of any one of embodiments A102-A104, wherein the administered combination of the burixafor and the ADRB2 inhibitor alters heteromer-specific properties of the subject's derived cell(s) containing the CXCR4-ADRB2 heteromer.

Embodiment A106. The method for suppressing, the method for treating, the pharmaceutical kit for use, or the pharmaceutical composition for use, of any one of embodiments A102-A105, wherein the administered combination of the burixafor and the ADRB2 inhibitor decreases cancer progression in the subject having cells containing said CXCR4-ADRB2 heteromer.

Embodiment A107. The method for suppressing, the method for treating, the pharmaceutical kit for use, or the pharmaceutical composition for use, of any one of embodiments A102-A106, wherein the CXCR4-ADRB2 heteromer is characterized as having the following characteristic: a combination of burixafor and an ADRB2 inhibitor decreases cancer progression of the subject derived cell(s) containing the CXCR4-ADRB2 heteromer.

Embodiment A108. The method for suppressing, the method for treating, the pharmaceutical kit for use, or the pharmaceutical composition for use, of any one of embodiments A102-A107, wherein the decreased cancer progression comprises a decrease in cancer progression of the subject's derived cell(s) containing said CXCR4-ADRB2 heteromer.

Embodiment A109. The method for suppressing, the method for treating, the pharmaceutical kit for use, or the pharmaceutical composition for use, of any one of embodiments A102-A108, wherein the decreased cancer progression comprises a decrease in cell proliferation of the subject's derived cell(s) containing said CXCR4-ADRB2 heteromer.

Embodiment A110. The method for suppressing, the method for treating, the pharmaceutical kit for use, or the pharmaceutical composition for use, of any one of embodiments A102-A109, wherein the decreased cancer progression comprises a decrease in cell migration of the subject's derived cell(s) containing said CXCR4-ADRB2 heteromer.

Embodiment A111. The method for suppressing, the method for treating, the pharmaceutical kit for use, or the pharmaceutical composition for use, of any one of embodiments A102-A110, wherein the decreased cancer progression comprises a decrease in metastasis of the subject's derived cell(s) containing said CXCR4-ADRB2 heteromer.

Embodiment A112. The method for suppressing, the method for treating, the pharmaceutical kit for use, or the pharmaceutical composition for use, of any one of embodiments A102-A111, wherein the decreased cancer progression comprises a decrease in angiogenesis of the subject's derived cell(s) containing said CXCR4-ADRB2 heteromer.

Embodiment A113. The method for suppressing, the method for treating, the pharmaceutical kit for use, or the pharmaceutical composition for use, of any one of embodiments A1-A112, wherein the burixafor is administered as a pharmaceutical composition comprising a pharmaceutically acceptable carrier.

Embodiment A114. The method for suppressing, the method for treating, the pharmaceutical kit for use, or the pharmaceutical composition for use, of any one of embodiments A1-A113, wherein the ADRB2 inhibitor is administered as a pharmaceutical composition comprising a pharmaceutically acceptable carrier.

Embodiment A115. The method for suppressing, the method for treating, the pharmaceutical kit for use, or the pharmaceutical composition for use, of any one of embodiments A1-A114, wherein the combination of the burixafor and the ADRB2 inhibitor is administered sequentially, concurrently, or simultaneously.

Embodiment A116. The method for suppressing, the method for treating, the pharmaceutical kit for use, or the pharmaceutical composition for use, of any one of embodiments A1-A115, wherein the combination of the burixafor and the ADRB2 inhibitor is administered as a pharmaceutical composition further comprising a pharmaceutically acceptable carrier.

Embodiment A117. The method for suppressing, the method for treating, the pharmaceutical kit for use, or the pharmaceutical composition for use, of any one of embodiments A1-A116, wherein the burixafor and the ADRB2 inhibitor are administered as a combination of pharmaceutical compositions.

Embodiment A118. The method for suppressing, the method for treating, the pharmaceutical kit for use, or the pharmaceutical composition for use, of embodiment A117, wherein the combination of pharmaceutical compositions comprises:
a) a pharmaceutical composition comprising the burixafor and a pharmaceutically acceptable carrier; and
b) a pharmaceutical composition comprising the ADRB2 inhibitor and a pharmaceutically acceptable carrier.

Embodiment A119. The method for suppressing, the method for treating, the pharmaceutical kit for use, or the pharmaceutical composition for use, of embodiment A117 or A118, wherein the combination of the pharmaceutical compositions is administered sequentially, concurrently, or simultaneously.

Embodiment A120. The method for suppressing, the method for treating, the pharmaceutical kit for use, or the pharmaceutical composition for use, of any one of embodiments A1-A119, wherein the cancer is a hematological cancer or a solid tumor.

Embodiment A121. The method for suppressing, the method for treating, the pharmaceutical kit for use, or the pharmaceutical composition for use, of embodiment A120, wherein the cancer is a hematological cancer.

Embodiment A122. The method for suppressing, the method for treating, the pharmaceutical kit for use, or the pharmaceutical composition for use, of embodiment A121, wherein the hematological cancer is selected from the group consisting of: a lymphoma, a leukemia, a myeloma, and a multiple myeloma.

Embodiment A123. The method for suppressing, the method for treating, the pharmaceutical kit for use, or the pharmaceutical composition for use, of embodiment A122, wherein the lymphoma is a B cell lymphoma, a T-cell lymphoma, or a NK cell lymphoma.

Embodiment A124. The method for suppressing, the method for treating, the pharmaceutical kit for use, or the pharmaceutical composition for use, of embodiment A122 or A123, wherein the lymphoma is a relapsed or refractory lymphoma.

Embodiment A125. The method for suppressing, the method for treating, the pharmaceutical kit for use, or the pharmaceutical composition for use, of any one of embodiments A122-A124, wherein the lymphoma is selected from the group consisting of: Hodgkin's lymphoma, non-Hodgkin's lymphoma, cutaneous B-cell lymphoma, activated B-cell lymphoma, Diffuse Large B-Cell Lymphoma (DLBCL), Burkitt lymphoma, mantle cell lymphoma (MCL), follicular lymphoma (FL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphoma (PTCL), cutaneous T-Cell lymphoma (CTCL), mantle zone lymphoma, and low grade follicular lymphoma.

Embodiment A126. The method for suppressing, the method for treating, the pharmaceutical kit for use, or the pharmaceutical composition for use, of embodiment A122, wherein the leukemia is:
a) an acute leukemia selected from the group consisting of: acute lymphocytic leukemia (ALL), T-cell acute lymphocytic leukemia (T-ALL), B-cell acute lymphoblastic leukemia, acute monocytic leukemia, acute promyelocytic leukemia, acute myelocytic leukemia (AML), acute myeloid leukemia, acute myelogenous leukemia and myeloblasts, promyeiocytic, myelomonocytic, monocytic, and erythroleukemia;
b) a chronic leukemia selected from the group consisting of: chronic myelocytic (granulocytic) leukemia, chronic myelogenous leukemia (chronic myeloid leukemia; CML), and chronic lymphocytic leukemia (CLL); or
c) chronic myelomonocytic leukemia (CMML), chronic eosinophilic leukemia, juvenile myelomonocytic leukemia (JMML), polycythemia vera, natural killer cell leukemia (NK leukemia), or hairy cell leukemia.

Embodiment A127. The method for suppressing, the method for treating, the pharmaceutical kit for use, or the pharmaceutical composition for use, of embodiment A120, wherein the cancer is a solid tumor.

Embodiment A128. The method for suppressing, the method for treating, the pharmaceutical kit for use, or the pharmaceutical composition for use, of embodiment A127, wherein the solid tumor is a benign tumor or a cancer.

Embodiment A129. The method for suppressing, the method for treating, the pharmaceutical kit for use, or the pharmaceutical composition for use, of embodiment A127, wherein the solid tumor is a carcinoma or a sarcoma.

Embodiment A130. The method for suppressing, the method for treating, the pharmaceutical kit for use, or the pharmaceutical composition for use, of embodiment A127, wherein the solid tumor is selected from the group consisting of: fibrosarcoma, myxosarcoma, liposarcoma, chondrosarcoma, osteosarcoma, synovioma, mesothelioma, malignant epithelioid mesothelioma, Ewing's tumor, leiomyosarcoma, rhabdomyosarcoma, stomach cancer, colorectal cancer, esophageal cancer, colon carcinoma, lymphoid malignancy, pancreatic cancer, pancreatic adenocarcinoma, pancreatic ductal adenocarcinoma, breast cancer, breast adenocarcinoma, breast ductal adenocarcinoma, lung cancer, small cell lung carcinoma, lung adenocarcinoma, adenosquamous lung carcinoma, alveolar rhabdomyosarcoma, ovarian cancer, ovarian clear cell adenocarcinoma, ovarian mucinous cystadenocarcinoma, ovarian serous adenocarcinoma, prostate cancer, hepatocellular carcinoma, soft tissue sarcomas, squamous cell carcinoma, basal cell carcinoma, adenocarcinoma, sweat gland carcinoma, medullary thyroid carcinoma, papillary thyroid carcinoma, thyroid gland carcinoma, pheochromocytomas sebaceous gland carcinoma, papillary carcinoma, papillary adenocarcinomas, medullary carcinoma, bronchogenic carcinoma, gastrointestinal cancer, gastric tubular adenocarcinoma, gastric adenosquamous carcinoma, kidney cancer, intrahepatic cholangiocarcinoma, renal cell carcinoma, hepatoma, bile duct carcinoma, choriocarcinoma, Wilms' tumor, uterine carcinosarcoma, endometrium adenocarcinoma, endometrial stromal sarcoma, endometrial carcinoma, cervical cancer, testicular tumor, seminoma, bladder carcinoma, melanoma, multiple myeloma, multiple endocrine neoplasia, CNS tumor, glioblastoma, astrocytoma, CNS lymphoma, germinoma, meduloblastoma, Schwannoma craniopharyogioma, ependymoma, pineaioma, hemangioblastoma, acoustic neuroma, oligodendroglioma, menangioma, neuroblastoma, retinoblastoma, and brain metastases.

Embodiment A131. The method for suppressing, the method for treating, the pharmaceutical kit for use, or the pharmaceutical composition for use, of embodiment A130, wherein the solid tumor is selected from the group consisting of: breast cancer, lung cancer, and hepatocellular carcinoma.

Embodiment A132. The method for suppressing, the method for treating, the pharmaceutical kit for use, or the pharmaceutical composition for use, of embodiment A131, wherein the solid tumor is breast cancer.

Embodiment A133. The method for suppressing, the method for treating, the pharmaceutical kit for use, or the pharmaceutical composition for use, of embodiment A131, wherein the solid tumor is lung cancer.

Embodiment A134. The method for suppressing, the method for treating, the pharmaceutical kit for use, or the pharmaceutical composition for use, of embodiment A131, wherein the solid tumor is hepatocellular carcinoma.

Embodiment A135. The method for suppressing, the method for treating, the pharmaceutical kit for use, or the pharmaceutical composition for use, of any one of embodiments A1-A9 or A17-A134, wherein the subject's biological sample is a biological fluid sample.

Embodiment A136. The method for suppressing, the method for treating, the pharmaceutical kit for use, or the pharmaceutical composition for use, of embodiment A135, wherein a liquid biopsy is performed on the biological fluid sample.

Embodiment A137. The method for suppressing, the method for treating, the pharmaceutical kit for use, or the pharmaceutical composition for use, of embodiment A135 or A136, wherein the biological fluid sample is a blood sample, a plasma sample, a saliva sample, a cerebral fluid sample, an eye fluid sample, or a urine sample.

Embodiment A138. The method for suppressing, the method for treating, the pharmaceutical kit for use, or the pharmaceutical composition for use, of any one of embodiments A1-A9 or A17-A137, wherein the subject's biological sample is a biological tissue sample.

Embodiment A139. The method for suppressing, the method for treating, the pharmaceutical kit for use, or the pharmaceutical composition for use, of embodiment A138, wherein a tissue sample assay is performed on the biological tissue sample.

Embodiment A140. The method for suppressing, the method for treating, the pharmaceutical kit for use, or the pharmaceutical composition for use, of embodiment A138 or A139, wherein the biological tissue sample is an organ tissue sample, a bone tissue sample, or a tumor tissue sample.

Embodiment A141. The method for suppressing, the method for treating, the pharmaceutical kit for use, or the pharmaceutical composition for use, of any one of embodiments A1-A140, wherein the method for treating cancer is a method for suppressing enhanced downstream signaling resulting from the CXCR4-ADRB2 heteromer.

Embodiment A142. The method for suppressing, the method for treating, the pharmaceutical kit for use, or the pharmaceutical composition for use, of any one of embodiments A1-A140, wherein the method for suppressing enhanced downstream signaling resulting from the CXCR4-ADRB2 heteromer is a method for treating cancer.

Embodiment A143. The method for suppressing, the method for treating, the pharmaceutical kit for use, or the pharmaceutical composition for use, of any one of embodiments A1-A9 or A17-A142, wherein progression of the cancer in the subject having said cell containing the CXCR4-ADRB2 heteromer is decreased in the range of 5-100% more upon administering to the cancer subject the combination of the burixafor and the ADRB2 inhibitor, relative to administering the single inhibitor of either the burixafor or the ADRB2 inhibitor.

Embodiment A144. The method for suppressing, the method for treating, the pharmaceutical kit for use, or the pharmaceutical composition for use, of any one of embodiments A1-A9 or A17-A143, wherein efficacy of the burixafor is increased in the range of 5-5000% when administered in combination with the ADRB2 inhibitor to the subject having said cell containing the CXCR4-ADRB2 heteromer, relative to efficacy of the burixafor when administered as a single inhibitor.

Embodiment A145. The method for suppressing, the method for treating, the pharmaceutical kit for use, or the pharmaceutical composition for use, of any one of embodiments A1-A9 or A17-A144, wherein efficacy of an ADRB2 inhibitor is increased in the range of 5-5000% when administered in combination with the burixafor to the subject having said cell containing the CXCR4-ADRB2 heteromer, relative to efficacy of the ADRB2 inhibitor when administered as a single inhibitor.

Embodiment A146. The method for suppressing, the method for treating, the pharmaceutical kit for use, or the pharmaceutical composition for use, of any one of embodiments A1-A9 or A17-A145, wherein the administering to the cancer subject the combination of the burixafor and the ADRB2 inhibitor suppresses the enhanced downstream signaling from said CXCR4-ADRB2 heteromer in said cancer subject in the range of between 5-2000 fold, relative to single inhibitor administration.

Embodiment A147. The method for suppressing, the method for treating, the pharmaceutical kit for use, or the pharmaceutical composition for use, of any one of embodiments A1-A9 or A17-A146, wherein the administering to the cancer subject the combination of the burixafor and the ADRB2 inhibitor suppresses the enhanced downstream signaling from said CXCR4-ADRB2 heteromer in said cancer subject in the range of between 5-2000 fold, relative to suppression of downstream signaling from either a CXCR4 protomer or an ADRB2 protomer in their respective individual protomer context.

Embodiment A148. The method for suppressing, the method for treating, the pharmaceutical kit for use, or the pharmaceutical composition for use, of any one of embodiments A17-A147, wherein the cell is a cancer cell.

Embodiment A149. The method for suppressing, the method for treating, the pharmaceutical kit for use, or the pharmaceutical composition for use, of any one of embodiments A17-A147, wherein the cell is a subject's cell.

Embodiment A150. The method for suppressing, the method for treating, the pharmaceutical kit for use, or the pharmaceutical composition for use, of embodiment A149, wherein the subject's cell is a cancer cell.

Embodiment A151. The method for suppressing, the method for treating, the pharmaceutical kit for use, or the pharmaceutical composition for use, of any one of embodiments A1-A150, wherein the subject is a cancer subject.

Embodiment A152. The method for suppressing, the method for treating, the pharmaceutical kit for use, or the pharmaceutical composition for use, of any one of embodiments A1-A151, wherein the subject is a patient.

Embodiment A153. A pharmaceutical composition, comprising:
a) a CXCR4 inhibitor, wherein the CXCR4 inhibitor is burixafor;
b) an ADRB2 inhibitor; and
c) a pharmaceutically acceptable carrier.

Embodiment A154. The method for suppressing, the method for treating, the pharmaceutical kit for use, the pharmaceutical composition for use, or the pharmaceutical composition, of any one of embodiments A1-A153, wherein the ADRB2 inhibitor is an antagonist of ADRB2, an inverse agonist of ADRB2, a partial antagonist of ADRB2, an allosteric modulator of ADRB2, an antibody of ADRB2, an antibody fragment of ADRB2, a ligand of ADRB2, or an antibody-drug conjugate.

Embodiment A155. The method for suppressing, the method for treating, the pharmaceutical kit for use, the pharmaceutical composition for use, or the pharmaceutical composition, of embodiment A154, wherein the ADRB2 inhibitor is an antagonist ADRB2.

Embodiment A156. The method for suppressing, the method for treating, the pharmaceutical kit for use, the pharmaceutical composition for use, or the pharmaceutical composition, of embodiment A154, wherein the ADRB2 inhibitor is an inverse agonist ADRB2.

Embodiment A157. The method for suppressing, the method for treating, the pharmaceutical kit for use, the pharmaceutical composition for use, or the pharmaceutical composition, of embodiment A154, wherein the ADRB2 inhibitor is a partial antagonist ADRB2.

Embodiment A158. The method for suppressing, the method for treating, the pharmaceutical kit for use, the pharmaceutical composition for use, or the pharmaceutical composition, of embodiment A154, wherein the ADRB2 inhibitor is an allosteric modulator of ADRB2.

Embodiment A159. The method for suppressing, the method for treating, the pharmaceutical kit for use, the pharmaceutical composition for use, or the pharmaceutical composition, of embodiment A154, wherein the ADRB2 inhibitor is an antibody of ADRB2.

Embodiment A160. The method for suppressing, the method for treating, the pharmaceutical kit for use, the pharmaceutical composition for use, or the pharmaceutical composition, of embodiment A154, wherein the ADRB2 inhibitor is an antibody fragment of ADRB2.

Embodiment A161. The method for suppressing, the method for treating, the pharmaceutical kit for use, the pharmaceutical composition for use, or the pharmaceutical composition, of embodiment A154, wherein the ADRB2 inhibitor is a ligand of ADRB2.

Embodiment A162. The method for suppressing, the method for treating, the pharmaceutical kit for use, the pharmaceutical composition for use, or the pharmaceutical composition, of embodiment A154, wherein the ADRB2 inhibitor is an antibody-drug conjugate.

Embodiment A163. The method for suppressing, the method for treating, the pharmaceutical kit for use, the pharmaceutical composition for use, or the pharmaceutical composition, of embodiment A154, wherein the ADRB2 inhibitor is selected from the group consisting of: Alprenolol, atenolol, betaxolol, bupranolol, butoxamine, carazolol, carvedilol, CGP 12177, cicloprolol, ICI 118551, ICYP, labetalol, levobetaxolol, levobunolol, LK 204-545, metoprolol, nadolol, NIHP, NIP, propafenone, propranolol, sotalol, SR59230A, and timolol.

Embodiment A164. The method for suppressing, the method for treating, the pharmaceutical kit for use, the pharmaceutical composition for use, or the pharmaceutical composition, of embodiment A163, wherein the ADRB2 inhibitor is carvedilol.

Embodiment A165. The method for suppressing, the method for treating, the pharmaceutical kit for use, the pharmaceutical composition for use, or the pharmaceutical composition, of any one of embodiments A1-A9 or A17-A164, wherein a therapeutically effective amount of the burixafor is administered to the subject.

Embodiment A166. The method for suppressing, the method for treating, the pharmaceutical kit for use, the pharmaceutical composition for use, or the pharmaceutical composition, of any one of embodiments A1-A9 or A17-A164, wherein a sub-therapeutically effective amount of the burixafor is administered to the subject.

Embodiment A167. The method for suppressing, the method for treating, the pharmaceutical kit for use, the pharmaceutical composition for use, or the pharmaceutical composition, of any one of embodiments A1-A9 or A17-A166, wherein a therapeutically effective amount of the ADRB2 inhibitor is administered to the subject.

Embodiment A168. The method for suppressing, the method for treating, the pharmaceutical kit for use, the pharmaceutical composition for use, or the pharmaceutical composition, of any one of embodiments A1-A9 or A17-A166, wherein a sub-therapeutically effective amount of the ADRB2 inhibitor is administered to the subject.

Embodiment A169. The method for suppressing, the method for treating, the pharmaceutical kit for use, the pharmaceutical composition for use, or the pharmaceutical composition, of any one of embodiments A1-A9 or A14-A168, wherein the method or use further comprises determining the expression level of the CXCR4 gene in the cell, in the subject, or in the sample obtained from the subject, wherein the cell, the subject, or the sample obtained from the subject is determined to have a CXCR4-expressing cancer if the expression level is greater than a reference level of the CXCR4 gene.

Embodiment A170. The method for suppressing, the method for treating, the pharmaceutical kit for use, the pharmaceutical composition for use, or the pharmaceutical composition, of embodiment A169, wherein the cancer is a CXCR4-expressing cancer.

Embodiment A171. The method for suppressing, the method for treating, the pharmaceutical kit for use, the pharmaceutical composition for use, or the pharmaceutical composition, of embodiment A169 or A170, wherein the CXCR4 expression level in the cell is greater than a reference level.

Embodiment A172. The method for suppressing, the method for treating, the pharmaceutical kit for use, the pharmaceutical composition for use, or the pharmaceutical composition, of embodiment A169 or A170, wherein the CXCR4 expression level in the subject is greater than a reference level.

Embodiment A173. The method for suppressing, the method for treating, the pharmaceutical kit for use, the pharmaceutical composition for use, or the pharmaceutical composition, of embodiment A169 or A170, wherein the CXCR4 expression level in the sample obtained from the subject is greater than a reference level.

Embodiment A174. The method for suppressing, the method for treating, the pharmaceutical kit for use, the pharmaceutical composition for use, or the pharmaceutical composition, of any one of embodiments A169-A173, upon determining the CXCR4 gene expression level is greater than the reference level, the method or use comprises administering a CXCR4 inhibitor and an ADRB2 inhibitor, wherein the CXCR4 inhibitor is burixafor.

Embodiment A175. The method for suppressing, the method for treating, the pharmaceutical kit for use, the pharmaceutical composition for use, or the pharmaceutical composition, of any one of embodiments A1-A9 or A14-A168, wherein the method or use further comprises determining the expression level of the ADRB2 gene in the cell, in the subject, or in the sample obtained from the subject, wherein the cell, the subject, or the sample obtained from the subject is determined to have a ADRB2-expressing cancer if the expression level is greater than a reference level of the ADRB2 gene.

Embodiment A176. The method for suppressing, the method for treating, the pharmaceutical kit for use, the pharmaceutical composition for use, or the pharmaceutical composition, of embodiment A175, wherein the cancer is an ADRB2-expressing cancer.

Embodiment A177. The method for suppressing, the method for treating, the pharmaceutical kit for use, the pharmaceutical composition for use, or the pharmaceutical composition, of embodiment A175 or A176, wherein the ADRB2 expression level in the cell is greater than a reference level.

Embodiment A178. The method for suppressing, the method for treating, the pharmaceutical kit for use, the pharmaceutical composition for use, or the pharmaceutical composition, of embodiment A175 or A176, wherein the ADRB2 expression level in the subject is greater than a reference level.

Embodiment A179. The method for suppressing, the method for treating, the pharmaceutical kit for use, the pharmaceutical composition for use, or the pharmaceutical composition, of embodiment A175 or A176, wherein the ADRB2 expression level in the sample obtained from the subject is greater than a reference level.

Embodiment A180. The method for suppressing, the method for treating, the pharmaceutical kit for use, the pharmaceutical composition for use, or the pharmaceutical composition, of any one of embodiments A175-179, upon determining the ADRB2 gene expression level is greater than the reference level, the method or use comprises administering a CXCR4 inhibitor and an ADRB2 inhibitor, wherein the CXCR4 inhibitor is burixafor.

Embodiment A181. The method for suppressing, the method for treating, the pharmaceutical kit for use, the pharmaceutical composition for use, or the pharmaceutical composition, of any one of embodiments A1-A9 or A14-A168, wherein the method or use further comprises determining the expression level of the CXCR4 gene and the ADRB2 gene in the cell, in the subject, or in the sample obtained from the subject, wherein the cell, the subject, or the sample obtained from the subject is determined to have a CXCR4-expressing and ADRB2-expressing cancer if the expression levels of the CXCR4 gene and the ADRB2 gene are greater than respective reference levels of the CXCR4 gene and the ADRB2 gene.

Embodiment A182. The method for suppressing, the method for treating, the pharmaceutical kit for use, the pharmaceutical composition for use, or the pharmaceutical composition, of embodiment A181, wherein the cancer is a CXCR4-expressing cancer and an ADRB2-expressing cancer.

Embodiment A183. The method for suppressing, the method for treating, the pharmaceutical kit for use, the pharmaceutical composition for use, or the pharmaceutical composition, of embodiment A181 or A182, wherein the CXCR4 expression level and the ADRB2 expression level in the cell are greater than respective reference levels.

Embodiment A184. The method for suppressing, the method for treating, the pharmaceutical kit for use, the pharmaceutical composition for use, or the pharmaceutical composition, of embodiment A181 or A182, wherein the CXCR4 expression level and the ADRB2 expression level in the subject are greater than respective reference levels.

Embodiment A185. The method for suppressing, the method for treating, the pharmaceutical kit for use, the pharmaceutical composition for use, or the pharmaceutical composition, of embodiment A181 or A182, wherein the CXCR4 expression level and the ADRB2 expression level the sample obtained from the subject are greater than respective reference levels.

Embodiment A186. The method for suppressing, the method for treating, the pharmaceutical kit for use, the pharmaceutical composition for use, or the pharmaceutical composition, of any one of embodiments A181-A185, wherein upon determining the CXCR4 gene and the ADRB2 gene expression levels are greater than the respective reference levels, the method or use comprises administering a CXCR4 inhibitor and an ADRB2 inhibitor, wherein the CXCR4 inhibitor is burixafor.

Embodiment A187. The method for suppressing, the method for treating, the pharmaceutical kit for use, the pharmaceutical composition for use, or the pharmaceutical composition, of any one of embodiments A1-A9 or A14-A168, wherein the method or use further comprises determining the expression level of the CXCR4 protein in the cell, in the subject, or in the sample obtained from the subject, wherein the cell, the subject, or the sample obtained from the subject is determined to have a CXCR4-expressing cancer if the expression level is greater than a reference level of the CXCR4 protein.

Embodiment A188. The method for suppressing, the method for treating, the pharmaceutical kit for use, the pharmaceutical composition for use, or the pharmaceutical composition, of embodiment A187, wherein the cancer is a CXCR4-expressing cancer.

Embodiment A189. The method for suppressing, the method for treating, the pharmaceutical kit for use, the pharmaceutical composition for use, or the pharmaceutical composition, of embodiment A187 or A188, wherein the CXCR4 expression level in the cell is greater than a reference level.

Embodiment A190. The method for suppressing, the method for treating, the pharmaceutical kit for use, the pharmaceutical composition for use, or the pharmaceutical composition, of embodiment A187 or A188, wherein the CXCR4 expression level in the subject is greater than a reference level.

Embodiment A191. The method for suppressing, the method for treating, the pharmaceutical kit for use, the pharmaceutical composition for use, or the pharmaceutical composition, of embodiment A187 or A188, wherein the CXCR4 expression level in the sample obtained from the subject is greater than a reference level.

Embodiment A192. The method for suppressing, the method for treating, the pharmaceutical kit for use, the pharmaceutical composition for use, or the pharmaceutical composition, of any one of embodiments A187-A191, upon determining the CXCR4 protein expression level is greater than the reference level, the method or use comprises administering a CXCR4 inhibitor and an ADRB2 inhibitor, wherein the CXCR4 inhibitor is burixafor.

Embodiment A193. The method for suppressing, the method for treating, the pharmaceutical kit for use, the pharmaceutical composition for use, or the pharmaceutical composition, of any one of embodiments A1-A9 or A14-A168, wherein the method or use further comprises determining the expression level of the ADRB2 protein in the cell, in the subject, or in the sample obtained from the subject, wherein the cell, the subject, or the sample obtained from the subject is determined to have a ADRB2-expressing cancer if the expression level is greater than a reference level of the ADRB2 protein.

Embodiment A194. The method for suppressing, the method for treating, the pharmaceutical kit for use, the pharmaceutical composition for use, or the pharmaceutical composition, of embodiment A193, wherein the cancer is an ADRB2-expressing cancer.

Embodiment A195. The method for suppressing, the method for treating, the pharmaceutical kit for use, the pharmaceutical composition for use, or the pharmaceutical composition, of embodiment A193 or A194, wherein the ADRB2 expression level in the cell is greater than a reference level.

Embodiment A196. The method for suppressing, the method for treating, the pharmaceutical kit for use, the pharmaceutical composition for use, or the pharmaceutical composition, of embodiment A193 or A194, wherein the ADRB2 expression level in the subject is greater than a reference level.

Embodiment A197. The method for suppressing, the method for treating, the pharmaceutical kit for use, the pharmaceutical composition for use, or the pharmaceutical composition, of embodiment A193 or A194, wherein the ADRB2 expression level in the sample obtained from the subject is greater than a reference level.

Embodiment A198. The method for suppressing, the method for treating, the pharmaceutical kit for use, the pharmaceutical composition for use, or the pharmaceutical composition, of any one of embodiments A193-A197, upon determining the ADRB2 protein expression level is greater than the reference level, the method or use comprises administering a CXCR4 inhibitor and an ADRB2 inhibitor, wherein the CXCR4 inhibitor is burixafor.

Embodiment A199. The method for suppressing, the method for treating, the pharmaceutical kit for use, the pharmaceutical composition for use, or the pharmaceutical composition, of any one of embodiments A1-A9 or A14-A168, wherein the method or use further comprises determining the expression level of the CXCR4 protein and the ADRB2 protein in the cell, in the subject, or in the sample obtained from the subject, wherein the cell, the subject, or the sample obtained from the subject is determined to have a CXCR4-expressing and ADRB2-expressing cancer if the expression levels of the CXCR4 protein and the ADRB2 protein are greater than respective reference levels of the CXCR4 protein and the ADRB2 protein.

Embodiment A200. The method for suppressing, the method for treating, the pharmaceutical kit for use, the pharmaceutical composition for use, or the pharmaceutical composition, of embodiment A199, wherein the cancer is a CXCR4-expressing cancer and an ADRB2-expressing cancer.

Embodiment A201. The method for suppressing, the method for treating, the pharmaceutical kit for use, the pharmaceutical composition for use, or the pharmaceutical composition, of embodiment A199 or A200, wherein the CXCR4 expression level and the ADRB2 expression level in the cell are greater than respective reference levels.

Embodiment A202. The method for suppressing, the method for treating, the pharmaceutical kit for use, the pharmaceutical composition for use, or the pharmaceutical composition, of embodiment A199 or A200, wherein the CXCR4 expression level and the ADRB2 expression level in the subject are greater than respective reference levels.

Embodiment A203. The method for suppressing, the method for treating, the pharmaceutical kit for use, the pharmaceutical composition for use, or the pharmaceutical composition, of embodiment A199 or A200, wherein the CXCR4 expression level and the ADRB2 expression level the sample obtained from the subject are greater than respective reference levels.

Embodiment A204. The method for suppressing, the method for treating, the pharmaceutical kit for use, the pharmaceutical composition for use, or the pharmaceutical composition, of any one of embodiments A199-A203, wherein upon determining the CXCR4 protein and the ADRB2 protein expression levels are greater than the respective reference levels, the method or use comprises administering a CXCR4 inhibitor and an ADRB2 inhibitor, wherein the CXCR4 inhibitor is burixafor.

Embodiment A205. The method for suppressing, the method for treating, the pharmaceutical kit for use, the pharmaceutical composition for use, or the pharmaceutical composition, of any one of embodiments A1-A204, wherein the ADRB2 inhibitor is carvedilol.

Embodiment A206. The method for suppressing, the method for treating, the pharmaceutical kit for use, the pharmaceutical composition for use, or the pharmaceutical composition, of any one of embodiments A1-A205, wherein the enhanced downstream signaling is an enhanced response to co-stimulation of the CXCR4-ADRB2 heteromer with a CXCR4 agonist and an ADRB2 agonist.

Embodiment A207. The method for suppressing, the method for treating, the pharmaceutical kit for use, the pharmaceutical composition for use, or the pharmaceutical composition, of any one of embodiments A1-A206, wherein the enhanced downstream signaling is an enhanced cancer progression in the subject having cell(s) containing said CXCR4-ADRB2 heteromer.

Embodiment A208. The method for suppressing, the method for treating, the pharmaceutical kit for use, the pharmaceutical composition for use, or the pharmaceutical composition, of embodiment A207, wherein the enhanced cancer progression is an enhanced cell proliferation in the subject having cell(s) containing said CXCR4-ADRB2 heteromer.

Embodiment A209. The method for suppressing, the method for treating, the pharmaceutical kit for use, the pharmaceutical composition for use, or the pharmaceutical composition, of embodiment A207 or A208, wherein the enhanced cancer progression is an enhanced cell migration in the subject having cell(s) containing said CXCR4-ADRB2 heteromer.

Embodiment A210. The method for suppressing, the method for treating, the pharmaceutical kit for use, the pharmaceutical composition for use, or the pharmaceutical composition, of any one of embodiments A207-A209, wherein the enhanced cancer progression is an enhanced metastasis in the subject having cell(s) containing said CXCR4-ADRB2 heteromer.

Embodiment A211. The method for suppressing, the method for treating, the pharmaceutical kit for use, the pharmaceutical composition for use, or the pharmaceutical composition, of any one of embodiments A207-A210, wherein the enhanced cancer progression is an enhanced tumor growth in the subject having cell(s) containing said CXCR4-ADRB2 heteromer.

Embodiment A212. The method for suppressing, the method for treating, the pharmaceutical kit for use, the pharmaceutical composition for use, or the pharmaceutical composition, of any one of embodiments A207-A211, wherein the enhanced cancer progression is an enhanced angiogenesis in the subject having cell(s) containing said CXCR4-ADRB2 heteromer.

Embodiment A213. The method for suppressing, the method for treating, the pharmaceutical kit for use, the pharmaceutical composition for use, or the pharmaceutical composition, of any one of embodiments A207-A212, wherein the cell(s) are cancer cell(s).

Embodiment A214. The method for suppressing, the method for treating, the pharmaceutical kit for use, the pharmaceutical composition for use, or the pharmaceutical composition, of any one of embodiments A207-A213, wherein the cell(s) are derived from a subject.

Embodiment A215. The method for suppressing, the method for treating, the pharmaceutical kit for use, the pharmaceutical composition for use, or the pharmaceutical composition, of any one of embodiments A207-A214, wherein the cell(s) are from a biological sample obtained from a subject.

### Materials and Methods

### Reagents

CXCL12 were purchased from R&D systems (Minneapolis, Minn., USA). Ulocuplumab and BKT140 were purchased from Creative Biolabs (Shirley, N.Y., USA) and Chem Scene (Monmouth Junction, N.J., USA), respectively. AMD3100 was obtained from Cayman Chemical Company (Ann Arbor, Mich., USA). AMD070 and LY2510924 were purchased from Medchem express (Princeton, N.J., USA). TG-0054 (Brixafor) was purchased from MedKoo Biosciences, Inc (Triangle Park, N.C., USA). Carvedilol was purchased from 4 Chem Laboratory (Korea). Information on drugs used is as follows: Recombinant Human SDF-1α (CXCL12) (PEPROTECH, Cat. 300-28A, Rocky Hill, N.J., USA), Salmeterol (Tocris, Cat. 4712, Minneapolis, Minn., USA), Carvedilol (Tocris, Cat. 2685, Minneapolis, Minn., USA), AMD3100 (Tocris, Cat. 3299, Minneapolis, Minn., USA), TG-0054 (MedKoo Biosciencex, Inc., Cat. 206522, Morrisville, N.C., USA), Bupranolol (Abcam, ab141132, Cambridge, CB2 0AX, UK), Labetalol (Prestwick Chemical Library®, Prestw-277), Alprenolol (Prestwick Chemical Library®, Prestw-250, Boulevard Gonthier d'Andernach Parc d'innovation 67400 ILLKIRCH—France), Carazolol (PubChem, Cat. 71739, Bethesda, Md., USA), Propafenone (Prestwick Chemical Library®, Prestw-499), and Timolol (Prestwick Chemical Library®, Prestw-948).

### Cell Culture

All cell lines were purchased from American Type Culture Collection (Manassas, Va., USA). A549, MDA-MB-231, U2OS, HL-60, U937 and RPMI 8226 cells were grown in RPMI medium 1640 (Thermo Fisher Scientific, Waltham, Mass., USA) supplemented with 10% fetal bovine serum (Gibco) and 1% penicillin-streptomycin (Gibco). All cell lines were grown at 37° C. in the presence of 5% CO₂.

### Construction of Stable Cell Lines

To establish stable Rluc and luc2P expressing cell lines as a control cell lines having hygromycin and blasticidin resistance, lentiviral stock (containing the packaged pLenti6-Rluc expression construct which inserted with Rluc gene in pLenti-CMV Hygro DEST (Addgene, #17454)), were produced by co-transfecting the ViraPower Lentiviral Packaging Mix (Invitrogen, K497500) and pLenti6-Rluc expression construct into 293FT producer cell line. Transduction of this lentiviral stock into A549 cell line was performed and followed by selection with hygromycin (100 μg/mL). To establish stable Rluc-luc2P expressing cell lines, lentiviral stock (containing the packaged pLenti6/V5-luc2P expression construct which inserted with Luc2P gene in pLenti6/V5-DEST Gateway™ Vector (Invitrogen, V49610)), were produced by co-transfecting the ViraPower Packaging Mix and pLenti6/V5-luc2P expression construct into 293FT producer cell line. Transduction of this lentiviral stock into A549-Rluc cell line was performed and followed by selection with blasticitin (5 μg/mL). Then clones resistant to antibiotics were selected and performed RT-qPCR and immunofluorescence to confirm the expression of the inserted gene, Rluc and luc2P.

To establish stable CXCR4 expressing cell lines, lentiviral stock (containing the packaged pLenti6-CXCR4 expression construct which inserted with CXCR4 gene in pLenti-CMV Hygro DEST (Addgene, #17454)), were produced by co-transfecting the ViraPower Lentiviral Packaging Mix (Invitrogen, K497500) and pLenti6-CXCR4 expression construct into 293FT producer cell line. Transduction of this lentiviral stock into A549 cell line was performed and followed by selection with hygromycin (100 μg/mL). To establish stable CXCR4-ADRB2 heteromer expressing cell lines, lentiviral stock (containing the packaged pLenti6/V5-ADRB2 expression construct which inserted with ADRB2 gene in pLenti6/V5-DEST Gateway™ Vector (Invitrogen, V49610)), were produced by co-transfecting the ViraPower Packaging Mix and pLenti6/V5-ADRB2 expression construct into 293FT producer cell line. Transduction of this lentiviral stock into A549-CXCR4 cell line was performed and followed by selection with hygromycin (100 μg/mL) and blasticitin (5 μg/mL). Then clones resistant to antibiotics were selected and performed RT-qPCR and immunofluorescence to confirm the expression of the inserted gene, CXCR4 and ADRB2. pLenti CMV Hygro DEST (w117-1) was a gift from Eric Campeau & Paul Kaufman (Addgene plasmid #17454) (Campeau, E., et al., (2009) A versatile viral system for expression and depletion of proteins in mammalian cells, PLoS One 4, e6529). CXCR4 cDNA was inserted into the lentiviral vector using LR recombination. Lentiviruses encoding CXCR4 were produced using ViraPower Lentiviral Expression Systems (Invitrogen).

To establish stable CXCR4 expressing cell lines in MDA-MB-231 breast cancer cell, lentiviral stock (containing the packaged pLenti6-CXCR4 expression construct which inserted with CXCR4 gene in pLenti-CMV Hygro DEST (Addgene, #17454)), were produced by co-transfecting the ViraPower Lentiviral Packaging Mix (Invitrogen, K497500) and pLenti6-CXCR4 expression construct into 293FT producer cell line. Transduction of this lentiviral stock into MDA-MB-231 cell line was performed and followed by selection with hygromycin (100 μg/mL). To establish stable cell lines expressing both CXCR4 and ADRB2 such that the cells contain CXCR4-ADRB2 heteromer, lentiviral stock (containing the packaged pLenti6/V5-ADRB2 expression construct which inserted with ADRB2 gene in pLenti6/V5-DEST Gateway™ Vector (Invitrogen, V49610)), were produced by co-transfecting the ViraPower Packaging Mix and pLenti6/V5-ADRB2 expression construct into 293FT producer cell line. Transduction of this lentiviral stock into MDA-MB-231-CXCR4 cell line was performed and followed by selection with hygromycin (100 μg/mL) blasticitin (5 μg/mL). Then clones resistant to antibiotics were selected and performed RT-qPCR and immunofluorescence to confirm the expression of the inserted gene, CXCR4 and ADRB2.

### Calcium Mobilization Assay

MDA-MB-231 human breast cancer cells were seeded at 20,000 cells per well in a black clear bottom 96-well plate (Corning Costar, #3340) in 100 μL of RPMI 1640 supplemented with 10% FBS. The next day, cells were co-transduced with 10 MOI of CXCR4 and 30 MOI of GPCRx. Adenoviruses encoding HA-VC were used to adjust the total amount of adenoviruses transduced. After 2 days, cells were treated with antagonist with indicated amounts and incubated with Cal 6 (FLIPR® Calcium 6 Assay Kit by Molecular Devices, Cat. R8191) for 2 hr. And then, cells were stimulated with indicated amounts of CXCL12, ADRB2 agonist, or CXCL12 and ADRB2 agonist. Calcium mobilization was measured using FlexStation 3 Multi-Mode Microplate Reader (Molecular Devices, Sunnyvale, Calif., USA). The results were normalized for base-line activity. Calcium mobilization was measured at 37° C. using an excitation wavelength of 490 nm and an emission wavelength of 525 nm and quantified by calculating the area-under-the-curve (AUC) of each graph. Data were normalized to CXCL12-stimulated calcium response in cells expressing CXCR4 alone. Data represent three independent experiments (mean±SEM).

Utilizing the Calcium mobilization assay, U937 (human myeloid leukemia cell) and HL-60 (human leukemia cell) cells were seeded at 100,000 cells per well in a black clear bottom 96-well plate (Corning Costar, #3340) in 100 μL of RPMI 1640 supplemented with 10% FBS. And centrifuge the cell at 100×g for 1 minute. Cells were stained with Cal 6 (FLIPR® Calcium 6 Assay Kit by Molecular Devices, Cat. R8191) diluted in assay buffer (Hank's balanced salt solution without phenol red) and incubated for 2 hr. Antagonists, if needed, were added 2 hours before agonist treatment. Cells were stimulated with indicated amounts of CXCR4 agonist (CXCL12, 200 nM), ADRB2 agonist (Formoterol, 10 μM). Calcium mobilization was measured using Flexstation 3 Multi-Mode Microplate Reader (Molecular Devices, Sunnyvale, Calif., USA). Intracellular Ca²⁺ was measured at 37° C. using an Excitation wavelength of 490 nm and an emission wavelength of 525 nm. The results were normalized for base-line activity. Calcium mobilization was quantified by calculating the area-under-the curve (AUC) of each graph. Data were normalized to CXCL12-stimulated calcium response in cells expressing CXCR4 alone. And IC₅₀ values were calculated using GraphPad Prism software.

### Western Blot Analysis

Cells were seeded in 6 well plates at a density of 5×10⁵ cells per well. After 16 hours of serum starvation, the cells were treated with 10 nM of SDF-1 (PEROTECH, #300-28A) and Salmeterol (TOCRIS, #4712) for 20 minutes for MDA-MB-231, MDA-MB-231-CXCR4, and MDA-MB-231-CXCR4-ADRB2 cells and 10 minutes for A549, A549-CXCR4, A549-CXCR4-ADRB2 cells. Thereafter, the cells were harvested using NP-40 lysis buffer (Thermo Fisher Scientific, #FNN0021) supplemented with phosphatase inhibitor cocktail (Roche, #4906845001) and protease inhibitor cocktail (Thermo Fisher Scientific, #A32953). Proteins of cell lysates concentrations were determined by the Bio-Rad protein assay (Bio-Rad, #5000006). 20 μg of protein samples were subjected to reducing SDS-PAGE and transferred to PVDF (Polyvinylidene difluoride) using dry transfer system (Thermo Fisher Scientific, #IB21001). The membranes were blocked with 5% skim milk (BD Difco, #232100) in Tris-Buffered Saline with 1% Tween-twenty for 1 hour and incubated with phospho-ERK1/2 Thr202/Tyr204 (Cell Signaling Technology, #4370) and total-ERK1/2 (Cell Signaling Technology, #4695) antibodies. Antigen-antibody complexes were detected by incubating with horseradish peroxidase coupled secondary antibodies (Cell Signaling Technology) followed by SuperSignal West Femto Maximum Sensitivity Substrate (Thermo Fisher Scientific, #34096). Western blot images were captured and analyzed by iBright Western Blot Imaging System (Thermo Fisher Scientific, #A32752).

Quantitative Polymerase Chain Reaction (qPCR)

Total RNA was extracted using TRIzol (Invitrogen) and cDNA was synthesized from 1 μg of total RNA after treatment with DNase I (Sigma). qPCR was conducted using SensiFAST SYBR kit (Bioline). Primer sequences are as follows:

ADRB2-F:
(SEQ ID NO: 1)
5′-CTCTTCCATCGTGTCCTTCTAC-3′,
ADRB2-R:
(SEQ ID NO: 2)
5′-AATCTTCTGGAGCTGCCTTT-3′;
CXCR4-F:
(SEQ ID NO: 3)
5′-CCACCATCTACTCCATCATCTTC-3′,
CXCR4-R:
(SEQ ID NO: 4)
5′-ACTTGTCCGTCATGCTTCTC-3′;
β-actin-F:
(SEQ ID NO: 5)
5′-GGAAATCGTGCGTGACATTAAG-3′,
β-actin-R:
(SEQ ID NO: 6)
5′-AGCTCGTAGCTCTTCTCCA-3′;

Threshold cycles (Ct) were calculated by QuantStudio 3 Real-Time PCR system (Thermo Fisher Scientific). Delta Ct (ΔCt) corresponds to the difference between CtSOI (sequence of interest) and Ct RS (reference sequence), a house keeping gene sequence usual; Delta Ct (ΔCt)=Ct(CXCR4 or ADRB2)−Ct (actin).

Construction of Adenoviral Vectors Containing GPCR cDNAs

Human GPCR cDNA clones were obtained from the Missouri S&T cDNA Resource Center (Rolla, Mo., USA). GPCR cDNAs were amplified by PCR and cloned into pDONR201 vector. Adenoviruses encoding GPCR were obtained through in vitro LR recombination between entry clones containing GPCR and pAdHTS vectors, and subsequent transfection into 293A cells using AdHTS system as described previously (Choi, E. W., et al., (2012) AdHTS: a high-throughput system for generating recombinant adenoviruses, J Biotechnol 162, 246-252; and Song, Y. B., et al., (2014)).

### Proliferation Assay

A549 double negative (RLuc-lucP) or A549-CXCR4-ADRB2 cell lines overexpressing both CXCR4 and ADRB2 plated at a density of 1×10⁴ cells/well in 96-well plates and incubated in culture medium for 24 hr to allow adherence. After washing, Serum free RPMI1640±SDF-1α with or without indicated drugs were added and the cells were incubated for 72 hr (96 hr post seeding) at 37° C., 5% CO₂. At the end of incubation, cell proliferation was assessed by using Prestoblue Cell Viability Reagent (Thermo Fisher Scientific, Cat. A13262) according to the manufacture's instruction. Briefly, cells were incubated in 1× Prestoblue Cell Viability Reagent for 1 hr at 37° C. Fluorescence was measured at 560 nm excitation and 590 nm emission using Varioskan LUX multimode microplate reader (Thermo Fisher Scientific). The amount of fluorescence detected was proportional to the number of cells within a well. Results are expressed as mean ratio (Fluorescence_{Drug}/Fluorescence_{Vehicle only}) from triplicate wells±SEM.

### Mouse Xenograft Model

Five week-old, female Balb/c-nu/nu mice were obtained from Envigo (France) and maintained in specific pathogen-free animal facility. All protocols for animal use and euthanasia were approved by the Qubest Bio (South Korea) Animal Experimental Ethics Committee based on the Animal Protection Act. The 1×10⁷ cells of A549, or A549-CXCR4-ADRB2 were suspended in 100 μL of phosphate-buffered saline (PBS) and were implanted by subcutaneously in axillary region between the clavicular and chest wall on the right side of the mouse. The tumor growth was monitored every third or fourth day by measuring the length (L) and width (W) of the tumor with an electronic caliper and calculating tumor volume in the basis of the following formula: Volume=0.5 LW².

### Anti-Tumor Effect Studies

For the antitumor efficacy test using CXCR4 inhibitors, A549-CXCR4-ADRB2 cell line overexpressing CXCR4 and ADRB2 in A549, lung cancer cell line was subcutaneously administered (1×10⁷ cell in 100 μL PBS/head) to BALB/c-nu. When Tumor size reached 50-100 mm³, groups of ten mice were administrated with vehicle (1% dimethylcellulose in PBS), AMD3100 (7.5 mpk, 22.5 mpk formulated in PBS), LY2510924 (3 mpk, 10 mpk formulated in PBS), AMD070 (10 mpk, 30 mpk formulated in 1% dimethylcellulose in PBS) or TG-0054 (10 mpk, 30 mpk formulated in PBS) or Carvedilol (20 mpk formulated in 1% dimethylcellulose in PBS). AMD3100, LY2510924 and TG-0054 were subcutaneously injected and AMD070 and Carvedilol were administrated orally once a day for 4 weeks (28 times in total). The tumor growth was monitored every third or fourth day by measuring the length (L) and width (W) of the tumor: Volume=0.5 LW².

### Orthotopic Model

MDA-MB-231 cells or MDA-MB-231-CXCR4-ADRB2 cells are orthotopically administered at Balb/c-nu/nu female mice (5×10⁶ cells/head (100 μL cells+100 μL Matrigel)) using FBS free media. Cells are treated with Matrigel™ (BD, 354248) prior to administration in the following manner. 1) Matrigel and the syringe and needle used for the administration also keep the condition of 10° C. until the administration. 2) Matrigel is mixed with the cell suspension at a ratio of 50:50, and needle size of 23 G is used to prevent cell destruction.

Place the needle point in the pre-administration site (between the nipple 2 and 3) and move it to the right or left to make a wide subcutaneous pocket. The Matrigel Mixture (matrigel: cell suspension=50:50) is injected into the subcutaneous pocket. The animals are placed in a breeding box after confirming that the liquid is not spilled at the injection site. When tumor size were reached at about 50-100 mm³, we treated with CXCR4 inhibitor or ADRB2 inhibitor alone or in combination at indicated dose daily for 4 weeks. The day of drug treatment is defined as Day 1. AMD3100 (2.5 mg/kg, 7.5 mg/kg in PBS), LY2510924 (1 mg/kg, 3 mg/kg in PBS), AMD070 (3 mg/kg, 10 mg/kg in 1% methylcellulose in PBS) and/or Carvedilol (30 mg/kg in 1% methylcellulose in PBS) was administered once a day for 4 weeks (28 times in total). AMD3100, LY2510924, and AMD070 were administered subcutaneously, and Carvedilol was administered orally. The size of the tumor was calculated by converting the tumor size to 100 on the first day of drug administration. The results are presented as the mean±standard deviation of 5 individuals.

### Antibody TR-FRET

Before the TR-FRET assay, 10,000 cells were plated in each well of 96-well half-area plate (Corning). After the overnight incubation in the 37° C. humidified incubator, both CXCR4 and ADRB2 were transiently overexpressed using adenoviral system (0.9-30 MOI of CXCR4-carrying adenovirus and 0.5-15 MOI of ADRB2-carrying adenovirus). The cells were washed twice with DPBS and fixed with 4% paraformaldehyde at room temperature for 10 minutes following the 48 h infection of each adenovirus. After washing the cells twice, to permeabilize the cells, DPBS containing 0.1% Triton X-100 was treated at room temperature for 10 minutes. The cells were then blocked with DPBS supplemented with 2% bovine serum albumin (Sigma-Aldrich, St. Louis, Mo., USA) at room temperature for 30 minutes, followed by incubation with both rabbit anti-CXCR4 antibody (#ab124824, abcam) and mouse anti-ADRB2 antibody (#sc271322, Santa Cruz Biotechnology) at room temperature for 3 hours. After washing the cells with DPBS for 4 times, goat anti-rabbit IgG labeled with terbium cryptate (Cisbio, Bedford, Mass., USA) and goat anti-mouse IgG labeled with Alexa Fluor 647 (Thermo Fisher) were treated at room temperature for 1 hour. The cells were then washed with DPBS for 4 times, followed by analysis using Varioskan LUX Multimode Microplate Reader (Thermo Fisher Scientific). The FRET ratio is simply calculated for each well as the ratio between the acceptor FRET signal at 520 nm and the donor emission at 620 nm.

FRET ratio=signal at 520 nm/signal at 620 nm×10000

The resulting data represents the FRET efficiency. The calculation of the ΔF % is based on the different FRET ratio.

ΔF %=(Ratioₛₐₘₚₗₑ−Ratio_{neg})/Ratio_{neg}×100

ΔF % takes into account the variation of the donor concentration and corresponds to a percentage of the FRET increase compared to a negative control. However, the ΔF % parameter does not allow the comparison of experiments that are not performed with the same donor-ligand concentrations.

### Ligand TR-FRET

A549 cells were seeded in 96-well plates (Corning, N.Y., USA #3688) at a density of 20,000 cells per well. CXCR4 and ADRB2 were transiently over-expressed in A549 cells by adenovirus infection followed by 48 h incubation at 37° C., 5% CO₂. CXCR4 expressing adenovirus was treated at MOI of 0, 0.1, 0.5, 1.25, 2.5, 5, 10 and 20, and ADRB2 expressing adenovirus was treated at three times higher MOI. Adenoviruses encoding HA-VC were used to adjust the total amount of adenoviruses transduced.

To characterize CXCR4:ADRB2 heteromers TR-FRET assay with labeled ligand was performed with fluorescently labeled Propranolol and terbium labeled TZ14011 (Cisbio, USA). 18 hours after infection with CXCR4 and ADRB2, the ligands were prepared separately in ice-cold Tris-KREBS buffer (20 mM Tris-HCl, 118 mM NaCl, 5.6 mM glucose, 1.2 mM KH₂PO₄, 1.2 mM MgSO₄, 4.7 mM KCl, 1.8 mM CaCl₂), pH 7.4) and kept on ice (by working below 15° C., all internalization phenomena are avoided). At this step, all ligand solutions were prepared at 5 times the desired final concentration with Tris-KREBS buffer. Cells were labeled with 10 nM TZ14011-tb, 20 nM Propranolol-g2 (Cisbio, USA #L0011GRE) and 10 mM unlabeled propranolol (PRESTWICK CHEMICAL, France) in 96-well plate by incubating for 18 hr at 4° C. in the dark and TR-FRET signal was measured with Varioskan LUX Multimode (Thermo Fisher Scientific, USA) plate reader. The preparations were excited at 334 nm and fluorescence emissions were measured at 620 nm (TZ14011-Tb) for the donor and at 520 nm (Propranolol-g2) depending on acceptor on a VARIOSKAN. Data analysis is the same as the Antibody TR-FRET analysis.

### Proximity Ligation Assay (PLA)

Unfold PLA was performed according to the manufacturer's protocol (Olink Proteomics, Uppsala, Sweden). Since the cells used in the assay are floating, all resuspension steps were performed by centrifugation at 2,000 rpm for 3 minutes. Each washing step was carried out twice using centrifugation between all reactions. Cultured cells were washed in 1 mL of DPBS (Thermo Fisher Scientific), followed by fixation of the cells in 4% paraformaldehyde at room temperature for 10 minutes. The cells were then permeabilized in DPBS containing 0.1% Triton X-100 and blocked in blocking solution at 37° C. for 1 hour. Blocking solution was then removed, and the cells were resuspended by primary antibody solution. Both mouse anti-CXCR4 antibody (#35-8800, Thermo Fisher Scientific) and rabbit anti-ADRB2 antibody (#PA5-33333, Thermo Fisher Scientific) were diluted in antibody diluent (1:200 and 1:2000, respectively). After the incubation at 37° C. for 1 hour, PLA probes were applied at a dilution of 1:50 diluted in antibody diluent. The probes were incubated with the cells at 37° C. for 1 hour. Digestion of the probes were performed at 37° C. for 1 hour, followed by ligation step at 37° C. for 30 minutes. Amplification mix was then treated to the cells at 37° C. for 100 minutes. Mounting of the cells with 20 μL of VECTASHIELD Antifade Mounting Medium with DAPI (Vector Laboratories). PLA images were acquired with IN Cell Analyzer 2500 (GE Healthcare).

All publications and patent applications mentioned in this specification are herein incorporated by reference in their entirety to the same extent as if each individual publication or patent application was specifically and individually indicated to be incorporated by reference.

While preferred embodiments have been shown and described herein, it will be obvious to those skilled in the art that such embodiments are provided by way of example only. It is intended that the following claims define the scope of the invention and that methods and structures within the scope of these claims and their equivalents be covered thereby.

What is claimed is:

1. A method for suppressing enhanced downstream signaling resulting from a CXCR4-ADRB2 heteromer in a cell of a subject suffering from cancer, the method comprising administering to the subject:
a) a CXCR4 inhibitor, wherein the CXCR4 inhibitor is burixafor; and
b) an ADRB2 inhibitor;
wherein:
i) the enhanced downstream signaling results from the CXCR4-ADRB2 heteromer; and
ii) the administered burixafor and ADRB2 inhibitor suppresses the enhanced downstream signaling from said CXCR4-ADRB2 heteromer in the cancer subject.

2. A method for treating cancer in a subject having a cell containing a CXCR4-ADRB2 heteromer, the method comprising administering to the subject:
a) a CXCR4 inhibitor, wherein the CXCR4 inhibitor is burixafor; and
b) an ADRB2 inhibitor;
wherein:
i) enhanced downstream signaling results from the CXCR4-ADRB2 heteromer; and
ii) the administered burixafor and ADRB2 inhibitor suppresses the enhanced downstream signaling from said CXCR4-ADRB2 heteromer in the cancer subject.

3. A method for treating cancer in a subject having a cell containing a CXCR4-ADRB2 heteromer, the method comprising:
a) determining whether the subject's cell contains a CXCR4-ADRB2 heteromer, wherein enhanced downstream signaling results from the CXCR4-ADRB2 heteromer; and
b) if the subject's cell contains said CXCR4-ADRB2 heteromer, then administering to the cancer subject:
i) a CXCR4 inhibitor, wherein the CXCR4 inhibitor is burixafor; and
ii) an ADRB2 inhibitor.

4. A method for treating cancer in a subject having a cell containing a CXCR4-ADRB2 heteromer, wherein enhanced downstream signaling results from the CXCR4-ADRB2 heteromer, the method comprising:
1) determining whether the subject has the cell containing the CXCR4-ADRB2 heteromer by: obtaining or having obtained a biological sample from the subject; and performing or having performed an assay on the biological sample to determine if:
i) the subject's cell contains said CXCR4-ADRB2 heteromer; or
ii) a combination of a CXCR4 inhibitor and an ADRB2 inhibitor: alters heteromer-specific properties or function of said CXCR4-ADRB2 heteromer in the subject's derived cell(s); alters heteromer-specific properties of the subject's derived cell(s) containing said CXCR4-ADRB2 heteromer; or decreases cancer progression in the subject having cells containing said CXCR4-ADRB2 heteromer; and
2) if the subject has a cell containing said CXCR4-ADRB2 heteromer, then administering to the cancer subject a combination of a CXCR4 inhibitor and an ADRB2 inhibitor, wherein the CXCR4 inhibitor is burixafor; and
3) if the subject does not have a cell containing said CXCR4-ADRB2 heteromer, then administering to the cancer subject a single inhibitor of either the burixafor or the ADRB2 inhibitor.

5. A method for treating cancer in a subject having a cell containing a CXCR4-ADRB2 heteromer, wherein enhanced downstream signaling results from the CXCR4-ADRB2 heteromer, the method comprising:
1) determining whether the subject has the cell containing the CXCR4-ADRB2 heteromer by: obtaining or having obtained a biological sample from the subject; and performing or having performed an assay on the biological sample to determine if:
i) the subject's cell contains said CXCR4-ADRB2 heteromer; or
ii) a combination of a CXCR4 inhibitor and an ADRB2 inhibitor: alters heteromer-specific properties or function of said CXCR4-ADRB2 heteromer in the subject's derived cell(s); alters heteromer-specific properties of the subject's derived cell(s) containing said CXCR4-ADRB2 heteromer; or decreases cancer progression in the subject having cells containing said CXCR4-ADRB2 heteromer; and
2) if the subject has a cell containing said CXCR4-ADRB2 heteromer, then administering to the cancer subject a combination of a CXCR4 inhibitor and an ADRB2 inhibitor, wherein the CXCR4 inhibitor is burixafor; and
3) if the subject does not have a cell containing said CXCR4-ADRB2 heteromer, then administering to the cancer subject a single inhibitor of either the burixafor or the ADRB2 inhibitor;
wherein:
a) progression of the cancer in the subject having said cell containing the CXCR4-ADRB2 heteromer is decreased in the range of 5-100% more upon administering to the cancer subject the combination of the burixafor and the ADRB2 inhibitor, relative to administering the single inhibitor of either the burixafor or the ADRB2 inhibitor;
b) efficacy of the burixafor is increased in the range of 5-2000% when administered in combination with the ADRB2 inhibitor to the subject having said cell containing the CXCR4-ADRB2 heteromer, relative to efficacy of the burixafor when administered as a single inhibitor; and/or
c) efficacy of the ADRB2 inhibitor is increased in the range of 5-2000% when administered in combination with the burixafor to the subject having said cell containing the CXCR4-ADRB2 heteromer, relative to efficacy of the ADRB2 inhibitor when administered as a single inhibitor.

6. A method for treating cancer in a subject having a cell containing a CXCR4-ADRB2 heteromer, wherein enhanced downstream signaling results from the CXCR4-ADRB2 heteromer, the method comprising:
1) determining whether the subject's cell contains the CXCR4-ADRB2 heteromer by: obtaining or having obtained a biological sample from the subject and performing or having performed an assay on the biological sample to determine if said CXCR4-ADRB2 heteromer is present in the subject's cell; wherein the assay performed on the biological sample is or comprises one or more of the following: a co-internalization assay, a colocalization assay, in situ hybridization, immunohistochemistry, immunoelectron microscopy, a proximity-based assay, a co-immunoprecipitation assay, enzyme-linked immunosorbent assay (ELISA), flow cytometry, RNAseq, RT-qPCR, expression level of CXCR4, expression level of ADRB2, expression level of CXCR4 and ADRB2, microarray, or a fluorescent animal assay; and
2) if the subject's cell contains said CXCR4-ADRB2 heteromer, then administering to the cancer subject a combination of a CXCR4 inhibitor and an ADRB2 inhibitor, wherein the CXCR4 inhibitor is burixafor; and
3) if the subject's cell does not contain said CXCR4-ADRB2 heteromer, then administering to the cancer subject a single inhibitor of either the burixafor or the ADRB2 inhibitor.

7. A method for treating cancer in a subject having a cell containing a CXCR4-ADRB2 heteromer, wherein enhanced downstream signaling results from the CXCR4-ADRB2 heteromer, the method comprising:
1) determining whether the subject's cell contains the CXCR4-ADRB2 heteromer by: obtaining or having obtained a biological sample from the subject and performing or having performed an assay on the biological sample to determine if said CXCR4-ADRB2 heteromer is present in the subject's cell; wherein the assay performed on the biological sample is or comprises one or more of the following: a co-internalization assay, a colocalization assay, in situ hybridization, immunohistochemistry, immunoelectron microscopy, a proximity-based assay, a co-immunoprecipitation assay, enzyme-linked immunosorbent assay (ELISA), flow cytometry, RNAseq, RT-qPCR, expression level of CXCR4, expression level of ADRB2, expression level of CXCR4 and ADRB2, microarray, or a fluorescent animal assay; and
2) if the subject's cell contains said CXCR4-ADRB2 heteromer, then administering to the cancer subject a combination of a CXCR4 inhibitor and an ADRB2 inhibitor, wherein the CXCR4 inhibitor is burixafor; and
3) if the subject's cell does not contain said CXCR4-ADRB2 heteromer, then administering to the cancer subject a single inhibitor of either the burixafor or the ADRB2 inhibitor;
wherein:
a) progression of the cancer in the subject having said cell containing the CXCR4-ADRB2 heteromer is decreased in the range of 5-100% more upon administering to the cancer subject the combination of the burixafor and the ADRB2 inhibitor, relative to administering the single inhibitor of either the burixafor or the ADRB2 inhibitor;
b) efficacy of the burixafor is increased in the range of 5-2000% when administered in combination with the ADRB2 inhibitor to the subject having said cell containing the CXCR4-ADRB2 heteromer, relative to efficacy of the burixafor when administered as a single inhibitor; and/or
c) efficacy of the ADRB2 inhibitor is increased in the range of 5-2000% when administered in combination with the burixafor to the subject having said cell containing the CXCR4-ADRB2 heteromer, relative to efficacy of the ADRB2 inhibitor when administered as a single inhibitor.

8. A pharmaceutical kit for use in treating cancer in a subject having a cell containing a CXCR4-ADRB2 heteromer, the pharmaceutical kit comprising:
a) a CXCR4 inhibitor, wherein the CXCR4 inhibitor is burixafor; and
b) an ADRB2 inhibitor;
wherein enhanced downstream signaling results from the CXCR4-ADRB2 heteromer.

9. A pharmaceutical composition for use in treating cancer in a subject having a cell containing a CXCR4-ADRB2 heteromer, the pharmaceutical composition comprising:
a) a CXCR4 inhibitor, wherein the CXCR4 inhibitor is burixafor;
b) an ADRB2 inhibitor; and
c) a pharmaceutically acceptable carrier;
wherein enhanced downstream signaling results from the CXCR4-ADRB2 heteromer.

10. A method for suppressing enhanced downstream signaling resulting from a CXCR4-ADRB2 heteromer in a cell, the method comprising administering to the cell:
a) a CXCR4 inhibitor, wherein the CXCR4 inhibitor is burixafor; and
b) an ADRB2 inhibitor;
wherein:
i) the enhanced downstream signaling results from the CXCR4-ADRB2 heteromer; and
ii) the contacting with the burixafor and the ADRB2 inhibitor suppresses the enhanced downstream signaling from said CXCR4-ADRB2 heteromer in the cell.

11. The method for suppressing of claim 10, wherein the method further comprises determining whether the cell contains the CXCR4-ADRB2 heteromer.

12. A pharmaceutical kit for use in suppressing enhanced downstream signaling resulting from a CXCR4-ADRB2 heteromer in a cell, the pharmaceutical kit comprising:
a) a CXCR4 inhibitor, wherein the CXCR4 inhibitor is burixafor; and
b) an ADRB2 inhibitor;
wherein enhanced downstream signaling results from the CXCR4-ADRB2 heteromer.

13. A pharmaceutical composition for use suppressing enhanced downstream signaling resulting from a CXCR4-ADRB2 heteromer in a cell, the pharmaceutical composition comprising:
a) a CXCR4 inhibitor, wherein the CXCR4 inhibitor is burixafor;
b) an ADRB2 inhibitor; and
c) a pharmaceutically acceptable carrier;
wherein enhanced downstream signaling results from the CXCR4-ADRB2 heteromer.

14. The method for suppressing of any one of claims 10-13, wherein the cell is a subject's cell.

15. The method for suppressing of claim 14, wherein the subject's cell is a cancer cell.

16. The method for suppressing of any one of claims 1-13, wherein the cell is a cancer cell.

17. The method for suppressing, the method for treating, the pharmaceutical kit for use, or the pharmaceutical composition for use, of any one of claims 1-9, wherein the method further comprises detecting the presence of the CXCR4-ADRB2 heteromer in the cancer subject.

18. The method for suppressing, the method for treating, the pharmaceutical kit for use, or the pharmaceutical composition for use, of any one of claims 1-9 or 17, wherein the method further comprises identifying the CXCR4-ADRB2 heteromer in the cancer subject.

19. The method for suppressing, the method for treating, the pharmaceutical kit for use, or the pharmaceutical composition for use, of any one of claims 1-9 or 17-18, wherein the method further comprises obtaining a biological sample from the subject.

20. The method for suppressing, the method for treating, the pharmaceutical kit for use, or the pharmaceutical composition for use, of any one of claims 1-9 or 17-19, wherein the method further comprises performing an assay on the biological sample obtained from said subject.

21. The method for suppressing, the method for treating, the pharmaceutical kit for use, or the pharmaceutical composition for use, of any one of claims 1-9 or 17-20, wherein the method further comprises:
i) obtaining or having obtained a biological sample from the cancer subject;
ii) conducting or having conducted a diagnostic assay to determine presence, identity, or presence and identity, of a CXCR4-ADRB2 heteromer in the obtained biological sample from the cancer subject; and
iii) selecting the ADRB2 inhibitor to administer in combination with burixafor to suppress the enhanced downstream signaling resulting from the CXCR4-ADRB2 heteromer.

22. The method for suppressing, the method for treating, the pharmaceutical kit for use, or the pharmaceutical composition for use, of any one of claims 1-9 or 17-21, wherein the method further comprises determining whether the subject's cell contains the CXCR4-ADRB2 heteromer, comprising performing an assay on a biological sample obtained from the subject.

23. The method for suppressing, the method for treating, the pharmaceutical kit for use, or the pharmaceutical composition for use, of any one of claims 1-9 or 17-22, wherein the method further comprises determining whether the subject's cell contains the CXCR4-ADRB2 heteromer, comprising obtaining a biological sample from the subject, and performing an assay on the biological sample obtained from said subject.

24. The method for suppressing, the method for treating, the pharmaceutical kit for use, or the pharmaceutical composition for use, of any one of claims 1-9 or 17-23, wherein the biological sample obtained from said subject contains the CXCR4-ADRB2 heteromer.

25. The method for suppressing, the method for treating, the pharmaceutical kit for use, or the pharmaceutical composition for use, of any one of claims 1-9 or 14-24, wherein the subject's cell contains the CXCR4-ADRB2 heteromer.

26. The method for suppressing, the method for treating, the pharmaceutical kit for use, or the pharmaceutical composition for use, of any one of claims 1-25, wherein the CXCR4-ADRB2 heteromer has two or more of the following characteristics:
1) the CXCR4-ADRB2 heteromer components colocalize and physically interact in the cell, either directly or via intermediate proteins acting as conduits for allosterism;
2) an enhanced downstream signaling results from the CXCR4-ADRB2 heteromer; and/or
3) a combination of burixafor and an ADRB2 inhibitor:
i) alters heteromer-specific properties of the CXCR4-ADRB2 heteromer in the cell;
ii) alters heteromer-specific function of the CXCR4-ADRB2 heteromer in the cell; and/or
iii) alters heteromer-specific properties of the cell containing the CXCR4-ADRB2 heteromer.

27. The method for suppressing, the method for treating, the pharmaceutical kit for use, or the pharmaceutical composition for use, of any one of claims 1-9 or 14-25, wherein the CXCR4-ADRB2 heteromer has two or more of the following characteristics:
1) the CXCR4-ADRB2 heteromer components colocalize and physically interact in the cell, either directly or via intermediate proteins acting as conduits for allosterism;
2) an enhanced downstream signaling results from the CXCR4-ADRB2 heteromer; and/or
3) a combination of burixafor and an ADRB2 inhibitor:
i) alters heteromer-specific properties of the CXCR4-ADRB2 heteromer in the subject's derived cell(s);
ii) alters heteromer-specific function of the CXCR4-ADRB2 heteromer in the subject's derived cell(s);
iii) alters heteromer-specific properties of the subject's derived cell(s) containing the CXCR4-ADRB2 heteromer; and/or
iv) decreases cancer progression in the subject having cells containing said CXCR4-ADRB2 heteromer.

28. The method for suppressing, the method for treating, the pharmaceutical kit for use, or the pharmaceutical composition for use, of any one of claims 1-27, wherein the CXCR4-ADRB2 heteromer is characterized as having the following characteristic: the CXCR4-ADRB2 heteromer components in the cell colocalize and physically interact, either directly or via intermediate proteins acting as conduits for allosterism.

29. The method for suppressing, the method for treating, the pharmaceutical kit for use, or the pharmaceutical composition for use, of any one of claims 1-28, wherein the CXCR4-ADRB2 heteromer components in the cell colocalize and physically interact, either directly or via intermediate proteins acting as conduits for allosterism, is determined by one or more of the following: a co-internalization assay, a colocalization assay, in situ hybridization, immunohistochemistry, immunoelectron microscopy, a proximity-based assay, a co-immunoprecipitation assay, enzyme-linked immunosorbent assay (ELISA), flow cytometry, RNAseq, RT-qPCR, expression level of CXCR4, expression level of ADRB2, expression level of CXCR4 and ADRB2, microarray, or a fluorescent animal assay.

30. The method for suppressing, the method for treating, the pharmaceutical kit for use, or the pharmaceutical composition for use, of any one of claims 1-29, wherein the proximity-based assay is, or comprises, resonance energy transfer (RET), bioluminescence RET (BRET), fluorescence RET (FRET), time-resolved fluorescence RET (TR-FRET), antibody-based FRET, ligand-based FRET, bimolecular fluorescence complementation (BiFC), or a proximity ligation assay (PLA).

31. The method for suppressing, the method for treating, the pharmaceutical kit for use, or the pharmaceutical composition for use, of claim 30, wherein the TR-FRET is a ligand-based TR-FRET.

32. The method for suppressing, the method for treating, the pharmaceutical kit for use, or the pharmaceutical composition for use, of claim 30, wherein the TR-FRET is an antibody-based TR-FRET.

33. The method for suppressing, the method for treating, the pharmaceutical kit for use, or the pharmaceutical composition for use, of any one of claims 1-30, wherein the CXCR4-ADRB2 heteromer components in the cell colocalize and physically interact, either directly or via intermediate proteins acting as conduits for allosterism, is determined by one or more of the following: a co-internalization assay, bimolecular fluorescence complementation (BiFC), RT-PCR, RT-qPCR, expression level of CXCR4, expression level of ADRB2, expression level of CXCR4 and ADRB2, or a proximity ligation assay (PLA).

34. The method for suppressing, the method for treating, the pharmaceutical kit for use, or the pharmaceutical composition for use, of claim 33, wherein the CXCR4-ADRB2 heteromer components in the cell colocalize and physically interact, either directly or via intermediate proteins acting as conduits for allosterism, is determined by a co-internalization assay.

35. The method for suppressing, the method for treating, the pharmaceutical kit for use, or the pharmaceutical composition for use, of claim 33, wherein the CXCR4-ADRB2 heteromer components in the cell colocalize and physically interact, either directly or via intermediate proteins acting as conduits for allosterism, is determined by bimolecular fluorescence complementation (BiFC).

36. The method for suppressing, the method for treating, the pharmaceutical kit for use, or the pharmaceutical composition for use, of claim 33, wherein the CXCR4-ADRB2 heteromer components in the cell colocalize and physically interact, either directly or via intermediate proteins acting as conduits for allosterism, is determined by a proximity ligation assay (PLA).

37. The method for suppressing, the method for treating, the pharmaceutical kit for use, or the pharmaceutical composition for use, of any one of claims 1-9 or 17-36, wherein the assay determines the presence of the CXCR4-ADRB2 heteromer in the biological sample obtained from said subject.

38. The method for suppressing, the method for treating, the pharmaceutical kit for use, or the pharmaceutical composition for use, of any one of claims 1-37, wherein the assay determines colocalization and interaction of the CXCR4 and ADRB2 components of the CXCR4-ADRB2 heteromer.

39. The method for suppressing, the method for treating, the pharmaceutical kit for use, or the pharmaceutical composition for use, of any one of claims 1-9 or 14-38, wherein the assay determines the presence of the CXCR4-ADRB2 heteromer in the subject's cell.

40. The method for suppressing, the method for treating, the pharmaceutical kit for use, or the pharmaceutical composition for use, of any one of claims 1-9 or 17-39, wherein the assay determines the presence of the CXCR4-ADRB2 heteromer in the biological sample obtained from said subject.

41. The method for suppressing, the method for treating, the pharmaceutical kit for use, or the pharmaceutical composition for use, of any one of claims 1-9 or 17-40, wherein the assay determines the presence of the CXCR4-ADRB2 heteromer in the subject.

42. The method for suppressing, the method for treating, the pharmaceutical kit for use, or the pharmaceutical composition for use, of any one of claims 1-41, wherein the CXCR4-ADRB2 heteromer is characterized as having the following characteristic: enhanced downstream signaling results from the CXCR4-ADRB2 heteromer.

43. The method for suppressing, the method for treating, the pharmaceutical kit for use, or the pharmaceutical composition for use, of any one of claims 1-9 or 14-42, wherein the enhanced downstream signaling results from the CXCR4-ADRB2 heteromer in said subject's cell.

44. The method for suppressing, the method for treating, the pharmaceutical kit for use, or the pharmaceutical composition for use, of any one of claims 1-9 or 14-43, wherein the enhanced downstream signaling results from the presence of the CXCR4-ADRB2 heteromer in said subject's cell.

45. The method for suppressing, the method for treating, the pharmaceutical kit for use, or the pharmaceutical composition for use, of any one of claims 1-44, wherein the CXCR4-ADRB2 heteromer produces the enhanced downstream signaling.

46. The method for suppressing, the method for treating, the pharmaceutical kit for use, or the pharmaceutical composition for use, of any one of claims 1-9 or 14-45, wherein the CXCR4-ADRB2 heteromer produces the enhanced downstream signaling in the subject's cell.

47. The method for suppressing, the method for treating, the pharmaceutical kit for use, or the pharmaceutical composition for use, of any one of claims 1-46, wherein the enhanced downstream signaling results from agonism of the CXCR4-ADRB2 heteromer.

48. The method for suppressing, the method for treating, the pharmaceutical kit for use, or the pharmaceutical composition for use, of any one of claims 1-47, wherein the enhanced downstream signaling results from CXCR4 agonism of the CXCR4-ADRB2 heteromer.

49. The method for suppressing, the method for treating, the pharmaceutical kit for use, or the pharmaceutical composition for use, of any one of claims 1-47, wherein the enhanced downstream signaling results from ADRB2 agonism of the CXCR4-ADRB2 heteromer.

50. The method for suppressing, the method for treating, the pharmaceutical kit for use, or the pharmaceutical composition for use, of any one of claims 1-47, wherein the enhanced downstream signaling results from CXCR4 agonism and ADRB2 agonism of the CXCR4-ADRB2 heteromer.

51. The method for suppressing, the method for treating, the pharmaceutical kit for use, or the pharmaceutical composition for use, of any one of claims 1-50, wherein the enhanced downstream signaling is downstream of the CXCR4, the ADRB2, or the CXCR4-ADRB2 heteromer.

52. The method for suppressing, the method for treating, the pharmaceutical kit for use, or the pharmaceutical composition for use, of claim 51, wherein the enhanced downstream signaling is downstream of the CXCR4.

53. The method for suppressing, the method for treating, the pharmaceutical kit for use, or the pharmaceutical composition for use, of claim 51, wherein the enhanced downstream signaling is downstream of the ADRB2.

54. The method for suppressing, the method for treating, the pharmaceutical kit for use, or the pharmaceutical composition for use, of claim 51, wherein the enhanced downstream signaling is downstream of the CXCR4-ADRB2 heteromer.

55. The method for suppressing, the method for treating, the pharmaceutical kit for use, or the pharmaceutical composition for use, of any one of claims 1-50, wherein the enhanced downstream signaling from the CXCR4-ADRB2 heteromer is relative to downstream signaling from a CXCR4 protomer or an ADRB2 protomer in their respective individual protomer context.

56. The method for suppressing, the method for treating, the pharmaceutical kit for use, or the pharmaceutical composition for use, of claim 55, wherein the enhanced downstream signaling from the CXCR4-ADRB2 heteromer is relative to downstream signaling from a CXCR4 protomer in an individual protomer context.

57. The method for suppressing, the method for treating, the pharmaceutical kit for use, or the pharmaceutical composition for use, of claim 55, wherein the enhanced downstream signaling from the CXCR4-ADRB2 heteromer is relative to downstream signaling from an ADRB2 protomer in an individual protomer context.

58. The method for suppressing, the method for treating, the pharmaceutical kit for use, or the pharmaceutical composition for use, of claim 55, wherein the enhanced downstream signaling from the CXCR4-ADRB2 heteromer is relative to downstream signaling from a CXCR4 protomer and an ADRB2 protomer in their respective individual protomer context.

59. The method for suppressing, the method for treating, the pharmaceutical kit for use, or the pharmaceutical composition for use, of any one of claims 1-58, wherein the enhanced downstream signaling is an enhanced amount of calcium mobilization.

60. The method for suppressing, the method for treating, the pharmaceutical kit for use, or the pharmaceutical composition for use, of claim 59, wherein the enhanced amount of calcium mobilization is determined by an intracellular Ca2+ assay.

61. The method for suppressing, the method for treating, the pharmaceutical kit for use, or the pharmaceutical composition for use, of any one of claims 1-60, wherein the enhanced downstream signaling from the CXCR4-ADRB2 heteromer is determined by an intracellular Ca2+ assay.

62. The method for suppressing, the method for treating, the pharmaceutical kit for use, or the pharmaceutical composition for use, of claim 61, wherein the intracellular Ca2+ assay is a calcium mobilization assay.

63. The method for suppressing, the method for treating, the pharmaceutical kit for use, or the pharmaceutical composition for use, of claim 62, wherein the calcium mobilization assay determines the CXCR4-ADRB2 heteromer produces the enhanced downstream signaling.

64. The method for suppressing, the method for treating, the pharmaceutical kit for use, or the pharmaceutical composition for use, of claim 62 or 63, wherein the calcium mobilization assay determines the enhanced downstream signaling results from the presence of the CXCR4-ADRB2 heteromer.

65. The method for suppressing, the method for treating, the pharmaceutical kit for use, or the pharmaceutical composition for use, of any one of claims 1-64, wherein the CXCR4-ADRB2 heteromer exhibits the enhanced amount of calcium mobilization, such that:
a) either the CXCR4 or the ADRB2 in an individual protomer context in the cell upon co-stimulation with CXCL12 and an ADRB2 agonist results in a calcium mobilization amount that is equal to or less than the sum of calcium mobilization amounts resulting from single agonist stimulation with either the CXCL12 or the ADRB2 agonist; and
b) the CXCR4-ADRB2 heteromer exhibits an enhanced calcium mobilization upon co-stimulation with the CXCL12 and the ADRB2 agonist relative to the sum of calcium mobilization amounts resulting from single agonist stimulation with either the CXCL12 or the ADRB2 agonist;
as determined via a calcium mobilization assay.

66. The method for suppressing, the method for treating, the pharmaceutical kit for use, or the pharmaceutical composition for use, of any one of claims 1-65, wherein:
i) the calcium mobilization from the protomer CXCR4 or ADRB2, in the individual protomer context in the cell, is non-synergistic, as determined via calcium mobilization assay; and
ii) the calcium mobilization from the CXCR4-ADRB2 heteromer in the cell is synergistic, as determined via a calcium mobilization assay.

67. The method for suppressing, the method for treating, the pharmaceutical kit for use, or the pharmaceutical composition for use, of any one of claims 1-66, wherein in the individual protomer context:
a) the individual protomer CXCR4 in the cell, in the absence of the individual protomer ADRB2; or
b) the individual protomer ADRB2 in the cell, in the absence of the individual protomer CXCR4;
upon co-stimulation with CXCL12 and an ADRB2 agonist results in a calcium mobilization amount that is equal to or less than the sum of calcium mobilization amounts resulting from single agonist stimulation with either the CXCL12 or the ADRB2 agonist, as determined via a calcium mobilization assay.

68. The method for suppressing, the method for treating, the pharmaceutical kit for use, or the pharmaceutical composition for use, of any one of claims 1-67, wherein in the individual protomer context, independently:
a) the individual protomer CXCR4 in the cell, in the absence of the individual protomer ADRB2; and
b) the individual protomer ADRB2 in the cell, in the absence of the individual protomer CXCR4;
upon co-stimulation with CXCL12 and an ADRB2 agonist results in a calcium mobilization amount that is equal to or less than the sum of calcium mobilization amounts resulting from single agonist stimulation with either the CXCL12 or the ADRB2 agonist, as determined via a calcium mobilization assay.

69. The method for suppressing, the method for treating, the pharmaceutical kit for use, or the pharmaceutical composition for use, of any one of claims 1-68, wherein the CXCR4-ADRB2 heteromer upon co-stimulation with the CXCL12 and the ADRB2 agonist results in a calcium mobilization amount that is greater than the sum of calcium mobilization amounts resulting from single agonist stimulation of said cells with either the CXCL12 or the ADRB2 agonist, as determined via a calcium mobilization assay.

70. The method for suppressing, the method for treating, the pharmaceutical kit for use, or the pharmaceutical composition for use, of any one of claims 1-69, wherein the calcium mobilization amount resulting from the co-stimulation of the CXCR4-ADRB2 heteromer is an enhanced amount of calcium mobilization, relative to the sum of calcium mobilizations resulting from single agonist stimulation of said CXCR4-ADRB2 heteromer, as determined via a calcium mobilization assay.

71. The method for suppressing, the method for treating, the pharmaceutical kit for use, or the pharmaceutical composition for use, of any one of claims 1-70, wherein the enhanced amount of calcium mobilization resulting from the CXCR4-ADRB2 heteromer is a calcium mobilization amount that, upon co-stimulation with CXCL12 and ADRB2 agonist, is greater than the sum of calcium mobilization amounts resulting from single agonist stimulation of said cells with either the CXCL12 or the ADRB2 agonist, as determined via a calcium mobilization assay.

72. The method for suppressing, the method for treating, the pharmaceutical kit for use, or the pharmaceutical composition for use, of claim 71, wherein the enhanced amount of calcium mobilization resulting from the CXCR4-ADRB2 heteromer is a calcium mobilization amount that, upon co-stimulation with the CXCL12 and the ADRB2 agonist, is at least 10% greater, at least 20% greater, at least 30% greater, at least 40% greater, at least 50% greater, at least 75% greater, or at least 90% greater, than the sum of calcium mobilization amounts resulting from single agonist stimulation of said cells with either the CXCL12 or the ADRB2 agonist, as determined via a calcium mobilization assay.

73. The method for suppressing, the method for treating, the pharmaceutical kit for use, or the pharmaceutical composition for use, of claim 71 or 72, wherein the enhanced amount of calcium mobilization resulting from the CXCR4-ADRB2 heteromer is a calcium mobilization amount that, upon co-stimulation with the CXCL12 and the ADRB2 agonist, is at least 100% greater than the sum of calcium mobilization amounts resulting from single agonist stimulation of said cells with either the CXCL12 or the ADRB2 agonist, as determined via a calcium mobilization assay.

74. The method for suppressing, the method for treating, the pharmaceutical kit for use, or the pharmaceutical composition for use, of any one of claims 71-73, wherein the enhanced amount of calcium mobilization is a synergistic amount of calcium mobilization.

75. The method for suppressing, the method for treating, the pharmaceutical kit for use, or the pharmaceutical composition for use, of claim 74, wherein the synergistic amount of calcium mobilization from the cells containing the CXCR4-ADRB2 heteromer is a calcium mobilization amount that, upon co-stimulation with the CXCL12 and the ADRB2 agonist, is at least 10% greater, at least 20% greater, at least 30% greater, at least 40% greater, at least 50% greater, at least 75% greater, or at least 90% greater, than the sum of calcium mobilization amounts resulting from single agonist stimulation of said cells with either the CXCL12 or the ADRB2 agonist, as determined via a calcium mobilization assay.

76. The method for suppressing, the method for treating, the pharmaceutical kit for use, or the pharmaceutical composition for use, of any one of claims 1-75, wherein the enhanced amount of calcium mobilization is characterized as follows:
a) either the CXCR4 or the ADRB2 in an individual protomer context in a cell, upon co-stimulation with CXCL12 and an ADRB2 agonist, results in a calcium mobilization amount that is equal to or less than the sum of calcium mobilization amounts resulting from single agonist stimulation with either the CXCL12 or the ADRB2 agonist; and
b) the CXCR4-ADRB2 heteromer exhibits an enhanced calcium mobilization upon co-stimulation with the CXCL12 and the ADRB2 agonist relative to the sum of calcium mobilization amounts resulting from single agonist stimulation with either the CXCL12 or the ADRB2 agonist;
as determined via a calcium mobilization assay.

77. The method for suppressing, the method for treating, the pharmaceutical kit for use, or the pharmaceutical composition for use, of any one of claims 1-76, wherein the CXCR4-ADRB2 heteromer is characterized as having the following characteristic: a greater amount of downstream ERK signaling results from co-stimulation of the CXCR4-ADRB2 heteromer with a CXCR4 agonist and an ADRB2 agonist, relative to the amount of downstream ERK signaling resulting from mono-stimulation of said CXCR4-ADRB2 heteromer with either the CXCR4 agonist or the ADRB2 agonist.

78. The method for suppressing, the method for treating, the pharmaceutical kit for use, or the pharmaceutical composition for use, of claim 77, wherein the amount of downstream ERK signaling resulting from co-stimulation with the CXCR4 agonist and the ADRB2 agonist is greater than the amount resulting from mono-stimulation with the CXCR4 agonist.

79. The method for suppressing, the method for treating, the pharmaceutical kit for use, or the pharmaceutical composition for use, of claim 78, wherein the amount of downstream ERK signaling resulting from co-stimulation with the CXCR4 agonist and the ADRB2 agonist is at least 5% greater than the amount resulting from mono-stimulation with the CXCR4 agonist.

80. The method for suppressing, the method for treating, the pharmaceutical kit for use, or the pharmaceutical composition for use, of claim 78, wherein the amount of downstream ERK signaling resulting from co-stimulation with the CXCR4 agonist and the ADRB2 agonist is at least 10%, at least 25%, at least 50%, at least 75%, or at least 90%, greater than the amount resulting from mono-stimulation with the CXCR4 agonist.

81. The method for suppressing, the method for treating, the pharmaceutical kit for use, or the pharmaceutical composition for use, of any one of claims 78-80, wherein the amount of downstream ERK signaling resulting from co-stimulation with the CXCR4 agonist and the ADRB2 agonist in the range of between 5-15%, 10-25%, 20-50%, 40-75%, or 60-100%, greater than the amount resulting from mono-stimulation with the CXCR4 agonist.

82. The method for suppressing, the method for treating, the pharmaceutical kit for use, or the pharmaceutical composition for use, of claim 77, wherein the amount of downstream ERK signaling resulting from co-stimulation with the CXCR4 agonist and the ADRB2 agonist is greater than the amount resulting from mono-stimulation with the ADRB2 agonist.

83. The method for suppressing, the method for treating, the pharmaceutical kit for use, or the pharmaceutical composition for use, of claim 82, wherein the amount of downstream ERK signaling resulting from co-stimulation with the CXCR4 agonist and the ADRB2 agonist is at least 5% greater than the amount resulting from mono-stimulation with the ADRB2 agonist.

84. The method for suppressing, the method for treating, the pharmaceutical kit for use, or the pharmaceutical composition for use, of claim 82, wherein the amount of downstream ERK signaling resulting from co-stimulation with the CXCR4 agonist and the ADRB2 agonist is at least 10%, at least 25%, at least 50%, at least 75%, or at least 90%, greater than the amount resulting from mono-stimulation with the ADRB2 agonist.

85. The method for suppressing, the method for treating, the pharmaceutical kit for use, or the pharmaceutical composition for use, of any one of claims 82-84, wherein the amount of downstream ERK signaling resulting from co-stimulation with the CXCR4 agonist and the ADRB2 agonist in the range of between 5-15%, 10-25%, 20-50%, 40-75%, or 60-100%, greater than the amount resulting from mono-stimulation with the ADRB2 agonist.

86. The method for suppressing, the method for treating, the pharmaceutical kit for use, or the pharmaceutical composition for use, of any one of claims 1-9 or 14-85, wherein the CXCR4-ADRB2 heteromer is characterized as having the following characteristic: a combination of burixafor and an ADRB2 inhibitor alters heteromer-specific properties of the CXCR4-ADRB2 heteromer in the subject's derived cell(s).

87. The method for suppressing, the method for treating, the pharmaceutical kit for use, or the pharmaceutical composition for use, of any one of claims 1-9 or 14-86, wherein the CXCR4-ADRB2 heteromer is characterized as having the following characteristic: a combination of burixafor and an ADRB2 inhibitor alters heteromer-specific function of the CXCR4-ADRB2 heteromer in the subject's derived cell(s).

88. The method for suppressing, the method for treating, the pharmaceutical kit for use, or the pharmaceutical composition for use, of any one of claims 1-9 or 14-87, wherein the CXCR4-ADRB2 heteromer is characterized as having the following characteristic: a combination of burixafor and an ADRB2 inhibitor alters heteromer-specific properties of the subject's derived cell(s) containing the CXCR4-ADRB2 heteromer.

89. The method for suppressing, the method for treating, the pharmaceutical kit for use, or the pharmaceutical composition for use, of any one of claims 1-9 or 14-88, wherein the CXCR4-ADRB2 heteromer is characterized as having the following characteristic: a combination of burixafor and an ADRB2 inhibitor decreases cancer progression of the subject derived cell(s) containing the CXCR4-ADRB2 heteromer.

90. The method for suppressing, the method for treating, the pharmaceutical kit for use, or the pharmaceutical composition for use, of any one of claims 1-89, wherein the administered combination of the burixafor and the ADRB2 inhibitor suppresses the enhanced downstream signaling from said CXCR4-ADRB2 heteromer.

91. The method for suppressing, the method for treating, the pharmaceutical kit for use, or the pharmaceutical composition for use, of any one of claims 1-90, wherein the administered combination of the burixafor and the ADRB2 inhibitor suppresses the enhanced downstream signaling from said CXCR4-ADRB2 heteromer in the cell.

92. The method for suppressing, the method for treating, the pharmaceutical kit for use, or the pharmaceutical composition for use, of any one of claims 1-9 or 17-91, wherein the administered combination of the burixafor and the ADRB2 inhibitor suppresses the enhanced downstream signaling from said CXCR4-ADRB2 heteromer in the cancer subject.

93. The method for suppressing, the method for treating, the pharmaceutical kit for use, or the pharmaceutical composition for use, of any one of claims 1-9 or 17-92, wherein the administered combination of the burixafor and the ADRB2 inhibitor suppresses the enhanced downstream signaling from said CXCR4-ADRB2 heteromer in the cell of the cancer subject.

94. The method for suppressing, the method for treating, the pharmaceutical kit for use, or the pharmaceutical composition for use, of any one of claims 1-93, wherein the CXCR4-ADRB2 heteromer components comprise individual protomers of CXCR4 and ADRB2.

95. The method for suppressing, the method for treating, the pharmaceutical kit for use, or the pharmaceutical composition for use, of any one of claims 1-94, wherein the cell containing the CXCR4 in an individual protomer context comprises the individual protomer CXCR4 in the presence or absence of the individual protomer ADRB2.

96. The method for suppressing, the method for treating, the pharmaceutical kit for use, or the pharmaceutical composition for use, of claim 95, wherein the cell containing the CXCR4 in an individual protomer context comprises said individual protomer CXCR4 in the absence of the individual protomer ADRB2.

97. The method for suppressing, the method for treating, the pharmaceutical kit for use, or the pharmaceutical composition for use, of claim 95, wherein the cell containing the CXCR4 in an individual protomer context comprises said individual protomer CXCR4 in the presence of the individual protomer ADRB2.

98. The method for suppressing, the method for treating, the pharmaceutical kit for use, or the pharmaceutical composition for use, of any one of claims 1-94, wherein the cell containing the ADRB2 in an individual protomer context comprises the individual protomer ADRB2 in the presence or absence of the individual protomer CXCR4.

99. The method for suppressing, the method for treating, the pharmaceutical kit for use, or the pharmaceutical composition for use, of claim 98, wherein the cell containing the ADRB2 in an individual protomer context comprises said individual protomer ADRB2 in the absence of the individual protomer CXCR4.

100. The method for suppressing, the method for treating, the pharmaceutical kit for use, or the pharmaceutical composition for use, of claim 98, wherein the cell containing the ADRB2 in an individual protomer context comprises said individual protomer ADRB2 in the presence of the individual protomer CXCR4.

101. The method for suppressing, the method for treating, the pharmaceutical kit for use, or the pharmaceutical composition for use, of any one of claims 1-100, wherein the administered combination of burixafor and an ADRB2 inhibitor:
i) alters heteromer-specific properties of the CXCR4-ADRB2 heteromer in the cell;
ii) alters heteromer-specific function of the CXCR4-ADRB2 heteromer in the cell; and/or
iii) alters heteromer-specific properties of the cell containing the CXCR4-ADRB2 heteromer.

102. The method for suppressing, the method for treating, the pharmaceutical kit for use, or the pharmaceutical composition for use, of any one of claims 1-9 or 14-101, wherein the administered combination of the burixafor and the ADRB2 inhibitor:
i) alters heteromer-specific properties of the CXCR4-ADRB2 heteromer in the subject's derived cell(s);
ii) alters heteromer-specific function of the CXCR4-ADRB2 heteromer in the subject's derived cell(s);
iii) alters heteromer-specific properties of the subject's derived cell(s) containing the CXCR4-ADRB2 heteromer; or
iv) decreases cancer progression in the subject having cells containing said CXCR4-ADRB2 heteromer.

103. The method for suppressing, the method for treating, the pharmaceutical kit for use, or the pharmaceutical composition for use, of claim 102, wherein the administered combination of the burixafor and the ADRB2 inhibitor alters heteromer-specific properties of the CXCR4-ADRB2 heteromer in the subject's derived cell(s).

104. The method for suppressing, the method for treating, the pharmaceutical kit for use, or the pharmaceutical composition for use, of claim 102 or 103, wherein the administered combination of the burixafor and the ADRB2 inhibitor alters heteromer-specific function of the CXCR4-ADRB2 heteromer in the subject's derived cell(s).

105. The method for suppressing, the method for treating, the pharmaceutical kit for use, or the pharmaceutical composition for use, of any one of claims 102-104, wherein the administered combination of the burixafor and the ADRB2 inhibitor alters heteromer-specific properties of the subject's derived cell(s) containing the CXCR4-ADRB2 heteromer.

106. The method for suppressing, the method for treating, the pharmaceutical kit for use, or the pharmaceutical composition for use, of any one of claims 102-105, wherein the administered combination of the burixafor and the ADRB2 inhibitor decreases cancer progression in the subject having cells containing said CXCR4-ADRB2 heteromer.

107. The method for suppressing, the method for treating, the pharmaceutical kit for use, or the pharmaceutical composition for use, of any one of claims 102-106, wherein the CXCR4-ADRB2 heteromer is characterized as having the following characteristic: a combination of burixafor and an ADRB2 inhibitor decreases cancer progression of the subject derived cell(s) containing the CXCR4-ADRB2 heteromer.

108. The method for suppressing, the method for treating, the pharmaceutical kit for use, or the pharmaceutical composition for use, of any one of claims 102-107, wherein the decreased cancer progression comprises a decrease in cancer progression of the subject's derived cell(s) containing said CXCR4-ADRB2 heteromer.

109. The method for suppressing, the method for treating, the pharmaceutical kit for use, or the pharmaceutical composition for use, of any one of claims 102-108, wherein the decreased cancer progression comprises a decrease in cell proliferation of the subject's derived cell(s) containing said CXCR4-ADRB2 heteromer.

110. The method for suppressing, the method for treating, the pharmaceutical kit for use, or the pharmaceutical composition for use, of any one of claims 102-109, wherein the decreased cancer progression comprises a decrease in cell migration of the subject's derived cell(s) containing said CXCR4-ADRB2 heteromer.

111. The method for suppressing, the method for treating, the pharmaceutical kit for use, or the pharmaceutical composition for use, of any one of claims 102-110, wherein the decreased cancer progression comprises a decrease in metastasis of the subject's derived cell(s) containing said CXCR4-ADRB2 heteromer.

112. The method for suppressing, the method for treating, the pharmaceutical kit for use, or the pharmaceutical composition for use, of any one of claims 102-111, wherein the decreased cancer progression comprises a decrease in angiogenesis of the subject's derived cell(s) containing said CXCR4-ADRB2 heteromer.

113. The method for suppressing, the method for treating, the pharmaceutical kit for use, or the pharmaceutical composition for use, of any one of claims 1-112, wherein the burixafor is administered as a pharmaceutical composition comprising a pharmaceutically acceptable carrier.

114. The method for suppressing, the method for treating, the pharmaceutical kit for use, or the pharmaceutical composition for use, of any one of claims 1-113, wherein the ADRB2 inhibitor is administered as a pharmaceutical composition comprising a pharmaceutically acceptable carrier.

115. The method for suppressing, the method for treating, the pharmaceutical kit for use, or the pharmaceutical composition for use, of any one of claims 1-114, wherein the combination of the burixafor and the ADRB2 inhibitor is administered sequentially, concurrently, or simultaneously.

116. The method for suppressing, the method for treating, the pharmaceutical kit for use, or the pharmaceutical composition for use, of any one of claims 1-115, wherein the combination of the burixafor and the ADRB2 inhibitor is administered as a pharmaceutical composition further comprising a pharmaceutically acceptable carrier.

117. The method for suppressing, the method for treating, the pharmaceutical kit for use, or the pharmaceutical composition for use, of any one of claims 1-116, wherein the burixafor and the ADRB2 inhibitor are administered as a combination of pharmaceutical compositions.

118. The method for suppressing, the method for treating, the pharmaceutical kit for use, or the pharmaceutical composition for use, of claim 117, wherein the combination of pharmaceutical compositions comprises:
a) a pharmaceutical composition comprising the burixafor and a pharmaceutically acceptable carrier; and
b) a pharmaceutical composition comprising the ADRB2 inhibitor and a pharmaceutically acceptable carrier.

119. The method for suppressing, the method for treating, the pharmaceutical kit for use, or the pharmaceutical composition for use, of claim 117 or 118, wherein the combination of the pharmaceutical compositions is administered sequentially, concurrently, or simultaneously.

120. The method for suppressing, the method for treating, the pharmaceutical kit for use, or the pharmaceutical composition for use, of any one of claims 1-119, wherein the cancer is a hematological cancer or a solid tumor.

121. The method for suppressing, the method for treating, the pharmaceutical kit for use, or the pharmaceutical composition for use, of claim 120, wherein the cancer is a hematological cancer.

122. The method for suppressing, the method for treating, the pharmaceutical kit for use, or the pharmaceutical composition for use, of claim 121, wherein the hematological cancer is selected from the group consisting of: a lymphoma, a leukemia, a myeloma, and a multiple myeloma.

123. The method for suppressing, the method for treating, the pharmaceutical kit for use, or the pharmaceutical composition for use, of claim 122, wherein the lymphoma is a B cell lymphoma, a T-cell lymphoma, or a NK cell lymphoma.

124. The method for suppressing, the method for treating, the pharmaceutical kit for use, or the pharmaceutical composition for use, of claim 122 or 123, wherein the lymphoma is a relapsed or refractory lymphoma.

125. The method for suppressing, the method for treating, the pharmaceutical kit for use, or the pharmaceutical composition for use, of any one of claims 122-124, wherein the lymphoma is selected from the group consisting of: Hodgkin's lymphoma, non-Hodgkin's lymphoma, cutaneous B-cell lymphoma, activated B-cell lymphoma, Diffuse Large B-Cell Lymphoma (DLBCL), Burkitt lymphoma, mantle cell lymphoma (MCL), follicular lymphoma (FL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphoma (PTCL), cutaneous T-Cell lymphoma (CTCL), mantle zone lymphoma, and low grade follicular lymphoma.

126. The method for suppressing, the method for treating, the pharmaceutical kit for use, or the pharmaceutical composition for use, of claim 122, wherein the leukemia is:
a) an acute leukemia selected from the group consisting of: acute lymphocytic leukemia (ALL), T-cell acute lymphocytic leukemia (T-ALL), B-cell acute lymphoblastic leukemia, acute monocytic leukemia, acute promyelocytic leukemia, acute myelocytic leukemia (AML), acute myeloid leukemia, acute myelogenous leukemia and myeloblasts, promyeiocytic, myelomonocytic, monocytic, and erythroleukemia;
b) a chronic leukemia selected from the group consisting of: chronic myelocytic (granulocytic) leukemia, chronic myelogenous leukemia (chronic myeloid leukemia; CML), and chronic lymphocytic leukemia (CLL); or
c) chronic myelomonocytic leukemia (CMML), chronic eosinophilic leukemia, juvenile myelomonocytic leukemia (JMML), polycythemia vera, natural killer cell leukemia (NK leukemia), or hairy cell leukemia.

127. The method for suppressing, the method for treating, the pharmaceutical kit for use, or the pharmaceutical composition for use, of claim 120, wherein the cancer is a solid tumor.

128. The method for suppressing, the method for treating, the pharmaceutical kit for use, or the pharmaceutical composition for use, of claim 127, wherein the solid tumor is a benign tumor or a cancer.

129. The method for suppressing, the method for treating, the pharmaceutical kit for use, or the pharmaceutical composition for use, of claim 127, wherein the solid tumor is a carcinoma or a sarcoma.

130. The method for suppressing, the method for treating, the pharmaceutical kit for use, or the pharmaceutical composition for use, of claim 127, wherein the solid tumor is selected from the group consisting of: fibrosarcoma, myxosarcoma, liposarcoma, chondrosarcoma, osteosarcoma, synovioma, mesothelioma, malignant epithelioid mesothelioma, Ewing's tumor, leiomyosarcoma, rhabdomyosarcoma, stomach cancer, colorectal cancer, esophageal cancer, colon carcinoma, lymphoid malignancy, pancreatic cancer, pancreatic adenocarcinoma, pancreatic ductal adenocarcinoma, breast cancer, breast adenocarcinoma, breast ductal adenocarcinoma, lung cancer, small cell lung carcinoma, lung adenocarcinoma, adenosquamous lung carcinoma, alveolar rhabdomyosarcoma, ovarian cancer, ovarian clear cell adenocarcinoma, ovarian mucinous cystadenocarcinoma, ovarian serous adenocarcinoma, prostate cancer, hepatocellular carcinoma, soft tissue sarcomas, squamous cell carcinoma, basal cell carcinoma, adenocarcinoma, sweat gland carcinoma, medullary thyroid carcinoma, papillary thyroid carcinoma, thyroid gland carcinoma, pheochromocytomas sebaceous gland carcinoma, papillary carcinoma, papillary adenocarcinomas, medullary carcinoma, bronchogenic carcinoma, gastrointestinal cancer, gastric tubular adenocarcinoma, gastric adenosquamous carcinoma, kidney cancer, intrahepatic cholangiocarcinoma, renal cell carcinoma, hepatoma, bile duct carcinoma, choriocarcinoma, Wilms' tumor, uterine carcinosarcoma, endometrium adenocarcinoma, endometrial stromal sarcoma, endometrial carcinoma, cervical cancer, testicular tumor, seminoma, bladder carcinoma, melanoma, multiple myeloma, multiple endocrine neoplasia, CNS tumor, glioblastoma, astrocytoma, CNS lymphoma, germinoma, meduloblastoma, Schwannoma craniopharyogioma, ependymoma, pineaioma, hemangioblastoma, acoustic neuroma, oligodendroglioma, menangioma, neuroblastoma, retinoblastoma, and brain metastases.

131. The method for suppressing, the method for treating, the pharmaceutical kit for use, or the pharmaceutical composition for use, of claim 130, wherein the solid tumor is selected from the group consisting of: breast cancer, lung cancer, and hepatocellular carcinoma.

132. The method for suppressing, the method for treating, the pharmaceutical kit for use, or the pharmaceutical composition for use, of claim 131, wherein the solid tumor is breast cancer.

133. The method for suppressing, the method for treating, the pharmaceutical kit for use, or the pharmaceutical composition for use, of claim 131, wherein the solid tumor is lung cancer.

134. The method for suppressing, the method for treating, the pharmaceutical kit for use, or the pharmaceutical composition for use, of claim 131, wherein the solid tumor is hepatocellular carcinoma.

135. The method for suppressing, the method for treating, the pharmaceutical kit for use, or the pharmaceutical composition for use, of any one of claims 1-9 or 17-134, wherein the subject's biological sample is a biological fluid sample.

136. The method for suppressing, the method for treating, the pharmaceutical kit for use, or the pharmaceutical composition for use, of claim 135, wherein a liquid biopsy is performed on the biological fluid sample.

137. The method for suppressing, the method for treating, the pharmaceutical kit for use, or the pharmaceutical composition for use, of claim 135 or 136, wherein the biological fluid sample is a blood sample, a plasma sample, a saliva sample, a cerebral fluid sample, an eye fluid sample, or a urine sample.

138. The method for suppressing, the method for treating, the pharmaceutical kit for use, or the pharmaceutical composition for use, of any one of claims 1-9 or 17-137, wherein the subject's biological sample is a biological tissue sample.

139. The method for suppressing, the method for treating, the pharmaceutical kit for use, or the pharmaceutical composition for use, of claim 138, wherein a tissue sample assay is performed on the biological tissue sample.

140. The method for suppressing, the method for treating, the pharmaceutical kit for use, or the pharmaceutical composition for use, of claim 138 or 139, wherein the biological tissue sample is an organ tissue sample, a bone tissue sample, or a tumor tissue sample.

141. The method for suppressing, the method for treating, the pharmaceutical kit for use, or the pharmaceutical composition for use, of any one of claims 1-140, wherein the method for treating cancer is a method for suppressing enhanced downstream signaling resulting from the CXCR4-ADRB2 heteromer.

142. The method for suppressing, the method for treating, the pharmaceutical kit for use, or the pharmaceutical composition for use, of any one of claims 1-140, wherein the method for suppressing enhanced downstream signaling resulting from the CXCR4-ADRB2 heteromer is a method for treating cancer.

143. The method for suppressing, the method for treating, the pharmaceutical kit for use, or the pharmaceutical composition for use, of any one of claims 1-9 or 17-142, wherein progression of the cancer in the subject having said cell containing the CXCR4-ADRB2 heteromer is decreased in the range of 5-100% more upon administering to the cancer subject the combination of the burixafor and the ADRB2 inhibitor, relative to administering the single inhibitor of either the burixafor or the ADRB2 inhibitor.

144. The method for suppressing, the method for treating, the pharmaceutical kit for use, or the pharmaceutical composition for use, of any one of claims 1-9 or 17-143, wherein efficacy of the burixafor is increased in the range of 5-5000% when administered in combination with the ADRB2 inhibitor to the subject having said cell containing the CXCR4-ADRB2 heteromer, relative to efficacy of the burixafor when administered as a single inhibitor.

145. The method for suppressing, the method for treating, the pharmaceutical kit for use, or the pharmaceutical composition for use, of any one of claims 1-9 or 17-144, wherein efficacy of an ADRB2 inhibitor is increased in the range of 5-5000% when administered in combination with the burixafor to the subject having said cell containing the CXCR4-ADRB2 heteromer, relative to efficacy of the ADRB2 inhibitor when administered as a single inhibitor.

146. The method for suppressing, the method for treating, the pharmaceutical kit for use, or the pharmaceutical composition for use, of any one of claims 1-9 or 17-145, wherein the administering to the cancer subject the combination of the burixafor and the ADRB2 inhibitor suppresses the enhanced downstream signaling from said CXCR4-ADRB2 heteromer in said cancer subject in the range of between 5-2000 fold, relative to single inhibitor administration.

147. The method for suppressing, the method for treating, the pharmaceutical kit for use, or the pharmaceutical composition for use, of any one of claims 1-9 or 17-146, wherein the administering to the cancer subject the combination of the burixafor and the ADRB2 inhibitor suppresses the enhanced downstream signaling from said CXCR4-ADRB2 heteromer in said cancer subject in the range of between 5-2000 fold, relative to suppression of downstream signaling from either a CXCR4 protomer or an ADRB2 protomer in their respective individual protomer context.

148. The method for suppressing, the method for treating, the pharmaceutical kit for use, or the pharmaceutical composition for use, of any one of claims 17-147, wherein the cell is a cancer cell.

149. The method for suppressing, the method for treating, the pharmaceutical kit for use, or the pharmaceutical composition for use, of any one of claims 17-147, wherein the cell is a subject's cell.

150. The method for suppressing, the method for treating, the pharmaceutical kit for use, or the pharmaceutical composition for use, of claim 149, wherein the subject's cell is a cancer cell.

151. The method for suppressing, the method for treating, the pharmaceutical kit for use, or the pharmaceutical composition for use, of any one of claims 1-150, wherein the subject is a cancer subject.

152. The method for suppressing, the method for treating, the pharmaceutical kit for use, or the pharmaceutical composition for use, of any one of claims 1-151, wherein the subject is a patient.

153. A pharmaceutical composition, comprising:
a) a CXCR4 inhibitor, wherein the CXCR4 inhibitor is burixafor;
b) an ADRB2 inhibitor; and
c) a pharmaceutically acceptable carrier.

154. The method for suppressing, the method for treating, the pharmaceutical kit for use, the pharmaceutical composition for use, or the pharmaceutical composition, of any one of claims 1-153, wherein the ADRB2 inhibitor is an antagonist of ADRB2, an inverse agonist of ADRB2, a partial antagonist of ADRB2, an allosteric modulator of ADRB2, an antibody of ADRB2, an antibody fragment of ADRB2, a ligand of ADRB2, or an antibody-drug conjugate.

155. The method for suppressing, the method for treating, the pharmaceutical kit for use, the pharmaceutical composition for use, or the pharmaceutical composition, of claim 154, wherein the ADRB2 inhibitor is an antagonist ADRB2.

156. The method for suppressing, the method for treating, the pharmaceutical kit for use, the pharmaceutical composition for use, or the pharmaceutical composition, of claim 154, wherein the ADRB2 inhibitor is an inverse agonist ADRB2.

157. The method for suppressing, the method for treating, the pharmaceutical kit for use, the pharmaceutical composition for use, or the pharmaceutical composition, of claim 154, wherein the ADRB2 inhibitor is a partial antagonist ADRB2.

158. The method for suppressing, the method for treating, the pharmaceutical kit for use, the pharmaceutical composition for use, or the pharmaceutical composition, of claim 154, wherein the ADRB2 inhibitor is an allosteric modulator of ADRB2.

159. The method for suppressing, the method for treating, the pharmaceutical kit for use, the pharmaceutical composition for use, or the pharmaceutical composition, of claim 154, wherein the ADRB2 inhibitor is an antibody of ADRB2.

160. The method for suppressing, the method for treating, the pharmaceutical kit for use, the pharmaceutical composition for use, or the pharmaceutical composition, of claim 154, wherein the ADRB2 inhibitor is an antibody fragment of ADRB2.

161. The method for suppressing, the method for treating, the pharmaceutical kit for use, the pharmaceutical composition for use, or the pharmaceutical composition, of claim 154, wherein the ADRB2 inhibitor is a ligand of ADRB2.

162. The method for suppressing, the method for treating, the pharmaceutical kit for use, the pharmaceutical composition for use, or the pharmaceutical composition, of claim 154, wherein the ADRB2 inhibitor is an antibody-drug conjugate.

163. The method for suppressing, the method for treating, the pharmaceutical kit for use, the pharmaceutical composition for use, or the pharmaceutical composition, of claim 154, wherein the ADRB2 inhibitor is selected from the group consisting of:
Alprenolol, atenolol, betaxolol, bupranolol, butoxamine, carazolol, carvedilol, CGP 12177, cicloprolol, ICI 118551, ICYP, labetalol, levobetaxolol, levobunolol, LK 204-545, metoprolol, nadolol, NIHP, NIP, propafenone, propranolol, sotalol, SR59230A, and timolol.

164. The method for suppressing, the method for treating, the pharmaceutical kit for use, the pharmaceutical composition for use, or the pharmaceutical composition, of claim 163, wherein the ADRB2 inhibitor is carvedilol.

165. The method for suppressing, the method for treating, the pharmaceutical kit for use, the pharmaceutical composition for use, or the pharmaceutical composition, of any one of claims 1-9 or 17-164, wherein a therapeutically effective amount of the burixafor is administered to the subject.

166. The method for suppressing, the method for treating, the pharmaceutical kit for use, the pharmaceutical composition for use, or the pharmaceutical composition, of any one of claims 1-9 or 17-164, wherein a sub-therapeutically effective amount of the burixafor is administered to the subject.

167. The method for suppressing, the method for treating, the pharmaceutical kit for use, the pharmaceutical composition for use, or the pharmaceutical composition, of any one of claims 1-9 or 17-166, wherein a therapeutically effective amount of the ADRB2 inhibitor is administered to the subject.

168. The method for suppressing, the method for treating, the pharmaceutical kit for use, the pharmaceutical composition for use, or the pharmaceutical composition, of any one of claims 1-9 or 17-166, wherein a sub-therapeutically effective amount of the ADRB2 inhibitor is administered to the subject.

169. The method for suppressing, the method for treating, the pharmaceutical kit for use, the pharmaceutical composition for use, or the pharmaceutical composition, of any one of claims 1-9 or 14-168, wherein the method or use further comprises determining the expression level of the CXCR4 gene in the cell, in the subject, or in the sample obtained from the subject, wherein the cell, the subject, or the sample obtained from the subject is determined to have a CXCR4-expressing cancer if the expression level is greater than a reference level of the CXCR4 gene.

170. The method for suppressing, the method for treating, the pharmaceutical kit for use, the pharmaceutical composition for use, or the pharmaceutical composition, of claim 169, wherein the cancer is a CXCR4-expressing cancer.

171. The method for suppressing, the method for treating, the pharmaceutical kit for use, the pharmaceutical composition for use, or the pharmaceutical composition, of claim 169 or 170, wherein the CXCR4 expression level in the cell is greater than a reference level.

172. The method for suppressing, the method for treating, the pharmaceutical kit for use, the pharmaceutical composition for use, or the pharmaceutical composition, of claim 169 or 170, wherein the CXCR4 expression level in the subject is greater than a reference level.

173. The method for suppressing, the method for treating, the pharmaceutical kit for use, the pharmaceutical composition for use, or the pharmaceutical composition, of claim 169 or 170, wherein the CXCR4 expression level in the sample obtained from the subject is greater than a reference level.

174. The method for suppressing, the method for treating, the pharmaceutical kit for use, the pharmaceutical composition for use, or the pharmaceutical composition, of any one of claims 169-173, upon determining the CXCR4 gene expression level is greater than the reference level, the method or use comprises administering a CXCR4 inhibitor and an ADRB2 inhibitor, wherein the CXCR4 inhibitor is burixafor.

175. The method for suppressing, the method for treating, the pharmaceutical kit for use, the pharmaceutical composition for use, or the pharmaceutical composition, of any one of claims 1-9 or 14-168, wherein the method or use further comprises determining the expression level of the ADRB2 gene in the cell, in the subject, or in the sample obtained from the subject, wherein the cell, the subject, or the sample obtained from the subject is determined to have a ADRB2-expressing cancer if the expression level is greater than a reference level of the ADRB2 gene.

176. The method for suppressing, the method for treating, the pharmaceutical kit for use, the pharmaceutical composition for use, or the pharmaceutical composition, of claim 175, wherein the cancer is an ADRB2-expressing cancer.

177. The method for suppressing, the method for treating, the pharmaceutical kit for use, the pharmaceutical composition for use, or the pharmaceutical composition, of claim 175 or 176, wherein the ADRB2 expression level in the cell is greater than a reference level.

178. The method for suppressing, the method for treating, the pharmaceutical kit for use, the pharmaceutical composition for use, or the pharmaceutical composition, of claim 175 or 176, wherein the ADRB2 expression level in the subject is greater than a reference level.

179. The method for suppressing, the method for treating, the pharmaceutical kit for use, the pharmaceutical composition for use, or the pharmaceutical composition, of claim 175 or 176, wherein the ADRB2 expression level in the sample obtained from the subject is greater than a reference level.

180. The method for suppressing, the method for treating, the pharmaceutical kit for use, the pharmaceutical composition for use, or the pharmaceutical composition, of any one of claims 175-179, upon determining the ADRB2 gene expression level is greater than the reference level, the method or use comprises administering a CXCR4 inhibitor and an ADRB2 inhibitor, wherein the CXCR4 inhibitor is burixafor.

181. The method for suppressing, the method for treating, the pharmaceutical kit for use, the pharmaceutical composition for use, or the pharmaceutical composition, of any one of claims 1-9 or 14-168, wherein the method or use further comprises determining the expression level of the CXCR4 gene and the ADRB2 gene in the cell, in the subject, or in the sample obtained from the subject, wherein the cell, the subject, or the sample obtained from the subject is determined to have a CXCR4-expressing and ADRB2-expressing cancer if the expression levels of the CXCR4 gene and the ADRB2 gene are greater than respective reference levels of the CXCR4 gene and the ADRB2 gene.

182. The method for suppressing, the method for treating, the pharmaceutical kit for use, the pharmaceutical composition for use, or the pharmaceutical composition, of claim 181, wherein the cancer is a CXCR4-expressing cancer and an ADRB2-expressing cancer.

183. The method for suppressing, the method for treating, the pharmaceutical kit for use, the pharmaceutical composition for use, or the pharmaceutical composition, of claim 181 or 182, wherein the CXCR4 expression level and the ADRB2 expression level in the cell are greater than respective reference levels.

184. The method for suppressing, the method for treating, the pharmaceutical kit for use, the pharmaceutical composition for use, or the pharmaceutical composition, of claim 181 or 182, wherein the CXCR4 expression level and the ADRB2 expression level in the subject are greater than respective reference levels.

185. The method for suppressing, the method for treating, the pharmaceutical kit for use, the pharmaceutical composition for use, or the pharmaceutical composition, of claim 181 or 182, wherein the CXCR4 expression level and the ADRB2 expression level the sample obtained from the subject are greater than respective reference levels.

186. The method for suppressing, the method for treating, the pharmaceutical kit for use, the pharmaceutical composition for use, or the pharmaceutical composition, of any one of claims 181-185, wherein upon determining the CXCR4 gene and the ADRB2 gene expression levels are greater than the respective reference levels, the method or use comprises administering a CXCR4 inhibitor and an ADRB2 inhibitor, wherein the CXCR4 inhibitor is burixafor.

187. The method for suppressing, the method for treating, the pharmaceutical kit for use, the pharmaceutical composition for use, or the pharmaceutical composition, of any one of claims 1-9 or 14-168, wherein the method or use further comprises determining the expression level of the CXCR4 protein in the cell, in the subject, or in the sample obtained from the subject, wherein the cell, the subject, or the sample obtained from the subject is determined to have a CXCR4-expressing cancer if the expression level is greater than a reference level of the CXCR4 protein.

188. The method for suppressing, the method for treating, the pharmaceutical kit for use, the pharmaceutical composition for use, or the pharmaceutical composition, of claim 187, wherein the cancer is a CXCR4-expressing cancer.

189. The method for suppressing, the method for treating, the pharmaceutical kit for use, the pharmaceutical composition for use, or the pharmaceutical composition, of claim 187 or 188, wherein the CXCR4 expression level in the cell is greater than a reference level.

190. The method for suppressing, the method for treating, the pharmaceutical kit for use, the pharmaceutical composition for use, or the pharmaceutical composition, of claim 187 or 188, wherein the CXCR4 expression level in the subject is greater than a reference level.

191. The method for suppressing, the method for treating, the pharmaceutical kit for use, the pharmaceutical composition for use, or the pharmaceutical composition, of claim 187 or 188, wherein the CXCR4 expression level in the sample obtained from the subject is greater than a reference level.

192. The method for suppressing, the method for treating, the pharmaceutical kit for use, the pharmaceutical composition for use, or the pharmaceutical composition, of any one of claims 187-191, upon determining the CXCR4 protein expression level is greater than the reference level, the method or use comprises administering a CXCR4 inhibitor and an ADRB2 inhibitor, wherein the CXCR4 inhibitor is burixafor.

193. The method for suppressing, the method for treating, the pharmaceutical kit for use, the pharmaceutical composition for use, or the pharmaceutical composition, of any one of claims 1-9 or 14-168, wherein the method or use further comprises determining the expression level of the ADRB2 protein in the cell, in the subject, or in the sample obtained from the subject, wherein the cell, the subject, or the sample obtained from the subject is determined to have a ADRB2-expressing cancer if the expression level is greater than a reference level of the ADRB2 protein.

194. The method for suppressing, the method for treating, the pharmaceutical kit for use, the pharmaceutical composition for use, or the pharmaceutical composition, of claim 193, wherein the cancer is an ADRB2-expressing cancer.

195. The method for suppressing, the method for treating, the pharmaceutical kit for use, the pharmaceutical composition for use, or the pharmaceutical composition, of claim 193 or 194, wherein the ADRB2 expression level in the cell is greater than a reference level.

196. The method for suppressing, the method for treating, the pharmaceutical kit for use, the pharmaceutical composition for use, or the pharmaceutical composition, of claim 193 or 194, wherein the ADRB2 expression level in the subject is greater than a reference level.

197. The method for suppressing, the method for treating, the pharmaceutical kit for use, the pharmaceutical composition for use, or the pharmaceutical composition, of claim 193 or 194, wherein the ADRB2 expression level in the sample obtained from the subject is greater than a reference level.

198. The method for suppressing, the method for treating, the pharmaceutical kit for use, the pharmaceutical composition for use, or the pharmaceutical composition, of any one of claims 193-197, upon determining the ADRB2 protein expression level is greater than the reference level, the method or use comprises administering a CXCR4 inhibitor and an ADRB2 inhibitor, wherein the CXCR4 inhibitor is burixafor.

199. The method for suppressing, the method for treating, the pharmaceutical kit for use, the pharmaceutical composition for use, or the pharmaceutical composition, of any one of claims 1-9 or 14-168, wherein the method or use further comprises determining the expression level of the CXCR4 protein and the ADRB2 protein in the cell, in the subject, or in the sample obtained from the subject, wherein the cell, the subject, or the sample obtained from the subject is determined to have a CXCR4-expressing and ADRB2-expressing cancer if the expression levels of the CXCR4 protein and the ADRB2 protein are greater than respective reference levels of the CXCR4 protein and the ADRB2 protein.

200. The method for suppressing, the method for treating, the pharmaceutical kit for use, the pharmaceutical composition for use, or the pharmaceutical composition, of claim 199, wherein the cancer is a CXCR4-expressing cancer and an ADRB2-expressing cancer.

201. The method for suppressing, the method for treating, the pharmaceutical kit for use, the pharmaceutical composition for use, or the pharmaceutical composition, of claim 199 or 200, wherein the CXCR4 expression level and the ADRB2 expression level in the cell are greater than respective reference levels.

202. The method for suppressing, the method for treating, the pharmaceutical kit for use, the pharmaceutical composition for use, or the pharmaceutical composition, of claim 199 or 200, wherein the CXCR4 expression level and the ADRB2 expression level in the subject are greater than respective reference levels.

203. The method for suppressing, the method for treating, the pharmaceutical kit for use, the pharmaceutical composition for use, or the pharmaceutical composition, of claim 199 or 200, wherein the CXCR4 expression level and the ADRB2 expression level the sample obtained from the subject are greater than respective reference levels.

204. The method for suppressing, the method for treating, the pharmaceutical kit for use, the pharmaceutical composition for use, or the pharmaceutical composition, of any one of claims 199-203, wherein upon determining the CXCR4 protein and the ADRB2 protein expression levels are greater than the respective reference levels, the method or use comprises administering a CXCR4 inhibitor and an ADRB2 inhibitor, wherein the CXCR4 inhibitor is burixafor.

205. The method for suppressing, the method for treating, the pharmaceutical kit for use, the pharmaceutical composition for use, or the pharmaceutical composition, of any one of claims 1-204, wherein the ADRB2 inhibitor is carvedilol.

206. The method for suppressing, the method for treating, the pharmaceutical kit for use, the pharmaceutical composition for use, or the pharmaceutical composition, of any one of claims 1-205, wherein the enhanced downstream signaling is an enhanced response to co-stimulation of the CXCR4-ADRB2 heteromer with a CXCR4 agonist and an ADRB2 agonist.

207. The method for suppressing, the method for treating, the pharmaceutical kit for use, the pharmaceutical composition for use, or the pharmaceutical composition, of any one of claims 1-206, wherein the enhanced downstream signaling is an enhanced cancer progression in the subject having cell(s) containing said CXCR4-ADRB2 heteromer.

208. The method for suppressing, the method for treating, the pharmaceutical kit for use, the pharmaceutical composition for use, or the pharmaceutical composition, of claim 207, wherein the enhanced cancer progression is an enhanced cell proliferation in the subject having cell(s) containing said CXCR4-ADRB2 heteromer.

209. The method for suppressing, the method for treating, the pharmaceutical kit for use, the pharmaceutical composition for use, or the pharmaceutical composition, of claim 207 or 208, wherein the enhanced cancer progression is an enhanced cell migration in the subject having cell(s) containing said CXCR4-ADRB2 heteromer.

210. The method for suppressing, the method for treating, the pharmaceutical kit for use, the pharmaceutical composition for use, or the pharmaceutical composition, of any one of claims 207-209, wherein the enhanced cancer progression is an enhanced metastasis in the subject having cell(s) containing said CXCR4-ADRB2 heteromer.

211. The method for suppressing, the method for treating, the pharmaceutical kit for use, the pharmaceutical composition for use, or the pharmaceutical composition, of any one of claims 207-210, wherein the enhanced cancer progression is an enhanced tumor growth in the subject having cell(s) containing said CXCR4-ADRB2 heteromer.

212. The method for suppressing, the method for treating, the pharmaceutical kit for use, the pharmaceutical composition for use, or the pharmaceutical composition, of any one of claims 207-211, wherein the enhanced cancer progression is an enhanced angiogenesis in the subject having cell(s) containing said CXCR4-ADRB2 heteromer.

213. The method for suppressing, the method for treating, the pharmaceutical kit for use, the pharmaceutical composition for use, or the pharmaceutical composition, of any one of claims 207-212, wherein the cell(s) are cancer cell(s).

214. The method for suppressing, the method for treating, the pharmaceutical kit for use, the pharmaceutical composition for use, or the pharmaceutical composition, of any one of claims 207-213, wherein the cell(s) are derived from a subject.

215. The method for suppressing, the method for treating, the pharmaceutical kit for use, the pharmaceutical composition for use, or the pharmaceutical composition, of any one of claims 207-214, wherein the cell(s) are from a biological sample obtained from a subject.


SEQUENCE LISTING (6 sequences)

SEQ ID NO: 1 - DNA, 22 bp, Artificial Sequence
  Primer DNA sequence of ADRB2-F
ctcttccatcgtgtccttctac

SEQ ID NO: 2 - DNA, 20 bp, Artificial Sequence
  Primer DNA sequence of ADRB2-R
aatcttctggagctgccttt

SEQ ID NO: 3 - DNA, 23 bp, Artificial Sequence
  Primer DNA sequence of CXCR4-F
ccaccatctactccatcatcttc

SEQ ID NO: 4 - DNA, 20 bp, Artificial Sequence
  Primer DNA sequence of CXCR4-R
acttgtccgtcatgcttctc

SEQ ID NO: 5 - DNA, 22 bp, Artificial Sequence
  Primer DNA sequence of Beta-actin-F
ggaaatcgtgcgtgacattaag

SEQ ID NO: 6 - DNA, 19 bp, Artificial Sequence
  Primer DNA sequence of Beta-actin-R
agctcgtagctcttctcca